SEQUENCE LISTING (97 sequences)

SEQ ID NO: 1 - DNA, 19 bp, Artificial Sequence
  Synthetic
gcttcactctcatgtcgtc

SEQ ID NO: 2 - DNA, 23 bp, Artificial Sequence
  Synthetic
gggcacgggcagcttgccggtgg

SEQ ID NO: 3 - DNA, 23 bp, Artificial Sequence
  Synthetic
gggcacgggcagcttgccgctgg

SEQ ID NO: 4 - DNA, 23 bp, Artificial Sequence
  Synthetic
gggcacgggcagcttgcccgtgg

SEQ ID NO: 5 - DNA, 23 bp, Artificial Sequence
  Synthetic
gggcacgggcagcttcccggtgg

SEQ ID NO: 6 - DNA, 17 bp, Artificial Sequence
  Synthetic
gcttcactctcatcgtc

SEQ ID NO: 7 - DNA, 12 bp, Artificial Sequence
  Synthetic
acttgtttaagt

SEQ ID NO: 8 - DNA, 16 bp, Artificial Sequence
  Synthetic
ggcaccgagtcggtgc

SEQ ID NO: 9 - protein, 8 aa, Artificial Sequence
  Synthetic
GPLGIAGQ

SEQ ID NO: 10 - DNA, 20 bp, Artificial Sequence
  Synthetic
gccaaattggacgaccctcg

SEQ ID NO: 11 - DNA, 20 bp, Artificial Sequence
  Synthetic
cgaggagacccccgtttcgg

SEQ ID NO: 12 - DNA, 20 bp, Artificial Sequence
  Synthetic
cccgccgccgccgtggctcg

SEQ ID NO: 13 - DNA, 20 bp, Artificial Sequence
  Synthetic
tgagctctacgagatccaca

SEQ ID NO: 14 - DNA, 17 bp, Artificial Sequence
  Synthetic
gcttcactctcatcgtc

SEQ ID NO: 15 - DNA, 17 bp, Artificial Sequence
  Synthetic
gcttcactctcatcgtc

SEQ ID NO: 16 - DNA, 19 bp, Artificial Sequence
  Synthetic
cccggagaagcgtagcaaa

SEQ ID NO: 17 - DNA, 20 bp, Artificial Sequence
  Synthetic
ccccggcacgcttatttttc

SEQ ID NO: 18 - DNA, 20 bp, Artificial Sequence
  Synthetic
cccggagaagcgtagcaaag

SEQ ID NO: 19 - DNA, 22 bp, Artificial Sequence
  Synthetic
aagatccccggcacgcttattt

SEQ ID NO: 20 - DNA, 36 bp, Artificial Sequence
  Synthetic
cgtattagctatctacaggagattttttcaaatgag

SEQ ID NO: 21 - DNA, 30 bp, Artificial Sequence
  Synthetic
gtagatagctaatacgattcttccgacgtg

SEQ ID NO: 22 - DNA, 33 bp, Artificial Sequence
  Synthetic
aaaaatagaaagctttgatagtgttgaaatttc

SEQ ID NO: 23 - DNA, 24 bp, Artificial Sequence
  Synthetic
ttgaaataatcttcttttaattgc

SEQ ID NO: 24 - DNA, 33 bp, Artificial Sequence
  Synthetic
gaggaaggtgtgcgataagaaatactcaatagg

SEQ ID NO: 25 - DNA, 18 bp, Artificial Sequence
  Synthetic
ttcttcttgggcataaac

SEQ ID NO: 26 - DNA, 24 bp, Artificial Sequence
  Synthetic
tattaacgcatgcggagtagatgc

SEQ ID NO: 27 - DNA, 24 bp, Artificial Sequence
  Synthetic
gggttttcttcaaataattgattg

SEQ ID NO: 28 - DNA, 31 bp, Artificial Sequence
  Synthetic
gttacttgcggaatgcgaaaaccagcatttc

SEQ ID NO: 29 - DNA, 33 bp, Artificial Sequence
  Synthetic
cgcattccgcaagtaacatatttgacctttgtc

SEQ ID NO: 30 - DNA, 35 bp, Artificial Sequence
  Synthetic
aacaaatcgatgcgtaaccgttaagcaattaaaag

SEQ ID NO: 31 - DNA, 21 bp, Artificial sequence
  Synthetic
ttgaagagtaaatcaacaatg

SEQ ID NO: 32 - DNA, 36 bp, Artificial Sequence
  Synthetic
gtatgtggactgcgaattagatattaatcgtttaag

SEQ ID NO: 33 - DNA, 18 bp, Artificial Sequence
  Synthetic
atgtctcttccattttgg

SEQ ID NO: 34 - DNA, 32 bp, Artificial Sequence
  Synthetic
taaatacgattgcaatgataaacttattcgag

SEQ ID NO: 35 - DNA, 18 bp, Artificial Sequence
  Synthetic
gtattcatgcgactatcc

SEQ ID NO: 36 - DNA, 25 bp, Artificial Sequence
  Synthetic
tgctaagtcttgccaagaaataggc

SEQ ID NO: 37 - DNA, 23 bp, Artificial Sequence
  Synthetic
atcattttacgaacatcataaac

SEQ ID NO: 38 - DNA, 25 bp, Artificial Sequence
  Synthetic
tacacttgcatgcggagagattcgc

SEQ ID NO: 39 - DNA, 21 bp, Artificial Sequence
  Synthetic
atttctgttttgaagaagttc

SEQ ID NO: 40 - DNA, 30 bp, Artificial Sequence
  Synthetic
ctaatgggtgcactggagaaattgtctggg

SEQ ID NO: 41 - DNA, 32 bp, Artificial Sequence
  Synthetic
ctccagtgcacccattagtttcgattagaggg

SEQ ID NO: 42 - DNA, 29 bp, Artificial Sequence
  Synthetic
aaaaagaaattgcgacaagcttattgctc

SEQ ID NO: 43 - DNA, 19 bp, Artificial Sequence
  Synthetic
ggtaaaattgactccttgg

SEQ ID NO: 44 - DNA, 37 bp, Artificial Sequence
  Synthetic
aaaagggtgctcgaagaagttaaaatccgttaaagag

SEQ ID NO: 45 - DNA, 30 bp, Artificial Sequence
  Synthetic
cttcgagcaccctttttccaccttagcaac

SEQ ID NO: 46 - DNA, 27 bp, Artificial Sequence
  Synthetic
ttttgagttatgcaacggtcgtaaacg

SEQ ID NO: 47 - DNA, 24 bp, Artificial Sequence
  Synthetic
agactatatttaggtagtttaatg

SEQ ID NO: 48 - DNA, 55 bp, Artificial Sequence
  Synthetic
aaaagcaccgactcggtgccactttttcaagttgataacggactagccttatttt

SEQ ID NO: 49 - DNA, 25 bp, Artificial Sequence
  Synthetic
aacttgctatttctagctctaaaac

SEQ ID NO: 50 - DNA, 54 bp, Artificial Sequence
  Synthetic
taatacgactcactataggtccaggggtcttactcctgttttagagctagaaat

SEQ ID NO: 51 - DNA, 55 bp, Artificial Sequence
  Synthetic
taatacgactcactatagcctccaaggagtaagaccccgttttagagctagaaat

SEQ ID NO: 52 - DNA, 54 bp, Artificial Sequence
  Synthetic
taatacgactcactatagcgccaaggagtagggcacagttttagagctagaaat

SEQ ID NO: 53 - DNA, 54 bp, Artificial Sequence
  Synthetic
taatacgactcactatagggccagaaggggctcacaagttttagagctagaaat

SEQ ID NO: 54 - DNA, 54 bp, Artificial Sequence
  Synthetic
taatacgactcactatagggacctcggggagagtgacgttttagagctagaaat

SEQ ID NO: 55 - DNA, 54 bp, Artificial Sequence
  Synthetic
taatacgactcactataggggagagtgaccggctcacgttttagagctagaaat

SEQ ID NO: 56 - DNA, 54 bp, Artificial Sequence
  Synthetic
taatacgactcactatagcccaagtcccgtcgtgaaggttttagagctagaaat

SEQ ID NO: 57 - DNA, 54 bp, Artificial Sequence
  Synthetic
taatacgactcactatagctccagttgtggcacttgcgttttagagctagaaat

SEQ ID NO: 58 - DNA, 53 bp, Artificial Sequence
  Synthetic
taatacgactcactatagggtggaggcccggaggccgttttagagctagaaat

SEQ ID NO: 59 - DNA, 54 bp, Artificial Sequence
  Synthetic
taatacgactcactatagggtggaggcccggaggccggttttagagctagaaat

SEQ ID NO: 60 - DNA, 54 bp, Artificial Sequence
  Synthetic
taatacgactcactatagtctgaagatccccggcctcgttttagagctagaaat

SEQ ID NO: 61 - DNA, 54 bp, Artificial Sequence
  Synthetic
taatacgactcactatagtgggtggaggcccggaggcgttttagagctagaaat

SEQ ID NO: 62 - DNA, 54 bp, Artificial Sequence
  Synthetic
taatacgactcactatagctgaagatccccggcctccgttttagagctagaaat

SEQ ID NO: 63 - DNA, 54 bp, Artificial Sequence
  Synthetic
taatacgactcactatagacaatgccacgcttctgccgttttagagctagaaat

SEQ ID NO: 64 - DNA, 54 bp, Artificial Sequence
  Synthetic
taatacgactcactatagcttcagaccttggcactgggttttagagctagaaat

SEQ ID NO: 65 - DNA, 54 bp, Artificial Sequence
  Synthetic
taatacgactcactatagggcacgggcagcttgccgggttttagagctagaaat

SEQ ID NO: 66 - DNA, 54 bp, Artificial Sequence
  Synthetic
taatacgactcactatagggcacgggcagcttgccgcgttttagagctagaaat

SEQ ID NO: 67 - DNA, 54 bp, Artificial Sequence
  Synthetic
taatacgactcactatagggcacgggcagcttgcccggttttagagctagaaat

SEQ ID NO: 68 - DNA, 54 bp, Artificial Sequence
  Synthetic
taatacgactcactatagggcacgggcagcttcccgggttttagagctagaaat

SEQ ID NO: 69 - DNA, 200 bp, Artificial Sequence
  Synthetic
tcttctaggtatgacaacgaatttggctacagcaacagggtggtggacctcatggcccacatggcctccaaggaggtgagcggctggcggctgttcaagaagattagctaagacccctggaccaccagccccagcaagagcacaagaggaagagagagaccctcactgctggggagtccctgcgacgatgagagtgaagc

SEQ ID NO: 70 - DNA, 183 bp, Artificial Sequence
  Synthetic
tcttctaggtatgacaacgaatttggctacagcaacagggtggtggacctcatggcccacatggcctccaaggaggtgagcggctggcggctgttcaagaagattagctaagacccctggaccaccagccccagcaagagcacaagaggaagagagagaccctcactgctggggagtccctgc

SEQ ID NO: 71 - DNA, 198 bp, Artificial Sequence
  Synthetic
tcttctaggtatgacaacgaatttggctacagcaacagggtggtggacctcatggcccacatggcctccaaggaggtgagcggctggcggctgttcaagaagattagctaagacccctggaccaccagccccagcaagagcacaagaggaagagagagaccctcactgctggggagtccctgcgacgatgagagtgaa

SEQ ID NO: 72 - DNA, 196 bp, Artificial Sequence
  Synthetic
tcttctaggtatgacaacgaatttggctacagcaacagggtggtggacctcatggcccacatggcctccaaggaggtgagcggctggcggctgttcaagaagattagctaagacccctggaccaccagccccagcaagagcacaagaggaagagagagaccctcactgctggggagtccctgcgacgatgagagtg

SEQ ID NO: 73 - DNA, 196 bp, Artificial Sequence
  Synthetic
cagctcagagccctgtggcggactacagggcctgcacagacggtcactcaaagaaagatgtccctgtgccctagctaatcttcttgaacagccgccagccgctcacctccttggcgatggcaaagggcttctccacctcgatcttgccgcagtctgcgatgatcacatccttcaggggtgacgatgagagtgaagc

SEQ ID NO: 74 - DNA, 179 bp, Artificial Sequence
  Synthetic
cagctcagagccctgtggcggactacagggcctgcacagacggtcactcaaagaaagatgtccctgtgccctagctaatcttcttgaacagccgccagccgctcacctccttggcgatggcaaagggcttctccacctcgatcttgccgcagtctgcgatgatcacatccttcaggggt

SEQ ID NO: 75 - DNA, 200 bp, Artificial Sequence
  Synthetic
gaggtcaaggaccgctgcaccctggcagagaagctggggggcagtgccgtcatctccctggagggcaagcctttggtgagcggctggcggctgttcaagaagattagctgagccccttctggccccctgcctggagcatctggcagccccacacctgcccttgggggttgcaggctgccccctgacgatgagagtgaagc

SEQ ID NO: 76 - DNA, 183 bp, Artificial Sequence
  Synthetic
gaggtcaaggaccgctgcaccctggcagagaagctggggggcagtgccgtcatctccctggagggcaagcctttggtgagcggctggcggctgttcaagaagattagctgagccccttctggccccctgcctggagcatctggcagccccacacctgcccttgggggttgcaggctgccccct

SEQ ID NO: 77 - DNA, 185 bp, Artificial Sequence
  Synthetic
ggaggctctgtacctggtgtgtggggaacgtggtttcttctacacacccaagtcccgtcgtgaagtggagaagcgtgtgagcggctggcggctgttcaagaagattagcaagcgtgacccgcaagtgccacaactggagctgggtggaggcccggaggccggggatcttcagaccttggcactgg

SEQ ID NO: 78 - DNA, 183 bp, Artificial Sequence
  Synthetic
acacccaagtcccgtcgtgaagtggaggacccgcaagtgccacaactggagctgggtggaggcccggagaagcgtgtgagcggctggcggctgttcaagaagattagcaagcgtgccggggatcttcagaccttggcactggaggttgcccggcagaagcgtggcattgtggatcagtgctgc

SEQ ID NO: 79 - DNA, 200 bp, Artificial Sequence
  Synthetic
acacccaagtcccgtcgtgaagtggaggacccgcaagtgccacaactggagctgggtggaggcccggagaagcgtgtgagcggctggcggctgttcaagaagattagcaagcgtgccggggatcttcagaccttggcactggaggttgcccggcagaagcgtggcattgtggatcagtgctgcgacgatgagagtgaagc

SEQ ID NO: 80 - DNA, 185 bp, Artificial Sequence
  Synthetic
gcaagtgccacaactggagctgggtggaggcccggaggccggggatcttcagaccttggcactggaggttaagcgtgtgagcggctggcggctgttcaagaagattagcaagcgtgcccggcagaagcgtggcattgtggatcagtgctgcaccagcatctgctccctctaccaactggagaact

SEQ ID NO: 81 - DNA, 200 bp, Artificial Sequence
  Synthetic
gacaacgaatttggctacagcaacagggtggtggacctcatggcccacatggcctccaaggagggtggcggccgtgaccacatggtccttcatgagtatgtaaatgctgctgggattacataagacccctggaccaccagccccagcaagagcacaagaggaagagagagaccctcactgctggacgatgagagtgaagc

SEQ ID NO: 82 - DNA, 183 bp, Artificial Sequence
  Synthetic
gacaacgaatttggctacagcaacagggtggtggacctcatggcccacatggcctccaaggagggtggcggccgtgaccacatggtccttcatgagtatgtaaatgctgctgggattacataagacccctggaccaccagccccagcaagagcacaagaggaagagagagaccctcactgctg

SEQ ID NO: 83 - DNA, 137 bp, Artificial Sequence
  Synthetic
gtgggaagagctgcaggaggtgaagctgggagtgtgggacctcggtgagagtgaccggctcaccggactactagaccgcggcctttctgggccatatctgtgagggagccaaggagcagggacgatgagagtgaagc

SEQ ID NO: 84 - DNA, 94 bp, Artificial Sequence
  Synthetic
acagatatggcccagaaaggccgcggtctagtagtccggtgagccggtcactgtccccgaggtcccacacacccagcgacgatgagagtgaagc

SEQ ID NO: 85 - DNA, 831 bp, Artificial Sequence
  Synthetic
taatacgactcactatagcttcactctcatcgtcggctttattcattgcagaggggtgggcggggagtggtggactcagttgcagtagttctccagttggtagagggagcagatgctggtgcagcactgatccacaatgccacgcttctgccgggcaacctccagtgccaaggtctgaagatccccggcacgcttatttttcattttgagtgtcacgtaggcttctatgcagttgatgaagatgtcaaactcattcatggccttgtagacacctttgtcttggagcttattaaaatcattcttcacctgctccactgccttgcttttattctcacaggggagaaatcgatgacagcgtcgcagctgtatccagagggtcttcagcttctctcccagggaattcaaatgctccttgatttctgggccatggttctctgcctggggcatcacttctaccaggtaaaacttgatcatttctgacaaggcttggcaacccaagtaacccttaaagtcctgcagtaaggaatctgtcagcagtatgttgtccagctggtccttcttttgaaagaaagtcttcacttgactgaaggcagccctcagctctcggagcatgtgggtctggctgactgggaagtgggtgcagttattgtcaccccggatggaatggcctttgctacgcttctccgggcctccacccagctccagttgtggcacttgcgggtcctccacttcacgacgggacttgggtgtgtagaagaaaccacgttccccacacaccaggtacagagcctccaccaggtgaggaccacaaaggtgctgtttgacaaaagc

SEQ ID NO: 86 - DNA, 814 bp, Artificial Sequence
  Synthetic
taatacgactcactataggctttattcattgcagaggggtgggcggggagtggtggactcagttgcagtagttctccagttggtagagggagcagatgctggtgcagcactgatccacaatgccacgcttctgccgggcaacctccagtgccaaggtctgaagatccccggcacgcttatttttcattttgagtgtcacgtaggcttctatgcagttgatgaagatgtcaaactcattcatggccttgtagacacctttgtcttggagcttattaaaatcattcttcacctgctccactgccttgcttttattctcacaggggagaaatcgatgacagcgtcgcagctgtatccagagggtcttcagcttctctcccagggaattcaaatgctccttgatttctgggccatggttctctgcctggggcatcacttctaccaggtaaaacttgatcatttctgacaaggcttggcaacccaagtaacccttaaagtcctgcagtaaggaatctgtcagcagtatgttgtccagctggtccttcttttgaaagaaagtcttcacttgactgaaggcagccctcagctctcggagcatgtgggtctggctgactgggaagtgggtgcagttattgtcaccccggatggaatggcctttgctacgcttctccgggcctccacccagctccagttgtggcacttgcgggtcctccacttcacgacgggacttgggtgtgtagaagaaaccacgttccccacacaccaggtacagagcctccaccaggtgaggaccacaaaggtgctgtttgacaaaagc

SEQ ID NO: 87 - DNA, 32 bp, Artificial Sequence
  Synthetic
taatacgactcactatagcttcactctcatcg

SEQ ID NO: 88 - DNA, 42 bp, Artificial Sequence
  Synthetic
taatacgactcactataggctttattcattgcagaggggtgg

SEQ ID NO: 89 - DNA, 21 bp, Artificial Sequence
  Synthetic
gcttttgtcaaacagcacctt

SEQ ID NO: 90 - DNA, 814 bp, Artificial Sequence
  Synthetic
gcttttgtcaaacagcacctttgtggtcctcacctggtggaggctctgtacctggtgtgtggggaacgtggtttcttctacacacccaagtcccgtcgtgaagtggaggacccgcaagtgccacaactggagctgggtggaggcccggagaagcgtagcaaaggccattccatccggggtgacaataactgcacccacttcccagtcagccagacccacatgctccgagagctgagggctgccttcagtcaagtgaagactttctttcaaaagaaggaccagctggacaacatactgctgacagattccttactgcaggactttaagggttacttgggttgccaagccttgtcagaaatgatcaagttttacctggtagaagtgatgccccaggcagagaaccatggcccagaaatcaaggagcatttgaattccctgggagagaagctgaagaccctctggatacagctgcgacgctgtcatcgatttctcccctgtgagaataaaagcaaggcagtggagcaggtgaagaatgattttaataagctccaagacaaaggtgtctacaaggccatgaatgagtttgacatcttcatcaactgcatagaagcctacgtgacactcaaaatgaaaaataagcgtgccggggatcttcagaccttggcactggaggttgcccggcagaagcgtggcattgtggatcagtgctgcaccagcatctgctccctctaccaactggagaactactgcaactgagtccaccactccccgcccacccctctgcaatgaataaagccgacgatgagagtgaagc

SEQ ID NO: 91 - DNA, 797 bp, Artificial Sequence
  Synthetic
gcttttgtcaaacagcacctttgtggtcctcacctggtggaggctctgtacctggtgtgtggggaacgtggtttcttctacacacccaagtcccgtcgtgaagtggaggacccgcaagtgccacaactggagctgggtggaggcccggagaagcgtagcaaaggccattccatccggggtgacaataactgcacccacttcccagtcagccagacccacatgctccgagagctgagggctgccttcagtcaagtgaagactttctttcaaaagaaggaccagctggacaacatactgctgacagattccttactgcaggactttaagggttacttgggttgccaagccttgtcagaaatgatcaagttttacctggtagaagtgatgccccaggcagagaaccatggcccagaaatcaaggagcatttgaattccctgggagagaagctgaagaccctctggatacagctgcgacgctgtcatcgatttctcccctgtgagaataaaagcaaggcagtggagcaggtgaagaatgattttaataagctccaagacaaaggtgtctacaaggccatgaatgagtttgacatcttcatcaactgcatagaagcctacgtgacactcaaaatgaaaaataagcgtgccggggatcttcagaccttggcactggaggttgcccggcagaagcgtggcattgtggatcagtgctgcaccagcatctgctccctctaccaactggagaactactgcaactgagtccaccactccccgcccacccctctgcaatgaataaagcc

SEQ ID NO: 92 - protein, 5 aa, Artificial Sequence
  Synthetic
GGGGS

SEQ ID NO: 93 - protein, 10 aa, Artificial Sequence
  Synthetic
GGGGSGGGGS

SEQ ID NO: 94 - protein, 15 aa, Artificial Sequence
  Synthetic
GGGGSGGGGSGGGGS

SEQ ID NO: 95 - protein, 30 aa, Artificial Sequence
  Synthetic
GGGGSGGGGSGGGGSGGGGSGGGGSGGGGS

SEQ ID NO: 96 - protein, 45 aa, Artificial Sequence
  Synthetic
GGGGSGGGGSGGGGSGGGGSGGGGSGGGGSGGGGSGGGGSGGGGS

SEQ ID NO: 97 - protein, 60 aa, Artificial Sequence
  Synthetic
GGGGSGGGGSGGGGSGGGGSGGGGSGGGGSGGGGSGGGGSGGGGSGGGGSGGGGSGGGGS

(19) United States
(12) Patent Application Publication
(10) Pub. No.: US 2022/0162649 A1
(43) Pub. Date: May 26, 2022

(54) NOVEL NUCLEIC ACID MODIFIERS

(71) Applicants: THE BROAD INSTITUTE, INC., Cambridge, MA (US); THE BRIGHAM AND WOMEN'S HOSPITAL, INC., Boston, MA (US)

(72) Inventors: Amit CHOUDHARY, Boston, MA (US); Donghyun LIM, Boston, MA (US); Praveen KOKKONDA, Boston, MA (US); Sophia LAI, Boston, MA (US); Miseon LEE, Cambridge, MA (US)

(21) Appl. No.: 17/601,052

(22) PCT Filed: Apr. 1, 2020
(86) PCT No.: PCT/US2020/026264

§ 371 Date: Oct. 1, 2021

Related U.S. Application Data
(60) Provisional application No. 62/827,797, filed on Apr. 1, 2019.
(60) Provisional application No. 62/851,616, filed on May 22, 2019.

(51) Int. Cl. C12N 15/90; C12N 9/22; C12N 9/96; C12N 15/62; C12N 15/11; C12N 15/115

(52) U.S. Cl. CPC C12N 15/907; C12N 2320/32; C12N 9/96; C12N 15/62; C12N 15/11; C12N 15/115; C12N 2330/31; C12N 2800/80; C12N 2840/002; C12N 2830/001; C12N 2310/3181; C12N 2310/351; C12N 2310/16; C12N 2310/3513; C12N 9/22

(57) ABSTRACT

The present inventions generally relate to site-specific delivery of nucleic acid modifiers and includes novel DNA-binding proteins and effectors that can be rapidly programmed to make site-specific DNA modifications. The present inventions also provide synthetic all-in-one genome editor (SAGE) systems comprising designer DNA sequence readers and a set of small molecules that induce double-strand breaks, enhance cellular permeability, inhibit NHEJ and activate HDR, as well as methods of using and delivering such systems.

### CROSS-REFERENCE TO RELATED APPLICATIONS

This application claims the benefit of U.S. Provisional Application No. 62/827,797 filed Apr. 1, 2019, and U.S. Provisional Application No. 62/851,616 filed May 22, 2019. The entire contents of the above-identified applications are hereby fully incorporated herein by reference.

### STATEMENT AS TO FEDERALLY FUNDED RESEARCH

This invention was made with government support under Grant No. AI126239 awarded by the National Institutes of Health, Grant No. N66001-17-2-4055 awarded by the Department of Defense, and Grant No. W911NF1610586 awarded by the Army Research Office. The government has certain rights in the invention.

### REFERENCE TO AN ELECTRONIC SEQUENCE LISTING

The contents of the electronic sequence listing (“BROD-4150WP_ST25.txt”; Size 26,519 bytes and it was created on Mar. 23, 2020) is herein incorporated by reference in its entirety.

### TECHNICAL FIELD

The subject matter disclosed herein generally relates to nucleic acid modifiers with novel DNA readers and effector components that can facilitate DNA repair by homology directed repair (HDR), which can be rapidly programmed to make site-specific DNA modifications. Among other aspects, the compositions provide features of Clustered Regularly Interspaced Short Palindromic Repeats (CRISPR) proteins, CRISPR systems, components thereof, peptide nucleic acid (PNA), nucleic acid molecules, vectors, involving the same and uses of all of the foregoing.

### BACKGROUND

Of the three elements of central dogma (Proteins, RNA, and DNA), nearly all therapeutic agents target proteins. Genome and RNA editing have ushered in an era where DNA and RNA can be potential targets, expanding the scope of therapeutic targets to both the coding and non-coding regions of the genome. However, the agents used to accomplish genome editing do not display attributes of a typical therapeutic agent, and in many cases, the activity of these agents are described as genome vandalism rather than genome editing. As such, there is much room to expand the repertoire of genome editors.

Technologies to genetically fuse protein domains to CRISPR-Cas9, an RNA-guided DNA endonuclease, have furnished transformative methods for base and epigenome editing, transcriptional control, and chromatin imaging, though these technologies are generally limited to fusions that are linear, polypeptidic, and located on Cas9 termini. By developing platforms for creating new fusions that are non-polypeptidic (e.g., nucleic acids), internally located on Cas9, and branched with a multivalent display, the field of CRISPR-Cas9 would be opened to a multitude of new and interesting applications. For example, precise genomic editing to a desired sequence requires efficient incorporation of exogenously supplied single-stranded oligonucleotide donor DNA (ssODN) at the DNA double-strand break induced by Cas9 via the homology-directed repair (HDR) pathway. However, most cells instead adopt the non-homologous end-joining (NHEJ) repair pathway, which results in unpredictable insertions and deletions of bases, with some deletions extending to up to several kilobases and generating pathogenic consequences. This could be solved by chemically linking ssODN to Cas9 to increase its local concentration around the target site, allowing enhanced incorporation of the desired sequence in the correct location. In another application, appending PEG chains to Cas9 may reduce the immunogenicity, which is a major concern given the recent discovery of antibodies against Cas9 in humans. Additionally, small-molecule inhibitors of the NHEJ pathway can enhance precision editing, but genome-wide NHEJ inhibition causes cytotoxicity that limits their utility. Further, local inhibition of the NHEJ pathway and/or local activation of HDR at the strand-break site can also tip the balance in favor of DNA recombination. There is also a need to improve homology-directed repair (HDR) efficiency. Increased efficiency of repair is highly desirable in disease models and therapies.

Precise genome targeting technologies are needed to enable systematic reverse engineering of causal genetic variations by allowing selective perturbation of individual genetic elements, as well as to advance synthetic biology, biotechnological, and medical applications. There remains a need for new genome engineering technologies.

Citation or identification of any document in this application is not an admission that such document is available as prior art to the present invention.

### SUMMARY

In certain example embodiments, the disclosure relates to an engineered, non-naturally occurring nucleic acid modifying system, comprising: an engineered, non-naturally occurring nucleic acid-guided molecule comprising a nucleic acid binding domain which complexes with a guide comprising a polynucleotide, one or more effector domains, and the guide, wherein the guide directs sequence specific binding of the nucleic acid-guided molecule to a target nucleic acid, and as to an analogous naturally-occurring nucleic acid-guided molecule, the engineered, non-naturally-occurring nucleic acid-guided molecule is truncated and the one or more effector domains is heterologous.

In certain embodiments, the one or more effector components comprise one or more single-stranded oligo donors (ssODNs). In certain embodiments, the one or more effector components comprise one or more NHEJ inhibitors. In embodiments, the inhibitor of NHEJ is an inhibitor of DNA ligase IV, KU70, or KU80, in embodiments, the inhibitor is an SCR7 or SCR6 analog. In certain embodiments, the one or more effector components comprise one or more HDR activators. In certain embodiments, the one or more effector components comprise a single-stranded oligo donor (ssODN), one or more NHEJ inhibitors, one or more HDR activators, or a combination thereof.

A composition comprising an engineered, non-naturally occurring nucleic acid-guided molecule comprising a nucleic acid binding domain which complexes with a guide comprising a polynucleotide, and one or more effector domains, wherein the guide directs sequence specific binding of the nucleic acid-guided molecule to a target nucleic acid, wherein as to an analogous naturally-occurring nucleic acid-guided molecule, the engineered, non-naturally-occurring nucleic acid-guided molecule is truncated. comprises an activator of homology-directed repair (HDR) and/or an inhibitor of non-homologous end joining (NHEJ) as disclosed herein.

NHEJ inhibitors in some embodiments may be selected from

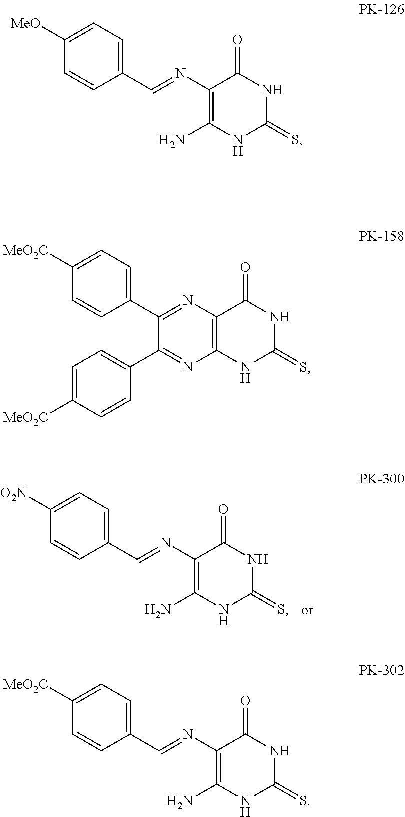 [SMILES: COc1ccc(/C=N/c2c(N)[nH]c(=S)[nH]c2=O)cc1 ; COC(=O)c1ccc(-c2nc3[nH]c(=S)[nH]c(=O)c3nc2-c2ccc(C(=O)OC)cc2)cc1 ; Nc1[nH]c(=S)[nH]c(=O)c1/N=C/c1ccc([N+](=O)[O-])cc1 ; COC(=O)c1ccc(/C=N/c2c(N)[nH]c(=S)[nH]c2=O)cc1]

The NHEJ inhibitor can comprise an SCR7 analog selected from:

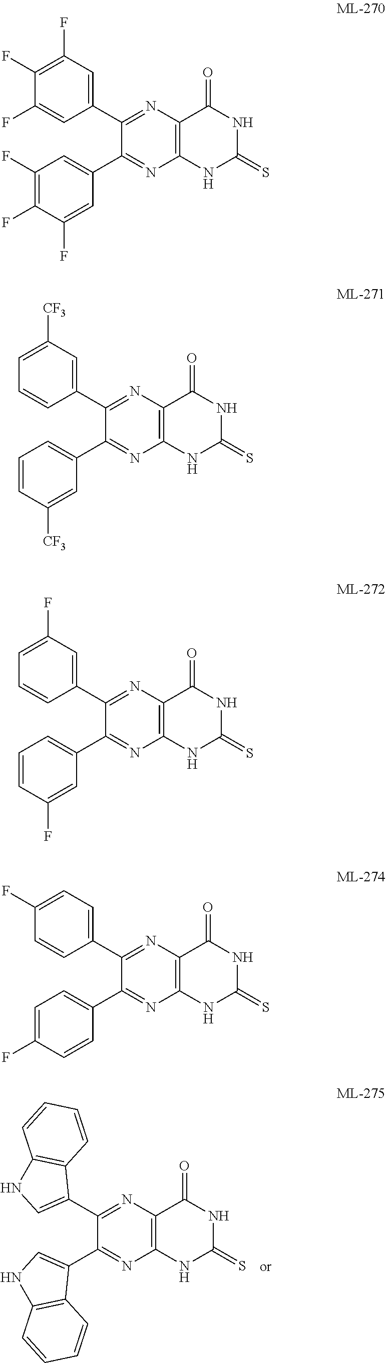 [SMILES: O=c1[nH]c(=S)[nH]c2nc(-c3cc(F)c(F)c(F)c3)c(-c3cc(F)c(F)c(F)c3)nc12 ; O=c1[nH]c(=S)[nH]c2nc(-c3cccc(C(F)(F)F)c3)c(-c3cccc(C(F)(F)F)c3)nc12 ; O=c1[nH]c(=S)[nH]c2nc(-c3cccc(F)c3)c(-c3cccc(F)c3)nc12 ; O=c1[nH]c(=S)[nH]c2nc(-c3ccc(F)cc3)c(-c3ccc(F)cc3)nc12 ; O=c1[nH]c(=S)[nH]c2nc(-c3c[nH]c4ccccc34)c(-c3c[nH]c4ccccc34)nc12]

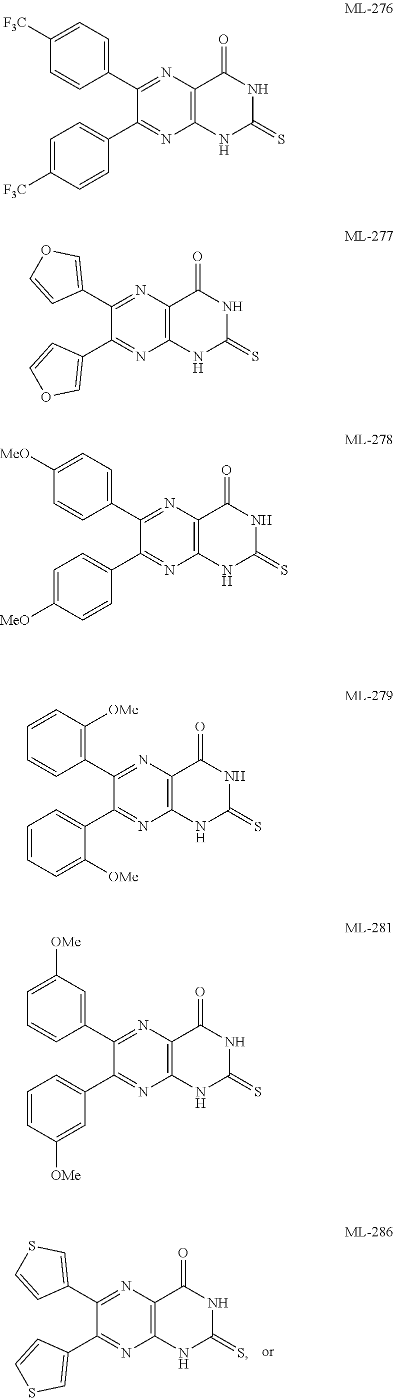

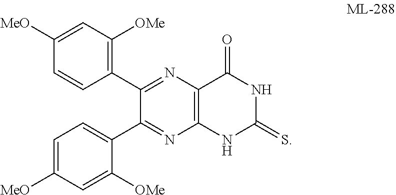 [SMILES: COc1ccc(-c2nc3[nH]c(=S)[nH]c(=O)c3nc2-c2ccc(OC)cc2OC)c(OC)c1]

HDR activators used in the compositions, systems, and complexes can be small molecules, is RS1 or stimulates RAD51. In embodiments, the HDR activators are selected from

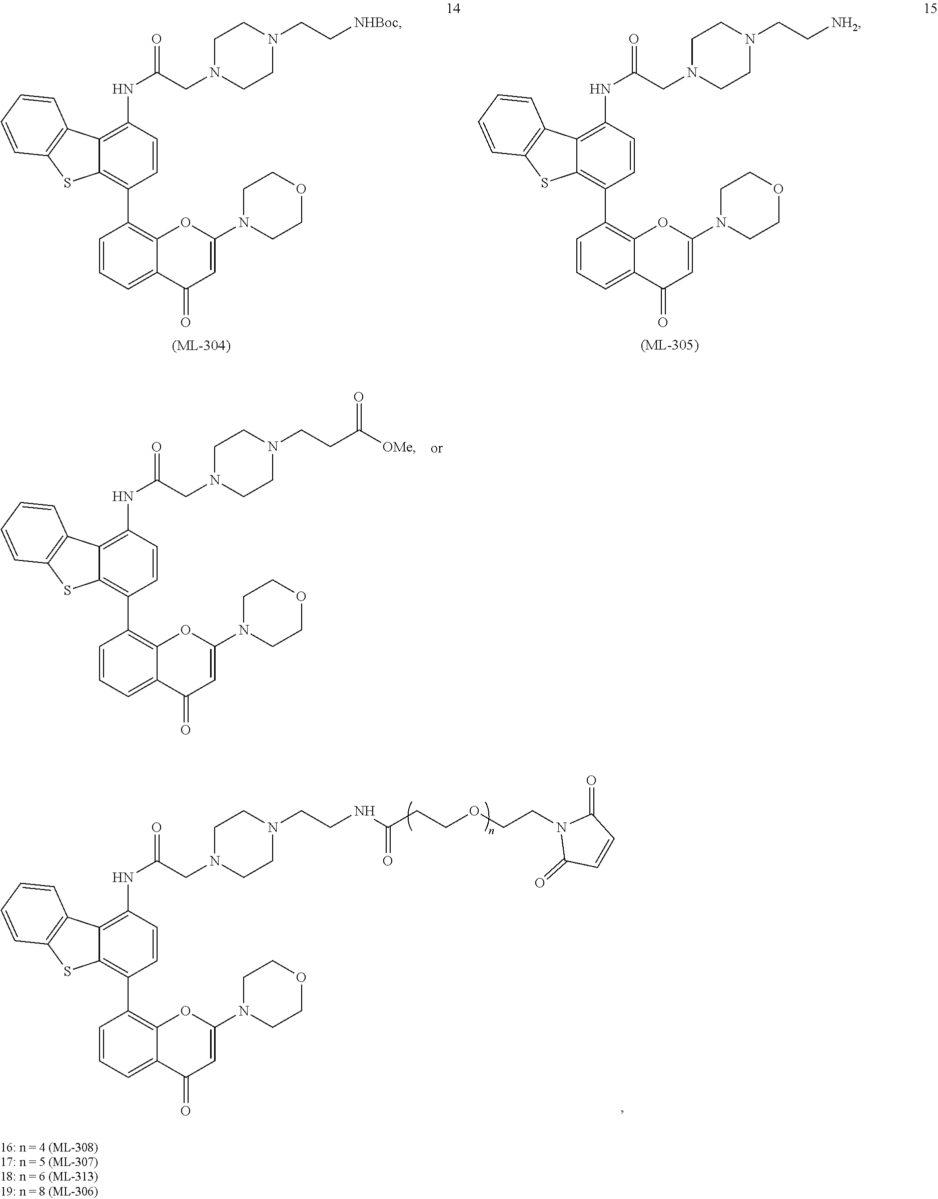

wherein n=4, 5, 6 or 8.

In embodiments, the engineered, non-naturally occurring complex comprises a p53 inhibitor, optionally α pifthrin, or an ATM kinase inhibitor, optionally KU-55933. In embodiments, the engineered, non-naturally occurring complex comprises a uracil DNA glycosylase inhibitor (UGI) or functional fragment thereof.

In embodiments, the nucleic acid binding domain is truncated, in embodiments the nucleic acid binding domain is truncated as to all or part of the NUC lobe of SpCas9 or SaCas9. In embodiments, the nucleic acid binding domain is truncated as to one or more of the RuvC I, RuvC II, RuvC III, HNH and PI domains of SpCas9, SaCas9, or AsCpf1. In embodiments, the nucleic acid binding domain comprises amino acids of the RuvC, bridge helix, REC, WED, phosphate lock loop (PLL), and PI domains of SpCas9, AsCpf1, or SaCas9. In embodiments, the nucleic acid binding domain lacks one or more of RuvCI, RuvCII, RuvCIII, NUC, PI, or BH.

In embodiments, the nucleic acid binding domain comprises amino acids of the RuvC, bridge helix, REC1, and PI domains of SpCas9 that interact with SpCas9 guide RNAs. In certain embodiments, the nucleic acid binding domain comprises binding residues which correspond to all or a subset of the following amino acids of SpCas9: Lys30, Lys33, Arg40, Lys44, Asn46, Glu57, Thr62, Arg69, Asn77, Leu101, Ser104, Phe105, Arg115, His116, Ile135, His160, Lys163, Arg165, Gly166, Tyr325, His328, Arg340, Phe351, Asp364, Gln402, Arg403, Thr404, Asn407, Arg447, Ile448, Leu455, Ser460, Arg467, Thr472, Ile473, Lys510, Tyr515, Trp659, Arg661, Met694, Gln695, His698, His721, Ala728, Lys742, Gln926, Val1009, Lys1097, Val1100, Gly1103, Thr1102, Phe1105, Ile1110, Tyr1113, Arg1122, Lys1123, Lys1124, Tyr1131, Glu1225, Ala1227, Gln1272, His1349, Ser1351, and Tyr1356. In embodiments, the nucleic acid binding domain further comprises binding residues which correspond to all or a subset of Ala59, Arg63, Arg66, Arg70, Arg74, Arg78, Lys50, Tyr515, Arg661, Gln926, and Val1009 of SpCas9, and/or further comprises binding residues which correspond to all or a subset of Leu169, Tyr450, Met495, Asn497, Trp659, Arg661, Met694, Gln695, His698, Ala728, Gln926, and Glu1108 of SpCas9. In embodiments, the nucleic acid binding domain lacks one or more amino acid positions K169, Y450, N497, R661, Q695, Q926, K810, K848, K1003, R1060, or D1135, or corresponding amino acids of an SpCas9 ortholog.

In embodiments, the nucleic acid binding domain comprises binding residues which correspond to all or a subset of the following amino acids of AsCpf1: Lys15, Arg18, Lys748, Gly753, His755, Gly756, Lys757, Asn759, His761, Arg790, Met806, Leu807, Asn808, Lys809, Lys810, Lys852, His856, Ile858, Arg863, Tyr940, Lys943, Asp966, His977, Lys1022 and Lys1029. In further embodiments, the nucleic acid binding domain further comprises binding residues which correspond to all or a subset of Tyr47, Lys51, Arg176, Arg192, Gly270, Gln286, Lys273, Lys307, Leu310, Lys369, Lys414, His 479, Asn515, Arg518, Lys530, Glu786, His872, Arg955, and Gln956 of AsCpf1 and/or all or a subset of Asn178, Ser186, Asn278, Arg301, Thr315, Ser376, Lys524, Lys603, Lys780, Gly783, Gln784, Arg951, Ile964, Lys965, Gnl1014, Phe1052, and Ala1053 of AsCpf1.

In embodiments, the nucleic acid binding domain comprises binding residues which correspond to all or a subset of the following amino acids of SaCas9: Asn47, Lys50, Arg54, Lys57, Arg58, Arg61, His62, His111, Lys114, Gly162, Val164, Arg165, Arg209, Glu213, Gly216, Ser219, Asn780, Arg781, Leu783, Leu788, Ser790, Arg792, Asn804, Lys867, Tyr868, Lys870, Lys878, Lys879, Lys881, Leu891, Tyr897, Arg901, and Lys906. The engineered, non-naturally occurring complex may comprise a nucleic acid binding domain that further comprises binding residues which correspond to all or a subset of Asn44, Arg48, Arg51, Arg55, Arg59, Arg60, Arg116, Gly117, Arg165, Gly166, Arg208, Arg209, Tyr211, Thr238, Tyr239, Lys248, Tyr256, Arg314, and Asn394, of SaCas9 and/or all or a subset of Tyr211, Trp229, Tyr230, Gly235, Arg245, Gly391, Thr392, Asn419, Leu446, Tyr651, and Arg654 of SaCas9.

In embodiments, the nucleic acid binding domain and the one or more effector domains are covalently linked. The linker may comprise a chemical linker, an amino acid linker, which may comprise Gly-Gly-Gly-Gly-Ser (GGGGS) (SEQ ID NO: 92). The linker may comprise PEG, and/or may be cleavable in vivo. In certain embodiments, the binding domain and one or more effector domains are non-covalently associated. In embodiments, the complex is inducible, or switchable.

In embodiments, the guide comprises RNA. The guide may comprise a nucleotide analog. The guide can comprise an aptamer that associates with one or more effector domains.

In certain embodiments, an engineered, non-naturally-occurring molecule is provided comprising a nucleic acid binding domain and one or more effector domains, and wherein as to an analogous naturally-occurring molecule, the engineered, non-naturally-occurring molecule is truncated and the one or more effector domains is heterologous. In embodiments, the nucleic acid-binding domain comprises at least five or more transcript activator-like effector (TALE) monomers and at least one or more half-monomers specifically ordered to a target locus of interest. The one or more monomers or half-monomers comprise one or more peptidomimetics, and/or may be further modified to be proteolytically and chemically stable. Further modifications may be provided, and may comprise one or more of stapling, side-chain cross-linking, and hydrogen-bond surrogating. The engineered molecule or complex may comprise one or more effector domain comprising one or more of a single-stranded nuclease, a double-stranded nuclease, a helicase, a methylase, a demethylase, an acetylase, a deacetylase, a deaminase, an integrase, a recombinase, of a cellular uptake activity associated domain. The one or more effector domains comprise a small molecule that induces single- or double-strand breaks in the nucleic acid target. The complex comprises one or more nuclear localization signals, which may be linked to the nucleic acid-binding domain, one or more effector domains.

The molecule may comprise a delivery enhancer, for example, a cellular permeability enhancer.

Guides used herein can comprise a guide which comprises a guide which directs sequence specific binding of the nucleic acid-guided molecule to a target nucleic acid. Guide molecules may comprise RNA, the RNA can be a single guide RNA (sgRNA). The guide nucleic acid in embodiments is in a duplex with a target nucleic acid. The target nucleic acid comprises chromosomal DNA, mitochondrial DNA, viral, bacterial, or fungal DNA or RNA.

Compositions may further comprise a recombination template. The recombination template is joined to the nucleic acid-binding domain by a cleavable linker.

Methods of repairing DNA damage in a cell or tissue, are provided, comprising contacting the damaged DNA of the cell or tissue with a complex or composition disclosed herein. DNA repair kits comprising the complexes or compositions described herein are also provided. Vector systems for delivering to a mammalian cell or tissue comprising the complex or compositions disclosed herein.

An engineered, non-naturally occurring nucleic acid modifying system, comprising an engineered, non-naturally occurring CRISPR/Cas protein; a guide nucleic acid, wherein the guide nucleic acid directs sequence specific binding of the CRISPR/Cas protein to a target nucleic acid; and one or more effector components, wherein the one or more effector components facilitate DNA repair by homology directed repair (HDR) are also disclosed. The systems may comprise one, two, or more ssODNs, one or more NHEJs, and/or one or more HDR activators disclosed herein. The CRISPR/Cas protein can comprise a CRISPR/Cas protein is selected from the group consisting of an engineered Cas9, Cpf1, Cas12b, Cas12c, Cas13a, Cas13b, Cas13c, and Cas13d protein. The CRISPR/Cas protein may comprise one or more engineered cysteine amino acids. In embodiments, the CRISPR/Cas protein is an SpCas9 protein comprising C80S and C574S mutations and one or more mutations selected from the group consisting of M1C, S204C, D435C, E532C, Q674C, Q826C, S867C, E945C, S1025C, E1026C, N1054C, E1068C, S1116C, K1153C, E1207C. The CRISPR/Cas may comprise two or more mutations comprising E532 C and E945C, or E532C and E1207C. The CRISPR/Cas protein can, in some embodiments, comprise a sortase recognition sequence Leu-Pro-Xxx-Thr-Gly, one or more unnatural amino acid p-Acetyl Phenylalanine (pAcF), or one or more unnatural amino acid comprising tetrazine.

In embodiments, the one or more effector components further comprise one or more adaptor oligonucleotides, wherein one adaptor oligonucleotide hybridizes with one ssODN. In embodiments, each adaptor oligonucleotide and the hybridizing ssODN have at least 13 overlapping nucleotides. The one or more effector components can in some embodiments, be linked to the CRISPR/Cas protein, which may be covalently linked. In embodiments, the one or more effector components are linked to the CRISPR/Cas protein via cysteines, sortase chemistry, or unnatural amino acids. The one or more effector components are linker modified, wherein the linker may comprise a maleimide group, PEG, or a poly-Gly peptide. In embodiments, one or more adaptor oligonucleotides are linked to the CRISPR/Cas protein via thiol-maleimide chemistry. The one or more adaptor oligonucleotides can comprise at least 10 nucleotides, at least 13 nucleotides, at least 15 nucleotides, or at least 17 nucleotides.

Methods for enhancing HDR at one or more target loci in a target cell are provided, comprising delivering the system of any of the systems or complexes disclosed herein to the target cell. Delivery to the target cell may be provided via electroporation, or lipid mediated delivery in some embodiments.

An engineered, non-naturally occurring nucleic acid modifying system, comprising a first engineered, non-naturally occurring DNA reader, wherein the first DNA reader binds a target nucleic acid; and a first effector component, wherein the first effector is a small molecule and modifies the target nucleic acid are provided. In embodiments, the first DNA reader is a peptide nucleic acid (PNA) polymer, or transcript activator-like effector (TALE). The systems can further comprise one or more NHEJ inhibitors and/or more HDR activators. The DNA reader may comprise a PNA polymer. The first effector component can comprise a small molecule synthetic nuclease, which can, in certain embodiments, be selected from the group consisting of diazofluorenes, nitracrines, metal complexes, enediyenes, methoxsalen derivatives, daunorubicin derivatives, and juglones. In embodiments, the small synthetic nuclease is selected from

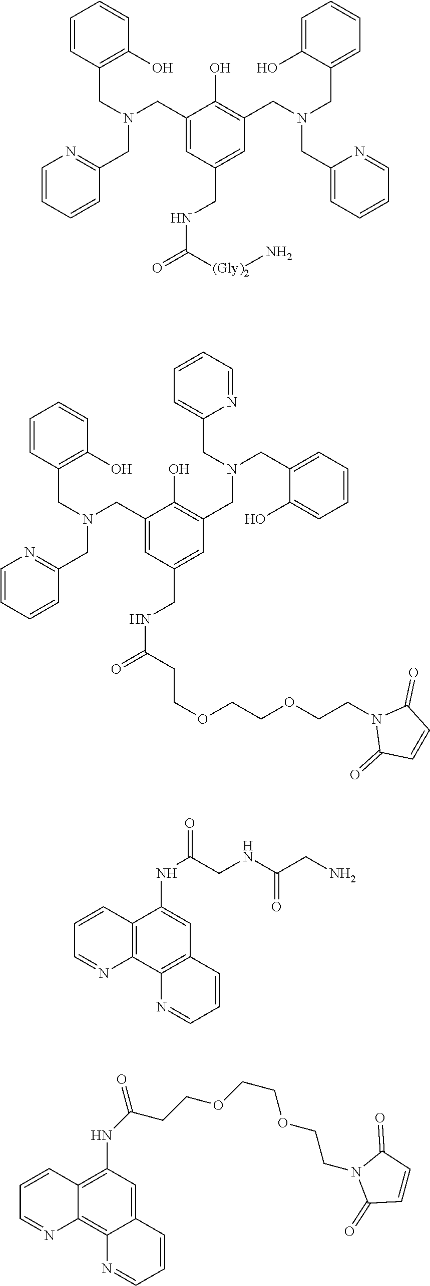

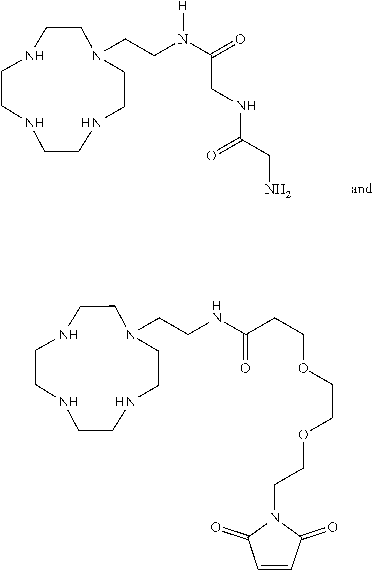 [SMILES: NCC(=O)NCC(=O)NCCN1CCNCCNCCNCC1 ; O=C(CCOCCOCCN1C(=O)C=CC1=O)NCCN1CCNCCNCCNCC1]

The small synthetic nuclease is, in some embodiments, a single strand breaking small molecule, or a double strand breaking small molecule. The first effector component can be linked to the first DNA reader, which may be covalently linked, comprises one or more maleimide, azide, or alkyne functional groups and the first DNA reader comprises a PEG linker comprising one or more thiol, alkyne, or azide functional groups. The systems can further comprise a second DNA reader and a second effector component, with a first effector component linked to the first DNA reader and the second effector component covalently linked to the second DNA reader, where both the first and second DNA readers are optionally PNA polymers.

In certain embodiments, the first effector component is an inactive small molecule synthetic nuclease and the second effector component is a trigger reagent, wherein the trigger reagent activates the small molecule synthetic nuclease. The first effector component can comprise Kinamycin C and the second effector component a reducing agent, or the first effector component can comprise dynemicin and the second effector component a reducing agent. The first effector component can comprise a first fragment of a reactive group of a small molecule synthetic nuclease and the second effector component a second fragment of the reactive group of the small molecule synthetic nuclease, wherein the small molecule synthetic nuclease is only active when the first fragment and the second fragment are together. The systems can comprise a third and fourth effector component. In embodiments, both the first and second DNA readers are PNA polymers, and the first, second, third, and fourth effector component are small molecule single strand breaking synthetic nucleases. In embodiments, the first and second synthetic nucleases are linked to the first PNA polymer, and the third and fourth synthetic nucleases are linked to the second PNA polymer. The systems can further comprise one or more NHEJ inhibitors and/or one or more HDR activators as described herein.

Methods of precise genome editing in a cell or tissue are provided, comprising delivering the systems provided herein to a cell or tissue. In embodiments, systems can be delivered using Poly(lactic co-glycolic acids) (PLGA) nanoparticles.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Development of SynGEM. (A) A SynGEM. (B) A HiBiT assay for HDR-mediated knock-in of the 33-nt DNA fragment. (C) Knock-in efficiencies by Cas9-adaptors compared to unlabeled wildtype Cas9 when a separate Cas9/ssODN system was used. (D) HDR-enhancement in U2OS cells, HEK-293FT cells, and MDA-MB-231 cells. (E-G) Sortase-mediated Cas9 labeling. (H) Small-molecule inhibitors of NHEJ pathway. (I) Demonstration of NHEJ inhibition by these small-molecules in the ddPCR assay. (J) Demonstration of HDR enhancement by NHEJ pathway inhibitors in the HiBiT assay.

FIG. 2—Schematic showing synthesis of ligand.

FIG. 3—Target molecule and synthesis scheme.

FIG. 4—Schematic showing synthesis of Phenanthroline-Gly.

FIG. 5—Schematic showing synthesis of cyclen-Gly.

FIG. 6—Schematic showing conjugation to Cas9-Cys-Mutants.

FIG. 7—Schematic showing conjugation to Cas-9-Cys-Mutants

FIG. 8—Schematic showing conjugation to Cas-9-Cys-Mutants

FIG. 9—Shows structures and cleavage data for compounds known to be able to cut a nucleotide strand.

FIG. 10—Synthetic scheme for SAGE compounds

FIG. 11A-11B—(11A) Synthetic scheme of SAGE compounds; (11B) Synthetic scheme of SAGE compounds.

FIG. 12A-12C—(12A) A modular design strategy to functionalize Cas9. (12B) Structure-guided selection of chemical labeling sites. (12C) ssODN is conjugated to Cas9 to promote HDR-mediated precision genome editing.

FIG. 13—Cas9-ssODN conjugation enhances HDR-mediated 33-nt HiBiT sequence knock-in efficiency at the GAPDH locus. (13A) Schematic of the separate Cas9/ssODN unconjugated system and Cas9-ssODN conjugates. (13B) Knock-in efficiencies by Cas9-adaptors compared to unlabeled wildtype (wt) Cas9 when a separate Cas9/ssODN system was used. (13C) Knock-in results in U2OS cells, (13D) HEK-293FT cells, and (13E) MDA-MB-231 cells. The panels on the left show luminescence intensities using the separate Cas9/ssODN system. The middle panels show luminescence intensities from Cas9-ssODN conjugates. The panels on the right show HDR fold-enhancement from the Cas9-ssODN conjugation. All data from biological replicates are shown. Error bars represent standard deviation.

FIG. 14—Cas9-ssODN conjugation promotes HDR in general. (A) Another GADPH-targeting gRNA was used for HiBiT knock-in. (B) The PPIB locus or (C) CFL1 locus was targeted for HiBiT knock-in. (D) The GFP11 sequence was inserted at the GAPDH locus. Either a separate Cas9/ssODN system (left panels) or a Cas9-ssODN conjugate (middle panels) was used to measure the fold knock-in enhancement (right panel). Unlabeled wt Cas9 and Cas9-adaptor labeled at residue 532 were used. All data from biological replicates are shown. Error bars represent standard deviation.

FIG. 15—Cas9-ssODN conjugation promotes HDR-mediated single-nucleotide exchange at the RBM20 locus in HEK-293FT cells. (A) One of the CG pairs at exon 9 or RBM20 gene is replaced by AT pair to generate a dilated cardiomyopathy model. 31 (B) ddPCR-based quantification of HDR and NHEJ frequencies with unlabeled wt Cas9 and Cas9-adaptor conjugates. ssODN contained adaptor-binding sequence. All data from biological replicates are shown (*p<0.05, **p<0.01, paired two-tailed t-test).

FIG. 16—Conjugation of a second ssODN to Cas9 further enhances HDR efficiency. (A) Schematic illustrating the production of Cas9 double-ssODN conjugates. (B) HiBiT sequence knock in at the GAPDH locus was detected in U2OS cells. (C), (D) Single-nucleotide exchange at the RBM20 locus was detected in HEK-293FT cells. Unlabeled wt Cas9 and Cas9-adaptor conjugates labeled at the indicated residues were used. RNP and ssODNs were used at a ratio of 1:2. All data from biological replicates are shown (*p<0.05, paired two-tailed t-test).

FIG. 17—Nucleic acid modifiers (SAGE). Shown are DNA strand breaking compounds for TALE and Cas9 conjugation.

FIG. 18—Nucleic acid modifiers (SAGE). Shown are NHEJ inhibitors/HDR activators, with SCR6 and its analogs shown at the top and middle and SCR7 and one of its analogs shown on the bottom.

FIG. 19—SCR7 and its analogs.

FIG. 20—HDR activators.

FIG. 21—Synthesis of northern (top) and southern part of Ku inhibitor (bottom).

FIG. 22—Schematic showing synthesis of new Ku inhibitor analog 15.

FIG. 23—CRISPR screen and inhibitors.

FIG. 24—Schematic illustrating synthesis of BRD9822.

FIG. 25—Schematic illustrating synthesis of BRD9822.

FIG. 26—Schematic illustrating synthesis of BRD7608.

FIG. 27—BRD7608-Biotin.

FIG. 28—Degradation domain modifications for spatio-temporal control of RNA-guided nucleases.

FIG. 29—Schematic illustrating synthesis of alcohol.

FIG. 30—Schematic illustrating synthesis of TFA salt.

FIG. 31—Schematic illustrating synthesis of acid.

FIG. 32—Schematic illustrating synthesis of acid.

FIG. 33—Schematic illustrating synthesis of dTAG47 (PK462).

FIG. 34—An exemplary modular design strategy to functionalize Cas9.

FIGS. 35A-35F—Pancreatic β-cell genome editing with Cas9-ssODN conjugates enabled the efficient secretion of exogenous peptides and proteins. (FIG. 35A) Schematic of the genome editing in INS1 locus of INS-1E cells to exploit insulin processing and secretion pathway. Engineered cells can secret exogenous gene product together with insulin. (FIG. 35B) INS-1E cells were engineered to secrete the 11-residue HiBiT peptide. Multiple gene insertion sites and DNA break sites were investigated. All data from two biological replicates are shown. (FIG. 35C) Glucose-stimulated HiBiT peptide secretion demonstrates the knock-in at the INS1 locus. All data from five technical replicates are shown. (FIG. 35D) INS-1E cells were engineered to secret IL-10. All data from three technical replicates are shown. Cas9-ssODN conjugates enhanced the secretion of (FIG. 35E) HiBiT peptide and (FIG. 35F) IL-10. All data from biological replicates are shown.

FIG. 36—A schematic showing methods according to certain examples embodiments.

FIGS. 37A-37B—Selection of Cas9 labeling sites based on crystal structures. (FIG. 37A) Structure of apo-Cas9 (PDB ID: 4CMP). Labeling sites are shown as spheres. Four other selected residues (1, 532, 1116, 1153) are not assigned at the structure possibly due to the high flexibility. It was assumed that those sites are surface-exposed based on the nucleic-acid-bound structures and/or high flexibility of the loops they belong to. (FIG. 37B) Structure of gRNA-bound Cas9 (PDB ID: 4ZT0). gRNA is shown. Labeling sites are shown as spheres. Only residue 558 is projected toward the interior of the protein, indicating that labeling at this site can inhibit the formation of the correct RNP structure. Cas9 exhibits a large conformational change, especially at the recognition (REC) lobe, upon gRNA binding (residues 204, 532, 558).

FIGS. 38A-38E—(FIG. 38A) Schematic of the exemplary site-specific labeling of Cas9 single-cysteine mutants by thiol-maleimide conjugation. (FIG. 38B) Biotin-maleimide was reacted with a cysteine on Cas9. The reaction mixture was subjected to pull-down by streptavidin beads to separate between unlabeled (Flow Thru) and biotinylated (Eluate) Cas9. Each fraction was analyzed by SDS-PAGE followed by Coomassie staining. (FIG. 38C) PEG-maleimide was reacted with a cysteine on Cas9. (FIG. 38D) The adaptor oligonucleotide with a 5′-maleimide group was reacted with a cysteine on Cas9. The degree of labeling was monitored through SDS-PAGE followed by Coomassie staining for PEG and DNA labeling. Because the 1153C and 1154C mutants did not give high conversion yields, they were not used for genome editing experiments. (SEQ ID NO: 1) (FIG. 38E) Retro-Diels-Alder reaction to obtain maleimide-modified DNA.

FIGS. 39A-39E—Schematic of the HiBiT assay to check the HDR-mediated knock-in of the 33-nt DNA fragment. (FIG. 39A) General gRNA and ssODN design strategy for HDR-based HiBiT sequence knock-in right before the stop codon of the gene of interest. (FIG. 39B) The knock-in results in the expression of a fusion protein having a C-terminal HiBiT tag, which is a small fragment of the NanoLuc luciferase. When an excess amount of the other fragment of NanoLuc (LgBiT) is supplied, a fully functional NanoLuc is reconstituted. The resulting luminescence signal is proportional to the HDR efficiency. (FIG. 39C) Design strategy for HiBiT knock-in at the GAPDH locus. gRNA 1 was used for genome editing in FIG. 2, and gRNA 2 was used in FIG. 14. (FIG. 39D) Design strategy for HiBiT knock-in at the PPIB locus or (FIG. 39E) the CFL1 locus.

FIG. 40—Electrophoretic mobility shift assay to check the binding between Cas9-adaptor conjugates and ssODN. When the ssODN contained the adaptor-binding sequence, the specific Cas9-ssODN complex was observed. In contrast, only non-specific binding patterns were observed when the ssODN did not have the corresponding sequence or when the unlabeled wildtype Cas9 (wt) was used. The ssODN for HiBiT knock-in at the GAPDH locus was used. Even though the lanes are not contiguous, they are all from a single gel.

FIGS. 41A-41B—GFP complementation assay to check the HDR-mediated insertion of the 57-nt GFP11 fragment. (FIG. 41A) In general, the GFP11 sequence was inserted right before the stop codon of a gene of interest through Cas9—and ssODN-mediated HDR. (FIG. 41B) Following genome editing, the gene of interest was expressed as a fusion with a C-terminal GFP11 tag. When the other fragment of GFP (GFP1-10) is supplied, a fully functional GFP is reconstituted, and the fluorescence signal can be detected.

FIG. 42—Schematic of the droplet digital PCR-based quantification of NHEJ and HDR. The reference probe was capable of binding to all alleles while the HDR probe bound only to the precisely edited allele. The NHEJ probe was a drop-off probe that was not capable of binding to the NHEJ-repaired allele. Each probe was labeled with a fluorophore-quencher pair. During the PCR, DNA-bound probes were hydrolyzed by the exonuclease activity of the DNA polymerase. Therefore, fluorophores and quenchers moved apart from each other, providing fluorescence signals.

FIGS. 43A-43B—Droplet digital PCR-based quantification of single-nucleotide exchange at the RBM20 locus using another gRNA-ssODN pair. (FIG. 43A) The relative location of the gRNA and ssODN in the context of the RBM20 genomic sequence. (FIG. 43B) Droplet digital PCR-based quantification of HDR and NHEJ frequencies with unlabeled wildtype Cas9 (wt) or Cas9-adaptor conjugates. The ssODN contained adaptor-binding sequence. All data points from two biological replicates are shown.

FIGS. 44A-44C—(FIG. 44A) Schematic of the eGFP knock-out assay to investigate the off-target profile of the Cas9-adaptor conjugate. The eGFP PEST gene stably expressed in U2OS cells was targeted by Cas9 RNP using on-target and off-target gRNAs. (FIG. 44B) Sequences of the gRNAs. Off-target sites were in light gray. PAM sequences in gray. (SEQ ID NOs: 2-5) (FIG. 44C) Results of the eGFP knock-out assay. Cells were nucleofected with 10 pmol of RNP and were incubated for 48 h followed by nuclei staining and fluorescence imaging. Unlabeled wildtype Cas9 (wt) and Cas9-adaptor labeled at residue 532 and 945 were used. Error bars represent standard deviation from≥ four technical replicates.

FIG. 45—Effect of the base-pairing length on the HDR-enhancing capability of the Cas9-ssODN conjugate. HiBiT sequence insertion was employed as a test HDR assay in U2OS.eGFP.PEST cells using the Cas9-adaptor labeled at residue 945. Luminescence was detected 24 h post transfection. All data points from three biological replicates are shown.

FIGS. 46A-46B—(FIG. 46A) Site-specific labeling of Cas9 mutants at two cysteine residues using thiol-maleimide conjugation. The degree of labeling was measured through SDS-PAGE followed by Coomassie staining. (SEQ ID NO: 6) (FIG. 46B) An electrophoretic mobility shift assay (EMSA) was performed using Cas9-adaptor conjugates and ssODN specific for GAPDH HiBiT tagging that contained the adaptor-binding sequence. The RNP and ssODN were used at a ratio of 1:2.

FIG. 47—Glucose-stimulated HiBiT peptide secretion from edited INS-1E cells in independent experiments. All data points from technical replicates are shown.

FIG. 48—IL-10 secretion from edited INS-1E cells in an independent experiment. All data points from technical replicates are shown.

FIG. 49A-FIG. 49B—Confirmation of IL-10 knock-in by PCR. FIG. 49A Primers specific for knock-in sequence were used. FIG. 49B Genomic DNA was extracted from cells exhibiting different IL-10 secretion levels, and PCR was performed using two different primer sets followed by agarose gel electrophoresis and ethidium bromide staining. Numbers in parentheses show IL-10 concentration form the cell culture supernatant. Correct incorporation of IL-10 was confirmed by Sanger sequencing.

FIG. 50A-FIG. 50E—Cas9-ssODN conjugate enhanced precision genome editing in INS-1E cells. FIG. 50A-50D Both HiBiT knock-in and IL-10 knock-in were promoted by Cas9-ssODN conjugation when two different gRNAs were tested. Unlabeled wildtype (wt) Cas9 and Cas9-adaptor labeled at residue 945 were used. All data from biological replicates are shown; FIG. 50E Electrophoretic mobility shift assay to check the binding between Cas9-adaptor conjugates and long ssODNs for IL-10 knock-in. The specific Cas9-ssODN complex was observed only when both Cas9 and ssODN contained the complementary adaptor sequences. The lanes are all from a single gel. Unlabeled wildtype (wt) Cas9 and Cas9-adaptor labeled at residue 945 were used.

FIG. 51—A Schematic showing an exemplary approach for editing INS1 gene.

FIG. 52—Selection of gRNA at different sites.

FIG. 53—Cas9-ssODN conjugates for HiBiT insertion.

FIG. 54—Results from glucose-stimulated peptide secretion.

The following detailed description, given by way of example, but not intended to limit the invention solely to the specific embodiments described, may best be understood in conjunction with the accompanying drawings.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboraotry Manual (1988) (Harlow and Lane, eds.): Antibodies A Laboraotry Manual, 2^{nd} edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to U.S. Provisional Application No. 62/575,948, filed Oct. 23, 2017, and U.S. Provisional Application No. 62/765,347 filed Aug. 20, 2018, and PCT/US2018/057182, entitled “Novel Nucleic Acid Modifiers,” filed Oct. 23, 2018,

All publications, published patent documents, and patent applications cited herein are hereby incorporated by reference to the same extent as though each individual publication, published patent document, or patent application was specifically and individually indicated as being incorporated by reference.

### Overview

The present disclosure provides a synthetic all-in-one genome editor (SAGE) comprising designer DNA sequence readers and a set of small molecules that induce double-strand breaks, enhance cellular permeability, inhibit NHEJ and activate HDR. The central problem of the CRISPR-system is the large size of the nuclease domains (>100 kDa). In SAGE, small molecules (<500 Da) preferably conduct the functions of these nuclease domains resulting in dramatic size reduction, which enhances cellular delivery and allows multiplexed genome editing on an unprecedented scale. The cellular delivery is further enhanced using small molecules that improve membrane permeability. Precise genome editing may comprise NHEJ inhibition and HDR activation locally at the site of the double-strand break, a feature missing from the current CRISPR-systems. In preferred embodiments, SAGE bears small molecules that activate HDR and suppress NHEJ locally at the genomic site of the double-strand breaks. SAGE's backbone, which may be made from synthetic polymer, and in certain embodiments is engineered to be resistant to degradation by proteases/nucleases, or harsh conditions of temperature, pH, and humidity. SAGE is fast acting since host does not synthesize/assemble its components (unlike CRISPR-system). Since SAGE components are synthetic polymers and small molecules, the infrastructure for their mass production is already in place. Further, SAGE provides a countermeasure for correcting unwanted genomic alteration in an organism or population.

Presented herein is a simple, scalable, and modular chemical platform for site-specific Cas9 labeling with a wide range of functional molecules. Multiple internal residues compatible with modification by thiol-maleimide reaction were identified without compromising the enzyme function. As model labels, small molecule (biotin) and medium-sized molecule (PEG) were efficiently linked to Cas9. In certain embodiments, short oligonucleotide handle is utilized as a universal anchoring point for any kind of oligonucleotide-containing functional molecules, making this platform amenable to nearly every type of desired conjugate. In embodiments, ssODN can be attached, which can increase HDR efficiency, and which can be displayed multivalently. The adaptor handle can hybridize to any type of cargos bearing the complementary DNA, providing methods for the practical application of genome engineering technology. It is also noteworthy that any types of knock-in (single nucleotide exchange, short DNA insertion, and long gene insertion) can be promoted by the chemically enhanced Cas9 constructs. Using the CRISPR-Cas9 and HDR-based genome editing, β-cells were precisely engineered and the precise knock-in strategy believed safer than conventional random gene integration methods using viral vectors that result in unpredictable genomic sequences. As a proof-of-concept, β-cells were produced that can secrete IL-10, and Cas9-ssODN conjugates were successfully used to enhance the precision genome editing opening up a new possibility of chemically enhanced Cas9.

In an aspect, the invention provides a composition comprising a nucleic acid modifier. In an aspect, the invention provides a composition for site specific delivery of a nucleic acid modifier. In one aspect, the invention provides an engineered, non-naturally occurring nucleic acid modifying system, comprising: (a) an engineered, non-naturally occurring CRISPR/Cas protein; (b) a guide nucleic acid, wherein the guide nucleic acid directs sequence specific binding of the CRISPR/Cas protein to a target nucleic acid; and (c) one or more effector components, wherein the one or more effector components facilitate DNA repair by HDR.

In an aspect, the SAGEs provide at a most basic level a molecule or molecules that bind target nucleic acid; and an effector component that modifies, directs breaks, or induces breaks in target nucleic acid. Advantageously the target nucleic acids can include DNA or RNA, for example chromosomal or mitochondrial DNA, viral, bacterial or fungal DNA or viral bacterial, or fungal RNA.

The one or more molecules that bind target nucleic acid comprise, in some embodiments, a nucleic acid binding domain, which in preferred embodiments is an engineered, non-naturally occurring CRISPR/Cas protein. In some embodiments, the CRISPR protein is truncated, in some embodiments, the CRISPR/Cas protein comprises one or more engineered amino acids or unnatural amino acids. The CRISPR/Cas proteins are in some embodiments an engineered Cas9, Cpf1, Cas12b, Cas12c, Cas13a, Cas13b, Cas13c, or Cas13d protein. The molecule that binds target nucleic acid may be provided with a guide nucleic acid that directs sequence specific binding of the CRISPR/Cas protein to a target nucleic acid.

In other embodiments, the one or molecules that bind target nucleic acid comprise at least five or more transcript activator-like effector (TALE) monomers and at least one or more half-monomers specifically ordered to a target locus of interest.

In embodiments, the one or more molecules that bind target nucleic acid are one or more engineered-non-naturally occurring DNA readers. In some embodiments, the DNA reader is a peptide nucleic acid (PNA) polymer or a TALE.

The effector component in embodiments may comprise one or more effector domains, which in some instances are a single strand nuclease, double strand nuclease, a helicase, a methylase, a demethylase, an acetylase, a deacetylase, a deaminase, an integrase, a recombinase or a cellular uptake activity associated domains.

The effector domain can comprise a small molecule that induces single or double strand breaks in the target nucleic acid. In some embodiments, the one or more effector components facilitate DNA repair by homology directed repair (HDR), and can be one or more single-stranded oligodonors (ssODNs), NHEJ inhibitors, or HDR activators.

In embodiments when a DNA reader is the molecule that binds a target nucleic acid, the effector component is a small molecule that can be a small molecule synthetic nuclease. The system with DNA readers may contain more than one DNA reader, preferably a PNA polymer. One or more effector components can be provided as more than one fragment that is only active when the fragments are together, e.g. split effector components.

In certain embodiments, the invention comprises the following modular components: (i) single- or double-strand breaker, (ii) NHEJ inhibitor, (iii) HDR activator, (iv) designer DNA-sequence reader, (v) nuclear localization sequence, (vi) enhancers of cellular permeability, (vii) p53 pathway inhibitor, and (viii) DNA glycosylase inhibitor.

The nuclease function may be effected by small-molecules such as

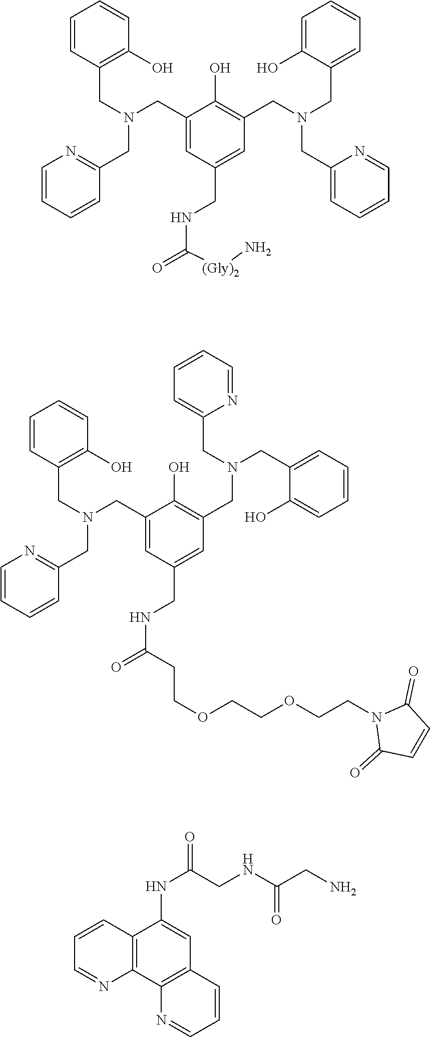

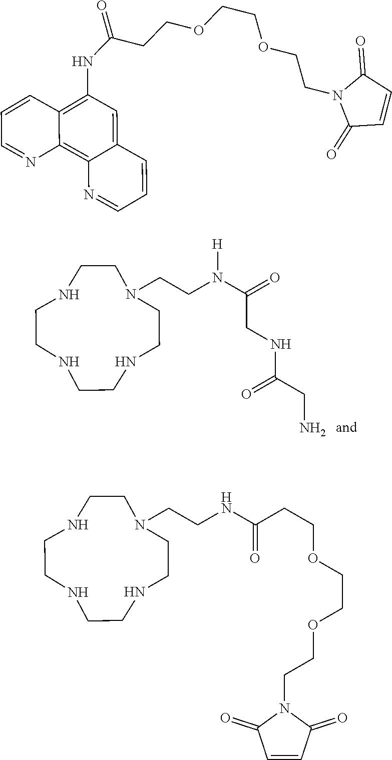 [SMILES: O=C(CCOCCOCCN1C(=O)C=CC1=O)Nc1cc2cccnc2c2ncccc12 ; NCC(=O)NCC(=O)NCCN1CCNCCNCCNCC1 ; O=C(CCOCCOCCN1C(=O)C=CC1=O)NCCN1CCNCCNCCNCC1]

NHEJ inhibition and HDR activation can be accomplished by appending small molecule inhibitors of NHEJ (e.g., SCR7 or SCR6 analogs) and small molecule activators or enhancers of HDR.

In an aspect, the invention provides a vector system for delivery of a nucleic acid modifier or delivery of a composition comprising a nucleic acid modifier to a mammalian cell or tissue.

In an aspect, the invention provides a nucleic acid modifying system comprising a nucleic acid modifier or a composition comprising a nucleic acid modifier.

In an aspect, the invention provides a particle delivery system for delivery of a nucleic acid modifier or delivery of a composition comprising a nucleic acid modifier to a mammalian cell or tissue. In certain embodiments, the particle delivery system is a nanoparticle delivery system comprised of polymers, which can comprise poly(lactic co-glycolic acids) (PLGA) polymers. In embodiments the particle delivery system comprises a hybrid virus capsid protein or hybrid viral outer protein, wherein the hybrid virus capsid or outer protein comprises a virus capsid or outer protein attached to at least a portion of a non-capsid protein or peptide. The genetic material of a virus is stored within a viral structure called the capsid. The capsid of certain viruses is enclosed in a membrane called the viral envelope. The viral envelope is made up of a lipid bilayer embedded with viral proteins including viral glycoproteins. As used herein, an “envelope protein” or “outer protein” means a protein exposed at the surface of a viral particle that is not a capsid protein. For example, envelope or outer proteins typically comprise proteins embedded in the envelope of the virus. Non-limiting examples of outer or envelope proteins include, without limit, gp41 and gp120 of HIV, hemagglutinin, neuraminidase and M2 proteins of influenza virus.

In one embodiment, the lipid, lipid particle or lipid layer of the delivery system further comprises a wild-type capsid protein.

In one embodiment, a weight ratio of hybrid capsid protein to wild-type capsid protein is from 1:10 to 1:1, for example, 1:1, 1:2, 1:3, 1:4, 1:5, 1:6, 1:7, 1:8, 1:9 and 1:10. Further delivery approaches can be used, as disclosed, for example, at [0546]-[0601] in PCT/US18/57182, incorporated herein by reference.

In an aspect, the invention provides a pharmaceutical composition comprising the particle delivery system or the delivery system or the virus particle of any one of the above embodiments or the cell of any one of the above embodiments.

In an aspect, the invention provides a method of repairing DNA damage in a cell or tissue, the method comprising contacting the damaged DNA of the cell or tissue with a nucleic acid modifier or a composition comprising a nucleic acid modifier. The invention provides a method of precise genome editing in a cell or tissue, comprising delivering the nucleic acid modifying system to the cell or tissue.

In one aspect, the invention provides a DNA repair kit comprising a nucleic acid modifier or a composition comprising a nucleic acid modifier.

Semi-Synthetic Genome Editor with Multifunctionality (SynGEM)

In embodiments, an engineered, non-naturally occurring composition is provided and includes i) an engineered, non-naturally occurring nucleic acid-guided molecule comprising a nucleic acid binding domain, and one or more effector domains. The composition can optionally be provided with a guide. In embodiments, the nucleic acid-guided molecule complexes with a guide that comprises a polynucleotide, and the composition can be provided as a complex with the guide. The guide can direct sequence specific binding of the nucleic acid-guided molecule to a target nucleic acid. Compared to an analogous naturally-occurring nucleic acid-guided molecule, such as site-specific guided nuclease, the engineered, non-naturally-occurring nucleic acid-guided complex may be truncated. In some embodiments, the nucleic acid-guided molecule is an engineered, non-naturally occurring CRISPR/Cas protein. In some embodiments, the one or more effector domains is heterologous. The nucleic acid binding domain and the one or more effector domains can be covalently linked or non-covalently associated. When the compositions are provided as a complex, the complexes can be inducible or switchable, which preferably occurs when the one or more effector domains are non-covalently associated.

In one aspect, the invention provides SynGEMs that enhance HDR at the double-strand break site. Multiple conjugation sites on engineered CRISPR/Cas proteins are identified that allow accommodation of molecular conjugation using novel, multivalent, or orthogonal conjugation chemistries without loss of activity. The capacities of Cas proteins can be augmented by bioactive small molecules. In certain embodiments, engineered Cas proteins can be mono-conjugated with ssODN, NHEJ inhibitors, or HDR activators. Complexes can be identified with a maximum enhancement of HDR. In certain embodiments, engineered CRISPR/Cas proteins can be multivalently conjugated with NHEJ inhibitors or HDR activators. In certain embodiments, engineered CRISPR/Cas proteins can be conjugated with ssODN, NHEJ inhibitors, and HDR activators using orthogonal conjugation chemistries. SynGEMs can be optimized for disease-specific ex vivo applications of interest to the members of somatic Cell Genome Editing (SCGE) Corsortia. SynGEMs allow precise genome edits while mitigating toxicity and mutagenesis arising from global NHEJ inhibition or HDR activation.

In an aspect, the invention provides a nucleic acid modifier which comprises a nucleic acid binding domain linked to an effector domain. The nucleic acid binding domain comprises one or more domains of a CRISPR protein which bind to a programmable system guide which directs complex formation of the nucleic acid modifier with the guide nucleic acid and the target nucleic acid. The nucleic acid binding domain in one embodiment does not contain a NUC lobe of a CRISPR protein, or the nucleic acid binding domain contains fewer than 50% of the amino acids of the naturally occurring CRISPR protein.

### Nucleic Acid Binding Domain

In an embodiment the nucleic acid modifier comprises Repeat Variable Diresidues (RVDs) of a TALE protein or a portion thereof linked to one or more effector domains. In an embodiment the nucleic acid modifier comprises the recognition (REC) lobe of a CRISPR protein linked to one or more effector domains. In an embodiment the nucleic acid modifier comprises domains/subdomains of Cas9 linked to one or more effector domains. In an embodiment the nucleic acid modifier comprises domains/subdomains of Cpf1 linked to one or more effector domains. In an embodiment the nucleic acid modifier comprises domains of a Cas13 protein linked to one or more effector domains.

In an embodiment of a nucleic acid modifier, the nucleic acid binding domain and the effector domain are linked by a linker comprising an inducible linker, a switchable linker, a chemical linker, PEG or (GGGGS)(SEQ ID NO:92) repeated 1-3 times, SEQ ID NOs: 92, 93 and 94, respectively.

In some general embodiments, the nucleic acid modifying protein is used for multiplex targeting comprises and/or is associated with one or more effector domains. In some more specific embodiments, the nucleic acid modifying protein used for multiplex targeting comprises one or more domains of a deadCas9 as defined herein elsewhere.

### CRISPR-Cas Proteins

In certain embodiments, the nucleic acid modifying protein is derived advantageously from a type II CRISPR system, preferably derived from Cas9. In some embodiments, one or more elements of a nucleic acid modifying system is derived from a particular organism comprising an endogenous CRISPR system, such as Streptococcus pyogenes. In preferred embodiments of the invention, the nucleic acid modifying system derives from a type II CRISPR system and the nucleic acid modifying protein comprises one or more domains of a Cas9, which catalyzes DNA cleavage. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof.

In some embodiments, the nucleic acid modifying protein has DNA cleavage activity, similar to Cas9. In some embodiments, the nucleic acid modifying protein directs cleavage of one or both strands at the location of a target sequence, such as within the target sequence and/or within the complement of the target sequence. In some embodiments, the nucleic acid modifying protein directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a nucleic acid modifying protein comprising one or more Cas9 domains that is mutated to with respect to a corresponding wild-type domains such that the nucleic acid modifying protein lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. As a further example, two or more catalytic domains of Cas9 (RuvC I, RuvC II, and RuvC III or the HNH domain) may be mutated to produce a nucleic acid modifying protein substantially lacking all DNA cleavage activity. In some embodiments, a D10A mutation is combined with one or more of H840A, N854A, or N863A mutations to produce a nucleic acid modifying protein substantially lacking all DNA cleavage activity. In some embodiments, a nucleic acid modifying protein is considered to substantially lack all DNA cleavage activity when the DNA cleavage activity of the protein is about no more than 25%, 10%, 5%, 1%, 0.1%, 0.01%, or less of the DNA cleavage activity of the protein comprising non-mutated form of the enzyme domains; an example can be when the DNA cleavage activity of the protein comprising the mutated enzyme domain is nil or negligible as compared with the protein comprising the non-mutated enzyme domain. Where the enzyme is not SpCas9, mutations may be made at any or all residues corresponding to positions 10, 762, 840, 854, 863 and/or 986 of SpCas9 (which may be ascertained for instance by standard sequence comparison tools). In particular, any or all of the following mutations are preferred in SpCas9: D10A, E762A, H840A, N854A, N863A and/or D986A; as well as conservative substitution for any of the replacement amino acids is also envisaged. The same (or conservative substitutions of these mutations) at corresponding positions in other Cas9s are also preferred. Particularly preferred are D10 and H840 in SpCas9. However, in other Cas9s, residues corresponding to SpCas9 D10 and H840 are also preferred. One or more domains belonging to orthologs of SpCas9 can be used in the practice of the invention. A Cas enzyme may be identified Cas9 as this can refer to the general class of enzymes that share homology to the biggest nuclease with multiple nuclease domains from the type II CRISPR system. Most preferably, the Cas9 enzyme is from, or is derived from, spCas9 (S. pyogenes Cas9) or saCas9 (S. aureus Cas9). StCas9″ refers to wild type Cas9 from S. thermophilus, the protein sequence of which is given in the SwissProt database under accession number G3ECR1. Similarly, S. pyogenes Cas9 or spCas9 is included in SwissProt under accession number Q99ZW2. By derived, Applicants mean that the derived enzyme is largely based, in the sense of having a high degree of sequence homology with, a wildtype enzyme, but that it has been mutated (modified) in some way as described herein. It will be appreciated that the terms Cas and CRISPR enzyme are generally used herein interchangeably, unless otherwise apparent. As mentioned above, many of the residue numberings used herein refer to the Cas9 enzyme from the type II CRISPR locus in Streptococcus pyogenes.

However, it will be appreciated that this invention includes many more Cas9s from other species of microbes, such as SpCas9, SaCa9, StCas9 and so forth. Enzymatic action by one or more domains of Cas9 derived from Streptococcus pyogenes or any closely related Cas9 generates double stranded breaks at target site sequences which hybridize to 20 nucleotides of the guide sequence and that have a protospacer-adjacent motif (PAM) sequence (examples include NGG/NRG or a PAM that can be determined as described herein) following the 20 nucleotides of the target sequence. CRISPR activity through one or more domains of Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defence in bacteria and archaea, Mole Cell 2010, Jan. 15; 37(1): 7. The type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA:tracrRNA complex directs Cas9 to the DNA target comprising, consisting essentially of, or consisting of the protospacer and the corresponding PAM via heteroduplex formation between the spacer region of the crRNA and the protospacer DNA. Finally, Cas9 mediates cleavage of target DNA upstream of PAM to create a DSB within the protospacer. A pre-crRNA array comprising, consisting essentially of, or consisting of a single spacer flanked by two direct repeats (DRs) is also encompassed by the term “tracr-mate sequences”). In certain embodiments, a nucleic acid modifying protein may be constitutively present or inducibly present or conditionally present or administered or delivered. nucleic acid modifying protein optimization may be used to enhance function or to develop new functions, one can generate chimeric nucleic acid modifying proteins. And one or more domains of Cas9 may be used as a generic DNA binding protein.

In an advantageous embodiment, the present invention encompasses effector proteins identified in a Type V CRISPR-Cas loci, e.g. a Cas12a (also referred to as Cpf1)-encoding loci denoted as subtype V-A. Presently, the subtype V-A loci encompasses cas1, cas2, a distinct gene denoted Cas12a and a CRISPR array. Cpf1 (CRISPR-associated protein Cas12a, subtype PREFRAN) is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in all Cas9 proteins, and the RuvC-like domain is contiguous in the Cas12a sequence, in contrast to Cas9 where it contains long inserts including the HNH domain. Accordingly, in particular embodiments, the nucleic acid modifying protein comprises a RuvC-like nuclease domain.

The Cas12a gene is found in several diverse bacterial genomes, typically in the same locus with cas1, cas2, and cas4 genes and a CRISPR cassette (for example, FNFX1_1431-FNFX1_1428 of Francisella cf. novicida Fx1). Thus, the layout of this novel CRISPR-Cas system appears to be similar to that of type II-B. Furthermore, similar to Cas9, the Cpf1 protein contains a readily identifiable C-terminal region that is homologous to the transposon ORF-B and includes an active RuvC-like nuclease, an arginine-rich region, and a Zn finger (absent in Cas9). However, unlike Cas9, Cas12a is also present in several genomes without a CRISPR-Cas context and its relatively high similarity with ORF-B suggests that it might be a transposon component. It was suggested that if this was a genuine CRISPR-Cas system and Cas12a is a functional analog of Cas9 it would be a novel CRISPR-Cas type, namely type V (See Annotation and Classification of CRISPR-Cas Systems. Makarova K S, Koonin EV. Methods Mol Biol. 2015; 1311:47-75). However, as described herein, Cas12a is denoted to be in subtype V-A to distinguish it from Cas12b which does not have an identical domain structure and is hence denoted to be in subtype V-B.

The nucleic acid-targeting system may be derived advantageously from a Type VI CRISPR system. In some embodiments, one or more elements of a nucleic acid-targeting system is derived from a particular organism comprising an endogenous RNA-targeting system. In particular embodiments, the Type VI RNA-targeting, the nucleic acid modifying protein comprises one or more domains of C2c2 (also referred to herein as Cas13a) Cas enzyme. In an embodiment of the invention, there is provided a nucleic acid modifying protein which comprises one or more domains of C2c2, wherein the amino acid sequence of the one or more domains have at least 80% sequence homology to the wild-type sequence of one of more domains of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2.

In an embodiment of the invention, the nucleic acid modifying protein comprises at least one HEPN domain, including but not limited to HEPN domains described herein, HEPN domains known in the art, and domains recognized to be HEPN domains by comparison to consensus sequences and motifs.

### Inducible Nucleases

The crystal structure of SaCas9 has been used to conduct structure-guided engineering generating the SaCas9-based activator system by creating a catalytically inactive version of SaCas9 (dSaCas9). Truncated CRISPR proteins of the invention generally comprise all or portions of nucleic acid binding domains of whole CRISPR proteins while nuclease functions are removed.

Accordingly, the invention includes binding domains that are homologous to nucleic acid binding domains of CRISPR proteins such as SpCas9, SaCas9, Cas12a and orthologs, and can be 60%, 70%, 80%, 90%, or 95% identical over the range of amino acid locations in common. Amino acid residues likely to be conserved between binding domains of the invention and the various CRISPR proteins include those identified for SpCas9, SaCas9, and Cas12a: Binding domains can resemble the complex of the Cas9 protein with crRNA and tracrRNA or sgRNA, and can comprise residues which correspond with respect to the binding of guide and target to amino acids of SaCas9, as provided in the table at pages 47-48 of International Patent Publication WO2019/135816, incorporated herein by reference, and as described in [0159]-[0166] of International Patent Publication WO2019/135816. Similarly, for SpCas9 at the Table International Patent Publication WO2019/135816 spanning pages 34-36 and description at [0136]-[0148], and Aspf1 at the Table of International Patent Publication WO2019/135816 at pp. 59-61 and as described at [0180]-[0187].

In embodiments, the nucleic acid binding domain comprises binding residues which correspond to all or a subset of the following amino acids of SpCas9: Lys30, Lys33, Arg40, Lys44, Asn46, Glu57, Thr62, Arg69, Asn77, Leu101, Ser104, Phe105, Arg115, His116, Ile135, His160, Lys163, Arg165, Gly166, Tyr325, His328, Arg340, Phe351, Asp364, Gln402, Arg403, Thr404, Asn407, Arg447, Ile448, Leu455, Ser460, Arg467, Thr472, Ile473, Lys510, Tyr515, Trp659, Arg661, Met694, Gln695, His698, His721, Ala728, Lys742, Gln926, Val1009, Lys1097, Val1100, Gly1103, Thr1102, Phe1105, Ile1110, Tyr1113, Arg1122, Lys1123, Lys1124, Tyr1131, Glu1225, Ala1227, Gln1272, His1349, Ser1351, and Tyr1356. In certain instances, the nucleic acid binding domain further comprises binding residues which correspond to all or a subset of Ala59, Arg63, Arg66, Arg70, Arg74, Arg78, Lys50, Tyr515, Arg661, Gln926, and Val1009 of SpCas9, and/or further comprises binding residues which correspond to all or a subset of Leul69, Tyr450, Met495, Asn497, Trp659, Arg661, Met694, Gln695, His698, Ala728, Gln926, and Glu1108 of SpCas9. In embodiments, the nucleic acid binding domain is truncated as to all or part of the NUC lobe of SpCas9. The nucleic acid binding domain may be truncated as to one or more of the RuvCI, RuvC II, RuvC III, HNH and PI domains of SpCas9.

In certain embodiments, the nucleic acid binding domain comprises amino acids of the RuvC, bridge helix, REC, WED, phosphate lock loop (PLL), and PI domains of SaCas9 that interact with SaCas9 guide RNAs. In embodiments, the nucleic acid binding domain comprises binding residues which correspond to all or a subset of the following amino acids of SaCas9: Asn47, Lys50, Arg54, Lys57, Arg58, Arg61, His62, His111, Lys114, Gly162, Val164, Arg165, Arg209, Glu213, Gly216, Ser219, Asn780, Arg781, Leu783, Leu788, Ser790, Arg792, Asn804, Lys867, Tyr868, Lys870, Lys878, Lys879, Lys881, Leu891, Tyr897, Arg901, and Lys906. The nucleic acid binding domain further comprises binding residues which correspond to all or a subset of Asn44, Arg48, Arg51, Arg55, Arg59, Arg60, Arg116, Gly117, Arg165, Gly166, Arg208, Arg209, Tyr211, Thr238, Tyr239, Lys248, Tyr256, Arg314, and Asn394, of SaCas9 and/or all or a subset of Tyr211, Trp229, Tyr230, Gly235, Arg245, Gly391, Thr392, Asn419, Leu446, Tyr651, and Arg654 of SaCas9. In some instances, the nucleic acid binding domain is truncated as to all or part of the NUC lobe of SaCas9. In cartain instances, nucleic acid binding domain is truncated as to one or more of the RuvCI, RuvC II, RuvC III, HNH, WED, and PI domains of SaCas9.

The engineered, non-naturally occurring complex may comprise the nucleic acid binding domain comprises amino acids of WED, REC1, REC2, PI, bridge helix, and RuvC domains of AsCpf1 that interact with AsCpf1 guide RNAs. Certain embodiments of the nucleic acid binding domain comprises binding residues which correspond to all or a subset of the following amino acids of AsCpf1: Lys15, Arg18, Lys748, Gly753, His755, Gly756, Lys757, Asn759, His761, Arg790, Met806, Leu807, Asn808, Lys809, Lys810, Lys852, His856, Ile858, Arg863, Tyr940, Lys943, Asp966, His977, Lys1022 and Lys1029. The nucleic acid binding domain may further comprise binding residues which correspond to all or a subset of Tyr47, Lys51, Arg176, Arg192, Gly270, Gln286, Lys273, Lys307, Leu310, Lys369, Lys414, His 479, Asn515, Arg518, Lys530, Glu786, His872, Arg955, and Gln956 of AsCpf1 and/or all or a subset of Asn178, Ser186, Asn278, Arg301, Thr315, Ser376, Lys524, Lys603, Lys780, Gly783, Gln784, Arg951, Ile964, Lys965, Gnl1014, Phe1052, and Ala1053 of AsCpf1. In embodiments, the nucleic acid binding domain is truncated as to all or part of the NUC lobe of AsCpf1. The nucleic acid binding domain can be truncated as to one or more of the WED-I, WED-II, WED-III, PI, RuvC I, RuvC II, RuvC III, Nuc, BH, and PI domains of AsCpf1.

The nucleic acid binding domain, in some embodiments, lacks one or more amino acid positions K169, Y450, N497, R661, Q695, Q926, K810, K848, K1003, R1060, or D1135, or corresponding amino acids of an SpCas9 ortholog. In some embodiments, the nucleic acid binding domain lacks one or more of RuvCI, RuvCII, RuvCIII, NUC, PI, or BH.

In certain embodiments, a nucleic acid binding domain is linked to one or more effector domains. In certain embodiments, the linkage is a covalent linkage. In certain embodiments, the linkage comprises members of a specific binding pair. In certain embodiments, the linkage comprises an inducible linkage. In certain embodiments the nucleic acid binding domain is associated with an effector domain through binding of the guide. For example, the effector domain can be covalently linked to the guide, attached to the guide through members of a specific binding pair, or by an inducible linkage. In certain embodiments, the effector domain is comprised in the DNA binding protein, for example where the DNA binding domain binds to a nucleic acid and by binding to the nucleic acid blocks transcription, or where the DNA binding domain is designed to interact with components of transcription or translation machinery.

SpCas9 is an RNA-guided nuclease from the microbial CRISPR-Cas system that can be targeted to specific genomic loci by single guide RNAs (sgRNAs). See, e.g., WO2015/089364. SpCas9 comprises a bilobed architecture composed of target recognition and nuclease lobes, accommodating a sgRNA:DNA duplex in a positively-charged groove at their interface. Whereas the recognition lobe is essential for sgRNA and DNA binding, the nuclease lobe contains the HNH and RuvC nuclease domains, which are properly positioned for the cleavage of complementary and non-complementary strands of the target DNA, respectively.

SpCas9 consists of two lobes, a recognition (REC) lobe and a nuclease (NUC) lobe. The REC lobe can be divided into three regions, a long u-helix referred to as Bridge helix (BH) (residues 60-93), the REC1 (residues 94-179 and 308-713), and REC2 (residues 180-307) domains. The NUC lobe consists of the RuvC (residues 1-59, 718-769, and 909-1098), HNH (residues 775-908), and PAM-interacting (PI) (residues 1099-1368) domains. The negatively-charged sgRNA:DNA hybrid duplex is accommodated in a positively-charged groove at the interface between the REC and NUC lobes. In the NUC lobe, the RuvC domain is assembled from the three split RuvC motifs (RuvC I-III), which interfaces with the PI domain to form a positively-charged surface that interacts with the 3′ tail of the sgRNA. The HNH domain lies in between the RuvC II-III motifs and forms only a few contacts with the rest of the protein.

The REC lobe is one of the least conserved regions across the three families of Cas9 within the Type II CRISPR system (IIA, IIB and IIC) and many Cas9s contain significantly shorter REC lobes. The REC lobe may be truncated. Consistent with the observation that the REC2 domain does not contact the bound sgRNA:DNA hybrid duplex, a Cas9 mutant lacking the REC2 domain (Δ175-307) has shown ˜50% of the wild-type Cas9 activity, indicating that the REC2 domain is not critical for DNA cleavage. The lower cleavage efficiency may be attributed in part to the reduced levels of Cas9 (Δ175-307) expression relative to that of the wild-type protein. In striking contrast, deletion of the crRNA repeat-interacting region (A97-150) or tracrRNA anti-repeat-interacting region (Δ312-409) of the REC1 domain abolished DNA cleavage activity, indicating that the recognition of the repeat:anti-repeat duplex by the REC1 domain is critical for Cas9 function.

The PAM-interacting (PI) domain confers PAM specificity: The NUC lobe contains the PI domain, which adopts an elongated structure comprising seven α-helices (α47-α53), a three-stranded antiparallel β-sheet (β18-β20), a five-stranded antiparallel β-sheet (β21-β23, β26 and β27), and two-stranded antiparallel 1-sheet (124 and 125). Similar to the REC lobe, the PI domain also represents a novel protein fold unique to the Cas9 family.

The RuvC domain targets the non-complementary strand DNA: The RuvC domain consists of a six-stranded mixed β-sheet (β1, β2, β5, β11, β14 and β17) flanked by α-helices (α34, α35 and α40-α46) and two additional two-stranded antiparallel β-sheets (β3/β4 and β5/β6). It shares structural similarity with retroviral integrase superfamily members characterized by an RNase H fold, such as Escherichia coli RuvC (PDB code 1HJR, 13% identity, root-mean-square deviation (rmsd) of 3.4 Å for 123 equivalent Cα atoms) (Ariyoshi et al., 1994) and Thermus thermophilus RuvC (PDB code 4LD0, 17% identity, rmsd of 3.4 Å for 129 equivalent Cu atoms) (Ariyoshi et al., 1994) and Thermus thermophilus RuvC (PDB code 4LD0, 17% identity, rmsd of 3.4 Å for 129 equivalent Cu atoms) (Gorecka et al., 2013). RuvC nucleases have four catalytic residues (e.g., Asp7, Glu70, His143 and Asp146 in T. thermophilus RuvC), and cleave Holliday junctions through a two-metal mechanism (Ariyoshi et al., 1994; Chen et al., 2013; Gorecka et al., 2013). Asp10 (Ala), Glu762, His983 and Asp986 of the Cas9 RuvC domain are located at positions similar to those of the catalytic residues of T. thermophilus RuvC, consistent with the previous results that the D10A mutation abolished cleavage of the non-complementary DNA strand and that Cas9 requires Mg2+ ions for cleavage activity (Gasiunas et al., 2012; Jinek et al., 2012). Moreover, alanine substitution of Glu762, His983 or Asp986 also converted Cas9 into nickases. Each nickase mutant was able to facilitate targeted double strand breaks using pairs of juxtaposed sgRNAs, as demonstrated with the D10A nickase previously (Ran et al., 2013). This combination of structural observations and mutational analysis suggest that the Cas9 RuvC domain cleaves the non-complementary strand of the target DNA through the two-metal mechanism previously observed for other retroviral integrase superfamily nucleases.

It is important to note that there are key structural dissimilarities between the Cas9 RuvC domain and RuvC nucleases, explaining their functional differences. Unlike the Cas9 RuvC domain, RuvC nucleases forms a dimer and recognize a Holliday junction (Gorecka et al., 2013). In addition to the conserved RNase H fold, the RuvC domain of Cas9 has additional structural elements involved in the interactions with the guide:DNA duplex (an end-capping loop between α43 and α44), and the PI domain/stem loop 3 (β-hairpin formed by β3 and β4).

The HNH domain targets the complementary strand DNA: The HNH domain comprises a two-stranded antiparallel β-sheet (β12 and β13) flanked by four u-helices (α36-α42). Likewise, it shares structural similarity with HNH endonucleases characterized by a ββα-metal fold, such as the phage T4 endonuclease VII (Endo VII) (Biertumpfel et al., 2007) (PDB code 2QNC, 8% identity, rmsd of 2.6 Å for 60 equivalent Cu atoms) and Vibrio vulnificus nuclease (Li et al., 2003) (PDB code 1OUP, 8% identity, rmsd of 2.9 Å for 78 equivalent Ca atoms). HNH nucleases have three catalytic residues (e.g., Asp40, His41, and Asn62 in Endo VII), and cleave nucleic acid substrates through a single-metal mechanism (Biertumpfel et al., 2007; Li et al., 2003). In the structure of the Endo VII N62D mutant in complex with a Holliday junction, a Mg2+ ion is coordinated by Asp40, Asp62, and oxygen atoms of the scissile phosphate group of the substrate, while His41 acts as a general base to activate a water molecule for catalysis. Asp839, His840, and Asn863 of the Cas9 HNH domain correspond to Asp40, His41, and Asn62 of Endo VII, respectively, consistent with the observation that His840 is critical for the cleavage of the complementary DNA strand (Gasiunas et al., 2012; Jinek et al., 2012). The N863A mutant functions as a nickase, indicating that Asn863 participates in catalysis. These observations suggest that the Cas9 HNH domain may cleave the complementary strand of the target DNA through a single-metal mechanism as observed for other HNH superfamily nucleases. However, in the present structure, Asn863 of Cas9 is located at a position different from that of Asn62 in Endo VII (Biertumpfel et al., 2007), whereas Asp839 and His840 (Ala) of Cas9 are located at positions similar to those of Asp40 and His41 of Endo VII, respectively. This might be due to the absence of divalent ions, such as Mg2+, in Applicants' crystallization solution, suggesting that Asn863 can point towards the active site and participate in catalysis. Whereas the HNH domain shares a ββα-metal fold with other HNN endonuclease, their overall structures are different, consistent with the differences in their substrate specificities.

Conserved arginines clustered on Bridge helix play a critical role in sgRNA:DNA interaction: the crRNA guide region is primarily recognized by the REC lobe. The backbone phosphate groups of the crRNA guide region (nucleotides 4-6 and 13-20) interact with the REC1 domain (Arg165, Gly166, Arg403, Asn407, Lys510, Tyr515 and Arg661) and Bridge helix (Ala59, Arg63, Arg66, Arg70, Arg71, Arg74 and Arg78) and the 2′-hydroxyl groups of C15, U16 and G19 hydrogen bond with Tyr450, Arg447/Ile448 and Thr404 in the REC1 domain, respectively. These observations suggested that the Watson-Crick faces of eight PAM-proximal nucleotides of the Cas9-bound sgRNA are exposed to the solvent, thus serving as a nucleation site for pairing with the target complementary strand. This is consistent with previous reports that the 10-12 bp PAM-proximal “seed” region is critical for Cas9-catalyzed DNA cleavage (Cong et al., 2013; Fu et al., 2013; Hsu et al., 2013; Jinek et al., 2012; Mali et al., 2013a; Pattanayak et al., 2013).

Mutational analysis demonstrated that the R66A, R70A and R74A mutations on Bridge helix markedly reduced DNA cleavage activities, highlighting the functional significance of the recognition of the sgRNA “seed” region by the Bridge helix. Although Arg78 and Arg165 also interact with the “seed” region, the R78A and R165A mutants showed only moderately decreased activities. These results may reflect that, whereas Arg66, Arg70 and Arg74 form bifurcated salt bridges with the sgRNA backbone, Arg78 and Arg165 form a single salt bridge with the sgRNA backbone. A cluster of arginine residues on the Bridge helix are highly conserved among Cas9 proteins in the Type II-A-C systems, suggesting that the Bridge helix is a universal structural feature of Cas9 proteins involved in recognition of the sgRNA and target DNA. This notion is supported by a previous observation that a strictly conserved arginine residue, equivalent to Arg70 of S. pyogenes Cas9, is essential for the function of Francisella novicida Cas9 in the Type II-B system (Sampson et al., 2013). Moreover, the alanine mutation of the repeat:anti-repeat duplex-interacting residues (Arg75 and Lys163) and stem loop 1-interacting residue (Arg69) resulted in decreased DNA cleavage activity, confirming the functional importance of the recognition of the repeat:anti-repeat duplex and stem loop 1 by Cas9.

The crRNA guide region is recognized by Cas9 in a sequence-independent manner except for the U16-Arg447 and G18-Arg71 interactions. This base-specific G18-Arg71 interaction may partly explain the observed preference of Cas9 for sgRNAs having guanines in the four PAM-proximal guide sequences (Wang et al., 2014).

The REC1 and RuvC domains facilitate RNA-guided DNA targeting: Cas9 recognizes the 20-bp DNA target site in a sequence-independent manner. The backbone phosphate groups of the target DNA (nucleotides 1′, 9′-11′, 13′, and 20′) interact with the REC1 (Asn497, Trp659, Arg661 and Gln695), RuvC (Gln926), and PI (Glu1108) domains. The C2′ atoms of the target DNA (nucleotides 5′, 7′, 8′, 11′, 19′, and 20′) form van der Waals interactions with the REC1 domain (Leul69, Tyr450, Met495, Met694 and His698) and RuvC domain (Ala728). These interactions are likely to contribute towards discriminating between DNA vs. RNA targets by Cas9. The terminal base pair of the guide:DNA duplex (G1:C20′) is recognized by the RuvC domain via end-capping interactions; the nucleobases of sgRNA G1 and target DNA C20′ interact with the side chains of Tyr1013 and Val1015, respectively, whereas the 2′-hydroxyl and phosphate groups of sgRNA G1 interact with Val1009 and Gln926, respectively. These end-capping interactions are consistent with the previous observation that Cas9 recognizes a 17-20-bp guide:DNA duplex, and that extended guide sequences are degraded in cells and do not contribute to improving sequence specificity (Mali et al., 2013a; Ran et al., 2013). Taken together, these structural findings explain the RNA-guided DNA targeting mechanism of Cas9.

In certain embodiments, the complex of nucleic acid binding domain with the guide resembles the complex of SpCas9 with crRNA and tracrRNA and/or the complex of SpCas9 with sgRNA. In an embodiment of the invention, the nucleic acid binding domain comprises residues which correspond with respect to binding of guide and target to amino acids of SpCas9 that interact with the guide and/or target. Such amino acids of SpCas9 that interact with guide and/or target include, without limitation, amino acids that interact with the portions of the guide such as stem loop 1, stem loop 3, and/or the repeat:antirepeat duplex, as well as the guide:target heteroduplex. Each of the residues of the nucleic acid binding domain may interact with the guide and/or the guide:target heteroduplex through the amino acid backbone, side chain, or both. Where the interaction is by the amino backbone, there is greater leeway to vary the amino acid side chain at that position. Also, the residues of the nucleic acid binding domain may interact with the sugar-phosphate backbone or a base of the guide or guide:target heteroduplex. With respect to the guide:target heteroduplex, interactions with the sugar-phosphate backbone are preferred which allows for unrestricted sequence variation of the target sequence and the targeting sequence of the guide.

As described elsewhere herein, guides of the invention can comprise ribonucleotides, deoxyribonucleotides, and nucleotide analogs, for example, there can be variation in the sugar-phosphate backbone with nucleic acid binding domains adjusted accordingly.

SaCas9 sgRNA-targetDNA complex: The sgRNA consists of the guide region (G1-C20), repeat region (G21-G34), tetraloop (G35-A38), anti-repeat region (C39-C54), stem loop 1 (A56-G68) and single-stranded linker (U69-U73), with A55 connecting the anti-repeat region and stem loop 1. See, e.g., WO2016/205759. No electron density was observed for U73 at the 3′ end, suggesting that U73 is disordered in the structure. The guide region (G1-C20) and the target DNA strand (dG1-dC20) form an RNA-DNA heteroduplex (referred to as a guide:target heteroduplex), whereas the target DNA strand (dC(−8)-dA(−1)) and the non-target DNA strand (dT1*-dG8*) form a PAM-containing duplex (referred to as a PAM duplex). The repeat (G21-G34) and anti-repeat (C39-C54) regions form a distorted duplex (referred to as a repeat:anti-repeat duplex) via 13 Watson-Crick base pairs. The unpaired nucleotides (C30, A43, U44 and C45) form an internal loop, which is stabilized by a hydrogen bonding-interaction between the 02 of U44 and the N4 of C45. The repeat:anti-repeat duplex is recognized by the REC and WED domains. Indeed, a GAU insertion into the repeat region, which would disrupt the internal loop, reduced the Cas9-mediated DNA cleavage, confirming the functional importance of the distorted structure of the repeat:anti-repeat duplex.

Stem loop 1 is formed via three Watson-Crick base pairs (G57:C67-C59:G65) and two non-canonical base pairs (A56:G68 and A60:A63). U64 does not base pair with A60, and is flipped out of the stem loop. The N1 and N6 of A63 hydrogen bond with the 2′ OH and N3 of A60, respectively. G68 stacks with G57:C67, with the G68 N2 interacting with the backbone phosphate group between A55 and A56. A55 adopts the syn conformation, and its adenine base stacks with U69. In addition, the N1 of A55 hydrogen bonds with the 2′ OH of G68, stabilizing the basal region of stem loop 1. An adenosine nucleotide immediately after the repeat:anti-repeat duplex is highly conserved among CRISPR-Cas9 systems, and equivalent adenosine A51 in the SpCas9 crRNA:tracrRNA also adopts the syn conformation (Anders et al., 2014; Nishimasu et al., 2014), suggesting conserved key roles of an adenosine connecting the repeat:anti-repeat duplex and stem loop 1.

The SpCas9 sgRNA contains three stem loops (stem loops 1-3), which interact with Cas9 and contribute to the complex formation (Nishimasu et al., 2014). The sgRNA lacking stem loops 2 and 3 supports the Cas9-catalyzed DNA cleavage in vitro but not in vivo, indicating the importance of stem loops 2 and 3 for the cleavage activity in vivo (Hsu et al., 2013; Jinek et al., 2012; Nishimasu et al., 2014). The nucleotide sequence of the SaCas9 sgRNA indicated that it contains two stem loops (stem loops 1 and 2) based on its nucleotide sequence. Truncation of putative stem loop 2 remarkably improved the quality of the crystals. As in SpCas9, the sgRNA lacking stem loop 2 supported Cas9-catalyzed DNA cleavage in vitro but not in vivo, suggesting that secondary structures on the 3′ tail of the SaCas9 sgRNA are important for in vivo function.

Tetraloop and stem loop 2 of the SpCas9 sgRNA are exposed to the solvent (Anders et al., 2014; Nishimasu et al., 2014). Thus, these two loops are available for the fusion of RNA aptamers, and the three components system consisting of (1) catalytically inactive SpCas9 (D10A/N863A) fused with a VP64 transcriptional activator domain, (2) a MS2 bacteriophage coat protein fused with p65 and HSF1 transcriptional activator domains, and (3) the engineered sgRNA fused to MS2-interacting RNA aptamers can induce the RNA-guided transcriptional activation of target endogenous loci (Konermann et al., 2015). To examine whether tetraloop and stem loop 2 of the SaCas9 sgRNA are available for the MS2-interacting aptamer fusion, Applicants co-expressed in HEK293F cells the three components, (1) dSpCas9 (D10A/N863A)-VP64 or dSaCas9 (D10A/N580A)-VP64, (2) its engineered sgRNA, and (3) MS2-p65-HSF1, and then monitored the transcriptional activation of two different endogenous genes (ASCL1 and MYOD1). The results showed that the dSaCas9-based activator induces the transcription activation of the ASCL1 and MYOD1 genes at levels comparable to those of the dSpCas9-based activator. These results indicate that the SaCas9 sgRNA has solvent-exposed stem loop 2, and demonstrate that the engineered SaCas9 sgRNA can recruit multiple MS2-fused proteins.

The guide:target heteroduplex is accommodated in the central channel between the REC and NUC lobes. The sugar-phosphate backbone of the PAM-distal region (A3-U6) of the sgRNA interacts with the REC lobe (Thr238, Tyr239, Lys248, Tyr256, Arg314, Asn394 and Gln414). In SpCas9 and SaCas9, the RNA-DNA base pairing in the 8 bp PAM-proximal “seed” region in the guide:target heteroduplex is critical for Cas9-catalyzed DNA cleavage (Hsu et al., 2013; Jinek et al., 2012; Ran et al., 2015). Consistent with this, the phosphate backbone of the sgRNA seed region (C13-C20) is extensively recognized by the bridge helix (Asn44, Arg48, Arg51, Arg55, Arg59 and Arg60) and the REC lobe (Arg116, Gly117, Arg165, Gly166, Asn169 and Arg209), as in the case of SpCas9. In addition, the 2′ OH groups of C15, U16, U17 and G19 interact with the REC lobe (Gly166, Arg208, Arg209 and Tyr211). These structural findings suggest that the sgRNA binds to SaCas9, with its seed region pre-ordered in an A-form conformation for base-paring with the target DNA strand, as proposed for SpCas9 (Jiang et al., 2015). In addition, the sugar-phosphate backbone of the target DNA strand interacts with the REC lobe (Tyr211, Trp229, Tyr230, Gly235, Arg245, Gly391, Thr392, and Asn419) and the RuvC domain (Leu446, Tyr651 and Arg654). Together, there structural findings explain the RNA-guided DNA targeting mechanism of SaCas9. Notably, the REC lobe of SaCas9 shares structural similarity with those of SpCas9 (PDB code 4UN3, 26% identity, rmsd of 1.9 Å for 177 equivalent Cu atoms) and AnCas9 (PDB ID 40GE, 16% identity, rmsd of 3.2 Å for 167 equivalent Cu atoms), indicating that the Cas9 orthologs recognize the guide:target heteroduplex in a similar manner.

Recognition Mechanism of the crRNA:tracrRNA Scaffolds

The repeat:anti-repeat duplex is recognized by the REC and WED domains, primarily through interactions between the sugar-phosphate backbone and protein. Consistent with the data showing that the sgRNA containing the fully-paired repeat:anti-repeat duplex fails to support Cas9-catalyzed DNA cleavage, the internal loop (C30, U44 and C45) is extensively recognized by the WED domain. The 2′ OH and 02 of C30 hydrogen bond with Tyr868 and Lys867, respectively, and the phosphate groups of U31, C45 and U46 interact with Lys870, Arg792 and Lys881, respectively. These structural observations explain the structure-dependent recognition of the repeat:anti-repeat duplex by SaCas9.

Stem loop 1 is recognized by the bridge helix and REC lobe. The phosphate backbone of stem loop 1 interact with the bridge helix (Arg47, Arg54, Arg55, Arg58 and Arg59) and the REC lobe (Arg209, Gly216 and Ser219). The 2′ OH of A63 hydrogen bonds with His64. The flipped-out U64 is recognized by Glu213 and Arg209 via hydrogen-bonding and stacking interactions, respectively. A55 is extensively recognized by the phosphate lock loop. The N6, N7 and 2′ OH of A55 hydrogen bond with Asn780/Arg781, Leu783 and Lys906, respectively. Lys57 interacts with the phosphate group between C54 and A55, and the side chain of Leu783 form hydrophobic contacts with the adenine bases of A55 and A56. The phosphate backbone of the linker region electrostatically interacts with the RuvC domain (Arg452, Lys459 and Arg774) and the phosphate lock loop (Arg781), and the guanine base of G80 stacks with the side chain of Arg47 on the bridge helix.

### Recognition Mechanism of the 5′-NNGRRT-3′ PAM

SaCas9 recognizes the 5′-NNGRRN-3′ PAM with a preference for a thymine base at the 6th position (Ran et al., 2015), which is distinct from the 5′-NGG-3′ PAM of SpCas9. In the present structures containing either the 5′-TTGAAT-3′ PAM or the 5′-TTGGGT-3′ PAM, the PAM duplex is sandwiched between the WED and PI domains, and the PAM in the non-target DNA strand is read out from the major groove side by the PI domain. dT1* and dT2* form no direct contact with the protein. Consistent with the observed requirement for the 3rd G in the 5′-NNGRRT-3′ PAM, the 06 and N7 of dG3* forms bidentate hydrogen bonds with the side chain of Arg1015, which is anchored via salt bridges with Glu993 in both complexes. In the 5′-TTGAAT-3′ PAM complex, the N7 atoms of dA4* and dA5* form direct and water-mediated hydrogen bonds with Asn985 and Asn985/Asn986/Arg991, respectively. In addition, the N6 of dA5* forms a water-mediated hydrogen bond with Asn985. Similarly, in the 5′-TTGGGT-3′ PAM complex, the N7 atoms of dG4* and dG5* form direct and water-mediated hydrogen bonds with Asn985 and Asn985/Asn986/Arg991, respectively. The 06 of dG5* forms a water-mediated hydrogen bond with Asn985. These structural findings explain the ability of SaCas9 to recognize the purine nucleotides at positions 4 and 5 in the 5′-NNGRRT-3′ PAM. The 04 of dT6* hydrogen bonds with Arg991, explaining the preference of SaCas9 to the 6th T in the 5′-NNGRRT-3′ PAM. Single alanine mutants of these PAM-interacting residues reduced cleavage activities in vivo, and double mutations abolished the activity, confirming the importance of Asn985, Asn986, Arg991, Glu993 and Arg1015 for PAM recognition. In addition, the phosphate backbone of the PAM duplex is recognized from the minor groove side by the WED domain (Tyr789, Tyr882, Lys886, Ans888, Ala889 and Leu909) in a manner distinct from SpCas9. Together, the structural and functional data reveal the mechanism of relaxed recognition of the 5′-NNGRRT-3′ PAM by SaCas9.

### Mechanism of Target DNA Unwinding

In the quaternary complex structure of SpCas9, Glu1108 and Ser1109 in the phosphate lock loop hydrogen bond with the phosphate group between dA(−1) and dT1 in the target DNA strand (referred to as +1 phosphate), and contribute to unwinding of the target DNA (Anders et al., 2014). The present structure revealed that SaCas9 also has the phosphate lock loop, although the phosphate lock loops of SaCas9 and SpCas9 share limited sequence similarity. In the present structure of SaCas9, the +1 phosphate between dA(−1) and dG1 in the target DNA strand hydrogen bonds with the main-chain amide groups of Asp786 and Thr787 and the side-chain Og atom of Thr787 in the phosphate lock loop. These interactions result in the rotation of the +1 phosphate, thereby facilitating base-pairing between dG1 in the target DNA strand and C20 in the sgRNA. Indeed, the SaCas9 T787A mutant showed reduced DNA cleavage activity, confirming the functional significance of Thr787 in the phosphate lock loop. Together, these data indicated that the molecular mechanism of the target DNA unwinding is conserved among SaCas9 and SpCas9.

### RuvC and HNH Nuclease Domains

The RuvC domain of SaCas9 has an RNase H fold, and shares structural similarity with those of SpCas9 (PDB code 4UN3, 25% identity, rmsd of 2.5 Å for 191 equivalent Ca atoms) and Actinomyces naeslundii Cas9 (AnCas9) (PDB code 40GE, 17% identity, rmsd of 3.0 Å for 170 equivalent Ca atoms). The catalytic residues of SaCas9 (Asp10, Glu477, His701 and Asp704) are located at positions similar to those of SpCas9 (Asp10, Glu762, His983 and Asp986) and AnCas9 (Asp17, Glu505, His736 and Asp739). The D10A, E477A, H701A and D704A mutants of SaCas9 showed almost no DNA cleavage activities. These observations indicated that the SaCas9 RuvC domain cleaves the non-target DNA strand through a two-metal ion mechanism as in other endonucleases of the RNase H superfamily (Gorecka et al., 2013).

The HNH domain of SaCas9 has an aab-metal fold, and shares structural similarity with those of SpCas9 (PDB code 4UN3, 27% identity, rmsd of 1.8 Å for 93 equivalent Ca atoms) and AnCas9 (PDB code 40GE, 18% identity, rmsd of 2.6 Å for 98 equivalent Ca atoms). The catalytic residues of SaCas9 (Asp556, His557 and Asn580) are located at positions similar to those of SpCas9 (Asp839, His840 and Asn863) and AnCas9 (Asp581, His582 and Asn606), although Asn863 is oriented away from the active site in the ternary and quaternary complex structures of SpCas9. The D556A, H557A and N580A mutants of SaCas9 showed almost no DNA cleavage activities). These observations indicated that the SaCas9 HNH domain cleaves the target DNA strand through a one-metal ion mechanism as in other aab-metal endonucleases (Biertumpfel et al., 2007).

A structural comparison of SaCas9 with SpCas9 and AnCas9 revealed that the RuvC and HNH domains are connected by α-helical linker, L1 and L2, and that there are notable differences in the relative arrangements between the two nuclease domains. A biochemical study suggested that the binding of the PAM duplex to SpCas9 facilitates the cleavage of the target DNA strand by the HNH domain (Sternberg et al., 2014). However, in the quaternary complex structures of SaCas9 and SpCas9, the HNH domains are located away from the cleavage site of the target DNA strand. A structural comparison of SaCas9 with Thermus thermophilus RuvC in complex with a Holliday junction substrate (Gorecka et al., 2013) indicated steric clashes between the L1 linker and the modeled non-target DNA strand bound to the active site of the SaCas9 RuvC domain. These observations suggested that the binding of the non-target DNA strand to the RuvC domain may contribute to triggering a conformational change in the L1, thereby bringing the HNH domain to the scissile phosphate group in the target DNA strand.

### Conserved Mechanism of RNA-Guided DNA Targeting

Previous structural studies revealed that SpCas9 undergoes conformational rearrangements upon guide RNA binding, to form the central channel between the REC and NUC lobes (Anders et al., 2014; Jinek et al., 2014; Nishimasu et al., 2014). In the absence of the guide RNA, SpCas9 adopts a closed conformation, where the active site of the HNH domain is covered by the RuvC domain. In contrast, the ternary and quaternary complex structures of SpCas9 adopt an open conformation and have the central channel, which accommodates the guide:target heteroduplex. The quaternary complex structure of SaCas9 adopts an open conformation and has the central channel, which accommodates the guide:target heteroduplex. Thus, the guide RNA-induced conformational rearrangement is conserved among SaCas9 and SpCas9.

The REC lobes of SaCas9 and SpCas9 (PDB code 4UN3) share structural similarity (25% identity, rmsd of 2.9 Å for 353 equivalent Ca atoms), and recognize the guide:target heteroduplex in a similar manner. In particular, the seed region of the sgRNA is commonly recognized by the arginine cluster on the bridge helix in SaCas9 and SpCas9. AnCas9 (PDB ID 40GE) also has a REC lobe similar to those of SaCas9 and SpCas9. These observations suggested that the recognition mechanism of the guide:target heteroduplex is conserved among Cas9 orthologs.

Structural Basis for the Orthogonal Recognition of sgRNA Scaffolds

Applicants made comparison of the quaternary complex structures of SaCas9 and SpCas9 revealing that the structurally diverse REC and WED domains recognize the distinct structural features of the repeat:anti-repeat duplex, allowing cognate sgRNAs to be distinguished in an orthogonal manner. The SpCas9 WED domain adopts a compact loop conformation (Nishimasu et al., 2014; Anders et al., 2014). In contrast, the SaCas9 WED domain has a new fold comprising a twisted five-stranded β-sheet flanked by four α-helices. The AnCas9 WED domain has yet a different fold containing three antiparallel P-hairpins (Jinek et al., 2014). These structural differences in the WED domains are consistent with variations in sgRNA scaffolds among CRISPR-Cas9 systems (Fonfara et al., 2014; Briner et al., 2014; Ran et al., 2015).

The REC lobe also contributes to the orthogonal recognition of sgRNA scaffolds. Although the REC lobes of SaCas9 and SpCas9 share structural similarity, the SpCas9 REC lobe has four characteristic insertions (Ins 1-4), which are absent in the SaCas9 REC lobe. Ins 2 (also known as the REC2 domain) forms no contact with the nucleic acids in the SpCas9 structures and is dispensable for DNA cleavage activity (Nishimasu et al., 2014), consistent with the absence of Ins2 in SaCas9. Ins 1 and 3 recognize the SpCas9-specific internal loop in the repeat:anti-repeat duplex, while in SaCas9 the WED domain recognizes the internal loop in the repeat:anti-repeat duplex, as described above. In addition, Ins 4 interacts with stem loop 1 of the SpCas9 sgRNA, which is shorter than that of the SaCas9 sgRNA. Together, these structural observations demonstrate that the Cas9 orthologs recognize their cognate sgRNA in an orthogonal manner, using a combination of the structurally diverse REC and WED domains.

### Structural Basis for the Distinct PAM Specificities

A structural comparison of SaCas9, SpCas9 and AnCas9 revealed that, despite lacking sequence homology, their PI domains share a similar protein fold. The PI domains consist of the Topo-homology domain, which comprises three-stranded anti-parallel β-sheet (β1-β3) flanked by several α helices, and the C-terminal domain, which comprises twisted six-stranded anti-parallel β-sheet (β4-β9) (the β7 in SpCas9 adopts a loop conformation). In both SaCas9 and SpCas9, the major groove of the PAM duplex is read out by the β5-β7 region in their PI domains. The 3rd G in the 5′-NNGRRT-3′ PAM is recognized by Arg1015 in SaCas9, and the 3rd G in the 5′-NGG-3′ PAM is recognized by Arg1335 in SpCas9 and in a similar manner. However, there are also notable differences in the PI domains of SaCas9 and SpCas9, consistent with their distinct PAM specificities. Arg1333 of SpCas9, which recognizes the 2nd G in the NGG PAM, is replaced with Pro1013 in SaCas9. In addition, SpCas9 lacks amino acid residues equivalent to Asn985/Asn986 (β5) and Arg991 (β6) of SaCas9, because the β5-β6 region of SpCas9 is shorter than that of SaCas9. Moreover, Asn985, Asn986, Arg991 and Arg1015 in SaCas9 are replaced with Asp1030, Thr1031, Lys1034 and Lys1061 in AnCas9, respectively, suggesting that the PAM for AnCas9 is different from those for SaCas9 and SpCas9. Together, these structural findings demonstrated that distinct PAM specificities of Cas9 orthologs are primarily defined by their structurally diverse PI domains. Accordingly these findings can be used in the engineering of the nucleic acid binding domains of the present compositions and complexes.

### Effector Domain

In certain embodiments, a nucleic acid binding domain is linked to one or more effector domains. Effector domains include, without limitation, a transcriptional activator, a transcriptional repressor, a recombinase, a transposase, a histone remodeler, a demethylase, a DNA methyltransferase, a cryptochrome, a light inducible/controllable domain, a chemically inducible/controllable domain, an epigenetic modifying domain, or a combination thereof. Effector domains further provide activities, such as locating proteins of the invention, non-limiting examples including cellular permeability enhancers or cell penetrating peptides, nuclear localization signals, nuclear export signals, capsid proteins, cell surface recognition such as ligands of cell surface receptors, and the like.

When there is more than one effector domain, the linkage to each binding domain can be the same or different. For example, in one non-limiting embodiment, a first linkage is covalent and a second linkage is inducible. In another non-limiting embodiment, a first linkage is covalent while a second linkage is covalent and cleavable. To illustrate, a first linkage can be, for example, to a cell penetrating peptide which is cleaved or otherwise dissociates from the nucleic acid binding domain upon or after entry into a cell wherein a second effector domain such as a NLS directs the protein to the cell nucleus.

In an embodiment the nucleic acid binding domain and the effector domain are linked by a cleavable or biodegradable linker.

The one or more effector domains can comprise one or more nucleases. In an embodiment, the one or more effector domain comprises a small molecule capable of inducing single- or double-stranded breaks.

In an embodiment, the one or more effector domains comprise one or more nuclear localization signals (NLSs). In an embodiment, the one or more effector domains comprise a cellular permeability enhancer. In an embodiment, the one or more effector domains comprises a recombination template.

### Local Inhibition of NHEJ and Enhancement of HDR

The invention provides improving HDR to accompany targeted cleavage of nucleic acids. Improvements in HDR can be accomplished by inhibition of NHEJ, enhancement of HDR, or both.

Several small molecule inhibitors of NHEJ pathway have been identified and their application to cells have modestly enhanced HDR. Similarly, multiple HDR activators increase HDR efficiency. However, the on-target toxicity of global NHEJ inhibition or global HDR activation in a cell severely limits the utility of such approaches. Ideally, NHEJ inhibition or HDR activation locally near the site of, e.g., a Cas9 mediated double strand break, is more efficient and safe. Such a targeted approach lowers the minimum efficacious dose of the inhibitors or activators and increases the maximum tolerated dose of the inhibitors or activators.

HDR activators may in some embodiments comprise

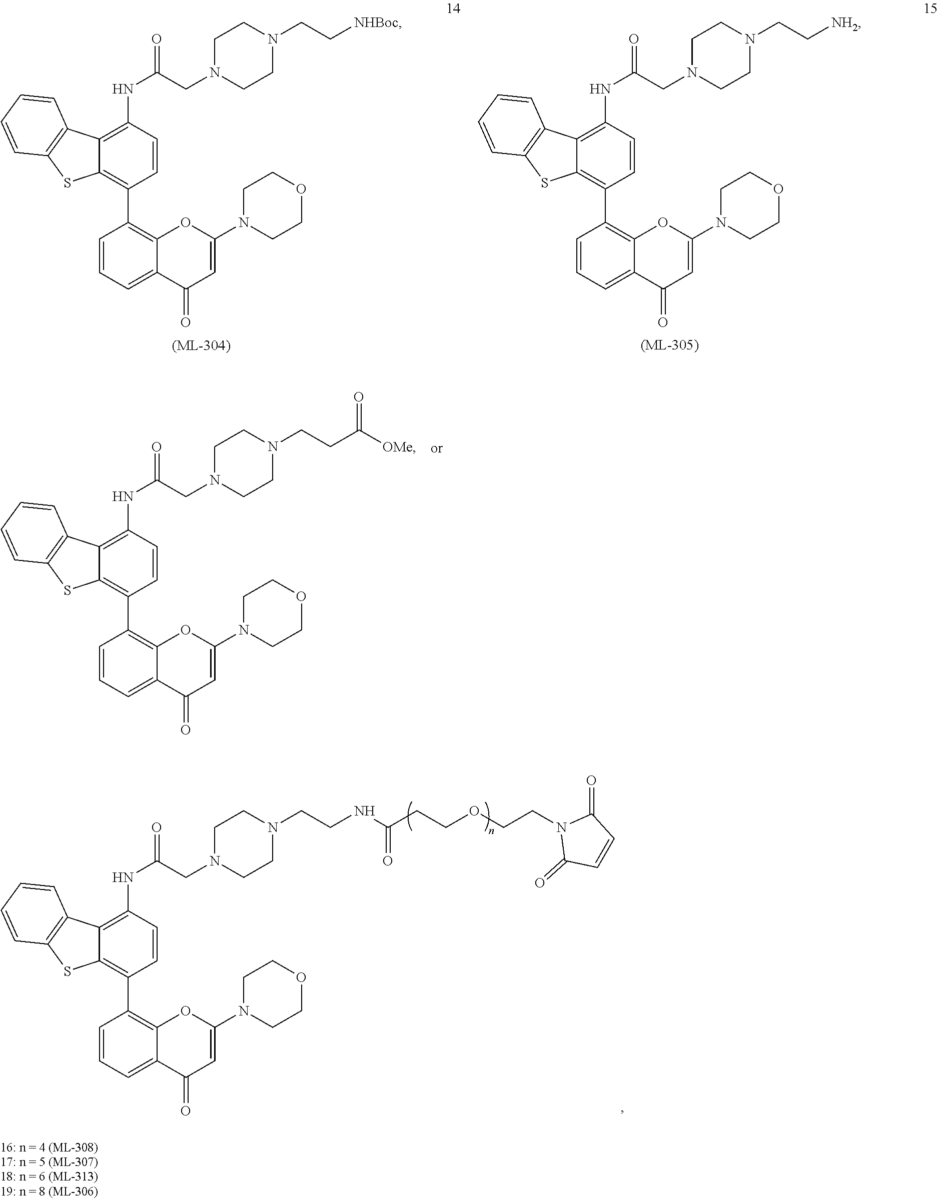

wherein n=4, 5, 6 or 8.

In embodiments, the NHEJ inhibitor is selected from

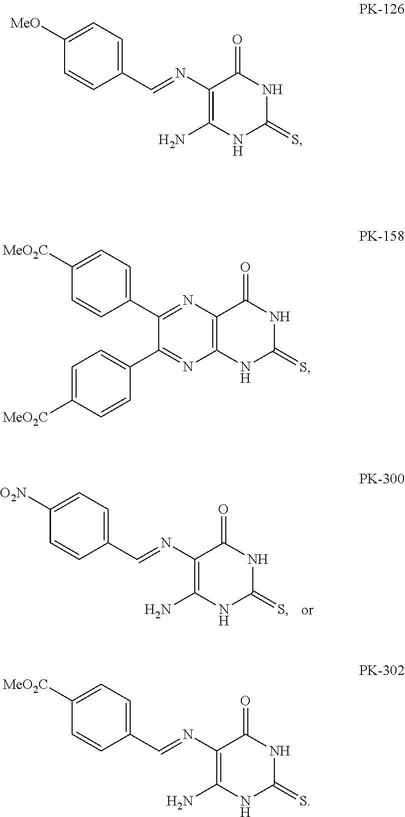 [SMILES: COc1ccc(/C=N/c2c(N)[nH]c(=S)[nH]c2=O)cc1 ; COC(=O)c1ccc(-c2nc3[nH]c(=S)[nH]c(=O)c3nc2-c2ccc(C(=O)OC)cc2)cc1 ; Nc1[nH]c(=S)[nH]c(=O)c1/N=C/c1ccc([N+](=O)[O-])cc1 ; COC(=O)c1ccc(/C=N/c2c(N)[nH]c(=S)[nH]c2=O)cc1]

In certain embodiments, the NHEJ inhibitor is selected from

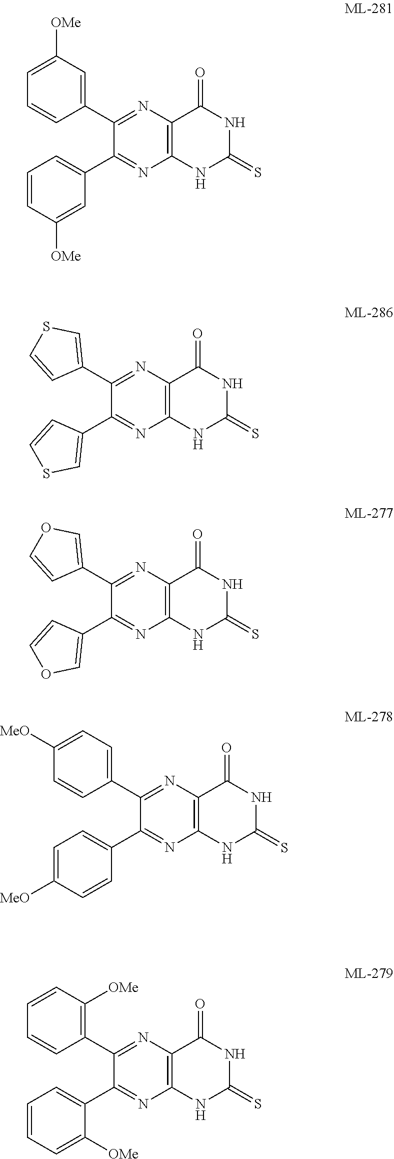 [SMILES: COc1cccc(-c2nc3[nH]c(=S)[nH]c(=O)c3nc2-c2cccc(OC)c2)c1 ; O=c1[nH]c(=S)[nH]c2nc(-c3ccsc3)c(-c3ccsc3)nc12 ; O=c1[nH]c(=S)[nH]c2nc(-c3ccoc3)c(-c3ccoc3)nc12 ; COc1ccc(-c2nc3[nH]c(=S)[nH]c(=O)c3nc2-c2ccc(OC)cc2)cc1 ; COc1ccccc1-c1nc2[nH]c(=S)[nH]c(=O)c2nc1-c1ccccc1OC]

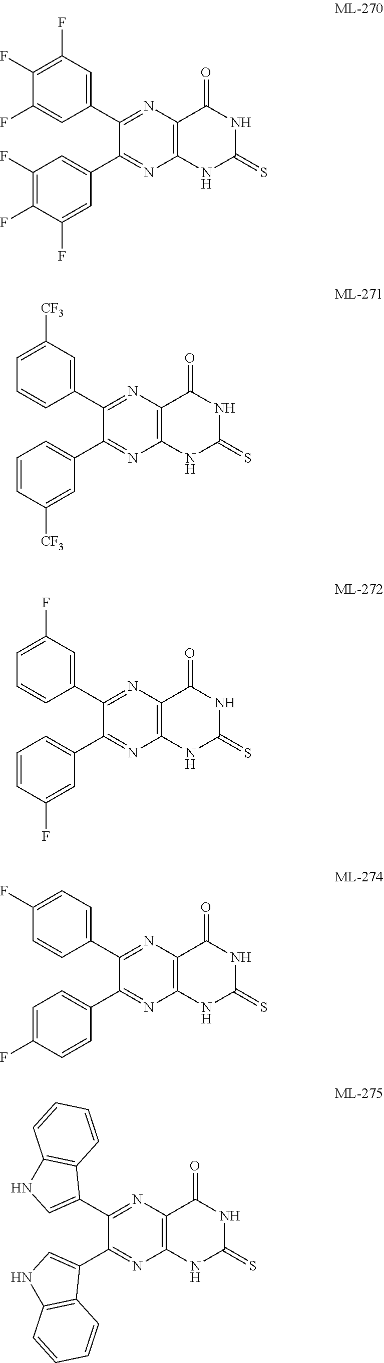

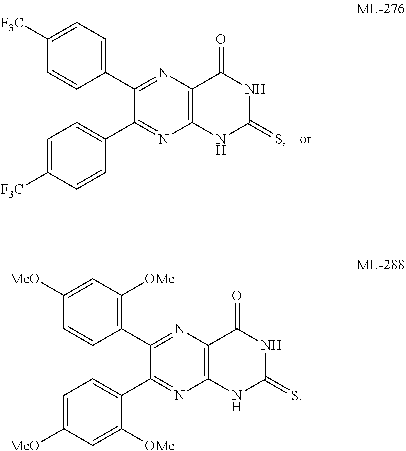 [SMILES: O=c1[nH]c(=S)[nH]c2nc(-c3ccc(C(F)(F)F)cc3)c(-c3ccc(C(F)(F)F)cc3)nc12 ; COc1ccc(-c2nc3[nH]c(=S)[nH]c(=O)c3nc2-c2ccc(OC)cc2OC)c(OC)c1]

### p5 Pathway Inhibition

Local inhibition of p53 pathway activation can increase the efficiency of precision genome editing in many primary cells where Cas9-induced double-strand breaks lead to apoptosis via activation of the p53 pathway. Haapaniemi, E.; Botla, S.; Persson, J.; Schmierer, B.; Taipale, J., CRISPR-Cas9 genome editing induces a p53-mediated DNA damage response. Nat Med 2018, 24 (7), 927-930; Ihry, et al., p53 inhibits CRISPR-Cas9 engineering in human pluripotent stem cells. Nat Med 2018, 24 (7), 939-946. In an embodiment, the genome editors overcome the predisposition of non-homologous end joining (NHEJ) repair which can lead to the p53 apoptosis pathway though use of ssODNs, NHEJ inhibitors and/or HDR activators. One exemplary p53 inhibitor small molecule is pifithrin-α (PFTα), a reversible inhibitor of p53-mediated apoptosis and p53-dependent gene transcription such as cyclin G, p21/waf1, and mdm2 expression which may be linked, associated or delivered to cells before, concurrent with, or after delivery of the synthetic base editors disclosed herein. Additional inhibitors can be an ATM kinase inhibitor, including, for example KU-55933.

### DNA Glycosylase Inhibition

Local inhibition of uracil DNA glycosylase would also be helpful for the development of efficient base editors. One important function of uracil-DNA glycosylases is to prevent mutagenesis by eliminating uracil from DNA molecules by cleaving the N-glycosylic bond and initiating the base-excision repair (BER) pathway. Komor, et al., Editing the Genome Without Double-Stranded DNA Breaks. Acs Chemical Biology 2018, 13 (2), 383-388. The Uracil Glycosylase Inhibitor (UGI) of Bacillus subtilis bacteriophage PBS1 or PBS2 is a small protein (9.5 kDa) which inhibits E. coli uracil-DNA glycosylase (UDG) as well as UDG from other species. In some embodiments, the UGI is provided separate from the SAGE, in others the UGI is provided appended or associate with the SAGE. Wang et al., Caell Res. 2017 10: 1289-1292, DOI 10.1038/cr.2017.111.

### Sortase-Mediated Ligation

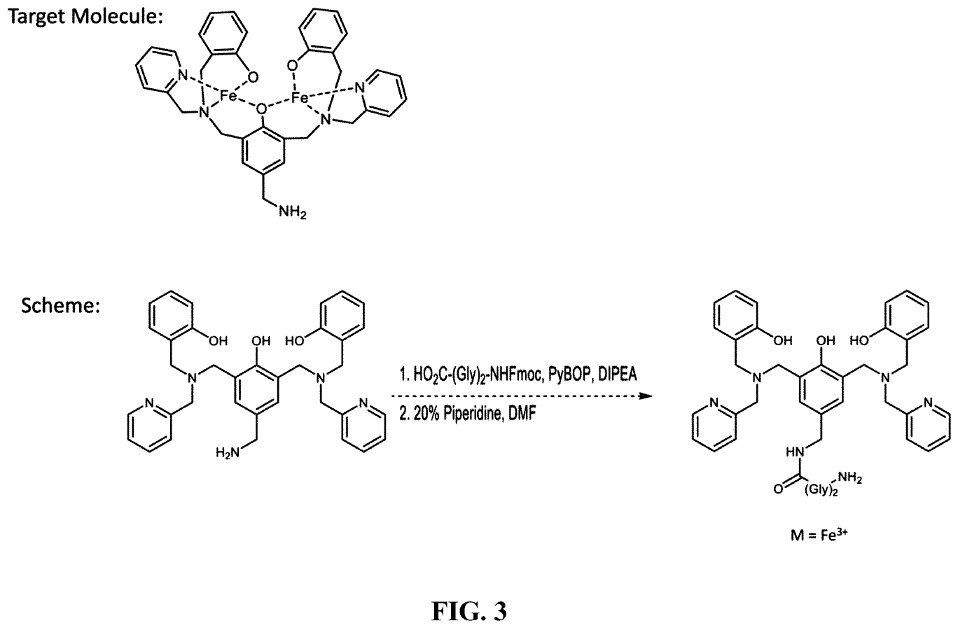

A method to inhibit NHEJ and activate HDR locally comprises linking an inhibitor of NHEJ and/or an activator of HDR to a nucleic acid targeting moiety. For example, a Cas9 nuclease can be engineered to accommodate a single or multiple sortase recognition sequences (Leu-Pro-Xxx-Thr-Gly (SEQ ID NO: 1), where Xxx is any amino acid) at which position effector moieties can be linked. Sortase is a transpeptidase that cleaves its recognition sequence between Thr-Gly, and ligates an acceptor peptide containing an N-terminal glycine to the newly formed Thr carboxylate (FIG. 3A). Engineering sortase recognition sequences onto Cas9 or other nucleic acid-targeting moiety allows site-specific conjugation of any chemical payload. Insertion sites can be regions previously validated as cut sites for split Cas9, particularly those for which the N and C fragments have been shown to have a high affinity for each other.

One way to validate insertion sites in Cas9 or other nucleic acid-targeting moiety as to tolerance to modification is by sortase-mediated ligation of the model substrate Gly-Gly-Gly-Lys(Biotin) (SEQ ID NO: 2). The biotin handle allows efficient detection of Cas9 modification by immunoblotting and facilitates enrichment of labeled protein through affinity purification with anti-biotin or streptavidin. Cas9 activity has been validated using an EGFP based screening assay, wherein a U2OS.EGFP cell line is exposed to Cas9 containing a guide RNA sequence targeting EGFP, leading to loss of EGFP fluorescence. Active biotin-ligated Cas9 proteins can be validated for in vivo efficacy. Using the positively charged transfection agent, such as RNAiMAX, biotin-ligated Cas9-sgRNA ribonucleoproteins can be transfected into U2OS.EGFP cell lines, comparing the loss of GFP fluorescence to the introduction of wtCas9.

Sortase-mediated ligation allows attachment to the surface of Cas9 or other nucleic acid targeting moiety many non-native chemicals that can enhance the activity and modulate the effects of Cas9. A particularly powerful example of this is in the local modulation of the NHEJ/HDR pathway in cells. Methods for inhibiting NHEJ to boost HDR are typically achieved through gene knockout of key NHEJ components such as DNA ligase IV, KU70, or KU80. Small molecule inhibitors of DNA ligase IV (SCR7; herein compound 1.21, also known as SCR7-G) have been described, but their cellular toxicity prohibits use at high concentration and may interfere with global, Cas9-independent DNA repair. Instead, Cas9-SortLoop proteins are used as a scaffold for multivalent display of NHEJ-inhibited compounds to control the spatial reach of their effects, enabling local enhancement of HDR.

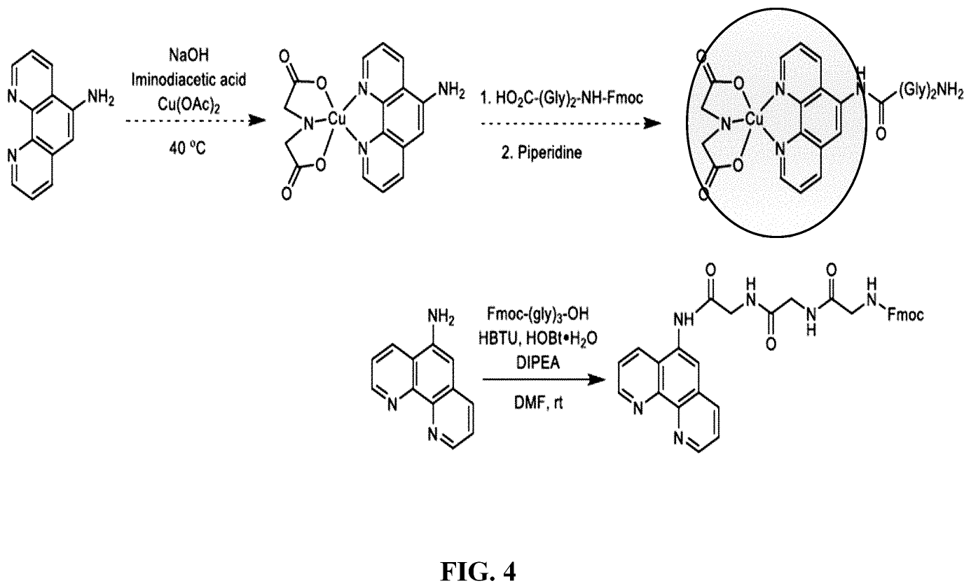

In an embodiment of the invention, small molecule inhibitors of NHEJ are linked to a poly-glycine tripeptide through PEG for sortase-mediated ligation (FIG. 4). Based on the reported structure-activity relationship of NHEJ inhibitor L189, SCR7 (structure as reported by Srivastava), and SCR7-G, rings 1, 2, and 3 are involved in the target-engagement while the presence of ring 4 increases the hindrance and thus helps to block the ligase more efficiently. Conjugation of a poly-glycine peptide with the para-carboxylic moiety in the ring 4 will retain activity. This method provides a simple and effective strategy to ligate Cas9 with NHEJ inhibitors to precisely enhance HDR pathway near the Cas9 target site while keeping the global DNA repair unaffected. In an embodiment of the invention, nucleic acid targeting moiety conjugates based on small molecule inhibitor of DNA-dependent protein kinase (DNA-PK) or heterodimeric Ku (KU70/KU80). KU-0060648 is one of the most potent KU-inhibitors, which can also be functionalized with poly-glycine and used for Cas9-functionalization.

In embodiments, conjugation via cysteine and unnatural amino acid mutagenesis will be high yielding, although conjugation via sortase may vary. Previously, conjugation chemistry was developed by generating two types of cysteines that differ widely in their reactivity in the presence of a catalyst. Briefly, one cysteine type is surrounded by arginine (called “Arg cysteine”), and the other cysteine is surrounded by aspartic acid (called “Asp cysteine”). By using polycarboxylates (e.g., citric acid, mellitic acid) that interact with arginines through salt-bridges and that can also act as a base catalyst, Applicants demonstrated substantial selective enhancement of “Arg-cysteine” reactivity over that of “Asp-cysteine.” Accordingly, in some embodiments, cysteines with disparate reactivity can be deployed in addition to, or instead of, sortase chemistry.

Increasing local NHEJ inhibitor molarity is also effective in vivo. For example, Cas9-NHEJ inhibitor can be complexed with sgRNA and delivered into appropriate patient-derived cells. The following table provides an exemplary list of mutations that can be rectified.

TABLE 1
Exemplary list of mutations to rectify

| | Harris ID/ | | | | |
|---|---|---|---|---|---|
| PKD type | Coriell ID | Gene | Exon | Mutation | Mutation call |
| Dominant | OX3502/− | PKD1 | 1 | C39Y | Highly likely |
| | | | | | pathogenic |
| Dominant | OX3504/− | PKD1 | 15 | E1929X | Definitely |
| | | | | | pathogenic |
| Recessive | OX3688 | PKHD1 | 3 | T36M | Definitely |
| | | | | | pathogenic |

The extent of HDR can be quantified using next-generation sequencing and data analysis platform that have been used previously.

In an embodiment, the same approach can be utilized to local enhance HDR without NHEJ inhibition. RAD51 is a protein involved in strand exchange and the search for homology regions during HDR repair. The phenylbenzamide RS1, discovered by high-throughput screening against a 10,000-compound library, was identified as a small-molecule RAD51-stimulator (FIG. 4C). RS1 has also been evaluated as a potent enhancer for Cas9-based genome editing, and has been shown to inhibit HIV-1 integration and decrease of viral replication. Thus, RS1-ligated Cas9 may be used to enhance HDR of Cas9-mediated repair. Docking analysis using a homology model of RAD51 showed that the terminal phenyl group at the benzylsulfamoyl handle on the phenylbenzamide scaffold is amenable for the attachment of a peptide linker incorporating N-terminal glycine residues, which could be ligated with the acyl intermediate formed between the threonine of cleaved Cas9-LPXT and sortase. The assays previously described can be used to assess the degree of local HDR enhancement.

In an embodiment the nucleic acid modifier comprises an effector domain, the effector domain comprising an activator of homology-directed repair (HDR) and/or an inhibitor of non-homologous end joining (NHEJ). In an embodiment the activator of HDR is a small molecule. In an embodiment the activator of HDR is an activator of RAD51. In an embodiment the activator of HDR is linked to the nucleic acid binding domain.

In an embodiment the nucleic acid modifier comprises an inhibitor of NHEJ, the inhibitor comprising a DNA ligase IV inhibitor. In an embodiment the inhibitor of NHEJ comprises a small molecule. In an embodiment the inhibitor of NHEJ is linked to the nucleic acid binding domain.

In an embodiment, the effector domain comprises a repressor domain, an activator domain, a transposase domain, an integrase domain, a recombinase domain, a resolvase domain, an invertase domain, a protease domain, a DNA methyltransferase domain, a DNA hydroxylmethylase domain, a DNA demethylase domain, a histone acetylase domain, a histone deacetylase domain or a cellular uptake activity associated domain.

In some embodiments, one or more effector domains may be associated with or tethered to CRISPR enzyme and/or may be associated with or tethered to modified guides via adaptor proteins. These can be used irrespective of the fact that the CRISPR enzyme may also be tethered to a virus outer protein or capsid or envelope, such as a VP2 domain or a capsid, via modified guides with aptamer RAN sequences that recognize correspond adaptor proteins.

In some embodiments, one or more effector domains comprise a transcriptional activator, repressor, a recombinase, a transposase, a histone remodeler, a demethylase, a DNA methyltransferase, a cryptochrome, a light inducible/controllable domain, a chemically inducible/controllable domain, an epigenetic modifying domain, or a combination thereof. Advantageously, the effector domain comprises an activator, repressor or nuclease.

In some embodiments, a effector domain can have methylase activity, demethylase activity, transcription activation activity, transcription repression activity, transcription release factor activity, histone modification activity, RNA cleavage activity or nucleic acid binding activity, or activity that a domain identified herein has.

Examples of activators include P65, a tetramer of the herpes simplex activation domain VP16, termed VP64, optimized use of VP64 for activation through modification of both the sgRNA design and addition of additional helper molecules, MS2, P65 and HSF1 in the system called the synergistic activation mediator (SAM) (Konermann et al, “Genome-scale transcriptional activation by an engineered CRISPR-Cas9 complex,” Nature 517(7536):583-8 (2015)); and examples of repressors include the KRAB (Kruppel-associated box) domain of Kox1 or SID domain (e.g. SID4X); and an example of a nuclease or nuclease domain suitable for a effector domain comprises Fok1.

Suitable effector domains for use in practice of the invention, such as activators, repressors or nucleases are also discussed in documents incorporated herein by reference, including the patents and patent publications herein-cited and incorporated herein by reference regarding general information on CRISPR-Cas Systems.

Miniature Genome Editor with Multifunctionality (MiniGEMs)

In one aspect, the invention provides an engineered, non-naturally occurring nucleic acid modifying system, comprising: a) a first engineered, non-naturally occurring DNA reader, wherein the first DNA reader binds a target nucleic acid; and b) a first effector component, wherein the first effector is a small molecule and modifies the target nucleic acid. The DNA reader can be a peptide nucleic acid (PNA) polymer, or transcript activator-like effector (TALE).

In some embodiments, the nucleic acid modifying systems utilizing a non-naturally occurring DNA reader such as a PNA polymer is referred to as a miniGEM. The miniGEMs disclosed can be ˜30% of the size of Cas9 guide RNA complex. The size reduction stems from the use of synthetic small-molecule effector components (<500 Da), in place of the large nuclease domains (>100 kDa) employed by Cas9. Further, in miniGEM, PNAs will act as high fidelity DNA readers as well as a scaffold for display of synthetic nucleases, further reducing the size compared to that of Cas9-guide RNA complex. This size reduction will allow facile delivery of multiple miniGEMs into a cell type of interest and may even allow highly multiplexed editing. The synthetic nature of miniGEM also lowers the cost of both mass production and storage that is often associated with protein/nucleic-acid based therapeutic agents. Thus, miniGEMs provide a novel platform to enhance cellular delivery and allow multiplexed precision genome editing on an unprecedented scale.

In some embodiments, the activity of synthetic nucleases can be masked using pro-drug strategies enabling tissue-specific activation of miniGEMs. Some synthetic nucleases require specific triggers and others can be split into two components, affording additional control of specificity and activity of miniGEM. Fourth, the synthetic nature of the editor allows display of additional functionalities. For example, effector components can comprise ssODNs, NHEJ inhibitors or HDR activators for precise genome edits can be utilized.

In some embodiments, the engineered nucleic acid modifying systems can be tuned for varying potencies, including low (>10 μM), medium (0.5-10 μM), and high (<1 nM) with single or double-strand cleavage activity.

### DNA Reader

The designer nucleic acid sequence readers include target nucleic acid binding molecules designed like CRISPR systems to recognize nucleic acid sequences using a programmable guide. In certain embodiments, the designer nucleic acid sequence readers comprise one or more peptide nucleic acids (PNAs) polymers. The nucleic acid sequence readers further include readers designed like Transcription Activator-Like Effectors (TALEs) to recognize DNA using two variable amino acid residues for each base being recognized. The invention employs peptidomimetics (e.g., unnatural amino acids, peptoids) and commonly employed chemistries for secondary structure pre-organization (e.g., “stapling,” side-chain crosslinking, hydrogen-bond surrogating) to miniaturize a TALE-like system providing nucleotide sequence readers that are proteolytically and chemically stable. In some embodiments, the nucleic acid binding domain may comprise at least five or more Transcription activator-like effector (TALE) monomers and at least one or more half-monomers specifically ordered to target the genomic locus of interest linked to at least one or more effector domains are further linked to a chemical or energy sensitive protein. This leads to a change in the sub-cellular localization of the entire polypeptide (i.e. transportation of the entire polypeptide from cytoplasm into the nucleus of the cells) upon the binding of a chemical or energy transfer to the chemical or energy sensitive protein. This transportation of the entire polypeptide from one sub-cellular compartments or organelles, in which its activity is sequestered due to lack of substrate for the effector domain, into another one in which the substrate is present would allow the entire polypeptide to come in contact with its desired substrate (i.e. genomic DNA in the mammalian nucleus) and result in activation or repression of target gene expression.

In certain embodiments, the sequence readers comprise or are engineered from zinc finger proteins, meganucleases, argonaute, or other nucleic acid binding domains.

### Peptide Nucleic Acids (PNAs)

In some embodiments, the DNA reader is a PNA. PNAs act as high fidelity DNA readers as well as a scaffold for display of synthetic nucleases, further reducing the size compared to that of Cas9-guide RNA complex. This size reduction will allow facile delivery of multiple miniGEMs into a cell type of interest and may even allow highly multiplexed editing. Advantageously, PNAs are resistant to degradation by proteases/nucleases. Second, the synthetic nuclease can be positioned anywhere along the PNA backbone allowing a way to introduce designer cuts—a feature extremely difficult to achieve with CRISPR-associated nucleases.

In some embodiments, templates for HDR can also be directly conjugated to the PNA backbone, enhancing their local concentration and improving the rate of genome integration at the desired site.

While multiple high-fidelity DNA readers exist, PNAs can be chosen for multiple reasons. First, PNAs are DNA analogs with neutral synthetic backbone in place of the negatively charged phosphodiester backbone of DNA. This neutral charge allows high-affinity binding to DNA compared to those attained by DNA/DNA or DNA/RNA hybrids. Second, next-generation PNAs (e.g., γPNA) are preorganized for binding to B-DNA in a sequence-unrestricted manner via Watson-Crick recognition. Third, the synthetic backbone of PNAs makes them resistant to proteases/nucleases. Fourth, a PNA/DNA mismatch is more destabilizing than a DNA/RNA mismatch, which could potentially reduce the off-target effects. Finally, efficient in vivo delivery of PNAs has been demonstrated for several disease systems by many groups.

In some embodiments, editors will induce four precisely spaced nicks on the genomic DNA, excising ˜20 base pairs fragment and leaving behind high-affinity “sticky ends.” Simultaneously, this editor will facilitate delivery of a high-concentration of an exogenous DNA (˜20 base pair) that will hybridize to the sticky ends and be inserted into the genome. Here the fact that the single-strand breaking small-molecules can be positioned at any site on the PNA will be leveraged, essentially allowing the introduction of any type of DNA break.

In some preferred embodiments, the nucleic acid modifying system can include two or more PNA molecules.

### Small Effector Component

Small effector components, can be in some embodiments, a small molecule synthetic nuclease, that in some embodiments is selected from the group consisting of diazofluorenes, nitracines, metal complexes, enediyenes, methoxsalen derivatives, daunorubicin derivatives and juglones. Embodiments can include a second, third or fourth effector component, which can be small molecule single strand breaking nucleases.

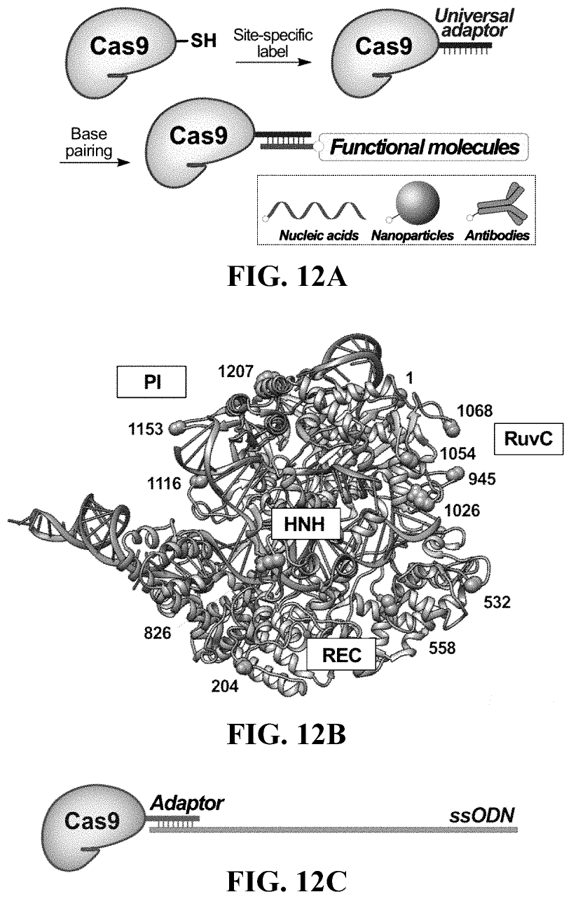

Single stranded oligo donors as described, one or more NHEJ inhibitors and/or HDR activators may be included, as well as p53 inhibitors, uracil DNA glycosylase inhibitors, as described herein. Conjugation of cargo is also provided as provided herein. In an embodiment, the cargo can include antibodies, nucleic acid molecules, nanoparticles, and other functional molecules utilizing a universal adaptor on the engineered genome editor that base pairs to functional molecules. See, e.g. FIG. 12A.

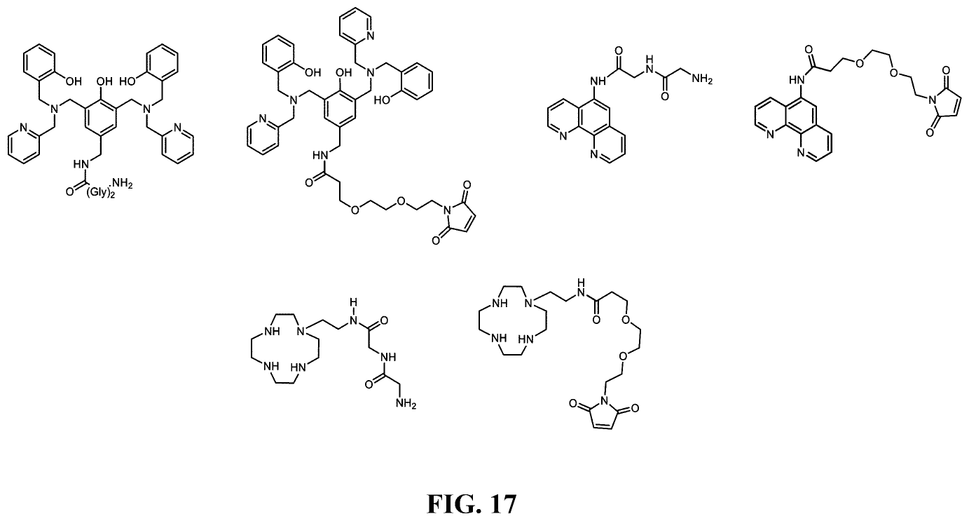

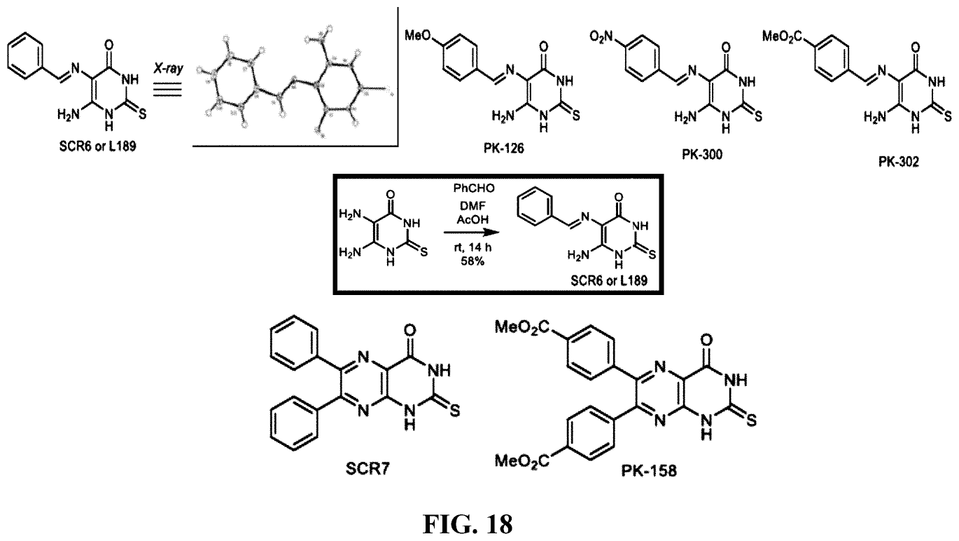

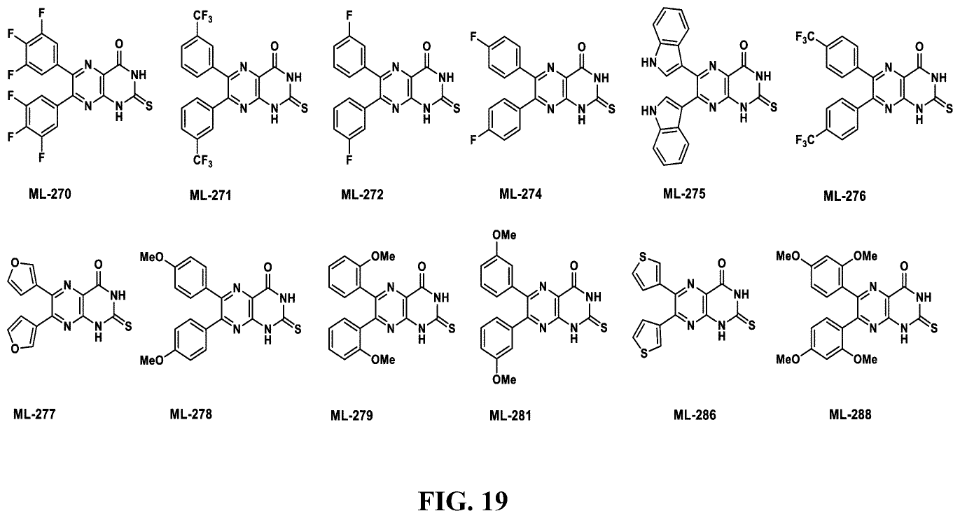

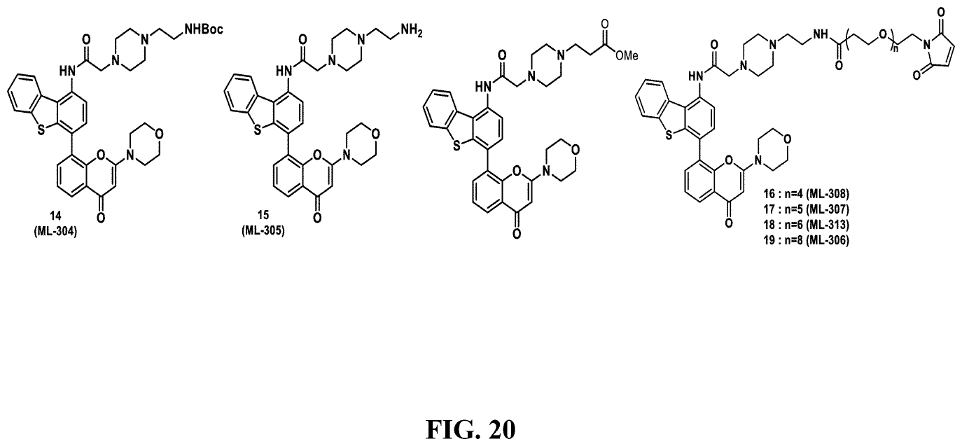

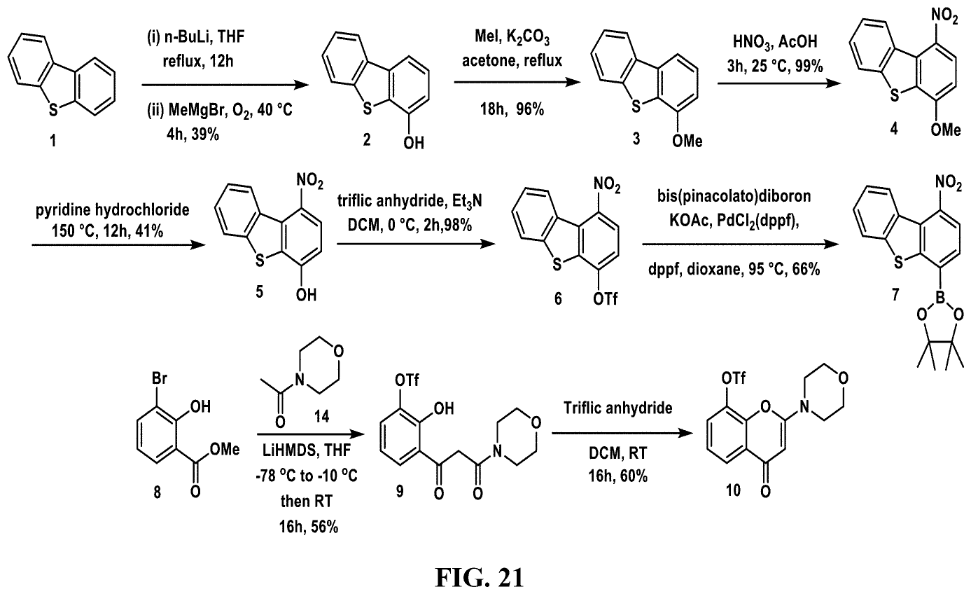

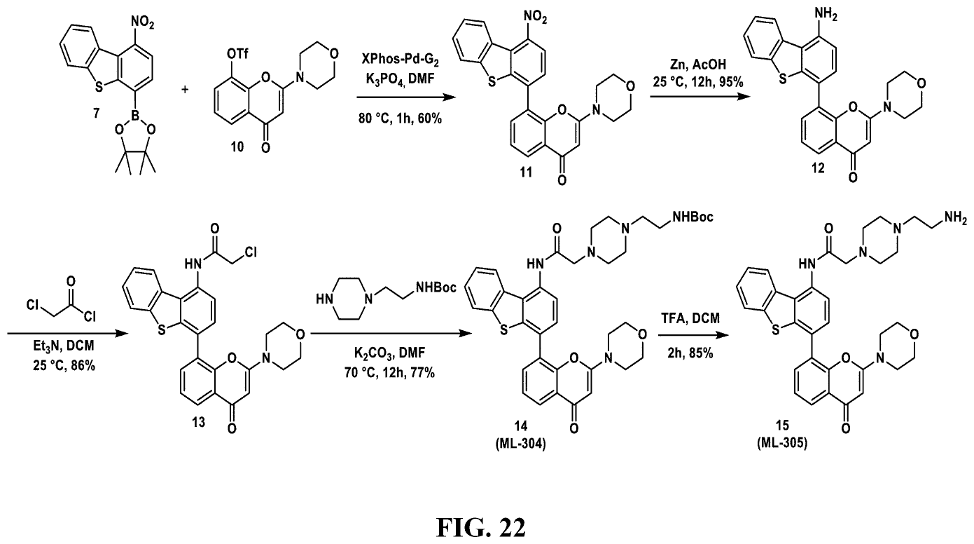

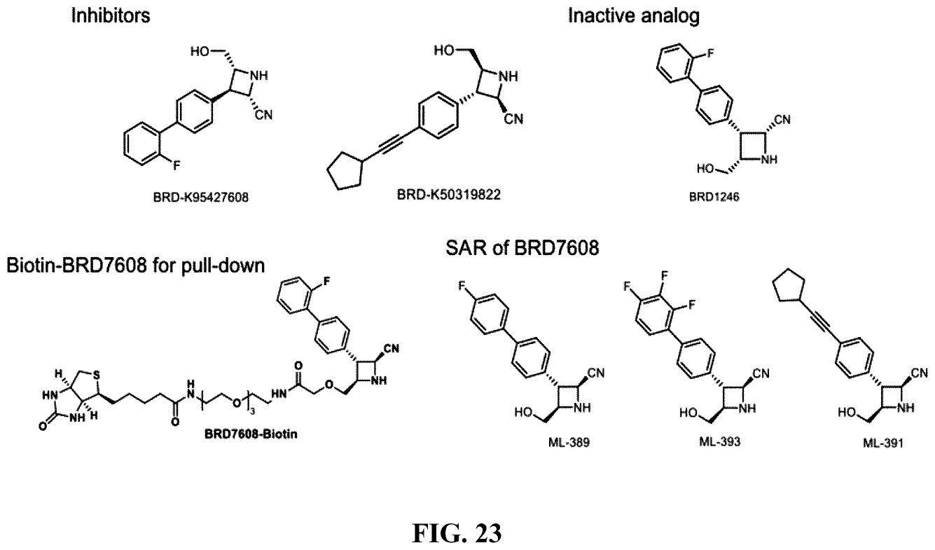

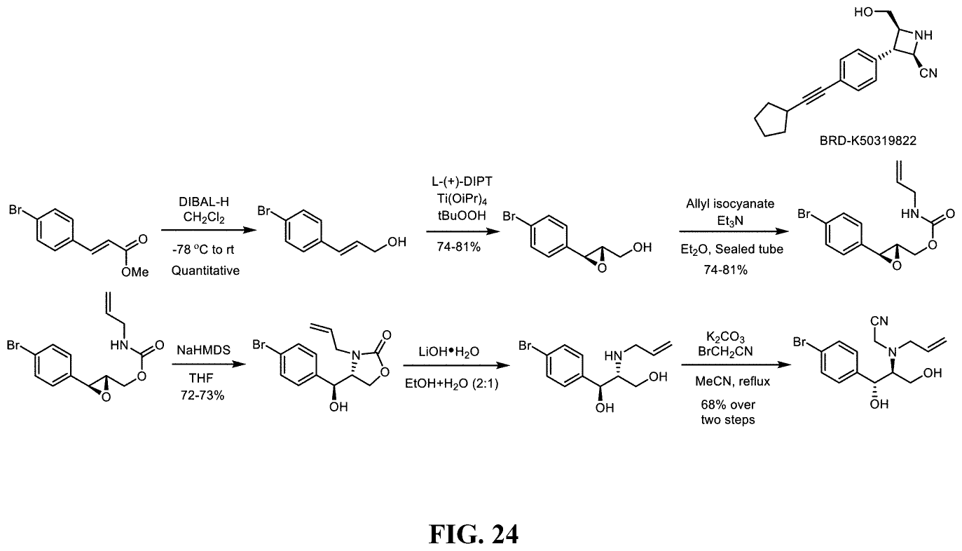

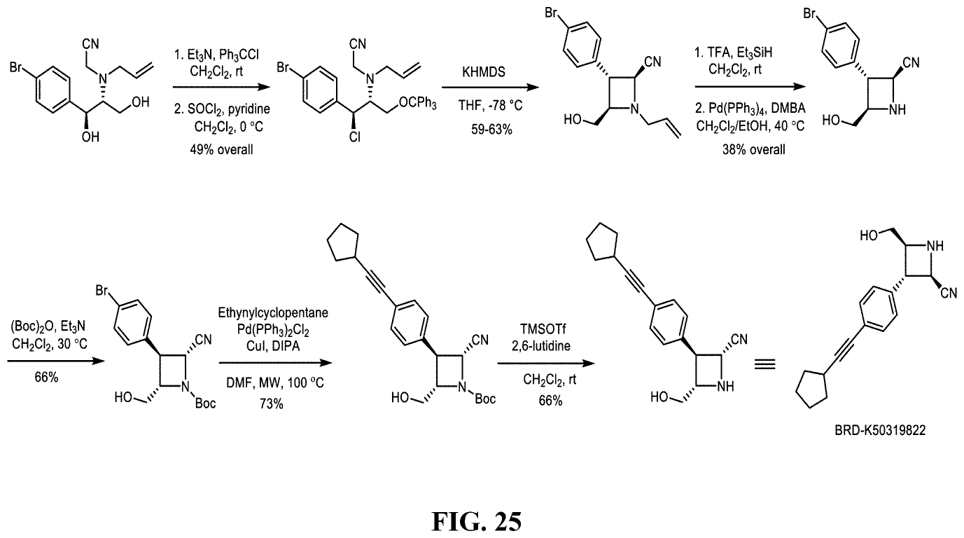

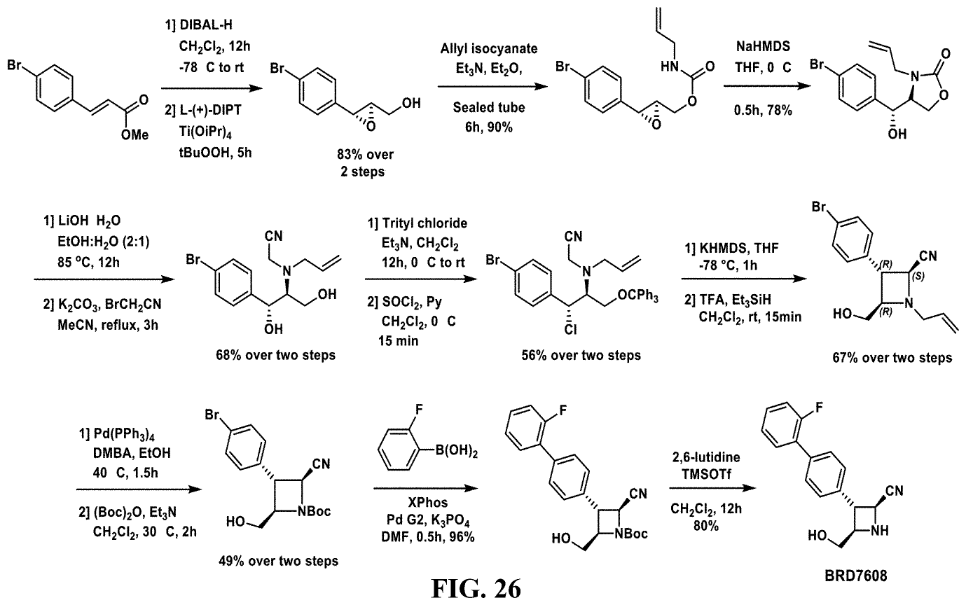

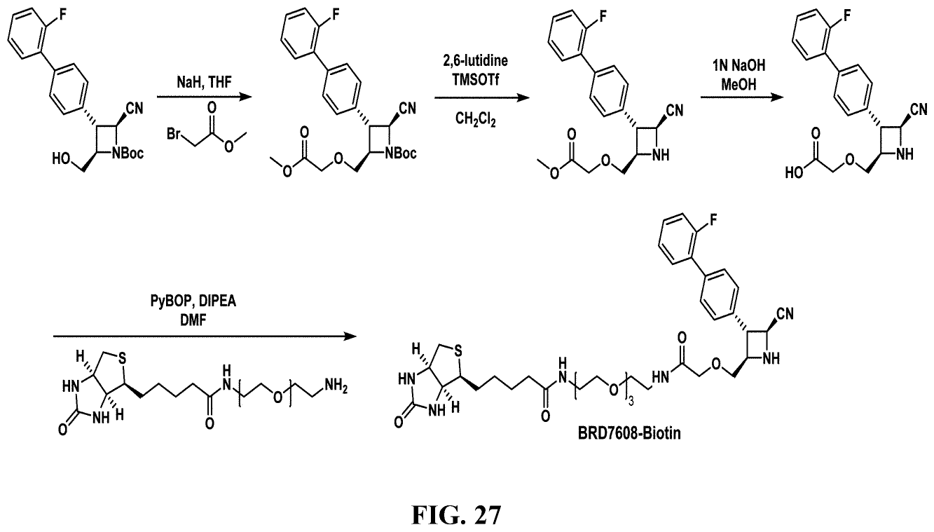

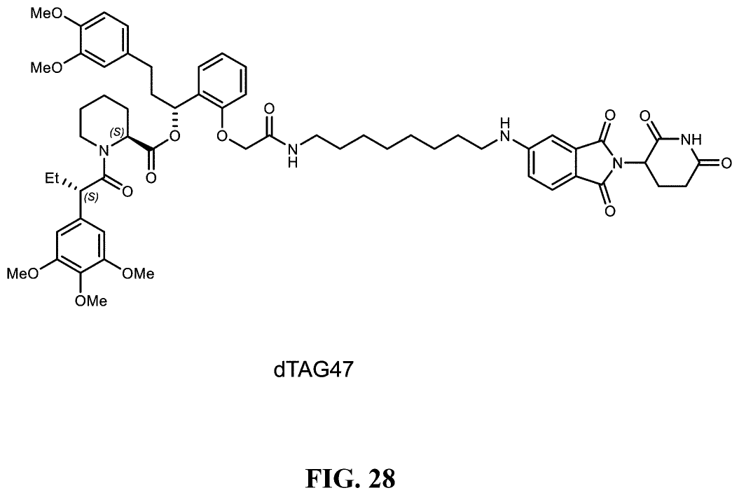

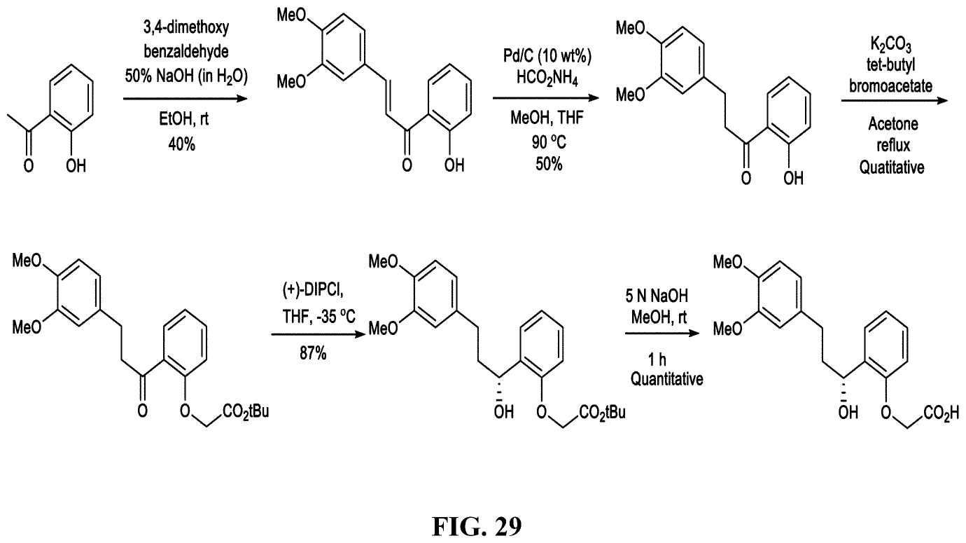

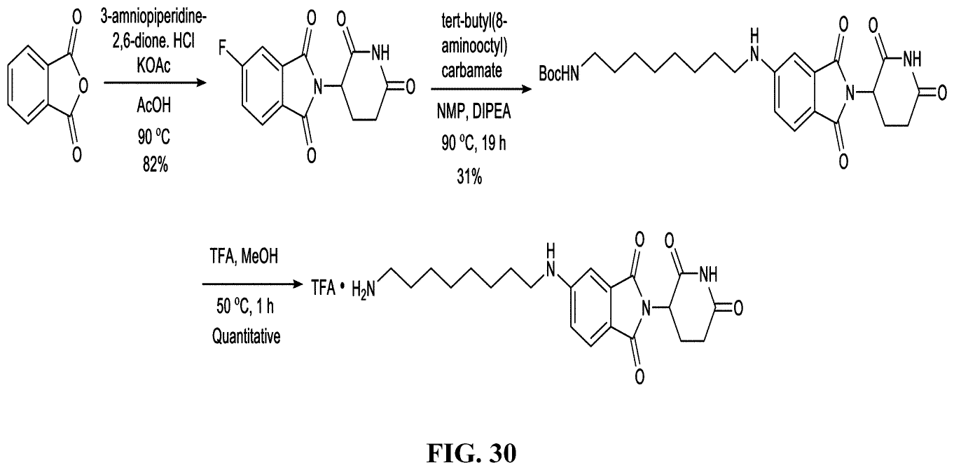

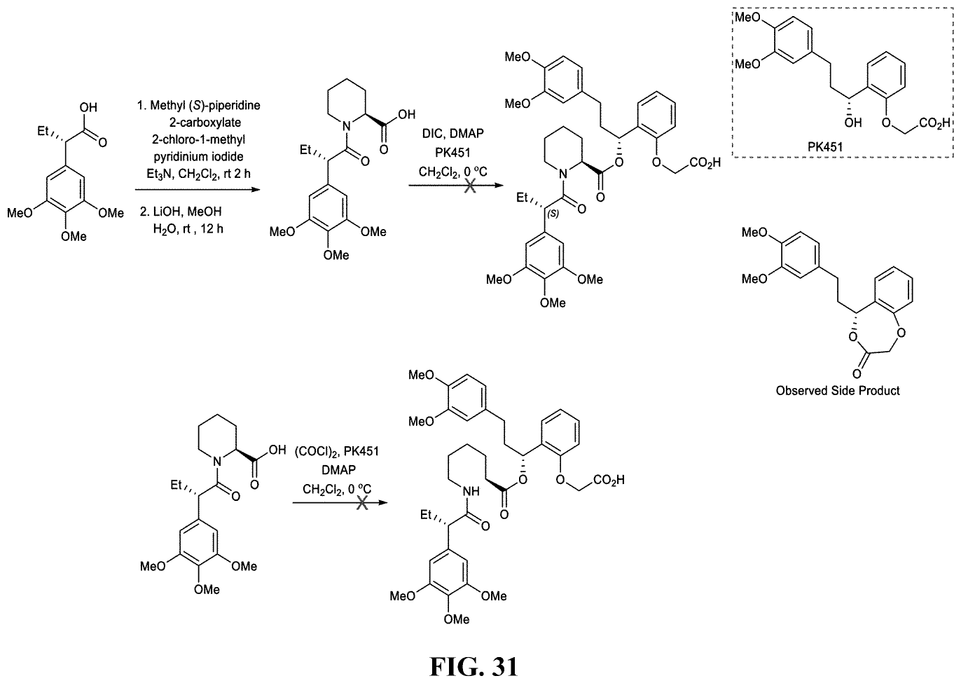

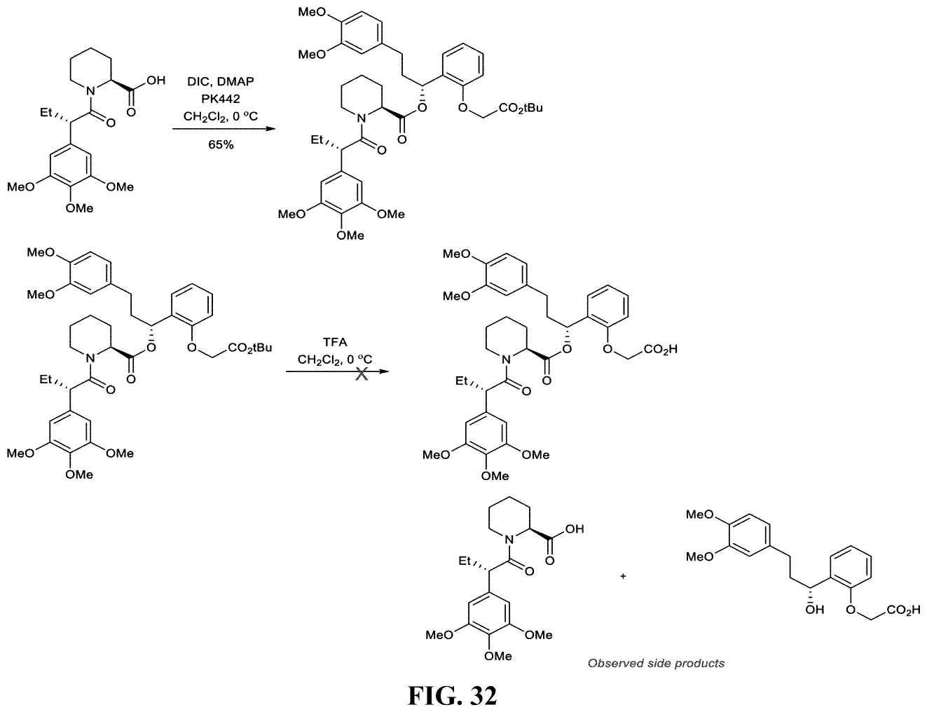

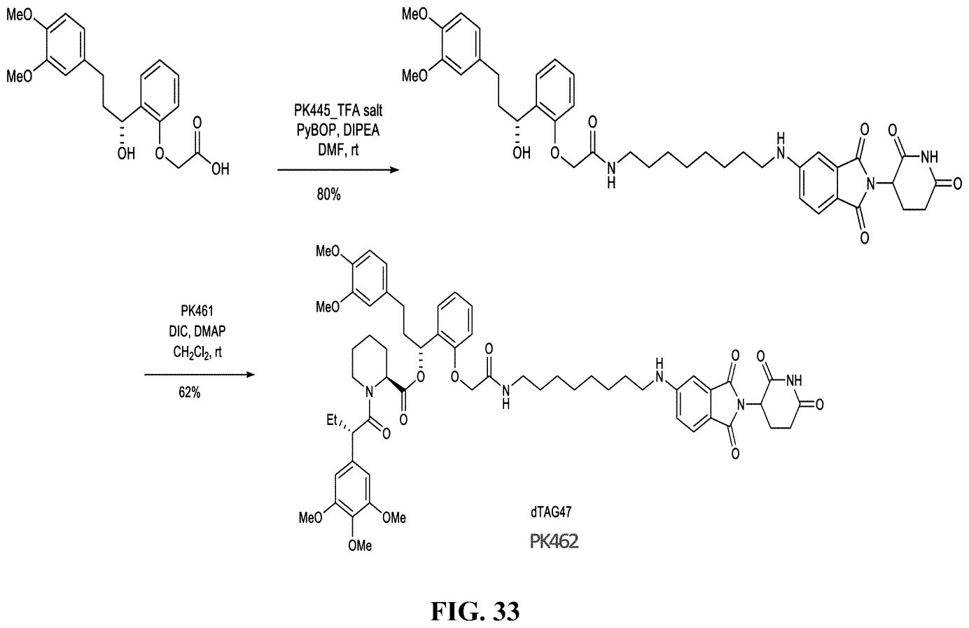

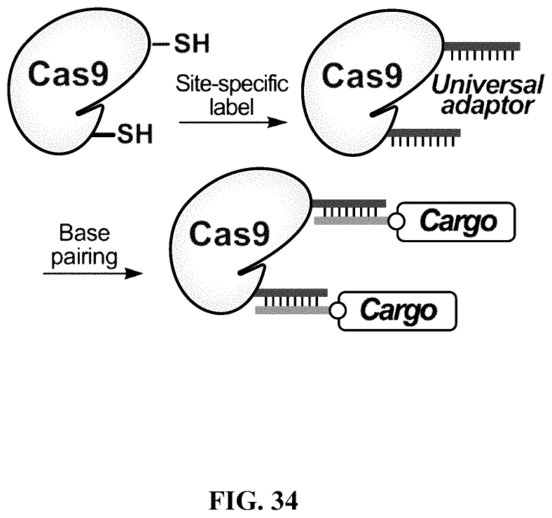

In some embodiments, a PNA can be conjugated with a double strand breaking small-molecule. In some embodiments, the systems provide two PNA molecules, each bearing a fragment of the split-small molecule nucleases. In some embodiments, the split-small molecule nucleases are metal complexes. It can be envisioned that the DNA acts as template for facilitating the coming together of two reactive components-a strategy that has been employed for DNA templated synthesis of molecules. Various small-molecule strand breakers and DNA modifiers have been described, and are contemplated for use in the currently described system, including diazofluorenes, nitracines metal complexes, enedyenes, DNA modifiers and others with diverse structures, including use as split molecule nucleases is described in PCT/2018/057182 at [0850]-[0856] and FIGS. 17A-19D, incorporated by reference.

### Conjugation of Strand-Breakers to PNA

In the PNA based genome editor that is envisioned, the PNA serves as the designer DNA reader that can be customized to target any desired genomic sequence while the DNA strand breaks will be induced by synthetic nucleases. In some embodiments, small molecules are covalently conjugated to the PNA. In some embodiments, the small molecule strand breaker can be covalently conjugated utilizing maleimide, azide or alkyne functional groups on the small molecules while installing a PEG linker with thiol, alkyne or azide functional handles on the PNA respectively to allow for efficient conjugation. By varying the length of the PEG linker, it is possible to effect the DNA cut close to or away from the PNA binding site, which provides additional flexibility in designing the DNA cut sites. To create staggered double stranded breaks on the DNA, two PNA molecules can be conjugated to single strand breakers at both N and C termini designed to bind the target DNA in a staggered fashion. In this manner, four staggered cuts in the DNA such that the donor DNA with complementary staggered ends can anneal to bring about precise genomic modification without involving DNA repair pathway.

HDR Enhancement Using miniGEMs.

NHEJ inhibitors and HDR activators can be displayed on the synthetic nucleic acid modifiers to enhance HDR as discussed. Simultaneous display of NHEJ inhibitors/HDR activators and DNA strand breakers requires multiple attachment sites on the PNA. The peptide backbone of the PNA provides such additional sites of attachment., including using functionalized PEG linkers (alkyne, azide, cyclooctyne etc.) that are commercially available can be employed for conjugation of NHEJ inhibitors at the (E≥ position. Functionalization of PNA at the (E≥ position by attachment of (R)-diethylene glycol miniPEG (MP) transforms a randomly folded PNA into a right handed helix providing right handed helical, R-MP(E≥PNA oligomers that hybridize to DNA and RNA with greater affinity and sequence selectivity than the parental PNA oligomers. Further, the miniPEG PNA has also been successfully used in ex vivo and in vivo studies for gene editing applications.

Guides that May be Used in the Present Invention

As used herein, the term “guide”, “crRNA” or “guide RNA” or “single guide RNA” or “sgRNA” or “one or more nucleic acid components” of a nucleic acid modifying protein comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid modifying system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence, and hence a nucleic acid-targeting guide may be selected to target any target nucleic acid sequence. In some embodiments, the target sequence may be DNA. In some embodiments, the target sequence may be any RNA sequence. In some embodiments, the target sequence may comprise both DNA and RNA, for example one or more DNA nucleotides with the rest being RNA, or one or more RNA nucleotides with the rest being DNA. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop corresponds to the tracr mate sequence, and the portion of the sequence 3′ of the loop corresponds to the tracr sequence.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In general, the nucleic acid modifying system may be as used in the foregoing documents, such as WO 2014/093622 (PCT/US2013/074667) and refers collectively to transcripts and other elements involved in the expression of or directing the activity of nucleic acid modifying-associated genes, including sequences encoding one or more domains of a Cas gene, for example, one of more domains of a Cas9 gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous nucleic acid modifying system), a guide sequence (also referred to as a “spacer” in the context of an endogenous nucleic acid modifying system), or “RNA(s)” as that term is herein used (e.g., RNA(s) to guide nucleic acid modifying protein, e.g. CRISPR RNA and transactivating (tracr) RNA or a single guide RNA (sgRNA) (chimeric RNA)) or other sequences and transcripts derived from a CRISPR locus. In general, a nucleic acid modifying system is characterized by elements that promote the formation of a nucleic acid modifying complex at the site of a target sequence (also referred to as a protospacer in the context of an endogenous nucleic acid modifying system). In the context of formation of a nucleic acid modifying complex, “target sequence” refers to a sequence to which a guide sequence is designed to have complementarity, where hybridization between a target sequence and a guide sequence promotes the formation of a nucleic acid modifying complex. The section of the guide sequence through which complementarity to the target sequence is important for cleavage activity is referred to herein as the seed sequence. A target sequence may comprise any polynucleotide, such as DNA or RNA polynucleotides. In some embodiments, a target sequence is located in the nucleus or cytoplasm of a cell, and may include nucleic acids in or from mitochondrial, organelles, vesicles, liposomes or particles present within the cell. In some embodiments, especially for non-nuclear uses, NLSs are not preferred. In some embodiments, a nucleic acid modifying system comprises one or more nuclear exports signals (NESs). In some embodiments, a nucleic acid modifying system comprises one or more NLSs and one or more NESs. In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used.

In embodiments of the invention the terms guide sequence and guide RNA, i.e. RNA capable of guiding Cas to a target genomic locus, are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a nucleic acid modifying protein to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 30 nucleotides long. The ability of a guide sequence to direct sequence-specific binding of a nucleic acid modifying protein to a target sequence may be assessed by any suitable assay. For example, the components of a nucleic acid modifying system sufficient to form a nucleic acid modifying complex, including the guide sequence to be tested and the nucleic acid modifying protein, may be provided to a host cell having the corresponding target sequence, such as by transfection with vectors encoding the components of the nucleic acid modifying sequence, followed by an assessment of preferential cleavage within the target sequence, such as by Surveyor assay as described herein. Similarly, cleavage of a target polynucleotide sequence may be evaluated in a test tube by providing the target sequence, components of a nucleic acid modifying complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments of nucleic acid modifying systems, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and advantageously tracr RNA is 30 or 50 nucleotides in length. However, an aspect of the invention is to reduce off-target interactions, e.g., reduce the guide interacting with a target sequence having low complementarity. Indeed, in the examples, it is shown that the invention involves mutations that result in the nucleic acid modifying system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In particularly preferred embodiments according to the invention, the guide RNA (capable of guiding nucleic acid modifying protein to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e. an sgRNA (arranged in a 5′ to 3′ orientation), or the tracr RNA may be a different RNA than the RNA containing the guide and tracr sequence. The tracr hybridizes to the tracr mate sequence and directs the nucleic acid modifying complex to the target sequence.

The methods according to the invention as described herein comprehend inducing one or more mutations in a eukaryotic cell (in vitro, i.e. in an isolated eukaryotic cell) as herein discussed comprising delivering to cell a vector as herein discussed. The mutation(s) can include the introduction, deletion, or substitution of one or more nucleotides at each target sequence of cell(s) via the guide(s). The mutations can include the introduction, deletion, or substitution of 1-75 nucleotides at each target sequence of said cell(s) via the guide(s) RNA(s) or sgRNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 40, 45, 50, 75, 100, 200, 300, 400 or 500 nucleotides at each target sequence of said cell(s) via the guide(s) RNA(s) or sgRNA(s).

For minimization of toxicity and off-target effect, it may be important to control the concentration of nucleic acid modifying protein mRNA and guide RNA delivered. Optimal concentrations of nucleic acid modifying protein mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, nucleic acid modifying nickase mRNA (for example nucleic acid modifying protein comprising one or more domains of S. pyogenes Cas9 with the D10A mutation) can be delivered with a pair of guide RNAs targeting a site of interest. Guide sequences and strategies to minimize toxicity and off-target effects can be as in WO 2014/093622 (PCT/US2013/074667); or, via mutation as herein.

Typically, in the context of an endogenous nucleic acid modifying system, formation of a nucleic acid modifying complex (comprising a guide sequence hybridized to a target sequence and complexed with one or more nucleic acid modifying proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a nucleic acid modifying complex, such as by hybridization along at least a portion of the tracr sequence to all or a portion of a tracr mate sequence that is operably linked to the guide sequence.

In some embodiments, guides of the invention comprise RNA. In certain embodiments, guides of the invention comprise DNA. In certain embodiments, guides of the invention comprise both RNA and DNA. In other words, guides of the invention may comprise both Ribonucleic acid (RNA) and/or Deoxyribonucleic acid (DNA). For areas where secondary structure is preferred or required, then Ribonucleic acid (RNA) is most useful. However, in other areas, such as a sequence complementary to the target sequence, then some or potentially all of the nucleotides may be Deoxyribonucleic acid (DNA). This may be designed subject to the functional requirements of the user. Blends of RNA to DNA may be about 100:0; 90:10; 80:20; 70:30; 60:40; 50:50; 40:60; 30:70; 20:80; 10:90; or 0:1000. Due to the utility of RNA secondary structure in some embodiments, the RNA:DNA ratio in the guide molecule may be 80:20; 70:30; 60:40; or 50:50. The Ribonucleic acid (RNA) and/or Deoxyribonucleic acid (DNA) may also be modified and so forth as described below.

In certain embodiments, guides of the invention comprise non-naturally occurring nucleic acids and/or non-naturally occurring nucleotides and/or nucleotide analogs, and/or chemically modified nucleotides (i.e. nucleotides comprising chemical modifications). Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′phosphorothioate (MS), or 2′-O-methyl 3′thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015; Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target DNA and one or more deoxyribonucleotides and/or nucleotide analogs in a region that binds to Cas9, Cpf1, or C2c1. In an embodiment of the invention, deoxyribonucleotides and/or nucleotide analogs are incorporated in engineered guide structures, such as, without limitation, 5′ and/or 3′ end, stem-loop regions, and the seed region. In certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemicially modified with 2′-O-methyl (M), 2′-O-methyl-3′-phosphorothioate (MS), S-constrained ethyl(cEt), or 2′-O-methyl-3′-thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemicially modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554)

In one aspect of the invention, the guide comprises a modified crRNA for Cpf1, having a 5′-handle and a guide segment further comprising a seed region and a 3′-terminus. In some embodiments, the modified guide can be used with a Cpf1 of any one of Acidaminococcus sp. BV3L6 Cpf1 (AsCpf1); Francisella tularensis subsp. Novicida U112 Cpf1 (FnCpf1); L. bacterium MC2017 Cpf1 (Lb3Cpf1); Butyrivibrio proteoclasticus Cpf1 (BpCpf1); Parcubacteria bacterium GWC2011_GWC2_44_17 Cpf1 (PbCpf1); Peregrinibacteria bacterium GW2011_GWA_33_10 Cpf1 (PeCpf1); Leptospira inadai Cpf1 (LiCpf1); Smithella sp. SC_K08D17 Cpf1 (SsCpf1); L. bacterium MA2020 Cpf1 (Lb2Cpf1); Porphyromonas crevioricanis Cpf1 (PcCpf1); Porphyromonas macacae Cpf1 (PmCpf1); Candidatus Methanoplasma termitum Cpf1 (CMtCpf1); Eubacterium eligens Cpf1 (EeCpf1); Moraxella bovoculi 237 Cpf1 (MbCpf1); Prevotella disiens Cpf1 (PdCpf1); or L. bacterium ND2006 Cpf1 (LbCpf1).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (melΨ), 5-methoxyuridine(5moU), inosine, 7-methylguanosine, 2′-O-methyl-3′-phosphorothioate (MS), S-constrained ethyl(cEt), phosphorothioate (PS), or 2′-O-methyl-3′-thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 or 10 nucleotides in the 3′-terminus are chemically modified. Such chemical modifications at the 3′-terminus of the Cpf1 CrRNA improve gene cutting efficiency (see Li, et al., Nature Biomedical Engineering, 2017, 1:0066). In a specific embodiment, 5 nucleotides in the 3′-terminus are replaced with 2′-fluoro analogues. In a specific embodiment, 10 nucleotides in the 3′-terminus are replaced with 2′-fluoro analogues. In a specific embodiment, 5 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the loop comprises 3, 4, or 5 nucleotides. In certain embodiments, the loop comprises the sequence of UCUU, UUUU, UAUU, or UGUU.

### Synthetically Linked Guides

In one aspect, the guide comprises a tracr sequence and a tracr mate sequence that are chemically linked or conjugated via a non-phosphodiester bond. In some embodiments, the tracr sequence and the tracr mate sequence are considered to be fused together or contiguous. In one aspect, the guide comprises a tracr sequence and a tracr mate sequence that are chemically linked or conjugated via a non-nucleotide loop. In some embodiments, the tracr and tracr mate sequences are joined via a non-phosphodiester covalent linker. Examples of the covalent linker include but are not limited to a chemical moiety selected from the group consisting of carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, the tracr and tracr mate sequences are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, the tracr or tracr mate sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once the tracr and the tracr mate sequences are functionalized, a covalent chemical bond or linkage can be formed between the two oligonucleotides. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, the tracr and tracr mate sequences can be chemically synthesized. The tracer and tracr mate alone/individually, synthesized together in the form of a fusion, or synthesized separately and chemically linked. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In some embodiments, the tracr and tracr mate sequences can be covalently linked using various bioconjugation reactions, loops, bridges, and non-nucleotide links via modifications of sugar, internucleotide phosphodiester bonds, purine and pyrimidine residues. Sletten et al., Angew. Chem. Int. Ed. (2009) 48:6974-6998; Manoharan, M. Curr. Opin. Chem. Biol. (2004) 8: 570-9; Behlke et al., Oligonucleotides (2008) 18: 305-19; Watts, et al., Drug. Discov. Today (2008) 13: 842-55; Shukla, et al., ChemMedChem (2010) 5: 328-49.

In some embodiments, the tracr and tracr mate sequences can be covalently linked using click chemistry. In some embodiments, the tracr and tracr mate sequences can be covalently linked using a triazole linker. In some embodiments, the tracr and tracr mate sequences can be covalently linked using Huisgen 1,3-dipolar cycloaddition reaction involving an alkyne and azide to yield a highly stable triazole linker (He et al., ChemBioChem (2015) 17: 1809-1812; WO 2016/186745). In some embodiments, the tracr and tracr mate sequences are covalently linked by ligating a 5′-hexyne tracrRNA and a 3′-azide crRNA. In some embodiments, either or both of the 5′-hexyne tracrRNA and a 3′-azide crRNA can be protected with 2′-acetoxyethl orthoester (2′-ACE) group, which can be subsequently removed using Dharmacon protocol (Scaringe et al., J. Am. Chem. Soc. (1998) 120: 11820-11821; Scaringe, Methods Enzymol. (2000) 317: 3-18).

In some embodiments, the tracr and tracr mate sequences can be covalently linked via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable spacers for purposes of this invention include, but are not limited to, polyethers (e.g., polyethylene glycols, polyalcohols, polypropylene glycol or mixtures of efhylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. The design of example linkers conjugating two RNA components are also described in WO 2004/015075.

The linker (e.g., a non-nucleotide loop) can be of any length. In some embodiments, the linker has a length equivalent to about 0-16 nucleotides. In some embodiments, the linker has a length equivalent to about 0-8 nucleotides. In some embodiments, the linker has a length equivalent to about 0-4 nucleotides. In some embodiments, the linker has a length equivalent to about 2 nucleotides. Example linker design is also described in WO2011/008730.

A typical nucleic acid modifying sgRNA comprises (in 5′ to 3′ direction): a guide sequence, a poly U tract, a first complimentary stretch (the “repeat”), a loop (tetraloop), a second complimentary stretch (the “anti-repeat” being complimentary to the repeat), a stem, and further stem loops and stems and a poly A (often poly U in RNA) tail (terminator). In preferred embodiments, certain aspects of guide architecture are retained, certain aspect of guide architecture cam be modified, for example by addition, subtraction, or substitution of features, whereas certain other aspects of guide architecture are maintained. Preferred locations for engineered sgRNA modifications, including but not limited to insertions, deletions, and substitutions include guide termini and regions of the sgRNA that are exposed when complexed with CRISPR protein and/or target, for example the tetraloop and/or loop2. Certain guide architecture and secondary structure may, as described herein, may utilized or encouraged in guides other than those specifically referred to as sgRNA.

In certain embodiments, guides of the invention comprise, for example are adapted or designed to include, one or more specific binding sites (e.g. comprising an aptamer or aptamer sequences such as MS2 or PP7, for example as described herein) for adaptor proteins. The adaptor proteins may comprise one or more effector domains (e.g. via fusion protein). When such a guide forms anucleic acid modifying complex (i.e. nucleic acid modifying protein binding to guide and target) the adaptor proteins bind and, the effector domain associated with the adaptor protein is positioned in a spatial orientation which is advantageous for the attributed function to be effective. For example, if the effector domain is a transcription activator (e.g. VP64 or p65), the transcription activator is placed in a spatial orientation which allows it to affect the transcription of the target. Likewise, a transcription repressor (e.g. KRAB) will be advantageously positioned to affect the transcription of the target and a nuclease (e.g. Fok1) will be advantageously positioned to cleave or partially cleave the target. Suitable examples of aptamer are described herein, for example below. Suitable examples of effector domains are also described herein.

The skilled person will understand that modifications to the guide which allow for binding of the adaptor+effector domain but not proper positioning of the adaptor+effector domain (e.g. due to steric hindrance within the three-dimensional structure of the nucleic acid modifying complex) are modifications which are not intended if the nucleic acid modifying complex is to be optimally formed or formed in a functional manner. In some embodiments, sub-optimal formation of the nucleic acid modifying complex may be useful. The one or more modified guide may be modified at the tetra loop, the stem loop 1, stem loop 2, or stem loop 3, as described herein, preferably at either the tetra loop or stem loop 2, and most preferably at both the tetra loop and stem loop 2.

The repeat:anti repeat duplex will be apparent from the secondary structure of the sgRNA. It may be typically a first complimentary stretch after (in 5′ to 3′ direction) the poly U tract and before the tetraloop; and a second complimentary stretch after (in 5′ to 3′ direction) the tetraloop and before the poly A tract. The first complimentary stretch (the “repeat”) is complimentary to the second complimentary stretch (the “anti-repeat”). As such, they Watson-Crick base pair to form a duplex of dsRNA when folded back on one another. As such, the anti-repeat sequence is the complimentary sequence of the repeat and in terms to A-U or C-G base pairing, but also in terms of the fact that the anti-repeat is in the reverse orientation due to the tetraloop.

In an embodiment of the invention, modification of guide architecture comprises replacing bases in stem loop 2. For example, in some embodiments, “actt” (“acuu” in RNA) and “aagt” (“aagu” in RNA) bases in stemloop2 are replaced with “cgcc” and “gcgg”. In some embodiments, “actt” and “aagt” bases in stemloop2 are replaced with complimentary GC-rich regions of 4 nucleotides. In some embodiments, the complimentary GC-rich regions of 4 nucleotides are “cgcc” and “gcgg” (both in 5′ to 3′ direction). In some embodiments, the complimentary GC-rich regions of 4 nucleotides are “gcgg” and “cgcc” (both in 5′ to 3′ direction). Other combination of C and G in the complimentary GC-rich regions of 4 nucleotides will be apparent including CCCC and GGGG.

In one aspect, the stem loop 2, e.g., “ACTTgtttAAGT” (SEQ ID NO: 7) can be replaced by any “XXXXgtttYYYY”, e.g., where XXXX and YYYY represent any complementary sets of nucleotides that together will base pair to each other to create a stem.

In one aspect, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-12 and Y2-12 (wherein X and Y represent any complementary set of nucleotides) may be contemplated. In one aspect, the stem made of the X and Y nucleotides, together with the “gttt,” will form a complete hairpin in the overall secondary structure; and, this may be advantageous and the amount of base pairs can be any amount that forms a complete hairpin. In one aspect, any complementary X:Y base pairing sequence (e.g., as to length) is tolerated, so long as the secondary structure of the entire sgRNA is preserved. In one aspect, the stem can be a form of X:Y base pairing that does not disrupt the secondary structure of the whole sgRNA in that it has a DR:tracr duplex, and 3 stem loops. In one aspect, the “gttt” tetraloop that connects ACTT and AAGT (or any alternative stem made of X:Y basepairs) can be any sequence of the same length (e.g., 4 basepair) or longer that does not interrupt the overall secondary structure of the sgRNA. In one aspect, the stem loop can be something that further lengthens stemloop2, e.g. can be MS2 aptamer. In one aspect, the stemloop3 “GGCACCGagtCGGTGC” (SEQ ID NO: 8) can likewise take on a “XXXXXXXXagtYYYYYYY” form, e.g., wherein X7 and Y7 represent any complementary sets of nucleotides that together will base pair to each other to create a stem. In one aspect, the stem comprises about 7 bp comprising complementary X and Y sequences, although stems of more or fewer bas epairs are also contemplated. In one aspect, the stem made of the X and Y nucleotides, together with the “agt”, will form a complete hairpin in the overall secondary structure. In one aspect, any complementary X:Y basepairing sequence is tolerated, so long as the secondary structure of the entire sgRNA is preserved. In one aspect, the stem can be a form of X:Y basepairing that doesn't disrupt the secondary structure of the whole sgRNA in that it has a DR:tracr duplex, and 3 stemloops. In one aspect, the “agt” sequence of the stemloop 3 can be extended or be replaced by an aptamer, e.g., a MS2 aptamer or sequence that otherwise generally preserves the architecture of stemloop3. In one aspect for alternative Stemloops 2 and/or 3, each X and Y pair can refer to any basepair. In one aspect, non-Watson Crick basepairing is contemplated, where such pairing otherwise generally preserves the architecture of the stemloop at that position. See herein for further discussion of aptamers.

In one aspect, the DR:tracrRNA duplex can be replaced with the form: gYYYYag(N)NNNNxxxxNNNN(AAN)uuRRRRu (using standard IUPAC nomenclature for nucleotides), wherein (N) and (AAN) represent part of the bulge in the duplex, and “xxxx” represents a linker sequence. NNNN on the direct repeat can be anything so long as it basepairs with the corresponding NNNN portion of the tracrRNA. In one aspect, the DR:tracrRNA duplex can be connected by a linker of any length (xxxx . . . ), any base composition, as long as it doesn't alter the overall structure.

In one aspect, the sgRNA structural requirement is to have a duplex and 3 stemloops. In most aspects, the actual sequence requirement for many of the particular base requirements are lax, in that the architecture of the DR:tracrRNA duplex should be preserved, but the sequence that creates the architecture, i.e., the stems, loops, bulges, etc., may be altered.

### Aptamers

In general, the guides are modified in a manner that provides specific binding sites (e.g. aptamers) for adaptor proteins comprising one or more effector domains (e.g. via fusion protein) to bind to. The modified guides are modified such that once the guides forms a DNA binding complex (i.e. nucleic acid modifying protein binding to guides and target) the adaptor proteins bind. The effector domain on the adaptor protein is positioned in a spatial orientation which is advantageous for the attributed function to be effective. For example, if the effector domain is a transcription activator (e.g. VP64 or p65), the transcription activator is placed in a spatial orientation which allows it to affect the transcription of the target. Likewise, a transcription repressor (e.g. KRAB) will be advantageously positioned to affect the transcription of the target and a nuclease (e.g. Fok1) will be advantageously positioned to cleave or partially cleave the target.

The skilled person will understand that modifications to the guide which allow for binding of the adaptor+effector domain but not proper positioning of the adaptor+effector domain (e.g. due to steric hindrance within the three dimensional structure of the nucleic acid modifying complex) are modifications which are not intended. The one or more modified guide may be modified at the tetra loop, the stem loop 1, stem loop 2, or stem loop 3, as appropriate. This is described herein, preferably at either the tetra loop or stem loop 2, and most preferably at both the tetra loop and stem loop 2.

As explained herein the effector domains may be, for example, one or more domains from the group consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g. light inducible). In some cases it is advantageous that additionally at least one NLS is provided. In some instances, it is advantageous to position the NLS at the N terminus. When more than one effector domain is included, the effector domains may be the same or different.

The guide may be designed to include multiple binding recognition sites (e.g. aptamers) specific to the same or different adaptor protein. The guide may be designed to bind to the promoter region −1000-+1 nucleic acids upstream of the transcription start site (i.e. TSS), preferably −200 nucleic acids. This positioning improves effector domains which affect gene activation (e.g. transcription activators) or gene inhibition (e.g. transcription repressors). The modified guide may be one or more modified guides targeted to one or more target loci (e.g. at least 1 guide, at least 2 guides, at least 5 guides, at least 10 guides, at least 20 guides, at least 30 guides, at least 50 guides) comprised in a composition. The guides may be gRNA or may comprise DNA as described herein.

MS2 and PP7 are examples of suitable aptamers and so their sequences may be incorporated into the guides. Thus, in some embodiments, the guide may comprise aptamer sequences such as MS2 or PP7, capable of binding to a nucleotide-binding protein. The nucleotide-binding protein may be fused to otherwise comprise a effector domain as described hereon. References is made here to Konermann et al. (Konermann et al., “Genome-scale transcription activation by an engineered CRISPR-Cas9 complex,” doi:10.1038/nature14136, incorporated herein by reference).

The adaptor protein may be any number of proteins that binds to an aptamer or recognition site introduced into the modified dead gRNA and which allows proper positioning of one or more effector domains, once the dead gRNA has been incorporated into the nucleic acid modifying complex, to affect the target with the attributed function. As explained in detail in this application such may be coat proteins, preferably bacteriophage coat proteins. The effector domains associated with such adaptor proteins (e.g. in the form of fusion protein) may include, for example, one or more domains from the group consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g. light inducible). Preferred domains are Fok1, VP64, P65, HSF1, MyoD1. In the event that the effector domain is a transcription activator or transcription repressor it is advantageous that additionally at least an NLS is provided and preferably at the N terminus. When more than one effector domain is included, the effector domains may be the same or different. The adaptor protein may utilize known linkers to attach such effector domains.

Examples of guide-aptamers-nucleotide-binding protein-effector domain arrangements include:

Guide—MS2 aptamer—MS2 RNA-binding protein—ED; or

Guide—PP7 aptamer—PP7 RNA-binding protein—ED.

where ED is a Effector domain such as VP64 activator, SID4x repressor, Fok1 nuclease, or as otherwise described herein.

One guide with a first aptamer/RNA-binding protein pair can be linked or fused to an activator, whilst a second guide with a second aptamer/RNA-binding protein pair can be linked or fused to a repressor. The guides are for different targets (loci), so this allows one gene to be activated and one repressed. For example, the following schematic shows such an approach:

Guide 1—MS2 aptamer—MS2 RNA-binding protein—VP64 activator; and
Guide 2—PP7 aptamer—PP7 RNA-binding protein—SID4x repressor.

The present invention also relates to orthogonal PP7/MS2 gene targeting. In this example, sgRNA targeting different loci are modified with distinct RNA loops in order to recruit MS2-VP64 or PP7-SID4X, which activate and repress their target loci, respectively. PP7 is the RNA-binding coat protein of the bacteriophage Pseudomonas. Like MS2, it binds a specific RNA sequence and secondary structure. The PP7 RNA-recognition motif is distinct from that of MS2. Consequently, PP7 and MS2 can be multiplexed to mediate distinct effects at different genomic loci simultaneously. For example, an sgRNA targeting locus A can be modified with MS2 loops, recruiting MS2-VP64 activators, while another sgRNA targeting locus B can be modified with PP7 loops, recruiting PP7-SID4X repressor domains. In the same cell, dCas9 can thus mediate orthogonal, locus-specific modifications. This principle can be extended to incorporate other orthogonal RNA-binding proteins such as Q-beta.

An alternative option for orthogonal repression includes incorporating non-coding RNA loops with transactive repressive function into the guide (either at similar positions to the MS2/PP7 loops integrated into the guide or at the 3′ terminus of the guide). For instance, guides were designed with non-coding (but known to be repressive) RNA loops (e.g. using the Alu repressor (in RNA) that interferes with RNA polymerase II in mammalian cells). The Alu RNA sequence was located: in place of the MS2 RNA sequences as used herein (e.g. at tetraloop and/or stem loop 2); and/or at 3′ terminus of the guide. This gives possible combinations of MS2, PP7 or Alu at the tetraloop and/or stemloop 2 positions, as well as, optionally, addition of Alu at the 3′ end of the guide (with or without a linker).

The use of two different aptamers (distinct RNA) allows an activator-adaptor protein fusion and a repressor-adaptor protein fusion to be used, with different guides, to activate expression of one gene, whilst repressing another. They, along with their different guides can be administered together, or substantially together, in a multiplexed approach. A large number of such modified guides can be used all at the same time, for example 10 or 20 or 30 and so forth, whilst only one (or at least a minimal number) of nucleic acid modifying proteins to be delivered, as a comparatively small number of nucleic acid modifying proteins can be used with a large number modified guides. The adaptor protein may be associated (preferably linked or fused to) one or more activators or one or more repressors. For example, the adaptor protein may be associated with a first activator and a second activator. The first and second activators may be the same, but they are preferably different activators. For example, one might be VP64, whilst the other might be p65, although these are just examples and other transcriptional activators are envisaged. Three or more or even four or more activators (or repressors) may be used, but package size may limit the number being higher than 5 different effector domains. Linkers are preferably used, over a direct fusion to the adaptor protein, where two or more effector domains are associated with the adaptor protein. Suitable linkers might include the GlySer linker.

It is also envisaged that the protein-guide complex as a whole may be associated with two or more effector domains. For example, there may be two or more effector domains associated with the nucleic acid modifying protein, or there may be two or more effector domains associated with the guide (via one or more adaptor proteins), or there may be one or more effector domains associated with the nucleic acid modifying protein and one or more effector domains associated with the guide (via one or more adaptor proteins).

The fusion between the adaptor protein and the activator or repressor may include a linker. For example, GlySer linkers GGGS can be used. They can be used in repeats of 3 ((GGGGS)3) (SEQ ID NO: 94) or 6, 9 or even 12 (SEQ ID NOs: 95, 96 and 97, respectively) or more, to provide suitable lengths, as required. Linkers can be used between the DNA binding protein and an effector domain (activator or repressor), or between the nucleic acid binding domain and the effector domain (activator or repressor). The linkers the user to engineer appropriate amounts of “mechanical flexibility”.

### Dead Guides: Guide RNAs Comprising a Dead Guide Sequence May be Used in the Present Invention

In one aspect, the invention provides guide sequences which are modified in a manner which allows for formation of the CRISPR complex and successful binding to the target, while at the same time, not allowing for successful nuclease activity (i.e. without nuclease activity/without indel activity). For matters of explanation such modified guide sequences are referred to as “dead guides” or “dead guide sequences”. These dead guides or dead guide sequences can be thought of as catalytically inactive or conformationally inactive with regard to nuclease activity. Nuclease activity may be measured using surveyor analysis or deep sequencing as commonly used in the art, preferably surveyor analysis. Similarly, dead guide sequences may not sufficiently engage in productive base pairing with respect to the ability to promote catalytic activity or to distinguish on-target and off-target binding activity. Briefly, the surveyor assay involves purifying and amplifying a CRISPR target site for a gene and forming heteroduplexes with primers amplifying the CRISPR target site. After re-anneal, the products are treated with SURVEYOR nuclease and SURVEYOR enhancer S (Transgenomics) following the manufacturer's recommended protocols, analyzed on gels, and quantified based upon relative band intensities.

Hence, in a related aspect, the invention provides a non-naturally occurring or engineered composition nucleic acid modifying system comprising a functional nucleic acid modifying protein as described herein, and guide RNA (gRNA) wherein the gRNA comprises a dead guide sequence whereby the gRNA is capable of hybridizing to a target sequence such that the nucleic acid modifying system is directed to a genomic locus of interest in a cell without detectable indel activity resultant from nuclease activity of nucleic acid modifying protein of the system as detected by a SURVEYOR assay. For shorthand purposes, a gRNA comprising a dead guide sequence whereby the gRNA is capable of hybridizing to a target sequence such that the nucleic acid modifying system is directed to a genomic locus of interest in a cell without detectable indel activity resultant from nuclease activity of a nucleic acid modifying protein of the system as detected by a SURVEYOR assay is herein termed a “dead gRNA”. It is to be understood that any of the gRNAs according to the invention as described herein elsewhere may be used as dead gRNAs/gRNAs comprising a dead guide sequence as described herein below. Any of the methods, products, compositions and uses as described herein elsewhere is equally applicable with the dead gRNAs/gRNAs comprising a dead guide sequence as further detailed below. By means of further guidance, the following particular aspects and embodiments are provided.

The ability of a dead guide sequence to direct sequence-specific binding of a nucleic acid modifying complex (nucleic acid modifying protein and guide) to a target sequence may be assessed by any suitable assay. For example, the components of a nucleic acid modifying system sufficient to form a nucleic acid modifying complex, including the dead guide sequence to be tested, may be provided to a host cell having the corresponding target sequence, such as by transfection with vectors encoding the components of the nucleic acid modifying sequence, followed by an assessment of preferential cleavage within the target sequence, such as by Surveyor assay as described herein. Similarly, cleavage of a target polynucleotide sequence may be evaluated in a test tube by providing the target sequence, components of a nucleic acid modifying complex, including the dead guide sequence to be tested and a control guide sequence different from the test dead guide sequence, and comparing binding or rate of cleavage at the target sequence between the test and control guide sequence reactions. Other assays are possible, and will occur to those skilled in the art. A dead guide sequence may be selected to target any target sequence. In some embodiments, the target sequence is a sequence within a genome of a cell.

As explained further herein, several structural parameters allow for a proper framework to arrive at such dead guides. Dead guide sequences are shorter than respective guide sequences which result in active nucleic acid modifying protein-specific indel formation. Dead guides are 5%, 10%, 20%, 30%, 40%, 50%, shorter than respective guides directed to the same nucleic acid modifying protein leading to active nucleic acid modifying protein-specific indel formation.

As explained below and known in the art, one aspect of gRNA—nucleic acid modifying protein specificity is the direct repeat sequence, which is to be appropriately linked to such guides. In particular, this implies that the direct repeat sequences are designed dependent on the origin of the nucleic acid modifying protein. Thus, structural data available for validated dead guide sequences may be used for designing nucleic acid modifying protein specific equivalents. Structural similarity between, e.g., the orthologous nuclease domains RuvC of two or more Cas9 effector proteins may be used to transfer design equivalent dead guides. Thus, the dead guide herein may be appropriately modified in length and sequence to reflect such Cas9 specific equivalents, allowing for formation of the nucleic acid modifying complex and successful binding to the target, while at the same time, not allowing for successful nuclease activity.

The use of dead guides in the context herein as well as the state of the art provides a surprising and unexpected platform for network biology and/or systems biology in both in vitro, ex vivo, and in vivo applications, allowing for multiplex gene targeting, and in particular bidirectional multiplex gene targeting. Prior to the use of dead guides, addressing multiple targets, for example for activation, repression and/or silencing of gene activity, has been challenging and in some cases not possible. With the use of dead guides, multiple targets, and thus multiple activities, may be addressed, for example, in the same cell, in the same animal, or in the same patient. Such multiplexing may occur at the same time or staggered for a desired timeframe.

For example, the dead guides now allow for the first time to use gRNA as a means for gene targeting, without the consequence of nuclease activity, while at the same time providing directed means for activation or repression. Guide RNA comprising a dead guide may be modified to further include elements in a manner which allow for activation or repression of gene activity, in particular protein adaptors (e.g. aptamers) as described herein elsewhere allowing for functional placement of gene effectors (e.g. activators or repressors of gene activity). One example is the incorporation of aptamers, as explained herein and in the state of the art. By engineering the gRNA comprising a dead guide to incorporate protein-interacting aptamers (Konermann et al., “Genome-scale transcription activation by an engineered CRISPR-Cas9 complex,” doi:10.1038/nature14136, incorporated herein by reference), one may assemble a synthetic transcription activation complex consisting of multiple distinct effector domains. Such may be modeled after natural transcription activation processes. For example, an aptamer, which selectively binds an effector (e.g. an activator or repressor; dimerized MS2 bacteriophage coat proteins as fusion proteins with an activator or repressor), or a protein which itself binds an effector (e.g. activator or repressor) may be appended to a dead gRNA tetraloop and/or a stem-loop 2. In the case of MS2, the fusion protein MS2-VP64 binds to the tetraloop and/or stem-loop 2 and in turn mediates transcriptional up-regulation, for example for Neurog2. Other transcriptional activators are, for example, VP64. P65, HSF1, and MyoD1. By mere example of this concept, replacement of the MS2 stem-loops with PP7-interacting stem-loops may be used to recruit repressive elements.

Thus, one aspect is a gRNA of the invention which comprises a dead guide, wherein the gRNA further comprises modifications which provide for gene activation or repression, as described herein. The dead gRNA may comprise one or more aptamers. The aptamers may be specific to gene effectors, gene activators or gene repressors. Alternatively, the aptamers may be specific to a protein which in turn is specific to and recruits/binds a specific gene effector, gene activator or gene repressor. If there are multiple sites for activator or repressor recruitment, it is preferred that the sites are specific to either activators or repressors. If there are multiple sites for activator or repressor binding, the sites may be specific to the same activators or same repressors. The sites may also be specific to different activators or different repressors. The gene effectors, gene activators, gene repressors may be present in the form of fusion proteins.

In an embodiment, the dead gRNA as described herein or the Cas9 CRISPR-Cas complex as described herein includes a non-naturally occurring or engineered composition comprising two or more adaptor proteins, wherein each protein is associated with one or more effector domains and wherein the adaptor protein binds to the distinct RNA sequence(s) inserted into the at least one loop of the dead gRNA.

Hence, an aspect provides a non-naturally occurring or engineered composition comprising a guide RNA (gRNA) comprising a dead guide sequence capable of hybridizing to a target sequence in a genomic locus of interest in a cell, wherein the dead guide sequence is as defined herein, a nucleic acid modifying protein comprising at least one or more nuclear localization sequences, wherein the nucleic acid modifying protein optionally comprises at least one mutation wherein at least one loop of the dead gRNA is modified by the insertion of distinct RNA sequence(s) that bind to one or more adaptor proteins, and wherein the adaptor protein is associated with one or more effector domains; or, wherein the dead gRNA is modified to have at least one non-coding functional loop, and wherein the composition comprises two or more adaptor proteins, wherein the each protein is associated with one or more effector domains.

In certain embodiments, the adaptor protein is a fusion protein comprising the effector domain, the fusion protein optionally comprising a linker between the adaptor protein and the effector domain, the linker optionally including a GlySer linker.

In certain embodiments, the at least one loop of the dead gRNA is not modified by the insertion of distinct RNA sequence(s) that bind to the two or more adaptor proteins.

In certain embodiments, the one or more effector domains associated with the adaptor protein is a transcriptional activation domain.

In certain embodiments, the one or more effector domains associated with the adaptor protein is a transcriptional activation domain comprising VP64, p65, MyoD1, HSF1, RTA or SET7/9.

In certain embodiments, the one or more effector domains associated with the adaptor protein is a transcriptional repressor domain.

In certain embodiments, the transcriptional repressor domain is a KRAB domain.

In certain embodiments, the transcriptional repressor domain is a NuE domain, NcoR domain, SID domain or a SID4X domain.

In certain embodiments, at least one of the one or more effector domains associated with the adaptor protein have one or more activities comprising methylase activity, demethylase activity, transcription activation activity, transcription repression activity, transcription release factor activity, histone modification activity, DNA integration activity RNA cleavage activity, DNA cleavage activity or nucleic acid binding activity.

In certain embodiments, the DNA cleavage activity is due to a Fok1 nuclease.

In certain embodiments, the dead gRNA is modified so that, after dead gRNA binds the adaptor protein and further binds to the nucleic acid modifying protein and target, the effector domain is in a spatial orientation allowing for the effector domain to function in its attributed function.

In certain embodiments, the at least one loop of the dead gRNA is tetra loop and/or loop2. In certain embodiments, the tetra loop and loop 2 of the dead gRNA are modified by the insertion of the distinct RNA sequence(s).

In certain embodiments, the insertion of distinct RNA sequence(s) that bind to one or more adaptor proteins is an aptamer sequence. In certain embodiments, the aptamer sequence is two or more aptamer sequences specific to the same adaptor protein. In certain embodiments, the aptamer sequence is two or more aptamer sequences specific to different adaptor protein.

In certain embodiments, the adaptor protein comprises MS2, PP7, Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s, PRR1.

In certain embodiments, the cell is a eukaryotic cell. In certain embodiments, the eukaryotic cell is a mammalian cell, optionally a mouse cell. In certain embodiments, the mammalian cell is a human cell.

In certain embodiments, a first adaptor protein is associated with a p65 domain and a second adaptor protein is associated with a HSF1 domain.

In certain embodiments, the composition comprises a nucleic acid modifying complex having at least three effector domains, at least one of which is associated with the nucleic acid modifying protein and at least two of which are associated with dead gRNA.

In certain embodiments, the composition further comprises a second gRNA, wherein the second gRNA is a live gRNA capable of hybridizing to a second target sequence such that a second nucleic acid modifying system is directed to a second genomic locus of interest in a cell with detectable indel activity at the second genomic locus resultant from nuclease activity of the nucleic acid modifying protein of the system.

In certain embodiments, the composition further comprises a plurality of dead gRNAs and/or a plurality of live gRNAs.

One aspect of the invention is to take advantage of the modularity and customizability of the gRNA scaffold to establish a series of gRNA scaffolds with different binding sites (in particular aptamers) for recruiting distinct types of effectors in an orthogonal manner. Again, for matters of example and illustration of the broader concept, replacement of the MS2 stem-loops with PP7-interacting stem-loops may be used to bind/recruit repressive elements, enabling multiplexed bidirectional transcriptional control. Thus, in general, gRNA comprising a dead guide may be employed to provide for multiplex transcriptional control and preferred bidirectional transcriptional control. This transcriptional control is most preferred of genes. For example, one or more gRNA comprising dead guide(s) may be employed in targeting the activation of one or more target genes. At the same time, one or more gRNA comprising dead guide(s) may be employed in targeting the repression of one or more target genes. Such a sequence may be applied in a variety of different combinations, for example the target genes are first repressed and then at an appropriate period other targets are activated, or select genes are repressed at the same time as select genes are activated, followed by further activation and/or repression. As a result, multiple components of one or more biological systems may advantageously be addressed together.

In an aspect, the invention provides nucleic acid molecule(s) encoding dead gRNA or the nucleic acid modifying complex or the composition as described herein.

In an aspect, the invention provides a vector system comprising: a nucleic acid molecule encoding dead guide RNA as defined herein. In certain embodiments, the vector system further comprises a nucleic acid molecule(s) encoding nucleic acid modifying protein. In certain embodiments, the vector system further comprises a nucleic acid molecule(s) encoding (live) gRNA. In certain embodiments, the nucleic acid molecule or the vector further comprises regulatory element(s) operable in a eukaryotic cell operably linked to the nucleic acid molecule encoding the guide sequence (gRNA) and/or the nucleic acid molecule encoding nucleic acid modifying protein and/or the optional nuclear localization sequence(s).

In another aspect, structural analysis may also be used to study interactions between the dead guide and the active nucleic acid modifying nuclease that enable DNA binding, but no DNA cutting. In this way amino acids or effector domains important for nuclease activity of nucleic acid modifying protein are determined. Modification of such amino acids allows for improved nucleic acid modifying protein used for gene editing.

A further aspect is combining the use of dead guides as explained herein with other applications of DNA modification, as explained herein as well as known in the art. For example, gRNA comprising dead guide(s) for targeted multiplex gene activation or repression or targeted multiplex bidirectional gene activation/repression may be combined with gRNA comprising guides which maintain nuclease activity, as explained herein. Such gRNA comprising guides which maintain nuclease activity may or may not further include modifications which allow for repression of gene activity (e.g. aptamers). Such gRNA comprising guides which maintain nuclease activity may or may not further include modifications which allow for activation of gene activity (e.g. aptamers). In such a manner, a further means for multiplex gene control is introduced (e.g. multiplex gene targeted activation without nuclease activity/without indel activity may be provided at the same time or in combination with gene targeted repression with nuclease activity).

For example, 1) using one or more gRNA (e.g. 1-50, 1-40, 1-30, 1-20, preferably 1-10, more preferably 1-5) comprising dead guide(s) targeted to one or more genes and further modified with appropriate aptamers for the recruitment of gene activators; 2) may be combined with one or more gRNA (e.g. 1-50, 1-40, 1-30, 1-20, preferably 1-10, more preferably 1-5) comprising dead guide(s) targeted to one or more genes and further modified with appropriate aptamers for the recruitment of gene repressors. 1) and/or 2) may then be combined with 3) one or more gRNA (e.g. 1-50, 1-40, 1-30, 1-20, preferably 1-10, more preferably 1-5) targeted to one or more genes. This combination can then be carried out in turn with 1)+2)+3) with 4) one or more gRNA (e.g. 1-50, 1-40, 1-30, 1-20, preferably 1-10, more preferably 1-5) targeted to one or more genes and further modified with appropriate aptamers for the recruitment of gene activators. This combination can then be carried in turn with 1)+2)+3) +4) with 5) one or more gRNA (e.g. 1-50, 1-40, 1-30, 1-20, preferably 1-10, more preferably 1-5) targeted to one or more genes and further modified with appropriate aptamers for the recruitment of gene repressors. As a result various uses and combinations are included in the invention. For example, combination 1)+2); combination 1)+3); combination 2)+3); combination 1)+2)+3); combination 1)+2)+3)+4); combination 1)+3)+4); combination 2)+3)+4); combination 1)+2)+4); combination 1)+2)+3)+4)+5); combination 1)+3)+4)+5); combination 2)+3)+4)+5); combination 1)+2)+4)+5); combination 1)+2)+3)+5); combination 1)+3)+5); combination 2)+3)+5); combination 1)+2)+5).

In an aspect, the invention provides an algorithm for designing, evaluating, or selecting a dead guide RNA targeting sequence (dead guide sequence) for guiding a nucleic acid modifying system to a target gene locus. In particular, it has been determined that dead guide RNA specificity relates to and can be optimized by varying i) GC content and ii) targeting sequence length. In an aspect, the invention provides an algorithm for designing or evaluating a dead guide RNA targeting sequence that minimizes off-target binding or interaction of the dead guide RNA. In an embodiment of the invention, the algorithm for selecting a dead guide RNA targeting sequence for directing a nucleic acid modifying system to a gene locus in an organism comprises a) locating one or more CRISPR motifs in the gene locus, analyzing the 20 nt sequence downstream of each CRISPR motif by i) determining the GC content of the sequence; and ii) determining whether there are off-target matches of the 15 downstream nucleotides nearest to the CRISPR motif in the genome of the organism, and c) selecting the 15 nucleotide sequence for use in a dead guide RNA if the GC content of the sequence is 70% or less and no off-target matches are identified. In an embodiment, the sequence is selected for a targeting sequence if the GC content is 60% or less. In certain embodiments, the sequence is selected for a targeting sequence if the GC content is 55% or less, 50% or less, 45% or less, 40% or less, 35% or less or 30% or less. In an embodiment, two or more sequences of the gene locus are analyzed and the sequence having the lowest GC content, or the next lowest GC content, or the next lowest GC content is selected. In an embodiment, the sequence is selected for a targeting sequence if no off-target matches are identified in the genome of the organism. In an embodiment, the targeting sequence is selected if no off-target matches are identified in regulatory sequences of the genome.

In an aspect, the invention provides a method of selecting a dead guide RNA targeting sequence for directing a functionalized nucleic acid modifying system to a gene locus in an organism, which comprises: a) locating one or more CRISPR motifs in the gene locus; b) analyzing the 20 nt sequence downstream of each CRISPR motif by: i) determining the GC content of the sequence; and ii) determining whether there are off-target matches of the first 15 nt of the sequence in the genome of the organism; c) selecting the sequence for use in a guide RNA if the GC content of the sequence is 70% or less and no off-target matches are identified. In an embodiment, the sequence is selected if the GC content is 50% or less. In an embodiment, the sequence is selected if the GC content is 40% or less. In an embodiment, the sequence is selected if the GC content is 30% or less. In an embodiment, two or more sequences are analyzed and the sequence having the lowest GC content is selected. In an embodiment, off-target matches are determined in regulatory sequences of the organism. In an embodiment, the gene locus is a regulatory region. An aspect provides a dead guide RNA comprising the targeting sequence selected according to the aforementioned methods.

In an aspect, the invention provides a dead guide RNA for targeting a functionalized nucleic acid modifying system to a gene locus in an organism. In an embodiment of the invention, the dead guide RNA comprises a targeting sequence wherein the CG content of the target sequence is 70% or less, and the first 15 nt of the targeting sequence does not match an off-target sequence downstream from a CRISPR motif in the regulatory sequence of another gene locus in the organism. In certain embodiments, the GC content of the targeting sequence 60% or less, 55% or less, 50% or less, 45% or less, 40% or less, 35% or less or 30% or less. In certain embodiments, the GC content of the targeting sequence is from 70% to 60% or from 60% to 50% or from 50% to 40% or from 40% to 30%. In an embodiment, the targeting sequence has the lowest CG content among potential targeting sequences of the locus.

In an embodiment of the invention, the first 15 nt of the dead guide match the target sequence. In another embodiment, first 14 nt of the dead guide match the target sequence. In another embodiment, the first 13 nt of the dead guide match the target sequence. In another embodiment first 12 nt of the dead guide match the target sequence. In another embodiment, first 11 nt of the dead guide match the target sequence. In another embodiment, the first 10 nt of the dead guide match the target sequence. In an embodiment of the invention the first 15 nt of the dead guide does not match an off-target sequence downstream from a CRISPR motif in the regulatory region of another gene locus. In other embodiments, the first 14 nt, or the first 13 nt of the dead guide, or the first 12 nt of the guide, or the first 11 nt of the dead guide, or the first 10 nt of the dead guide, does not match an off-target sequence downstream from a CRISPR motif in the regulatory region of another gene locus. In other embodiments, the first 15 nt, or 14 nt, or 13 nt, or 12 nt, or 11 nt of the dead guide do not match an off-target sequence downstream from a CRISPR motif in the genome.

In certain embodiments, the dead guide RNA includes additional nucleotides at the 3′-end that do not match the target sequence. Thus, a dead guide RNA that includes the first 15 nt, or 14 nt, or 13 nt, or 12 nt, or 11 nt downstream of a CRISPR motif can be extended in length at the 3′ end to 12 nt, 13 nt, 14 nt, 15 nt, 16 nt, 17 nt, 18 nt, 19 nt, 20 nt, or longer.

The invention provides a method for directing a nucleic acid modifying system, including but not limited to a dead Cas9 (dCas9) or functionalized nucleic acid modifying system (which may comprise a functionalized nucleic acid modifying protein or functionalized guide) to a gene locus. In an aspect, the invention provides a method for selecting a dead guide RNA targeting sequence and directing a functionalized nucleic acid modifying system to a gene locus in an organism. In an aspect, the invention provides a method for selecting a dead guide RNA targeting sequence and effecting gene regulation of a target gene locus by a functionalized nucleic acid modifying system. In certain embodiments, the method is used to effect target gene regulation while minimizing off-target effects. In an aspect, the invention provides a method for selecting two or more dead guide RNA targeting sequences and effecting gene regulation of two or more target gene loci by a functionalized nucleic acid modifying system. In certain embodiments, the method is used to effect regulation of two or more target gene loci while minimizing off-target effects.

In an aspect, the invention provides a method of selecting a dead guide RNA targeting sequence for directing a functionalized nucleic acid modifying protein to a gene locus in an organism, which comprises: a) locating one or more CRISPR motifs in the gene locus; b) analyzing the sequence downstream of each CRISPR motif by: i) selecting 10 to 15 nt adjacent to the CRISPR motif, ii) determining the GC content of the sequence; and c) selecting the 10 to 15 nt sequence as a targeting sequence for use in a guide RNA if the GC content of the sequence is 40% or more. In an embodiment, the sequence is selected if the GC content is 50% or more. In an embodiment, the sequence is selected if the GC content is 60% or more. In an embodiment, the sequence is selected if the GC content is 70% or more. In an embodiment, two or more sequences are analyzed and the sequence having the highest GC content is selected. In an embodiment, the method further comprises adding nucleotides to the 3′ end of the selected sequence which do not match the sequence downstream of the CRISPR motif. An aspect provides a dead guide RNA comprising the targeting sequence selected according to the aforementioned methods.

In an aspect, the invention provides a dead guide RNA for directing a functionalized nucleic acid modifying system to a gene locus in an organism wherein the targeting sequence of the dead guide RNA consists of 10 to 15 nucleotides adjacent to the CRISPR motif of the gene locus, wherein the CG content of the target sequence is 50% or more. In certain embodiments, the dead guide RNA further comprises nucleotides added to the 3′ end of the targeting sequence which do not match the sequence downstream of the CRISPR motif of the gene locus.

In an aspect, the invention provides for a single effector to be directed to one or more, or two or more gene loci. In certain embodiments, the effector is associated with one or more domains of a Cas9, and one or more, or two or more selected dead guide RNAs are used to direct the Cas9-associated effector to one or more, or two or more selected target gene loci. In certain embodiments, the effector is associated with one or more, or two or more selected dead guide RNAs, each selected dead guide RNA, when complexed with a nucleic acid modifying protein, causing its associated effector to localize to the dead guide RNA target. One non-limiting example of such nucleic acid modifying systems modulates activity of one or more, or two or more gene loci subject to regulation by the same transcription factor.

In an aspect, the invention provides for two or more effectors to be directed to one or more gene loci. In certain embodiments, two or more dead guide RNAs are employed, each of the two or more effectors being associated with a selected dead guide RNA, with each of the two or more effectors being localized to the selected target of its dead guide RNA. One non-limiting example of such nucleic acid modifying systems modulates activity of one or more, or two or more gene loci subject to regulation by different transcription factors. Thus, in one non-limiting embodiment, two or more transcription factors are localized to different regulatory sequences of a single gene. In another non-limiting embodiment, two or more transcription factors are localized to different regulatory sequences of different genes. In certain embodiments, one transcription factor is an activator. In certain embodiments, one transcription factor is an inhibitor. In certain embodiments, one transcription factor is an activator and another transcription factor is an inhibitor. In certain embodiments, gene loci expressing different components of the same regulatory pathway are regulated. In certain embodiments, gene loci expressing components of different regulatory pathways are regulated.

In an aspect, the invention also provides a method and algorithm for designing and selecting dead guide RNAs that are specific for target DNA cleavage or target binding and gene regulation mediated by a nucleic acid modifying system. In certain embodiments, the nucleic acid modifying system provides orthogonal gene control using an active nucleic acid modifying protein which cleaves target DNA at one gene locus while at the same time binds to and promotes regulation of another gene locus.

In an aspect, the invention provides an method of selecting a dead guide RNA targeting sequence for directing a functionalized nucleic acid modifying protein to a gene locus in an organism, without cleavage, which comprises a) locating one or more CRISPR motifs in the gene locus; b) analyzing the sequence downstream of each CRISPR motif by i) selecting 10 to 15 nt adjacent to the CRISPR motif, ii) determining the GC content of the sequence, and c) selecting the 10 to 15 nt sequence as a targeting sequence for use in a dead guide RNA if the GC content of the sequence is 30% more, 40% or more. In certain embodiments, the GC content of the targeting sequence is 35% or more, 40% or more, 45% or more, 50% or more, 55% or more, 60% or more, 65% or more, or 70% or more. In certain embodiments, the GC content of the targeting sequence is from 30% to 40% or from 40% to 50% or from 50% to 60% or from 60% to 70%. In an embodiment of the invention, two or more sequences in a gene locus are analyzed and the sequence having the highest GC content is selected.

In an embodiment of the invention, the portion of the targeting sequence in which GC content is evaluated is 10 to 15 contiguous nucleotides of the 15 target nucleotides nearest to the PAM. In an embodiment of the invention, the portion of the guide in which GC content is considered is the 10 to 11 nucleotides or 11 to 12 nucleotides or 12 to 13 nucleotides or 13, or 14, or 15 contiguous nucleotides of the 15 nucleotides nearest to the PAM.

In an aspect, the invention further provides an algorithm for identifying dead guide RNAs which promote nucleic acid modifying system gene locus cleavage while avoiding functional activation or inhibition. It is observed that increased GC content in dead guide RNAs of 16 to 20 nucleotides coincides with increased DNA cleavage and reduced functional activation.

It is also demonstrated herein that efficiency of functionalized nucleic acid modifying protein can be increased by addition of nucleotides to the 3′ end of a guide RNA which do not match a target sequence downstream of the CRISPR motif. For example, of dead guide RNA 11 to 15 nt in length, shorter guides may be less likely to promote target cleavage, but are also less efficient at promoting nucleic acid modifying system binding and functional control. In certain embodiments, addition of nucleotides that don't match the target sequence to the 3′ end of the dead guide RNA increase activation efficiency while not increasing undesired target cleavage. In an aspect, the invention also provides a method and algorithm for identifying improved dead guide RNAs that effectively promote nucleic acid modifying system function in DNA binding and gene regulation while not promoting DNA cleavage. Thus, in certain embodiments, the invention provides a dead guide RNA that includes the first 15 nt, or 14 nt, or 13 nt, or 12 nt, or 11 nt downstream of a CRISPR motif and is extended in length at the 3′ end by nucleotides that mismatch the target to 12 nt, 13 nt, 14 nt, 15 nt, 16 nt, 17 nt, 18 nt, 19 nt, 20 nt, or longer.

In an aspect, the invention provides a method for effecting selective orthogonal gene control. As will be appreciated from the disclosure herein, dead guide selection according to the invention, taking into account guide length and GC content, provides effective and selective transcription control by a functional nucleic acid modifying system, for example to regulate transcription of a gene locus by activation or inhibition and minimize off-target effects. Accordingly, by providing effective regulation of individual target loci, the invention also provides effective orthogonal regulation of two or more target loci.

In certain embodiments, orthogonal gene control is by activation or inhibition of two or more target loci. In certain embodiments, orthogonal gene control is by activation or inhibition of one or more target locus and cleavage of one or more target locus.

In one aspect, the invention provides a cell comprising a non-naturally occurring nucleic acid modifying system comprising one or more dead guide RNAs disclosed or made according to a method or algorithm described herein wherein the expression of one or more gene products has been altered. In an embodiment of the invention, the expression in the cell of two or more gene products has been altered. The invention also provides a cell line from such a cell.

In one aspect, the invention provides a multicellular organism comprising one or more cells comprising a non-naturally occurring nucleic acid modifying system comprising one or more dead guide RNAs disclosed or made according to a method or algorithm described herein. In one aspect, the invention provides a product from a cell, cell line, or multicellular organism comprising a non-naturally occurring nucleic acid modifying system comprising one or more dead guide RNAs disclosed or made according to a method or algorithm described herein.

A further aspect of this invention is the use of gRNA comprising dead guide(s) as described herein, optionally in combination with gRNA comprising guide(s) as described herein or in the state of the art, in combination with systems e.g. cells, transgenic animals, transgenic mice, inducible transgenic animals, inducible transgenic mice) which are engineered for either overexpression of nucleic acid modifying protein or preferably knock in nucleic acid modifying protein. As a result a single system (e.g. transgenic animal, cell) can serve as a basis for multiplex gene modifications in systems/network biology. On account of the dead guides, this is now possible in both in vitro, ex vivo, and in vivo.

For example, once the nucleic acid modifying composition is provided for, one or more dead gRNAs may be provided to direct multiplex gene regulation, and preferably multiplex bidirectional gene regulation. The one or more dead gRNAs may be provided in a spatially and temporally appropriate manner if necessary or desired (for example tissue specific induction). On account that the transgenic/inducible system is provided for (e.g. expressed) in the cell, tissue, animal of interest, both gRNAs comprising dead guides or gRNAs comprising guides are equally effective. In the same manner, a further aspect of this invention is the use of gRNA comprising dead guide(s) as described herein, optionally in combination with gRNA comprising guide(s) as described herein or in the state of the art, in combination with systems (e.g. cells, transgenic animals, transgenic mice, inducible transgenic animals, inducible transgenic mice) which are engineered for knockout nucleic acid modifying protein.

As a result, the combination of dead guides as described herein with DNA modification applications described herein and DNA modifications applications known in the art results in a highly efficient and accurate means for multiplex screening of systems (e.g. network biology). Such screening allows, for example, identification of specific combinations of gene activities for identifying genes responsible for diseases (e.g. on/off combinations), in particular gene related diseases. A preferred application of such screening is cancer. In the same manner, screening for treatment for such diseases is included in the invention. Cells or animals may be exposed to aberrant conditions resulting in disease or disease like effects. Candidate compositions may be provided and screened for an effect in the desired multiplex environment. For example, a patient's cancer cells may be screened for which gene combinations will cause them to die, and then use this information to establish appropriate therapies.

In one aspect, the invention provides a kit comprising one or more of the components described herein. The kit may include dead guides as described herein with or without guides as described herein.

The structural information provided herein allows for interrogation of dead gRNA interaction with the target DNA and the nucleic acid modifying protein permitting engineering or alteration of dead gRNA structure to optimize functionality of the entire nucleic acid modifying system. For example, loops of the dead gRNA may be extended, without colliding with the nucleic acid modifying protein by the insertion of adaptor proteins that can bind to RNA. These adaptor proteins can further recruit effector proteins or fusions which comprise one or more effector domains.

In some preferred embodiments, the effector domain is a transcriptional activation domain, preferably VP64. In some embodiments, the effector domain is a transcription repression domain, preferably KRAB. In some embodiments, the transcription repression domain is SID, or concatemers of SID (e.g. SID4X). In some embodiments, the effector domain is an epigenetic modifying domain, such that an epigenetic modifying enzyme is provided. In some embodiments, the effector domain is an activation domain, which may be the P65 activation domain.

An aspect of the invention is that the above elements are comprised in a single composition or comprised in individual compositions. These compositions may advantageously be applied to a host to elicit a functional effect on the genomic level.

In general, the dead gRNA are modified in a manner that provides specific binding sites (e.g. aptamers) for adaptor proteins comprising one or more effector domains (e.g. via fusion protein) to bind to. The modified dead gRNA are modified such that once the dead gRNA forms a nucleic acid modifying complex (i.e. nucleic acid modifying protein binding to dead gRNA and target) the adaptor proteins bind and, the effector domain on the adaptor protein is positioned in a spatial orientation which is advantageous for the attributed function to be effective. For example, if the effector domain is a transcription activator (e.g. VP64 or p65), the transcription activator is placed in a spatial orientation which allows it to affect the transcription of the target. Likewise, a transcription repressor will be advantageously positioned to affect the transcription of the target and a nuclease (e.g. Fok1) will be advantageously positioned to cleave or partially cleave the target.

The skilled person will understand that modifications to the dead gRNA which allow for binding of the adaptor+effector domain but not proper positioning of the adaptor+effector domain (e.g. due to steric hindrance within the three dimensional structure of the nucleic acid modifying complex) are modifications which are not intended. The one or more modified dead gRNA may be modified at the tetra loop, the stem loop 1, stem loop 2, or stem loop 3, as described herein, preferably at either the tetra loop or stem loop 2, and most preferably at both the tetra loop and stem loop 2.

As explained herein the effector domains may be, for example, one or more domains from the group consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g. light inducible). In some cases it is advantageous that additionally at least one NLS is provided. In some instances, it is advantageous to position the NLS at the N terminus. When more than one effector domain is included, the effector domains may be the same or different.

The dead gRNA may be designed to include multiple binding recognition sites (e.g. aptamers) specific to the same or different adaptor protein. The dead gRNA may be designed to bind to the promoter region −1000-+1 nucleic acids upstream of the transcription start site (i.e. TSS), preferably −200 nucleic acids. This positioning improves effector domains which affect gene activation (e.g. transcription activators) or gene inhibition (e.g. transcription repressors). The modified dead gRNA may be one or more modified dead gRNAs targeted to one or more target loci (e.g. at least 1 gRNA, at least 2 gRNA, at least 5 gRNA, at least 10 gRNA, at least 20 gRNA, at least 30 gRNA, at least 50 gRNA) comprised in a composition.

The adaptor protein may be any number of proteins that binds to an aptamer or recognition site introduced into the modified dead gRNA and which allows proper positioning of one or more effector domains, once the dead gRNA has been incorporated into the nucleic acid modifying complex, to affect the target with the attributed function. As explained in detail in this application such may be coat proteins, preferably bacteriophage coat proteins. The effector domains associated with such adaptor proteins (e.g. in the form of fusion protein) may include, for example, one or more domains from the group consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g. light inducible). Preferred domains are Fok1, VP64, P65, HSF1, MyoD1. In the event that the effector domain is a transcription activator or transcription repressor it is advantageous that additionally at least an NLS is provided and preferably at the N terminus. When more than one effector domain is included, the effector domains may be the same or different. The adaptor protein may utilize known linkers to attach such effector domains.

Thus, the modified dead gRNA, the (inactivated) nucleic acid modifying protein (with or without effector domains), and the binding protein with one or more effector domains, may each individually be comprised in a composition and administered to a host individually or collectively. Alternatively, these components may be provided in a single composition for administration to a host. Administration to a host may be performed via viral vectors known to the skilled person or described herein for delivery to a host (e.g. lentiviral vector, adenoviral vector, AAV vector). As explained herein, use of different selection markers (e.g. for lentiviral gRNA selection) and concentration of gRNA (e.g. dependent on whether multiple gRNAs are used) may be advantageous for eliciting an improved effect.

On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different effector domains to elicit one or more genomic locus events. The compositions may be applied in a wide variety of methods for screening in libraries in cells and functional modeling in vivo (e.g. gene activation of lincRNA and identification of function; gain-of-function modeling; loss-of-function modeling; the use the compositions of the invention to establish cell lines and transgenic animals for optimization and screening purposes).

The current invention comprehends the use of the compositions of the current invention to establish and utilize conditional or inducible nucleic acid modifying transgenic cell/animals, which are not believed prior to the present invention or application. For example, the target cell comprises nucleic acid modifying protein conditionally or inducibly (e.g. in the form of Cre dependent constructs) and/or the adaptor protein conditionally or inducibly and, on expression of a vector introduced into the target cell, the vector expresses that which induces or gives rise to the condition of nucleic acid modifying protein expression and/or adaptor expression in the target cell. By applying the teaching and compositions of the current invention with the known method of creating a nucleic acid modifying complex, inducible genomic events affected by effector domains are also an aspect of the current invention. One example of this is the creation of a nucleic acid modifying protein knock-in/conditional transgenic animal (e.g. mouse comprising e.g. a Lox-Stop-polyA-Lox(LSL) cassette) and subsequent delivery of one or more compositions providing one or more modified dead gRNA (e.g. −200 nucleotides to TSS of a target gene of interest for gene activation purposes) as described herein (e.g. modified dead gRNA with one or more aptamers recognized by coat proteins, e.g. MS2), one or more adaptor proteins as described herein (MS2 binding protein linked to one or more VP64) and means for inducing the conditional animal (e.g. Cre recombinase for rendering nucleic acid modifying protein expression inducible). Alternatively, the adaptor protein may be provided as a conditional or inducible element with a conditional or inducible nucleic acid modifying protein to provide an effective model for screening purposes, which advantageously only requires minimal design and administration of specific dead gRNAs for a broad number of applications.

In another aspect the dead guides are further modified to improve specificity. Protected dead guides may be synthesized, whereby secondary structure is introduced into the 3′ end of the dead guide to improve its specificity. A protected guide RNA (pgRNA) comprises a guide sequence capable of hybridizing to a target sequence in a genomic locus of interest in a cell and a protector strand, wherein the protector strand is optionally complementary to the guide sequence and wherein the guide sequence may in part be hybridizable to the protector strand. The pgRNA optionally includes an extension sequence. The thermodynamics of the pgRNA-target DNA hybridization is determined by the number of bases complementary between the guide RNA and target DNA. By employing ‘thermodynamic protection’, specificity of dead gRNA can be improved by adding a protector sequence. For example, one method adds a complementary protector strand of varying lengths to the 3′ end of the guide sequence within the dead gRNA. As a result, the protector strand is bound to at least a portion of the dead gRNA and provides for a protected gRNA (pgRNA). In turn, the dead gRNA references herein may be easily protected using the described embodiments, resulting in pgRNA. The protector strand can be either a separate RNA transcript or strand or a chimeric version joined to the 3′ end of the dead gRNA guide sequence.

### Tandem Guides and Uses in a Multiplex (Tandem) Targeting Approach

The inventors have shown that nucleic acid modifying compositions as defined herein can employ more than one RNA guide without losing activity. This enables the use of the nucleic acid modifying proteins, systems or complexes as defined herein for targeting multiple DNA targets, genes or gene loci, with a single enzyme, system or complex as defined herein. The guide RNAs may be tandemly arranged, optionally separated by a nucleotide sequence such as a direct repeat as defined herein. The position of the different guide RNAs is the tandem does not influence the activity. It is noted that the terms “nucleic acid modifying system” and “nucleic acid modifying complex” are used interchangeably. Also the terms “protein” or “nucleic acid modifying protein” can be used interchangeably. In preferred embodiments, said nucleic acid modifying protein comprises one or more domains of a Cas9, or other Cas protein, in particular, a truncated Cas protein, or one or more domains of any one of the modified or mutated variants thereof described herein elsewhere.

In one aspect, the invention provides a non-naturally occurring or engineered nucleic acid modifying protein comprising one or more domains of a CRISPR enzyme, preferably a class 2 CRISPR enzyme, preferably a Type V or VI CRISPR enzyme as described herein, such as without limitation Cas9 as described herein elsewhere, used for tandem or multiplex targeting. It is to be understood that the nucleic acid modifying protein nucleic acid modifying enzymes, complexes, or systems according to the invention as described herein elsewhere may be used in such an approach. Any of the methods, products, compositions and uses as described herein elsewhere are equally applicable with the multiplex or tandem targeting approach further detailed below. By means of further guidance, the following particular aspects and embodiments are provided.

In one aspect, the invention provides for the use of a nucleic acid modifying protein, complex or system as defined herein for targeting multiple gene loci. In one embodiment, this can be established by using multiple (tandem or multiplex) guide RNA (gRNA) sequences.

In one aspect, the invention provides methods for using one or more elements of a nucleic acid modifying protein, complex or system as defined herein for tandem or multiplex targeting, wherein said nucleic acid modifying system comprises multiple guide RNA sequences. Preferably, said gRNA sequences are separated by a nucleotide sequence, such as a direct repeat as defined herein elsewhere.

The nucleic acid modifying protein, system or complex as defined herein provides an effective means for modifying multiple target polynucleotides. The nucleic acid modifying protein, system or complex as defined herein has a wide variety of utility including modifying (e.g., deleting, inserting, translocating, inactivating, activating) one or more target polynucleotides in a multiplicity of cell types. As such the nucleic acid modifying protein, system or complex as defined herein of the invention has a broad spectrum of applications in, e.g., gene therapy, drug screening, disease diagnosis, and prognosis, including targeting multiple gene loci within a single nucleic acid modifying system.

In one aspect, the invention provides a nucleic acid modifying composition, system or complex as defined herein, i.e. a nucleic acid modifying complex having a nucleic acid modifying composition associated therewith, and multiple guide RNAs that target multiple nucleic acid molecules such as DNA molecules, whereby each of said multiple guide RNAs specifically targets its corresponding nucleic acid molecule, e.g., DNA molecule. Each nucleic acid molecule target, e.g., DNA molecule can encode a gene product or encompass a gene locus. Using multiple guide RNAs hence enables the targeting of multiple gene loci or multiple genes. In some embodiments the nucleic acid modifying protein may cleave the DNA molecule encoding the gene product. In some embodiments expression of the gene product is altered. The nucleic acid modifying protein and the guide RNAs do not naturally occur together. The invention comprehends the guide RNAs comprising tandemly arranged guide sequences. The invention further comprehends coding sequences for the DNA binding protein being codon optimized for expression in a eukaryotic cell. In a preferred embodiment the eukaryotic cell is a mammalian cell, a plant cell or a yeast cell and in a more preferred embodiment the mammalian cell is a human cell. Expression of the gene product may be decreased. The nucleic acid modifying protein may form part of a nucleic acid modifying system or complex, which further comprises tandemly arranged guide RNAs (gRNAs) comprising a series of 2, 3, 4, 5, 6, 7, 8, 9, 10, 15, 25, 25, 30, or more than 30 guide sequences, each capable of specifically hybridizing to a target sequence in a genomic locus of interest in a cell. In some embodiments, the functional nucleic acid modifying system or complex binds to the multiple target sequences. In some embodiments, the functional nucleic acid modifying system or complex may edit the multiple target sequences, e.g., the target sequences may comprise a genomic locus, and in some embodiments there may be an alteration of gene expression. In some embodiments, the functional nucleic acid modifying system or complex may comprise further effector domains. In some embodiments, the invention provides a method for altering or modifying expression of multiple gene products. The method may comprise introducing into a cell containing said target nucleic acids, e.g., DNA molecules, or containing and expressing target nucleic acid, e.g., DNA molecules; for instance, the target nucleic acids may encode gene products or provide for expression of gene products (e.g., regulatory sequences).

In preferred embodiments the nucleic acid modifying composition used for multiplex targeting comprises one or more domains of a Cas9, or the nucleic acid modifying system or complex comprises one or more domains of a Cas9. In some embodiments, the nucleic acid modifying protein used for multiplex targeting comprises one or more domains of AsCas9, or the nucleic acid modifying system or complex used for multiplex targeting comprises one or more domains of an AsCas9. In some embodiments, the nucleic acid modifying protein comprises one or more domains of an LbCas9, or the nucleic acid modifying system or complex comprises one or more domains of LbCas9. In some embodiments, the nucleic acid modifying protein used for multiplex targeting cleaves both strands of DNA to produce a double strand break (DSB). In some embodiments, the nucleic acid modifying protein used for multiplex targeting is a nickase. In some embodiments, the nucleic acid modifying protein used for multiplex targeting is a dual nickase.

In some general embodiments, the nucleic acid modifying protein used for multiplex targeting comprises and/or is associated with one or more effector domains. In some more specific embodiments, the nucleic acid modifying protein used for multiplex targeting comprises one or more domains of a deadCas9 as defined herein elsewhere.

In an aspect, the present invention provides a means for delivering the nucleic acid modifying protein, system or complex for use in multiple targeting as defined herein or the polynucleotides defined herein. Non-limiting examples of such delivery means are e.g. particle(s) delivering component(s) of the complex, vector(s) comprising the polynucleotide(s) discussed herein (e.g., encoding the nucleic acid modifying protein, providing the nucleotides encoding the nucleic acid modifying complex). In some embodiments, the vector may be a plasmid or a viral vector such as AAV, or lentivirus. Transient transfection with plasmids, e.g., into HEK cells may be advantageous, especially given the size limitations of AAV and that while Cas9 fits into AAV, one may reach an upper limit with additional guide RNAs.

Also provided is a model that constitutively expresses the nucleic acid modifying protein, complex or system as used herein for use in multiplex targeting. The organism may be transgenic and may have been transfected with the present vectors or may be the offspring of an organism so transfected. In a further aspect, the present invention provides compositions comprising the nucleic acid modifying protein, system and complex as defined herein or the polynucleotides or vectors described herein. Also provides are nucleic acid modifying systems or complexes comprising multiple guide RNAs, preferably in a tandemly arranged format. Said different guide RNAs may be separated by nucleotide sequences such as direct repeats.

Also provided is a method of treating a subject, e.g., a subject in need thereof, comprising inducing gene editing by transforming the subject with the polynucleotide encoding the nucleic acid modifying system or complex or any of polynucleotides or vectors described herein and administering them to the subject. A suitable repair template may also be provided, for example delivered by a vector comprising said repair template. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises the nucleic acid modifying protein, complex or system comprising multiple guide RNAs, preferably tandemly arranged. Where any treatment is occurring ex vivo, for example in a cell culture, then it will be appreciated that the term ‘subject’ may be replaced by the phrase “cell or cell culture.”

Compositions comprising nucleic acid modifying composition, complex or system comprising multiple guide RNAs, preferably tandemly arranged, or the polynucleotide or vector encoding or comprising said nucleic acid modifying protein, complex or system comprising multiple guide RNAs, preferably tandemly arranged, for use in the methods of treatment as defined herein elsewhere are also provided. A kit of parts may be provided including such compositions. Use of said composition in the manufacture of a medicament for such methods of treatment are also provided. Use of a nucleic acid modifying system in screening is also provided by the present invention, e.g., gain of function screens. Cells which are artificially forced to overexpress a gene are be able to down regulate the gene over time (re-establishing equilibrium) e.g. by negative feedback loops. By the time the screen starts the unregulated gene might be reduced again. Using an inducible nucleic acid modifying activator allows one to induce transcription right before the screen and therefore minimizes the chance of false negative hits. Accordingly, by use of the instant invention in screening, e.g., gain of function screens, the chance of false negative results may be minimized.

In one aspect, the invention provides an engineered, non-naturally occurring nucleic acid modifying system comprising a nucleic acid modifying protein and multiple guide RNAs that each specifically target a DNA molecule encoding a gene product in a cell, whereby the multiple guide RNAs each target their specific DNA molecule encoding the gene product and the nucleic acid modifying protein cleaves the target DNA molecule encoding the gene product, whereby expression of the gene product is altered; and, wherein the nucleic acid modifying protein and the guide RNAs do not naturally occur together. The invention comprehends the multiple guide RNAs comprising multiple guide sequences, preferably separated by a nucleotide sequence such as a direct repeat and optionally fused to a tracr sequence. In an embodiment of the invention the nucleic acid modifying protein comprises one or more domains of a type II or V or VI CRISPR-Cas protein, and in a more preferred embodiment the nucleic acid modifying protein comprises one or more domains of a Cas9 protein. The invention further comprehends a nucleic acid modifying protein being codon optimized for expression in a eukaryotic cell. In a preferred embodiment the eukaryotic cell is a mammalian cell and in a more preferred embodiment the mammalian cell is a human cell. In a further embodiment of the invention, the expression of the gene product is decreased.

In another aspect, the invention provides an engineered, non-naturally occurring vector system comprising one or more vectors comprising a first regulatory element operably linked to the multiple nucleic acid modifying system guide RNAs that each specifically target a DNA molecule encoding a gene product and a second regulatory element operably linked coding for a nucleic acid modifying protein. Both regulatory elements may be located on the same vector or on different vectors of the system. The multiple guide RNAs target the multiple DNA molecules encoding the multiple gene products in a cell and the nucleic acid modifying protein may cleave the multiple DNA molecules encoding the gene products (it may cleave one or both strands or have substantially no nuclease activity), whereby expression of the multiple gene products is altered; and, wherein the nucleic acid modifying protein and the multiple guide RNAs do not naturally occur together. In a preferred embodiment the nucleic acid modifying protein comprises one or more domains of a Cas9 protein, optionally codon optimized for expression in a eukaryotic cell. In a preferred embodiment the eukaryotic cell is a mammalian cell, a plant cell or a yeast cell and in a more preferred embodiment the mammalian cell is a human cell. In a further embodiment of the invention, the expression of each of the multiple gene products is altered, preferably decreased.

In one aspect, the invention provides a vector system comprising one or more vectors. In some embodiments, the system comprises: (a) a first regulatory element operably linked to a direct repeat sequence and one or more insertion sites for inserting one or more guide sequences up- or downstream (whichever applicable) of the direct repeat sequence, wherein when expressed, the one or more guide sequence(s) direct(s) sequence-specific binding of the nucleic acid modifying complex to the one or more target sequence(s) in a eukaryotic cell, wherein the nucleic acid modifying complex comprises a nucleic acid modifying protein complexed with the one or more guide sequence(s) that is hybridized to the one or more target sequence(s); and (b) a second regulatory element operably linked to protein-coding sequence encoding said nucleic acid modifying protein, preferably comprising at least one nuclear localization sequence and/or at least one NES; wherein components (a) and (b) are located on the same or different vectors of the system. Where applicable, a tracr sequence may also be provided. In some embodiments, component (a) further comprises two or more guide sequences operably linked to the first regulatory element, wherein when expressed, each of the two or more guide sequences direct sequence specific binding of a nucleic acid modifying complex to a different target sequence in a eukaryotic cell. In some embodiments, the nucleic acid modifying complex comprises one or more nuclear localization sequences and/or one or more NES of sufficient strength to drive accumulation of said nucleic acid modifying complex in a detectable amount in or out of the nucleus of a eukaryotic cell. In some embodiments, the first regulatory element is a polymerase III promoter. In some embodiments, the second regulatory element is a polymerase II promoter. In some embodiments, each of the guide sequences is at least 16, 17, 18, 19, 20, 25 nucleotides, or between 16-30, or between 16-25, or between 16-20 nucleotides in length.

Recombinant expression vectors can comprise the polynucleotides encoding the nucleic acid modifying protein, system or complex for use in multiple targeting as defined herein in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

In some embodiments, a host cell is transiently or non-transiently transfected with one or more vectors comprising the polynucleotides encoding the nucleic acid modifying protein, system or complex for use in multiple targeting as defined herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art and exemplified herein elsewhere. Cell lines are available from a variety of sources known to those with skill in the art (see, e.g., the American Type Culture Collection (ATCC) (Manassas, Va.)). In some embodiments, a cell transfected with one or more vectors comprising the polynucleotides encoding the nucleic acid modifying protein, system or complex for use in multiple targeting as defined herein is used to establish a new cell line comprising one or more vector-derived sequences. In some embodiments, a cell transiently transfected with the components of a nucleic acid modifying system or complex for use in multiple targeting as described herein (such as by transient transfection of one or more vectors, or transfection with RNA), and modified through the activity of a nucleic acid modifying system or complex, is used to establish a new cell line comprising cells containing the modification but lacking any other exogenous sequence. In some embodiments, cells transiently or non-transiently transfected with one or more vectors comprising the polynucleotides encoding the nucleic acid modifying protein, system or complex for use in multiple targeting as defined herein, or cell lines derived from such cells are used in assessing one or more test compounds.

The term “regulatory element” is as defined herein elsewhere.

Advantageous vectors include lentiviruses and adeno-associated viruses, and types of such vectors can also be selected for targeting particular types of cells.

In one aspect, the invention provides a eukaryotic host cell comprising (a) a first regulatory element operably linked to a direct repeat sequence and one or more insertion sites for inserting one or more guide RNA sequences up- or downstream (whichever applicable) of the direct repeat sequence, wherein when expressed, the guide sequence(s) direct(s) sequence-specific binding of the nucleic acid modifying complex to the respective target sequence(s) in a eukaryotic cell, wherein the nucleic acid modifying complex comprises a nucleic acid modifying protein complexed with the one or more guide sequence(s) that is hybridized to the respective target sequence(s); and/or (b) a second regulatory element operably linked to an enzyme-coding sequence encoding said nucleic acid modifying protein comprising preferably at least one nuclear localization sequence and/or NES. In some embodiments, the host cell comprises components (a) and (b). Where applicable, a tracr sequence may also be provided. In some embodiments, component (a), component (b), or components (a) and (b) are stably integrated into a genome of the host eukaryotic cell. In some embodiments, component (a) further comprises two or more guide sequences operably linked to the first regulatory element, and optionally separated by a direct repeat, wherein when expressed, each of the two or more guide sequences direct sequence specific binding of a nucleic acid modifying complex to a different target sequence in a eukaryotic cell. In some embodiments, the nucleic acid modifying protein comprises one or more nuclear localization sequences and/or nuclear export sequences or NES of sufficient strength to drive accumulation of said nucleic acid modifying protein in a detectable amount in and/or out of the nucleus of a eukaryotic cell.

In some embodiments, the nucleic acid modifying protein comprises one or more domains of a Cas enzyme that is a type V or VI CRISPR system enzyme. In some embodiments, the Cas enzyme is a Cas9 enzyme. In some embodiments, the Cas9 enzyme is derived from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, or Porphyromonas macacae Cas9, and may include further alterations or mutations of the Cas9 as defined herein elsewhere, and can be a chimeric Cas9. In some embodiments, the Cas9 enzyme is codon-optimized for expression in a eukaryotic cell. In some embodiments, the CRISPR enzyme directs cleavage of one or two strands at the location of the target sequence. In some embodiments, the first regulatory element is a polymerase III promoter. In some embodiments, the second regulatory element is a polymerase II promoter. In some embodiments, the one or more guide sequence(s) is (are each) at least 16, 17, 18, 19, 20, 25 nucleotides, or between 16-30, or between 16-25, or between 16-20 nucleotides in length and can be as described elsewhere herein. When multiple guide RNAs are used, they are preferably separated by a direct repeat sequence. In an aspect, the invention provides a non-human eukaryotic organism; preferably a multicellular eukaryotic organism, comprising a eukaryotic host cell according to any of the described embodiments. In other aspects, the invention provides a eukaryotic organism; preferably a multicellular eukaryotic organism, comprising a eukaryotic host cell according to any of the described embodiments. The organism in some embodiments of these aspects may be an animal; for example, a mammal. Also, the organism may be an arthropod such as an insect. The organism also may be a plant. Further, the organism may be a fungus.

In one aspect, the invention provides a kit comprising one or more of the components described herein. In some embodiments, the kit comprises a vector system and instructions for using the kit. In some embodiments, the vector system comprises (a) a first regulatory element operably linked to a direct repeat sequence and one or more insertion sites for inserting one or more guide sequences up- or downstream (whichever applicable) of the direct repeat sequence, wherein when expressed, the guide sequence directs sequence-specific binding of a nucleic acid modifying complex to a target sequence in a eukaryotic cell, wherein the nucleic acid modifying complex comprises a nucleic acid modifying protein comprising a nucleic acid binding protein complexed with the guide sequence that is hybridized to the target sequence; and/or (b) a second regulatory element operably linked to an protein-coding sequence encoding said nucleic acid modifying protein comprising a nuclear localization sequence. Where applicable, a tracr sequence may also be provided. In some embodiments, the kit comprises components (a) and (b) located on the same or different vectors of the system. In some embodiments, component (a) further comprises two or more guide sequences operably linked to the first regulatory element, wherein when expressed, each of the two or more guide sequences direct sequence specific binding of a nucleic acid modifying complex to a different target sequence in a eukaryotic cell. In some embodiments, the nucleic acid modifying protein comprises one or more nuclear localization sequences of sufficient strength to drive accumulation of said nucleic acid modifying protein in a detectable amount in the nucleus of a eukaryotic cell. In some embodiments, the nucleic acid modifying protein comprises one or more domains of a type V or VI CRISPR system enzyme. In some embodiments, the CRISPR enzyme is a Cas9 enzyme. In some embodiments, the Cas9 enzyme is derived from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, or Porphyromonas macacae Cas9 (e.g., modified to have or be associated with at least one DD), and may include further alteration or mutation of the Cas9, and can be a chimeric Cas9. In some embodiments, the DD-CRISPR enzyme is codon-optimized for expression in a eukaryotic cell. In some embodiments, the DD-CRISPR enzyme directs cleavage of one or two strands at the location of the target sequence. In some embodiments, the DD-CRISPR enzyme lacks or substantially DNA strand cleavage activity (e.g., no more than 5% nuclease activity as compared with a wild type enzyme or enzyme not having the mutation or alteration that decreases nuclease activity). In some embodiments, the first regulatory element is a polymerase III promoter. In some embodiments, the second regulatory element is a polymerase II promoter. In some embodiments, the guide sequence is at least 16, 17, 18, 19, 20, 25 nucleotides, or between 16-30, or between 16-25, or between 16-20 nucleotides in length.

In one aspect, the invention provides a method of modifying multiple target polynucleotides in a host cell such as a eukaryotic cell. In some embodiments, the method comprises allowing a nucleic acid modifying complex to bind to multiple target polynucleotides, e.g., to effect cleavage of said multiple target polynucleotides, thereby modifying multiple target polynucleotides, wherein the nucleic acid modifying complex comprises a nucleic acid modifying protein complexed with multiple guide sequences each of the being hybridized to a specific target sequence within said target polynucleotide, wherein said multiple guide sequences are linked to a direct repeat sequence. Where applicable, a tracr sequence may also be provided (e.g. to provide a single guide RNA, sgRNA). In some embodiments, said cleavage comprises cleaving one or two strands at the location of each of the target sequence by said nucleic acid modifying protein. In some embodiments, said cleavage results in decreased transcription of the multiple target genes. In some embodiments, the method further comprises repairing one or more of said cleaved target polynucleotide by homologous recombination with an exogenous template polynucleotide, wherein said repair results in a mutation comprising an insertion, deletion, or substitution of one or more nucleotides of one or more of said target polynucleotides. In some embodiments, said mutation results in one or more amino acid changes in a protein expressed from a gene comprising one or more of the target sequence(s). In some embodiments, the method further comprises delivering one or more vectors to said eukaryotic cell, wherein the one or more vectors drive expression of one or more of. the nucleic acid modifying protein and the multiple guide RNA sequence linked to a direct repeat sequence. Where applicable, a tracr sequence may also be provided. In some embodiments, said vectors are delivered to the eukaryotic cell in a subject. In some embodiments, said modifying takes place in said eukaryotic cell in a cell culture. In some embodiments, the method further comprises isolating said eukaryotic cell from a subject prior to said modifying. In some embodiments, the method further comprises returning said eukaryotic cell and/or cells derived therefrom to said subject.

In one aspect, the invention provides a method of modifying expression of multiple polynucleotides in a eukaryotic cell. In some embodiments, the method comprises allowing a nucleic acid modifying complex to bind to multiple polynucleotides such that said binding results in increased or decreased expression of said polynucleotides; wherein the nucleic acid modifying complex comprises a nucleic acid modifying protein complexed with multiple guide sequences each specifically hybridized to its own target sequence within said polynucleotide, wherein said guide sequences are linked to a direct repeat sequence. Where applicable, a tracr sequence may also be provided. In some embodiments, the method further comprises delivering one or more vectors to said eukaryotic cells, wherein the one or more vectors drive expression of one or more of: the nucleic acid modifying protein and the multiple guide sequences linked to the direct repeat sequences. Where applicable, a tracr sequence may also be provided.

In one aspect, the invention provides a recombinant polynucleotide comprising multiple guide RNA sequences up- or downstream (whichever applicable) of a direct repeat sequence, wherein each of the guide sequences when expressed directs sequence-specific binding of a nucleic acid modifying complex to its corresponding target sequence present in a eukaryotic cell. In some embodiments, the target sequence is a viral sequence present in a eukaryotic cell. Where applicable, a tracr sequence may also be provided. In some embodiments, the target sequence is a proto-oncogene or an oncogene.

Aspects of the invention encompass a non-naturally occurring or engineered composition that may comprise a guide RNA (gRNA) comprising a guide sequence capable of hybridizing to a target sequence in a genomic locus of interest in a cell and a nucleic acid modifying protein as defined herein that may comprise at least one or more nuclear localization sequences.

An aspect of the invention encompasses methods of modifying a genomic locus of interest to change gene expression in a cell by introducing into the cell any of the compositions described herein.

An aspect of the invention is that the above elements are comprised in a single composition or comprised in individual compositions. These compositions may advantageously be applied to a host to elicit a functional effect on the genomic level.

As used herein, the term “guide RNA” or “gRNA” has the leaning as used herein elsewhere and comprises any polynucleotide sequence having sufficient complementarity with a target nucleic acid sequence to hybridize with the target nucleic acid sequence and direct sequence-specific binding of a nucleic acid-targeting complex to the target nucleic acid sequence. Each gRNA may be designed to include multiple binding recognition sites (e.g., aptamers) specific to the same or different adaptor protein. Each gRNA may be designed to bind to the promoter region −1000-+1 nucleic acids upstream of the transcription start site (i.e. TSS), preferably −200 nucleic acids. This positioning improves effector domains which affect gene activation (e.g., transcription activators) or gene inhibition (e.g., transcription repressors). The modified gRNA may be one or more modified gRNAs targeted to one or more target loci (e.g., at least 1 gRNA, at least 2 gRNA, at least 5 gRNA, at least 10 gRNA, at least 20 gRNA, at least 30 g RNA, at least 50 gRNA) comprised in a composition. Said multiple gRNA sequences can be tandemly arranged and are preferably separated by a direct repeat.

Thus, gRNA, the nucleic acid modifying protein as defined herein may each individually be comprised in a composition and administered to a host individually or collectively. Alternatively, these components may be provided in a single composition for administration to a host. Administration to a host may be performed via viral vectors known to the skilled person or described herein for delivery to a host (e.g., lentiviral vector, adenoviral vector, AAV vector). As explained herein, use of different selection markers (e.g., for lentiviral sgRNA selection) and concentration of gRNA (e.g., dependent on whether multiple gRNAs are used) may be advantageous for eliciting an improved effect. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different effector domains to elicit one or more genomic locus events. The compositions may be applied in a wide variety of methods for screening in libraries in cells and functional modeling in vivo (e.g., gene activation of lincRNA and identification of function; gain-of-function modeling; loss-of-function modeling; the use the compositions of the invention to establish cell lines and transgenic animals for optimization and screening purposes).

In certain embodiments, effector domains are linked directly to guides. For example, a SNAP-tag is an engineered methyltransferase that can be reacted with guides that carry 06-benzylguanine derivatives.

The current invention comprehends the use of the compositions of the current invention to establish and utilize conditional or inducible nucleic acid modifying transgenic cell/animals; see, e.g., Platt et al., Cell (2014), 159(2): 440-455, or PCT patent publications cited herein, such as WO 2014/093622 (PCT/US2013/074667). For example, cells or animals such as non-human animals, e.g., vertebrates or mammals, such as rodents, e.g., mice, rats, or other laboratory or field animals, e.g., cats, dogs, sheep, etc., may be ‘knock-in’ whereby the animal conditionally or inducibly expresses nucleic acid modifying protein akin to Platt et al. The target cell or animal thus comprises the nucleic acid modifying protein comprising one or more domains of a Cas protein conditionally or inducibly (e.g., in the form of Cre dependent constructs), on expression of a vector introduced into the target cell, the vector expresses that which induces or gives rise to the condition of the nucleic acid modifying protein expression in the target cell. By applying the teaching and compositions as defined herein with the known method of creating a nucleic acid modifying complex, inducible genomic events are also an aspect of the current invention. Examples of such inducible events have been described herein elsewhere.

### Genetic Modifications

In some embodiments, phenotypic alteration is preferably the result of genome modification when a genetic disease is targeted, especially in methods of therapy and preferably where a repair template is provided to correct or alter the phenotype.

In some embodiments diseases that may be targeted include those concerned with disease-causing splice defects.

In some embodiments, cellular targets include Hemopoietic Stem/Progenitor Cells (CD34+); Beta cells, stem cells, alpha cells, Human T cells; and Eye (retinal cells)—for example photoreceptor precursor cells.

In some embodiments Gene targets include: Human Beta Globin—HBB (for treating Sickle Cell Anemia, including by stimulating gene-conversion (using closely related HBD gene as an endogenous template)); CD3 (T-Cells); and CEP920—retina (eye), c-peptide. In one particular embodiment, the systems disclosed herein are used for insertion of a polynucleotide encoding a protein into a plynucleotide encoding a secretory protein in a cell. The precise genome editing of the SAGE disclosed herein allow for in-frame insertion of polynucleotides in the exon of the polynucleotide sequence of a secretory protein, allowing the inserted polynucleotide to be expressed, and optionally secreted, as described in the examples.

In some embodiments disease targets also include: cancer; Sickle Cell Anemia (based on a point mutation); HBV, HIV; Beta-Thalassemia; and ophthalmic or ocular disease—for example Leber Congenital Amaurosis (LCA)-causing Splice Defect.

In some embodiments delivery methods include: Cationic Lipid Mediated “direct” delivery of Enzyme-Guide complex (RiboNucleoProtein) and electroporation of plasmid DNA.

Methods, products and uses described herein may be used for non-therapeutic purposes. Furthermore, any of the methods described herein may be applied in vitro and ex vivo.

In another embodiment, the nucleic acid modifying protein is delivered into the cell as a protein. In another and particularly preferred embodiment, the nucleic acid modifying protein is delivered into the cell as a protein or as a nucleotide sequence encoding it. Delivery to the cell as a protein may include delivery of a Ribonucleoprotein (RNP) complex, where the protein is complexed with the multiple guides.

In an aspect, host cells and cell lines modified by or comprising the compositions, systems or modified enzymes of present invention are provided, including stem cells, and progeny thereof.

In an aspect, methods of cellular therapy are provided, where, for example, a single cell or a population of cells is sampled or cultured, wherein that cell or cells is or has been modified ex vivo as described herein, and is then re-introduced (sampled cells) or introduced (cultured cells) into the organism. Stem cells, whether embryonic or induce pluripotent or totipotent stem cells, are also particularly preferred in this regard. But, of course, in vivo embodiments are also envisaged.

Inventive methods can further comprise delivery of templates, such as repair templates, which may be dsODN or ssODN, see below. Delivery of templates may be via the cotemporaneous or separate from delivery of any or all the nucleic acid modifying protein or guide RNAs and via the same delivery mechanism or different. In some embodiments, it is preferred that the template is delivered together with the guide RNAs and, preferably, also the nucleic acid modifying protein. An example may be an AAV vector where the nucleic acid modifying protein comprises one or more domains of a CRISPR Cas protein, as described herein, for example, one or more domains of AsCas9 or LbCas9.

Inventive methods can further comprise: (a) delivering to the cell a double-stranded oligodeoxynucleotide (dsODN) comprising overhangs complimentary to the overhangs created by said double strand break, wherein said dsODN is integrated into the locus of interest; or—(b) delivering to the cell a single-stranded oligodeoxynucleotide (ssODN), wherein said ssODN acts as a template for homology directed repair of said double strand break. Inventive methods can be for the prevention or treatment of disease in an individual, optionally wherein said disease is caused by a defect in said locus of interest. Inventive methods can be conducted in vivo in the individual or ex vivo on a cell taken from the individual, optionally wherein said cell is returned to the individual.

The invention also comprehends products obtained from using nucleic acid modifying protein or nucleic acid modifying enzyme or nucleic acid modifying protein comprising a nucleic acid binding domain, which comprises one or more domains of a Cas9 enzyme or nucleic acid modifying system or nucleic acid modifying complex for use in tandem or multiple targeting as defined herein.

### Escorted Guides for the Nucleic Acid Modifying System According to the Invention

In one aspect the invention provides escorted nucleic acid modifying systems or complexes, especially such a system involving an escorted nucleic acid modifying system guide. By “escorted” is meant that the nucleic acid modifying system or complex or guide is delivered to a selected time or place within a cell, so that activity of the nucleic acid modifying system or complex or guide is spatially or temporally controlled. For example, the activity and destination of the nucleic acid modifying system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted nucleic acid modifying systems or complexes have a gRNA with a functional structure designed to improve gRNA structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, provided herein is a gRNA modified, e.g., by one or more aptamer(s) designed to improve gRNA delivery, including delivery across the cellular membrane, to intracellular compartments, or into the nucleus. Such a structure can include, either in addition to the one or more aptamer(s) or without such one or more aptamer(s), moiety(ies) so as to render the guide deliverable, inducible or responsive to a selected effector. The invention accordingly comprehends an gRNA that responds to normal or pathological physiological conditions, including without limitation pH, hypoxia, 02 concentration, temperature, protein concentration, enzymatic concentration, lipid structure, light exposure, mechanical disruption (e.g. ultrasound waves), magnetic fields, electric fields, or electromagnetic radiation.

An aspect of the invention provides non-naturally occurring or engineered composition comprising an escorted guide RNA (egRNA) comprising:
an RNA guide sequence capable of hybridizing to a target sequence in a genomic locus of interest in a cell; and,
an escort RNA aptamer sequence, wherein the escort aptamer has binding affinity for an aptamer ligand on or in the cell, or the escort aptamer is responsive to a localized aptamer effector on or in the cell, wherein the presence of the aptamer ligand or effector on or in the cell is spatially or temporally restricted.

The escort aptamer may for example change conformation in response to an interaction with the aptamer ligand or effector in the cell.

The escort aptamer may have specific binding affinity for the aptamer ligand.

The aptamer ligand may be localized in a location or compartment of the cell, for example on or in a membrane of the cell. Binding of the escort aptamer to the aptamer ligand may accordingly direct the egRNA to a location of interest in the cell, such as the interior of the cell by way of binding to an aptamer ligand that is a cell surface ligand. In this way, a variety of spatially restricted locations within the cell may be targeted, such as the cell nucleus or mitochondria.

Once intended alterations have been introduced, such as by editing intended copies of a gene in the genome of a cell, continued nucleic acid modifying protein expression in that cell is no longer necessary. Indeed, sustained expression would be undesirable in certain casein case of off-target effects at unintended genomic sites, etc. Thus time-limited expression would be useful. Inducible expression offers one approach, but in addition Applicants have engineered a Self-Inactivating nucleic acid modifying system that relies on the use of a non-coding guide target sequence within the nucleic acid modifying vector itself. Thus, after expression begins, the nucleic acid modifying system will lead to its own destruction, but before destruction is complete it will have time to edit the genomic copies of the target gene (which, with a normal point mutation in a diploid cell, requires at most two edits). Simply, the self inactivating nucleic acid modifying system includes additional RNA (i.e., guide RNA) that targets the coding sequence for the nucleic acid modifying protein itself or that targets one or more non-coding guide target sequences complementary to unique sequences present in one or more of the following: (a) within the promoter driving expression of the non-coding RNA elements, (b) within the promoter driving expression of the nucleic acid modifying protein gene, (c) within 100 bp of the ATG translational start codon in the nucleic acid modifying protein coding sequence, (d) within the inverted terminal repeat (iTR) of a viral delivery vector, e.g., in an AAV genome.

The egRNA may include an RNA aptamer linking sequence, operably linking the escort RNA sequence to the RNA guide sequence.

In embodiments, the egRNA may include one or more photolabile bonds or non-naturally occurring residues.

In one aspect, the escort RNA aptamer sequence may be complementary to a target miRNA, which may or may not be present within a cell, so that only when the target miRNA is present is there binding of the escort RNA aptamer sequence to the target miRNA which results in cleavage of the egRNA by an RNA-induced silencing complex (RISC) within the cell.

In embodiments, the escort RNA aptamer sequence may for example be from 10 to 200 nucleotides in length, and the egRNA may include more than one escort RNA aptamer sequence.

It is to be understood that any of the RNA guide sequences as described herein elsewhere can be used in the egRNA described herein. In certain embodiments of the invention, the guide RNA or mature crRNA comprises, consists essentially of, or consists of a direct repeat sequence and a guide sequence or spacer sequence. In certain embodiments, the guide RNA or mature crRNA comprises, consists essentially of, or consists of a direct repeat sequence linked to a guide sequence or spacer sequence. In certain embodiments the guide RNA or mature crRNA comprises 19 nts of partial direct repeat followed by 23-25 nt of guide sequence or spacer sequence. In certain embodiments, the effector protein is a nucleic acid modifying protein comprising one or more domains of a FnCas9 effector protein and requires at least 16 nt of guide sequence to achieve detectable DNA cleavage and a minimum of 17 nt of guide sequence to achieve efficient DNA cleavage in vitro. In certain embodiments, the direct repeat sequence is located upstream (i.e., 5′) from the guide sequence or spacer sequence. In a preferred embodiment the seed sequence (i.e. the sequence essential critical for recognition and/or hybridization to the sequence at the target locus) of the FnCas9 guide RNA is approximately within the first 5 nt on the 5′ end of the guide sequence or spacer sequence.

The egRNA may be included in a non-naturally occurring or engineered nucleic acid modifying complex composition, together with a nucleic acid modifying protein which may include at least one mutation, for example a mutation so that the nucleic acid modifying protein has no more than 5% of the nuclease activity of a nucleic acid modifying protein not having the at least one mutation, for example having a diminished nuclease activity of at least 97%, or 100% as compared with the nucleic acid modifying protein not having the at least one mutation. The nucleic acid modifying protein may also include one or more nuclear localization sequences. Mutated nucleic acid modifying protein having modulated activity such as diminished nuclease activity are described herein elsewhere.

The engineered nucleic acid modifying composition may be provided in a cell, such as a eukaryotic cell, a mammalian cell, or a human cell.

In embodiments, the compositions described herein comprise a nucleic acid modifying complex having at least three effector domains, at least one of which is associated with nucleic acid modifying protein and at least two of which are associated with egRNA.

The compositions described herein may be used to introduce a genomic locus event in a host cell, such as an eukaryotic cell, in particular a mammalian cell, or a non-human eukaryote, in particular a non-human mammal such as a mouse, in vivo. The genomic locus event may comprise affecting gene activation, gene inhibition, or cleavage in a locus. The compositions described herein may also be used to modify a genomic locus of interest to change gene expression in a cell. Methods of introducing a genomic locus event in a host cell using the nucleic acid modifying protein provided herein are described herein in detail elsewhere. Delivery of the composition may for example be by way of delivery of a nucleic acid molecule(s) coding for the composition, which nucleic acid molecule(s) is operatively linked to regulatory sequence(s), and expression of the nucleic acid molecule(s) in vivo, for example by way of a lentivirus, an adenovirus, or an AAV.

The present invention provides compositions and methods by which gRNA-mediated gene editing activity can be adapted. The invention provides gRNA secondary structures that improve cutting efficiency by increasing gRNA and/or increasing the amount of RNA delivered into the cell. The gRNA may include light labile or inducible nucleotides.

To increase the effectiveness of gRNA, for example gRNA delivered with viral or non-viral technologies, Applicants added secondary structures into the gRNA that enhance its stability and improve gene editing. Separately, to overcome the lack of effective delivery, Applicants modified gRNAs with cell penetrating RNA aptamers; the aptamers bind to cell surface receptors and promote the entry of gRNAs into cells. Notably, the cell-penetrating aptamers can be designed to target specific cell receptors, in order to mediate cell-specific delivery. Applicants also have created guides that are inducible. In an embodiment the binding of the nucleic acid binding domain to a target nucleic acid is inducible. In an embodiment, the target nucleic acid comprises chromosomal DNA, mitochondrial DNA, viral DNA or RNA, bacterial DNA, or fungal DNA.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm2. In a preferred embodiment, a stimulation paradigm of as low as 0.25 sec every 15 sec should result in maximal activation.

Cells involved in the practice of the present invention may be a prokaryotic cell or a eukaryotic cell, advantageously an animal cell a plant cell or a yeast cell, more advantageously a mammalian cell.

The chemical or energy sensitive guide may undergo a conformational change upon induction by the binding of a chemical source or by the energy allowing it act as a guide and have the nucleic acid modifying system or complex function. The invention can involve applying the chemical source or energy so as to have the guide function and the nucleic acid modifying system or complex function; and optionally further determining that the expression of the genomic locus is altered.

There are several different designs of this chemical inducible system: 1. ABI-PYL based system inducible by Abscisic Acid (ABA) (see, e.g., http://stke.sciencemag.org/cgi/content/abstract/sigtrans; 4/164/rs2), 2. FKBP-FRB based system inducible by rapamycin (or related chemicals based on rapamycin) (see, e.g., http://www.nature.com/nmeth/journal/v2/n6/full/nmeth763.html), 3. GID1-GAI based system inducible by Gibberellin (GA) (see, e.g., http://www.nature.com/nchembio/journal/v8/n5/full/nchembio.922.html).

Another system contemplated by the present invention is a chemical inducible system based on change in sub-cellular localization. Applicants also developed a system in which the polypeptide include a nucleic acid binding domain comprising at least five or more Transcription activator-like effector (TALE) monomers and at least one or more half-monomers specifically ordered to target the genomic locus of interest linked to at least one or more effector domains are further linked to a chemical or energy sensitive protein. This protein will lead to a change in the sub-cellular localization of the entire polypeptide (i.e. transportation of the entire polypeptide from cytoplasm into the nucleus of the cells) upon the binding of a chemical or energy transfer to the chemical or energy sensitive protein. This transportation of the entire polypeptide from one sub-cellular compartments or organelles, in which its activity is sequestered due to lack of substrate for the effector domain, into another one in which the substrate is present would allow the entire polypeptide to come in contact with its desired substrate (i.e. genomic DNA in the mammalian nucleus) and result in activation or repression of target gene expression.

This type of system could also be used to induce the cleavage of a genomic locus of interest in a cell when the effector domain is a nuclease.

A chemical inducible system can be an estrogen receptor (ER) based system inducible by 4-hydroxytamoxifen (40HT) (see, e.g., http://www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., http://www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the nucleic acid modifying complex or system, and the binding will induce the change of sub-cellular localization of the polypeptide, leading to the entire polypeptide entering the nucleus of cells. Once inside the nucleus, the guide protein and the other components of the nucleic acid modifying complex will be active and modulating target gene expression in cells.

This type of system could also be used to induce the cleavage of a genomic locus of interest in a cell; and, in this regard, it is noted that the nucleic acid modifying protein is a nuclease. The light could be generated with a laser or other forms of energy sources. The heat could be generated by raise of temperature results from an energy source, or from nano-particles that release heat after absorbing energy from an energy source delivered in the form of radio-wave.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc., as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100 mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz’ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

The rapid transcriptional response and endogenous targeting of the instant invention make for an ideal system for the study of transcriptional dynamics. For example, the instant invention may be used to study the dynamics of variant production upon induced expression of a target gene. On the other end of the transcription cycle, mRNA degradation studies are often performed in response to a strong extracellular stimulus, causing expression level changes in a plethora of genes. The instant invention may be utilized to reversibly induce transcription of an endogenous target, after which point stimulation may be stopped and the degradation kinetics of the unique target may be tracked.

The temporal precision of the instant invention may provide the power to time genetic regulation in concert with experimental interventions. For example, targets with suspected involvement in long-term potentiation (LTP) may be modulated in organotypic or dissociated neuronal cultures, but only during stimulus to induce LTP, so as to avoid interfering with the normal development of the cells. Similarly, in cellular models exhibiting disease phenotypes, targets suspected to be involved in the effectiveness of a particular therapy may be modulated only during treatment. Conversely, genetic targets may be modulated only during a pathological stimulus. Any number of experiments in which timing of genetic cues to external experimental stimuli is of relevance may potentially benefit from the utility of the instant invention.

The in vivo context offers equally rich opportunities for the instant invention to control gene expression. Photoinducibility provides the potential for spatial precision. Taking advantage of the development of optrode technology, a stimulating fiber optic lead may be placed in a precise brain region. Stimulation region size may then be tuned by light intensity. This may be done in conjunction with the delivery of the nucleic acid modifying system or complex of the invention, or, in the case of transgenic nucleic acid modifying protein expressing animals, guide RNA of the invention may be delivered and the optrode technology can allow for the modulation of gene expression in precise brain regions. A transparent nucleic acid modifying protein expressing organism, can have guide RNA of the invention administered to it and then there can be extremely precise laser induced local gene expression changes.

A culture medium for culturing host cells includes a medium commonly used for tissue culture, such as M199-earle base, Eagle MEM (E-MEM), Dulbecco MEM (DMEM), SC-UCM102, UP-SFM (GIBCO BRL), EX-CELL302 (Nichirei), EX-CELL293-S(Nichirei), TFBM-01 (Nichirei), ASF104, among others. Suitable culture media for specific cell types may be found at the American Type Culture Collection (ATCC) or the European Collection of Cell Cultures (ECACC). Culture media may be supplemented with amino acids such as L-glutamine, salts, anti-fungal or anti-bacterial agents such as Fungizone®, penicillin-streptomycin, animal serum, and the like. The cell culture medium may optionally be serum-free.

The invention may also offer valuable temporal precision in vivo. The invention may be used to alter gene expression during a particular stage of development. The invention may be used to time a genetic cue to a particular experimental window. For example, genes implicated in learning may be overexpressed or repressed only during the learning stimulus in a precise region of the intact rodent or primate brain. Further, the invention may be used to induce gene expression changes only during particular stages of disease development. For example, an oncogene may be overexpressed only once a tumor reaches a particular size or metastatic stage. Conversely, proteins suspected in the development of Alzheimer's may be knocked down only at defined time points in the animal's life and within a particular brain region. Although these examples do not exhaustively list the potential applications of the invention, they highlight some of the areas in which the invention may be a powerful technology.

Protected Guides: Enzymes According to the Invention can be Used in Combination with Protected Guide RNAs

In one aspect, an object of the current invention is to further enhance the specificity of nucleic acid modifying protein given individual guide RNAs through thermodynamic tuning of the binding specificity of the guide RNA to target DNA. This is a general approach of introducing mismatches, elongation or truncation of the guide sequence to increase/decrease the number of complimentary bases vs. mismatched bases shared between a genomic target and its potential off-target loci, in order to give thermodynamic advantage to targeted genomic loci over genomic off-targets.

In one aspect, the invention provides for the guide sequence being modified by secondary structure to increase the specificity of the nucleic acid modifying system and whereby the secondary structure can protect against exonuclease activity and allow for 3′ additions to the guide sequence.

In one aspect, the invention provides for hybridizing a “protector RNA” to a guide sequence, wherein the “protector RNA” is an RNA strand complementary to the 5′ end of the guide RNA (gRNA), to thereby generate a partially double-stranded gRNA. In an embodiment of the invention, protecting the mismatched bases with a perfectly complementary protector sequence decreases the likelihood of target DNA binding to the mismatched base pairs at the 3′ end. In embodiments of the invention, additional sequences comprising an extended length may also be present.

Guide RNA (gRNA) extensions matching the genomic target provide gRNA protection and enhance specificity. Extension of the gRNA with matching sequence distal to the end of the spacer seed for individual genomic targets is envisaged to provide enhanced specificity. Matching gRNA extensions that enhance specificity have been observed in cells without truncation. Prediction of gRNA structure accompanying these stable length extensions has shown that stable forms arise from protective states, where the extension forms a closed loop with the gRNA seed due to complimentary sequences in the spacer extension and the spacer seed. These results demonstrate that the protected guide concept also includes sequences matching the genomic target sequence distal of the 20 mer spacer-binding region. Thermodynamic prediction can be used to predict completely matching or partially matching guide extensions that result in protected gRNA states. This extends the concept of protected gRNAs to interaction between X and Z, where X will generally be of length 17-20 nt and Z is of length 1-30 nt. Thermodynamic prediction can be used to determine the optimal extension state for Z, potentially introducing small numbers of mismatches in Z to promote the formation of protected conformations between X and Z. Throughout the present application, the terms “X” and seed length (SL) are used interchangeably with the term exposed length (EpL) which denotes the number of nucleotides available for target DNA to bind; the terms “Y” and protector length (PL) are used interchangeably to represent the length of the protector; and the terms “Z”, “E”, “E′” and EL are used interchangeably to correspond to the term extended length (E×L) which represents the number of nucleotides by which the target sequence is extended.

An extension sequence which corresponds to the extended length (E×L) may optionally be attached directly to the guide sequence at the 3′ end of the protected guide sequence. The extension sequence may be 2 to 12 nucleotides in length. Preferably E×L may be denoted as 0, 2, 4, 6, 8, 10 or 12 nucleotides in length. In a preferred embodiment the E×L is denoted as 0 or 4 nucleotides in length. In a more preferred embodiment the E×L is 4 nucleotides in length. The extension sequence may or may not be complementary to the target sequence.

An extension sequence may further optionally be attached directly to the guide sequence at the 5′ end of the protected guide sequence as well as to the 3′ end of a protecting sequence. As a result, the extension sequence serves as a linking sequence between the protected sequence and the protecting sequence. Without wishing to be bound by theory, such a link may position the protecting sequence near the protected sequence for improved binding of the protecting sequence to the protected sequence.

Addition of gRNA mismatches to the distal end of the gRNA can demonstrate enhanced specificity. The introduction of unprotected distal mismatches in Y or extension of the gRNA with distal mismatches (Z) can demonstrate enhanced specificity. This concept as mentioned is tied to X, Y, and Z components used in protected gRNAs. The unprotected mismatch concept may be further generalized to the concepts of X, Y, and Z described for protected guide RNAs.

In one aspect, the invention provides for enhanced nucleic acid modifying protein specificity wherein the double stranded 3′ end of the protected guide RNA (pgRNA) allows for two possible outcomes: (1) the guide RNA-protector RNA to guide RNA-target DNA strand exchange will occur and the guide will fully bind the target, or (2) the guide RNA will fail to fully bind the target and because nucleic acid modifying protein target cleavage is a multiple step kinetic reaction that requires guide RNA:target DNA binding to activate protein-catalyzed DSBs, wherein protein cleavage does not occur if the guide RNA does not properly bind. According to particular embodiments, the protected guide RNA improves specificity of target binding as compared to a unprotected guide system. According to particular embodiments the protected modified guide RNA improves stability as compared to an unmodified guide system. According to particular embodiments the protector sequence has a length between 3 and 120 nucleotides and comprises 3 or more contiguous nucleotides complementary to another sequence of guide or protector. According to particular embodiments, the protector sequence forms a hairpin. According to particular embodiments the guide RNA further comprises a protected sequence and an exposed sequence. According to particular embodiments the exposed sequence is 1 to 19 nucleotides. More particularly, the exposed sequence is at least 75%, at least 90% or about 100% complementary to the target sequence. According to particular embodiments the guide sequence is at least 90% or about 100% complementary to the protector strand. According to particular embodiments the guide sequence is at least 75%, at least 90% or about 100% complementary to the target sequence. According to particular embodiments, the guide RNA further comprises an extension sequence. More particularly, the extension sequence is operably linked to the 3′ end of the protected guide sequence, and optionally directly linked to the 3′ end of the protected guide sequence. According to particular embodiments the extension sequence is 1-12 nucleotides. According to particular embodiments the extension sequence is operably linked to the guide sequence at the 3′ end of the protected guide sequence and the 5′ end of the protector strand and optionally directly linked to the 3′ end of the protected guide sequence and the 3′ end of the protector strand, wherein the extension sequence is a linking sequence between the protected sequence and the protector strand. According to particular embodiments the extension sequence is 100% not complementary to the protector strand, optionally at least 95%, at least 90%, at least 80%, at least 70%, at least 60%, or at least 50% not complementary to the protector strand. According to particular embodiments the guide sequence further comprises mismatches appended to the end of the guide sequence, wherein the mismatches thermodynamically optimize specificity.

In one aspect, the invention provides an engineered, non-naturally occurring nucleic acid modifying system comprising a nucleic acid modifying protein and a protected guide RNA that targets a DNA molecule encoding a gene product in a cell, whereby the protected guide RNA targets the DNA molecule encoding the gene product and the nucleic acid modifying protein cleaves the DNA molecule encoding the gene product, whereby expression of the gene product is altered; and, wherein the nucleic acid modifying protein and the protected guide RNA do not naturally occur together. The invention comprehends the protected guide RNA comprising a guide sequence fused 3′ to a direct repeat sequence. The invention further comprehends the nucleic acid modifying protein being codon optimized for expression in a Eukaryotic cell. In a preferred embodiment the Eukaryotic cell is a mammalian cell, a plant cell or a yeast cell and in a more preferred embodiment the mammalian cell is a human cell. In a further embodiment of the invention, the expression of the gene product is decreased. In some embodiments, the nucleic acid modifying protein comprises one or more domains of a Acidaminococcus sp. BV3L6, Lachnospiraceae bacterium or Francisella Novicida Cas9, and may include mutated Cas9 derived from these organisms. The protein may comprise one or more domains of a Cas9 homolog or ortholog. In some embodiments, the nucleotide sequence encoding the nucleic acid modifying protein is codon-optimized for expression in a eukaryotic cell. In some embodiments, the nucleic acid modifying protein directs cleavage of one or two strands at the location of the target sequence. In some embodiments, the first regulatory element is a polymerase III promoter. In some embodiments, the second regulatory element is a polymerase II promoter. In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

Advantageous vectors include lentiviruses and adeno-associated viruses, and types of such vectors can also be selected for targeting particular types of cells.

In one aspect, the invention provides a eukaryotic host cell comprising (a) a first regulatory element operably linked to a direct repeat sequence and one or more insertion sites for inserting one or more guide sequences downstream of the direct repeat sequence, wherein when expressed, the guide sequence directs sequence-specific binding of a nucleic acid modifying complex to a target sequence in a eukaryotic cell, wherein the nucleic acid modifying complex comprises a nucleic acid modifying protein complexed with the guide RNA comprising the guide sequence that is hybridized to the target sequence and/or (b) a second regulatory element operably linked to an protein-coding sequence encoding said nucleic acid modifying protein comprising a nuclear localization sequence. In some embodiments, the host cell comprises components (a) and (b). In some embodiments, component (a), component (b), or components (a) and (b) are stably integrated into a genome of the host eukaryotic cell. In some embodiments, component (a) further comprises two or more guide sequences operably linked to the first regulatory element, wherein when expressed, each of the two or more guide sequences direct sequence specific binding of a nucleic acid modifying complex to a different target sequence in a eukaryotic cell. In some embodiments, the nucleic acid modifying protein directs cleavage of one or two strands at the location of the target sequence. In some embodiments, the nucleic acid modifying protein lacks DNA strand cleavage activity. In some embodiments, the first regulatory element is a polymerase III promoter. In some embodiments, the second regulatory element is a polymerase II promoter.

In an aspect, the invention provides a non-human eukaryotic organism; preferably a multicellular eukaryotic organism, comprising a eukaryotic host cell according to any of the described embodiments. In other aspects, the invention provides a eukaryotic organism; preferably a multicellular eukaryotic organism, comprising a eukaryotic host cell according to any of the described embodiments. The organism in some embodiments of these aspects may be an animal; for example a mammal. Also, the organism may be an arthropod such as an insect. The organism also may be a plant or a yeast. Further, the organism may be a fungus.

In one aspect, the invention provides a kit comprising one or more of the components described herein above. In some embodiments, the kit comprises a vector system and instructions for using the kit. In some embodiments, the vector system comprises (a) a first regulatory element operably linked to a direct repeat sequence and one or more insertion sites for inserting one or more guide sequences downstream of the direct repeat sequence, wherein when expressed, the guide sequence directs sequence-specific binding of a nucleic acid modifying complex to a target sequence in a eukaryotic cell, wherein the nucleic acid modifying complex comprises a nucleic acid modifying protein complexed with the protected guide RNA comprising the guide sequence that is hybridized to the target sequence and/or (b) a second regulatory element operably linked to an enzyme-coding sequence encoding said nucleic acid modifying protein comprising a nuclear localization sequence. In some embodiments, the kit comprises components (a) and (b) located on the same or different vectors of the system. In some embodiments, component (a) further comprises two or more guide sequences operably linked to the first regulatory element, wherein when expressed, each of the two or more guide sequences direct sequence specific binding of a nucleic acid modifying complex to a different target sequence in a eukaryotic cell. In some embodiments, the nucleic acid modifying protein comprises one or more nuclear localization sequences of sufficient strength to drive accumulation of said nucleic acid modifying protein in a detectable amount in the nucleus of a eukaryotic cell. In some embodiments, the nucleic acid modifying protein comprises one or more domains of a Acidaminococcus sp. BV3L6, Lachnospiraceae bacterium MA2020 or Francisella tularensis 1 Novicida Cas9 or mutated Cas9 derived from these organisms. The nucleic acid modifying protein may comprise one or more domains from a Cas9 homolog or ortholog. In some embodiments, the nucleic acid modifying protein is codon-optimized for expression in a eukaryotic cell. In some embodiments, the nucleic acid modifying protein directs cleavage of one or two strands at the location of the target sequence. In some embodiments, the nucleic acid modifying protein lacks DNA strand cleavage activity. In some embodiments, the first regulatory element is a polymerase III promoter. In some embodiments, the second regulatory element is a polymerase II promoter.

In one aspect, the invention provides a method of modifying a target polynucleotide in a eukaryotic cell. In some embodiments, the method comprises allowing a nucleic acid modifying complex to bind to the target polynucleotide to effect cleavage of said target polynucleotide thereby modifying the target polynucleotide, wherein the nucleic acid modifying complex comprises a nucleic acid modifying protein complexed with protected guide RNA comprising a guide sequence hybridized to a target sequence within said target polynucleotide. In some embodiments, said cleavage comprises cleaving one or two strands at the location of the target sequence by said nucleic acid modifying protein. In some embodiments, said cleavage results in decreased transcription of a target gene. In some embodiments, the method further comprises repairing said cleaved target polynucleotide by non-homologous end joining (NHEJ)-based gene insertion mechanisms, more particularly with an exogenous template polynucleotide, wherein said repair results in a mutation comprising an insertion, deletion, or substitution of one or more nucleotides of said target polynucleotide. In some embodiments, said mutation results in one or more amino acid changes in a protein expressed from a gene comprising the target sequence. In some embodiments, the method further comprises delivering one or more vectors to said eukaryotic cell, wherein the one or more vectors drive expression of one or more of: the nucleic acid modifying protein, the protected guide RNA comprising the guide sequence linked to direct repeat sequence. In some embodiments, said vectors are delivered to the eukaryotic cell in a subject. In some embodiments, said modifying takes place in said eukaryotic cell in a cell culture. In some embodiments, the method further comprises isolating said eukaryotic cell from a subject prior to said modifying. In some embodiments, the method further comprises returning said eukaryotic cell and/or cells derived therefrom to said subject.

In one aspect, the invention provides a method of modifying expression of a polynucleotide in a eukaryotic cell. In some embodiments, the method comprises allowing a nucleic acid modifying complex to bind to the polynucleotide such that said binding results in increased or decreased expression of said polynucleotide; wherein the CRISPR complex comprises a nucleic acid modifying protein complexed with a protected guide RNA comprising a guide sequence hybridized to a target sequence within said polynucleotide. In some embodiments, the method further comprises delivering one or more vectors to said eukaryotic cells, wherein the one or more vectors drive expression of one or more of: the nucleic acid modifying protein and the protected guide RNA.

In one aspect, the invention provides a method of generating a model eukaryotic cell comprising a mutated disease gene. In some embodiments, a disease gene is any gene associated an increase in the risk of having or developing a disease. In some embodiments, the method comprises (a) introducing one or more vectors into a eukaryotic cell, wherein the one or more vectors drive expression of one or more of: a nucleic acid modifying protein and a protected guide RNA comprising a guide sequence linked to a direct repeat sequence; and (b) allowing a nucleic acid modifying complex to bind to a target polynucleotide to effect cleavage of the target polynucleotide within said disease gene, wherein the nucleic acid modifying complex comprises the nucleic acid modifying protein complexed with the guide RNA comprising the sequence that is hybridized to the target sequence within the target polynucleotide, thereby generating a model eukaryotic cell comprising a mutated disease gene. In some embodiments, said cleavage comprises cleaving one or two strands at the location of the target sequence by said nucleic acid modifying protein. In some embodiments, said cleavage results in decreased transcription of a target gene. In some embodiments, the method further comprises repairing said cleaved target polynucleotide by non-homologous end joining (NHEJ)-based gene insertion mechanisms with an exogenous template polynucleotide, wherein said repair results in a mutation comprising an insertion, deletion, or substitution of one or more nucleotides of said target polynucleotide. In some embodiments, said mutation results in one or more amino acid changes in a protein expression from a gene comprising the target sequence.

In one aspect, the invention provides a method for developing a biologically active agent that modulates a cell signaling event associated with a disease gene. In some embodiments, a disease gene is any gene associated an increase in the risk of having or developing a disease. In some embodiments, the method comprises (a) contacting a test compound with a model cell of any one of the described embodiments; and (b) detecting a change in a readout that is indicative of a reduction or an augmentation of a cell signaling event associated with said mutation in said disease gene, thereby developing said biologically active agent that modulates said cell signaling event associated with said disease gene.

In one aspect, the invention provides a recombinant polynucleotide comprising a protected guide sequence downstream of a direct repeat sequence, wherein the protected guide sequence when expressed directs sequence-specific binding of a nucleic acid modifying complex to a corresponding target sequence present in a eukaryotic cell. In some embodiments, the target sequence is a viral sequence present in a eukaryotic cell. In some embodiments, the target sequence is a proto-oncogene or an oncogene.

In one aspect the invention provides for a method of selecting one or more cell(s) by introducing one or more mutations in a gene in the one or more cell (s), the method comprising: introducing one or more vectors into the cell (s), wherein the one or more vectors drive expression of one or more of: a nucleic acid modifying protein, a protected guide RNA comprising a guide sequence, and an editing template; wherein the editing template comprises the one or more mutations that abolish nucleic acid modifying protein cleavage; allowing non-homologous end joining (NHEJ)-based gene insertion mechanisms of the editing template with the target polynucleotide in the cell(s) to be selected; allowing a nucleic acid modifying complex to bind to a target polynucleotide to effect cleavage of the target polynucleotide within said gene, wherein the nucleic acid modifying complex comprises the nucleic acid modifying protein complexed with the protected guide RNA comprising a guide sequence that is hybridized to the target sequence within the target polynucleotide, wherein binding of the nucleic acid modifying complex to the target polynucleotide induces cell death, thereby allowing one or more cell(s) in which one or more mutations have been introduced to be selected. In a preferred embodiment of the invention the cell to be selected may be a eukaryotic cell. Aspects of the invention allow for selection of specific cells without requiring a selection marker or a two-step process that may include a counter-selection system.

With respect to mutations of the nucleic acid modifying protein, when the protein does not comprise one or more domains of FnCas9, mutations may be as described herein elsewhere; conservative substitution for any of the replacement amino acids is also envisaged. In an aspect the invention provides as to any or each or all embodiments herein-discussed wherein the CRISPR enzyme comprises at least one or more, or at least two or more mutations, wherein the at least one or more mutation or the at least two or more mutations are selected from those described herein elsewhere.

In a further aspect, the invention involves a computer-assisted method for identifying or designing potential compounds to fit within or bind to nucleic acid modifying system or a functional portion thereof or vice versa (a computer-assisted method for identifying or designing potential nucleic acid modifying systems or a functional portion thereof for binding to desired compounds) or a computer-assisted method for identifying or designing potential nucleic acid modifying systems (e.g., with regard to predicting areas of the nucleic acid modifying system to be able to be manipulated—for instance, based on crystal structure data or based on data of Cas9 orthologs, or with respect to where a functional group such as an activator or repressor can be attached to the CRISPR-Cas9 system, or as to Cas9 truncations or as to designing nickases), said method comprising: using a computer system, e.g., a programmed computer comprising a processor, a data storage system, an input device, and an output device, the steps of:

(a) inputting into the programmed computer through said input device data comprising the three-dimensional co-ordinates of a subset of the atoms from or pertaining to the CRISPR-Cas9 crystal structure, e.g., in the CRISPR-Cas9 system binding domain or alternatively or additionally in domains that vary based on variance among Cas9 orthologs or as to Cas9s or as to nickases or as to functional groups, optionally with structural information from CRISPR-Cas9 system complex(es), thereby generating a data set;

(b) comparing, using said processor, said data set to a computer database of structures stored in said computer data storage system, e.g., structures of compounds that bind or putatively bind or that are desired to bind to a CRISPR-Cas9 system or as to Cas9 orthologs (e.g., as Cas9s or as to domains or regions that vary amongst Cas9 orthologs) or as to the CRISPR-Cas9 crystal structure or as to nickases or as to functional groups;

(c) selecting from said database, using computer methods, structure(s)—e.g., CRISPR-Cas9 structures that may bind to desired structures, desired structures that may bind to certain CRISPR-Cas9 structures, portions of the CRISPR-Cas9 system that may be manipulated, e.g., based on data from other portions of the CRISPR-Cas9 crystal structure and/or from Cas9 orthologs, truncated Cas9s, novel nickases or particular functional groups, or positions for attaching functional groups or functional-group-CRISPR-Cas9 systems;

(d) constructing, using computer methods, a model of the selected structure(s); and

(e) outputting to said output device the selected structure(s);

and optionally synthesizing one or more of the selected structure(s);

and further optionally testing said synthesized selected structure(s) as or in a nucleic acid modifying system;

or, said method comprising: providing the co-ordinates of at least two atoms of the CRISPR-Cas9 crystal structure, e.g., at least two atoms of the herein Crystal Structure Table of the CRISPR-Cas9 crystal structure or co-ordinates of at least a sub-domain of the CRISPR-Cas9 crystal structure (“selected co-ordinates”), providing the structure of a candidate comprising a binding molecule or of portions of the CRISPR-Cas9 system that may be manipulated, e.g., based on data from other portions of the CRISPR-Cas9 crystal structure and/or from Cas9 orthologs, or the structure of functional groups, and fitting the structure of the candidate to the selected co-ordinates, to thereby obtain product data comprising CRISPR-Cas9 structures that may bind to desired structures, desired structures that may bind to certain CRISPR-Cas9 structures, portions of the CRISPR-Cas9 system that may be manipulated, truncated Cas9s, novel nickases, or particular functional groups, or positions for attaching functional groups or functional-group-CRISPR-Cas9 systems, with output thereof, and optionally synthesizing compound(s) from said product data and further optionally comprising testing said synthesized compound(s) as or in a nucleic acid modifying system.

The testing can comprise analyzing the nucleic acid modifying system resulting from said synthesized selected structure(s), e.g., with respect to binding, or performing a desired function.

The output in the foregoing methods can comprise data transmission, e.g., transmission of information via telecommunication, telephone, video conference, mass communication, e.g., presentation such as a computer presentation (e.g. POWERPOINT), internet, email, documentary communication such as a computer program (e.g. WORD) document and the like. Accordingly, the invention also comprehends computer readable media containing: atomic co-ordinate data according to the herein-referenced Crystal Structure, said data defining the three-dimensional structure of CRISPR-Cas9 or at least one sub-domain thereof, or structure factor data for CRISPR-Cas9, said structure factor data being derivable from the atomic co-ordinate data of herein-referenced Crystal Structure. The computer readable media can also contain any data of the foregoing methods. The invention further comprehends methods a computer system for generating or performing rational design as in the foregoing methods containing either: atomic co-ordinate data according to herein-referenced Crystal Structure, said data defining the three-dimensional structure of CRISPR-Cas9 or at least one sub-domain thereof, or structure factor data for CRISPR-Cas9, said structure factor data being derivable from the atomic co-ordinate data of herein-referenced Crystal Structure. The invention further comprehends a method of doing business comprising providing to a user the computer system or the media or the three dimensional structure of CRISPR-Cas9 or at least one sub-domain thereof, or structure factor data for CRISPR-Cas9, said structure set forth in and said structure factor data being derivable from the atomic co-ordinate data of herein-referenced Crystal Structure, or the herein computer media or a herein data transmission.

A “binding site” or an “active site” comprises or consists essentially of or consists of a site (such as an atom, a functional group of an amino acid residue or a plurality of such atoms and/or groups) in a binding cavity or region, which may bind to a compound such as a nucleic acid molecule, which is/are involved in binding.

By “fitting”, is meant determining by automatic, or semi-automatic means, interactions between one or more atoms of a candidate molecule and at least one atom of a structure of the invention, and calculating the extent to which such interactions are stable. Interactions include attraction and repulsion, brought about by charge, steric considerations and the like. Various computer-based methods for fitting are described further

By “root mean square (or rms) deviation”, is meant the square root of the arithmetic mean of the squares of the deviations from the mean.

By a “computer system”, is meant the hardware means, software means and data storage means used to analyze atomic coordinate data. The minimum hardware means of the computer-based systems of the present invention typically comprises a central processing unit (CPU), input means, output means and data storage means. Desirably a display or monitor is provided to visualize structure data. The data storage means may be RAM or means for accessing computer readable media of the invention. Examples of such systems are computer and tablet devices running Unix, Windows or Apple operating systems.

By “computer readable media”, is meant any medium or media, which can be read and accessed directly or indirectly by a computer e.g., so that the media is suitable for use in the above-mentioned computer system. Such media include, but are not limited to: magnetic storage media such as floppy discs, hard disc storage medium and magnetic tape; optical storage media such as optical discs or CD-ROM; electrical storage media such as RAM and ROM; thumb drive devices; cloud storage devices and hybrids of these categories such as magnetic/optical storage media.

The invention comprehends the use of the protected guides described herein above in the optimized functional nucleic acid modifying systems described herein.

### Targeting and Delivery

With regard to targeting moieties, mention is made of Deshpande et al, “Current trends in the use of liposomes for tumor targeting,” Nanomedicine (Lond). 8(9), doi:10.2217/nnm.13.118 (2013), and the documents it cites, all of which are incorporated herein by reference. Mention is also made of WO/2016/027264, and the documents it cites, all of which are incorporated herein by reference. And mention is made of Lorenzer et al, “Going beyond the liver: Progress and challenges of targeted delivery of siRNA therapeutics,” Journal of Controlled Release, 203: 1-15 (2015), and the documents it cites, all of which are incorporated herein by reference.

An actively targeting lipid particle or nanoparticle or liposome or lipid bilayer delivery system (generally as to embodiments of the invention, “lipid entity of the invention” delivery systems) are contemplated for use with the engineered compositions and complexes described herein. The lipid entities are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these aspects are within the ambit of the skilled artisan. In the field of active targeting, there are a number of cell-, e.g., tumor-, specific targeting ligands.

Also as to active targeting, with regard to targeting cell surface receptors such as cancer cell surface receptors, targeting ligands on liposomes can provide attachment of liposomes to cells, e.g., vascular cells, via a noninternalizing epitope; and, this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid by layers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirous or AAV. Tf is a monomeric serum glycoprotein of approximately 80 KDa involved in the transport of iron throughout the body. Tf binds to the TfR and translocates into cells via receptor-mediated endocytosis. The expression of TfR is can be higher in certain cells, such as tumor cells (as compared with normal cells and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Lipid entities of the invention can be multifunctional, i.e., employ more than one targeting moiety such as CPP, along with Tf; a bifunctional system; e.g., a combination of Tf and poly-L-arginine which can provide transport across the endothelium of the blood-brain barrier. EGFR, is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody(or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm. With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid by layer). The microenvironment of a cell mass such as a tumor microenvironment can be targeted; for instance, it may be advantageous to target cell mass vasculature, such as the the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG such as APRPG-PEG-modified. VCAM, the vascular endothelium plays a key role in the pathogenesis of inflammation, thrombosis and atherosclerosis. CAMs are involved in inflammatory disorders, including cancer, and are a logical target, E- and P-selectins, VCAM-1 and ICAMs. Can be used to target a lipid entity of the invention., e.g., with PEGylation. Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. αβ-integrins or integrins are a group of transmembrane glycoprotein receptors that mediate attachment between a cell and its surrounding tissues or extracellular matrix. Integrins contain two distinct chains (heterodimers) called α- and β-subunits. The tumor tissue-specific expression of integrin receptors can be been utilized for targeted delivery in the invention, e.g., whereby the targeting moiety can be an RGD peptide such as a cyclic RGD. Aptamers are ssDNA or RNA oligonucleotides that impart high affinity and specific recognition of the target molecules by electrostatic interactions, hydrogen bonding and hydro phobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer). The targeting moiety can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)). Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes. The invention also comprehends redox-triggered delivery: The difference in redox potential between normal and inflamed or tumor tissues, and between the intra- and extra-cellular environments has been exploited for delivery; e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment. Enzymes can also be used as a trigger to release payload. Enzymes, including MMPs (e.g. MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln) (SEQ ID NO: 9) can be incorporated into a linker, and can have antibody targeting, e.g., antibody 2C5. The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe2O3, e.g., those that are less than 10 nm in size. Targeted delivery can be then by exposure to a magnetic field.

Also as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA, cholesteryl-GALA and PEG-GALA may show a highly efficient endosomal release; a pore-forming protein listeriolysin 0 may provide an endosomal escape mechanism; and, histidine-rich peptides have the ability to fuse with the endosomal membrane, resulting in pore formation, and can buffer the proton pump causing membrane lysis.

Also as to active targeting, cell-penetrating peptides (CPPs) facilitate uptake of macromolecules through cellular membranes and, thus, enhance the delivery of CPP-modified molecules inside the cell. CPPs can be split into two classes: amphipathic helical peptides, such as transportan and MAP, where lysine residues are major contributors to the positive charge; and Arg-rich peptides, such as TATp, Antennapedia or penetratin. TATp is a transcription-activating factor with 86 amino acids that contains a highly basic (two Lys and six Arg among nine residues) protein transduction domain, which brings about nuclear localization and RNA binding. Other CPPs that have been used for the modification of liposomes include the following: the minimal protein transduction domain of Antennapedia, a Drosophilia homeoprotein, called penetratin, which is a 16-mer peptide (residues 43-58) present in the third helix of the homeodomain; a 27-amino acid-long chimeric CPP, containing the peptide sequence from the amino terminus of the neuropeptide galanin bound via the Lys residue, mastoparan, a wasp venom peptide; VP22, a major structural component of HSV-1 facilitating intracellular transport and transportan (18-mer) amphipathic model peptide that translocates plasma membranes of mast cells and endothelial cells by both energy-dependent and -independent mechanisms. The invention comprehends a lipid entity of the invention modified with CPP(s), for intracellular delivery that may proceed via energy dependent macropinocytosis followed by endosomal escape. The invention further comprehends organelle-specific targeting. A lipid entity of the invention surface-functionalized with the triphenylphosphonium (TPP) moiety or a lipid entity of the invention with a lipophilic cation, rhodamine 123 can be effective in delivery of cargo to mitochondria. DOPE/sphingomyelin/stearyl-octa-arginine can delivers cargos to the mitochondrial interior via membrane fusion. A lipid entity of the invention surface modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

An embodiment of the invention includes the particle delivery system comprising an actively targeting lipid particle or nanoparticle or liposome or lipid bilayer delivery system; or comprising a lipid particle or nanoparticle or liposome or lipid bilayer comprising a targeting moiety whereby there is active targeting or wherein the targeting moiety is an actively targeting moiety. A targeting moiety can be one or more targeting moieties, and a targeting moiety can be for any desired type of targeting such as, e.g., to target a cell such as any herein-mentioned; or to target an organelle such as any herein-mentioned; or for targeting a response such as to a physical condition such as heat, energy, ultrasound, light, pH, chemical such as enzymatic, or magnetic stimuli; or to target to achieve a particular outcome such as delivery of payload to a particular location, such as by cell penetration.

Exemplary targeting moieties are disclosed in PCT/US2018/057182 at [0492]-[0500]. It should be understood that as to each possible targeting or active targeting moiety herein-discussed, there is an aspect of the invention wherein the delivery system comprises such a targeting or active targeting moiety. Likewise, the disclosure provides exemplary targeting moieties that can be used in the practice of the invention an as to each an aspect of the invention provides a delivery system that comprises such a targeting moiety.

In an embodiment of the particle delivery system, the protein comprises a nucleic acid modifying protein.

In some embodiments a non-capsid protein or protein that is not a virus outer protein or a virus envelope (sometimes herein shorthanded as “non-capsid protein”), such as a nucleic acid modifying protein, can have one or more functional moiety(ies) thereon, such as a moiety for targeting or locating, such as an NLS or NES, or an activator or repressor.

In an embodiment of the particle delivery system, a nucleic acid modifying protein can comprise a tag.

In an aspect, the invention provides a virus particle comprising a capsid or outer protein having one or more hybrid virus capsid or outer proteins comprising the virus capsid or outer protein attached to at least a portion of a non-capsid protein or a nucleic acid modifying protein.

In an aspect, the invention provides an in vitro method of delivery comprising contacting the particle delivery system with a cell, optionally a eukaryotic cell, whereby there is delivery into the cell of constituents of the delivery

In one embodiment, the liposome of the particle delivery system comprises a CRISPR system component.

In one aspect, the invention provides a delivery system comprising one or more hybrid virus capsid proteins in combination with a lipid particle, wherein the hybrid virus capsid protein comprises at least a portion of a virus capsid protein attached to at least a portion of a non-capsid protein.

In an aspect, the invention provides an in vitro, a research or study method of delivery comprising contacting the particle delivery system with a cell, optionally a eukaryotic cell, whereby there is delivery into the cell of constituents of the delivery system, obtaining data or results from the contacting, and transmitting the data or results.

In an aspect, the invention provides a cell from or of an in vitro method of delivery, wherein the method comprises contacting the particle delivery system with a cell, optionally a eukaryotic cell, whereby there is delivery into the cell of constituents of the delivery system, and optionally obtaining data or results from the contacting, and transmitting the data or results.

In an aspect, the invention provides a cell from or of an in vitro method of delivery, wherein the method comprises contacting the particle delivery system with a cell, optionally a eukaryotic cell, whereby there is delivery into the cell of constituents of the delivery system, and optionally obtaining data or results from the contacting, and transmitting the data or results; and wherein the cell product is altered compared to the cell not contacted with the delivery system, for example altered from that which would have been wild type of the cell but for the contacting.

In an embodiment, the cell product is non-human or animal.

In one aspect, the invention provides a particle delivery system comprising a composite virus particle, wherein the composite virus particle comprises a lipid, a virus capsid protein, and at least a portion of a non-capsid protein or peptide. The non-capsid peptide or protein can have a molecular weight of up to one megadalton.

Lipid particles, liposomes, nucleic-acid lipid particles, viral delivery, and particle delivery for use in the present systems are as described in PCT/US2018/057182 at [0511]-[0727].

### Supercharged Proteins

Supercharged proteins are a class of engineered or naturally occurring proteins with unusually high positive or negative net theoretical charge and may be employed in delivery of CRISPR Cas system(s) or component(s) thereof or nucleic acid molecule(s) coding therefor. Both super-negatively and super-positively charged proteins exhibit a remarkable ability to withstand thermally or chemically induced aggregation. Super-positively charged proteins are also able to penetrate mammalian cells. Associating cargo with these proteins, such as plasmid DNA, RNA, or other proteins, can enable the functional delivery of these macromolecules into mammalian cells both in vitro and in vivo. David Liu's lab reported the creation and characterization of supercharged proteins in 2007 (Lawrence et al., 2007, Journal of the American Chemical Society 129, 10110-10112).

The non-viral delivery of RNA and plasmid DNA into mammalian cells are valuable both for research and therapeutic applications (Akinc et al., 2010, Nat. Biotech. 26, 561-569). Purified +36 GFP protein (or other super-positively charged protein) is mixed with RNAs in the appropriate serum-free media and allowed to complex prior addition to cells. Inclusion of serum at this stage inhibits formation of the supercharged protein-RNA complexes and reduces the effectiveness of the treatment. Protoclos have been found to be effective for a variety of cell lines (McNaughton et al., 2009, Proc. Natl. Acad. Sci. USA 106, 6111-6116)

See also, e.g., McNaughton et al., Proc. Natl. Acad. Sci. USA 106, 6111-6116 (2009); Cronican et al., ACS Chemical Biology 5, 747-752 (2010); Cronican et al., Chemistry & Biology 18, 833-838 (2011); Thompson et al., Methods in Enzymology 503, 293-319 (2012); Thompson, D. B., et al., Chemistry & Biology 19 (7), 831-843 (2012). The methods of the super charged proteins may be used and/or adapted for delivery of the CRISPR Cas system of the present invention. These systems of Dr. Lui and documents herein in conjunction with herein teaching can be employed in the delivery of CRISPR Cas system(s) or component(s) thereof or nucleic acid molecule(s) coding therefor.

### Cell Penetrating Peptides (CPPs)

In yet another embodiment, cell penetrating peptides (CPPs) are contemplated for the delivery of the CRISPR Cas system. CPPs are short peptides that facilitate cellular uptake of various molecular cargo (from nanosize particles to small chemical molecules and large fragments of DNA). The term “cargo” as used herein includes but is not limited to the group consisting of therapeutic agents, diagnostic probes, peptides, nucleic acids, antisense oligonucleotides, plasmids, proteins, particles, including nanoparticles, liposomes, chromophores, small molecules and radioactive materials. In aspects of the invention, the cargo may also comprise any component of the CRISPR Cas system or the entire functional CRISPR Cas system. Aspects of the present invention further provide methods for delivering a desired cargo into a subject comprising: (a) preparing a complex comprising the cell penetrating peptide of the present invention and a desired cargo, and (b) orally, intraarticularly, intraperitoneally, intrathecally, intrarterially, intranasally, intraparenchymally, subcutaneously, intramuscularly, intravenously, dermally, intrarectally, or topically administering the complex to a subject. The cargo is associated with the peptides either through chemical linkage via covalent bonds or through non-covalent interactions.

The function of the CPPs are to deliver the cargo into cells, a process that commonly occurs through endocytosis with the cargo delivered to the endosomes of living mammalian cells. Cell-penetrating peptides are of different sizes, amino acid sequences, and charges but all CPPs have one distinct characteristic, which is the ability to translocate the plasma membrane and facilitate the delivery of various molecular cargoes to the cytoplasm or an organelle. CPP translocation may be classified into three main entry mechanisms: direct penetration in the membrane, endocytosis-mediated entry, and translocation through the formation of a transitory structure. CPPs have found numerous applications in medicine as drug delivery agents in the treatment of different diseases including cancer and virus inhibitors, as well as contrast agents for cell labeling. Examples of the latter include acting as a carrier for GFP, MRI contrast agents, or quantum dots. CPPs hold great potential as in vitro and in vivo delivery vectors for use in research and medicine. CPPs typically have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. One of the initial CPPs discovered was the trans-activating transcriptional activator (Tat) from Human Immunodeficiency Virus 1 (HIV-1) which was found to be efficiently taken up from the surrounding media by numerous cell types in culture. Since then, the number of known CPPs has expanded considerably and small molecule synthetic analogues with more effective protein transduction properties have been generated. CPPs include but are not limited to Penetratin, Tat (48-60), Transportan, and (R-AhX-R4) (Ahx=aminohexanoyl).

U.S. Pat. No. 8,372,951, provides a CPP derived from eosinophil cationic protein (ECP) which exhibits highly cell-penetrating efficiency and low toxicity. Aspects of delivering the CPP with its cargo into a vertebrate subject are also provided. Further aspects of CPPs and their delivery are described in U.S. Pat. Nos. 8,575,305; 8,614,194 and 8,044,019. CPPs can be used to deliver the CRISPR-Cas system or components thereof. That CPPs can be employed to deliver the CRISPR-Cas system or components thereof is also provided in the manuscript “Gene disruption by cell-penetrating peptide-mediated delivery of Cas9 protein and guide RNA”, by Suresh Ramakrishna, Abu-Bonsrah Kwaku Dad, Jagadish Beloor, et al. Genome Res. 2014 Apr. 2. [Epub ahead of print], incorporated by reference in its entirety, wherein it is demonstrated that treatment with CPP-conjugated recombinant Cas9 protein and CPP-complexed guide RNAs lead to endogenous gene disruptions in human cell lines. In the paper the Cas9 protein was conjugated to CPP via a thioether bond, whereas the guide RNA was complexed with CPP, forming condensed, positively charged particles. It was shown that simultaneous and sequential treatment of human cells, including embryonic stem cells, dermal fibroblasts, HEK293T cells, HeLa cells, and embryonic carcinoma cells, with the modified Cas9 and guide RNA led to efficient gene disruptions with reduced off-target mutations relative to plasmid transfections.

Schwarze et al. demonstrated that intraperitoneal injection of the 120-kilodalton (3-galactosidase protein, fused to the protein transduction domain from the human immunodeficiency virus TAT protein, results in delivery of the biologically active fusion protein to all tissues in mice, including the brain. Schwarze et al., 1999, In Vivo Protein Transduction: Delivery of a Biologically Active Protein into the Mouse, Science 285:1569

Silvio et al. delivered a novel peptide inhibitor of CK2 phosphorylation to tumor cells by linkage to cell penetrating peptide Tat (48-68; GRKKRRQRRRPPQ). Silvio et al., 2004, Antitumor Effect of a Novel Proapoptotic Peptide that Impairs the Phosphorylation by the Protein Kinase 2 (Casein Kinase 2), Cancer Res. 64:7127.

Jo et al. developed recombinant cell-penetrating (CP) forms of suppressor of cytokine signaling 3 (SOCS3) for intracellular delivery to counteract SEB-, LPS- and ConA-induced inflammation and found that CP-SOCS3 ws distributed in multiple organs and persisted in leukocytes and lymphocytes. Jo et al., 2005, Intracellular protein therapy with SOCS3 inhibits inflammation and apoptosis, Nat. Medicine, 11:892.

Kamei et al. produced penetratin analogs indicating that chain length, hydrophobicity, and amphipathicity of the CPPs, as well as their basicity, contribute to their absorption-enhancing efficiency. It was further demonstrated that modified CPPs could be designed that had the capacity to complex with insulin and enhance insulin absorption to a greater extent that the original penetrating. Kamei et al., 2013, Determination of the Optimal Cell-Penetrating Peptide Sequence for Intestinal Insulin Delivery Based on Molecular Orbital Analysis with Self-Organizing Maps, J. Pharm. Sci. 102:469.

These and further examples are set forth in the table below, Table 2.

Peptides and proteins delivered by cell-penetrating peptides.

| CPP | Cargo | Formulation | Assay/result |
|---|---|---|---|
| Tat | β-galactosidase | Covalent | Tissue distribution of β-galactosidase in mice |
| | | conjugation | following IP administration. |
| Tat | P15 | Covalent | Apoptosis in various tumor cell lines and |
| | | conjugation | regression of tumor size upon intratumoral |
| | | | injections to mice. |
| FGF4-derived | suppressor of | Covalent | Uptake into mouse macrophage cells and |
| peptide | cytokine signaling | conjugation | suppression of the production of inflammatory |
| | (SOCS3) | | cytokines in mice following IP administration. |
| R9 | c-Myc, Sox2, Oct4, | Covalent | Induction of fibroblasts from human newborn |
| | Klf4 | conjugation | into pluripotent stem cells. |
| Pep-1 | Various peptides | Physical | Uptake of cargo peptide or protein in cells of |
| | and proteins | complexation | various cell culture models. |
| Penetratin | Insulin, GLP-1, | Physical | Cargo plasma concentration following nasal |
| | exendin-4 | complexation | or intestinal loop administration to rats. |
| PenetraMax | Insulin | Physical | Insulin plasma concentration following |
| | | complexation | intestinal loop administration to rats. |
| Tat | Bcl-x1 | Covalent | Brain distribution of Bcl-x1 and reduction of |
| | | conjugation | cerebral infarction. |
| Tat | NR2B9c | Covalent | Brain concentration of NR2B9c in rats and |
| | | conjugation | reduction of cerebral infarction in mice |
| | | | following IP administration. |
| Tat | GDNF | Covalent | Brain concentration of GDNF and reduction |
| | | conjugation | of cerebral infarction following intravenous |
| | | | administration to mice. |

### Inducible Systems

In an aspect the invention provides a (non-naturally occurring or engineered) inducible nucleic acid modifying protein according to the invention as described herein (nucleic acid modifying system), comprising: a first nucleic acid modifying protein fusion construct attached to a first half of an inducible dimer and a second nucleic acid modifying protein fusion construct attached to a second half of the inducible dimer, wherein the first nucleic acid modifying protein fusion construct is operably linked to one or more nuclear localization signals, wherein the second nucleic acid modifying protein protein fusion construct is operably linked to one or more nuclear export signals, wherein contact with an inducer energy source brings the first and second halves of the inducible dimer together, wherein bringing the first and second halves of the inducible dimer together allows the first and second nucleic acid modifying protein fusion constructs to constitute a functional nucleic acid modifying protein (optionally wherein the nucleic acid modifying system comprises a guide RNA (gRNA) comprising a guide sequence capable of hybridizing to a target sequence in a genomic locus of interest in a cell, and wherein the functional nucleic acid modifying system binds to the target sequence and, optionally, edits the genomic locus to alter gene expression).

In an aspect of the invention in the inducible nucleic acid modifying system, the inducible dimer is or comprises or consists essentially of or consists of an inducible heterodimer. In an aspect, in inducible nucleic acid modifying system, the first half or a first portion or a first fragment of the inducible heterodimer is or comprises or consists of or consists essentially of an FKBP, optionally FKBP12. In an aspect of the invention, in the inducible nucleic acid modifying system, the second half or a second portion or a second fragment of the inducible heterodimer is or comprises or consists of or consists essentially of FRB. In an aspect of the invention, in the inducible nucleic acid modifying system, the arrangement of the first nucleic acid modifying protein fusion construct is or comprises or consists of or consists essentially of N′ terminal nucleic acid modifying protein part-FRB-NES. In an aspect of the invention, in the inducible nucleic acid modifying system, the arrangement of the first nucleic acid modifying protein fusion construct is or comprises or consists of or consists essentially of NES-N′ terminal nucleic acid modifying protein part-FRB-NES. In an aspect of the invention, in the inducible nucleic acid modifying system, the arrangement of the second nucleic acid modifying protein fusion construct is or comprises or consists essentially of or consists of C′ terminal nucleic acid modifying protein part-FKBP-NLS. In an aspect the invention provides in the inducible nucleic acid modifying system, the arrangement of the second nucleic acid modifying protein fusion construct is or comprises or consists of or consists essentially of NLS-C′ terminal nucleic acid modifying protein part-FKBP-NLS. In an aspect, in inducible nucleic acid modifying system there can be a linker that separates the nucleic acid modifying protein part from the half or portion or fragment of the inducible dimer. In an aspect, in the inducible nucleic acid modifying system, the inducer energy source is or comprises or consists essentially of or consists of rapamycin. In an aspect, in inducible nucleic acid modifying system, the inducible dimer is an inducible homodimer. In an aspect, in an inducible nucleic acid modifying system, the nucleic acid modifying protein comprises one or more domains of a AsCpf1, LbCpf1 or FnCpf1.

In an aspect, the invention provides a (non-naturally occurring or engineered) inducible nucleic acid modifying system, comprising: a first nucleic acid modifying protein fusion construct attached to a first half of an inducible heterodimer and a second nucleic acid modifying protein fusion construct attached to a second half of the inducible heterodimer, wherein the first nucleic acid modifying protein fusion construct is operably linked to one or more nuclear localization signals, wherein the second nucleic acid modifying protein fusion construct is operably linked to a nuclear export signal, wherein contact with an inducer energy source brings the first and second halves of the inducible heterodimer together, wherein bringing the first and second halves of the inducible heterodimer together allows the first and second nucleic acid modifying protein fusion constructs to constitute a functional nucleic acid modifying protein (optionally wherein the nucleic acid modifying system comprises a guide RNA (gRNA) comprising a guide sequence capable of hybridizing to a target sequence in a genomic locus of interest in a cell, and wherein the functional nucleic acid modifying system edits the genomic locus to alter gene expression).

Accordingly, the invention comprehends inter alia homodimers as well as heterodimers, dead-nucleic acid modifying protein or nucleic acid modifying protein having essentially no nuclease activity, e.g., through mutation, systems or complexes wherein there is one or more NLS and/or one or more NES; effector domain(s) linked to split nucleic acid modifying protein; methods, including methods of treatment, and uses.

An inducer energy source may be considered to be simply an inducer or a dimerizing agent. The term ‘inducer energy source’ is used herein throughout for consistency. The inducer energy source (or inducer) acts to reconstitute the enzyme. In some embodiments, the inducer energy source brings the two parts of the enzyme together through the action of the two halves of the inducible dimer. The two halves of the inducible dimer therefore are brought tougher in the presence of the inducer energy source. The two halves of the dimer will not form into the dimer (dimerize) without the inducer energy source.

Thus, the two halves of the inducible dimer cooperate with the inducer energy source to dimerize the dimer. This in turn reconstitutes the nucleic acid modifying protein by bringing the first and second parts of the nucleic acid modifying protein together.

The nucleic acid modifying protein fusion constructs each comprise one part of the split nucleic acid modifying protein. These are fused, preferably via a linker such as a GlySer linker described herein, to one of the two halves of the dimer. The two halves of the dimer may be substantially the same two monomers that together that form the homodimer, or they may be different monomers that together form the heterodimer. As such, the two monomers can be thought of as one half of the full dimer.

The nucleic acid modifying protein is split in the sense that the two parts of the nucleic acid modifying protein substantially comprise a functioning nucleic acid modifying protein. That nucleic acid modifying protein may function as a genome editing enzyme (when forming a complex with the target DNA and the guide), such as a nickase or a nuclease (cleaving both strands of the DNA), or it may be a dead-nucleic acid modifying protein which is essentially a DNA-binding protein with very little or no catalytic activity, due to typically mutation(s) in its catalytic domains.

The two parts of the split nucleic acid modifying protein can be thought of as the N′ terminal part and the C′ terminal part of the split nucleic acid modifying protein. The fusion is typically at the split point of the nucleic acid modifying protein. In other words, the C′ terminal of the N′ terminal part of the split nucleic acid modifying protein is fused to one of the dimer halves, whilst the N′ terminal of the C′ terminal part is fused to the other dimer half.

The nucleic acid modifying protein does not have to be split in the sense that the break is newly created. The split point is typically designed in silico and cloned into the constructs. Together, the two parts of the split nucleic acid modifying protein, the N′ terminal and C′ terminal parts, form a full nucleic acid modifying protein, comprising preferably at least 70% or more of the wildtype amino acids (or nucleotides encoding them), preferably at least 80% or more, preferably at least 90% or more, preferably at least 95% or more, and most preferably at least 99% or more of the wildtype amino acids (or nucleotides encoding them). Some trimming may be possible, and mutants are envisaged. Non-functional domains may be removed entirely. What is important is that the two parts may be brought together and that the desired nucleic acid modifying protein function is restored or reconstituted.

The dimer may be a homodimer or a heterodimer.

One or more, preferably two, NLSs may be used in operable linkage to the first nucleic acid modifying protein construct. One or more, preferably two, NESs may be used in operable linkage to the first nucleic acid modifying protein construct. The NLSs and/or the NESs preferably flank the split nucleic acid modifying protein-dimer (i.e., half dimer) fusion, i.e., one NLS may be positioned at the N′ terminal of the first nucleic acid modifying protein construct and one NLS may be at the C′ terminal of the first nucleic acid modifying protein construct. Similarly, one NES may be positioned at the N′ terminal of the second nucleic acid modifying construct and one NES may be at the C′ terminal of the second nucleic acid modifying protein construct. Where reference is made to N′ or C′ terminals, it will be appreciated that these correspond to 5′ ad 3′ ends in the corresponding nucleotide sequence.

A preferred arrangement is that the first nucleic acid modifying protein construct is arranged 5′-NLS-(N′ terminal nucleic acid modifying protein part)-linker-(first half of the dimer)-NLS-3′. A preferred arrangement is that the second nucleic acid modifying protein construct is arranged 5′-NES-(second half of the dimer)-linker-(C′ terminal nucleic acid modifying protein part)-NES-3′. A suitable promoter is preferably upstream of each of these constructs. The two constructs may be delivered separately or together.

In some embodiments, one or all of the NES(s) in operable linkage to the second nucleic acid modifying protein construct may be swapped out for an NLS. However, this may be typically not preferred and, in other embodiments, the localization signal in operable linkage to the second nucleic acid modifying protein construct is one or more NES(s).

It will also be appreciated that the NES may be operably linked to the N′ terminal fragment of the split nucleic acid modifying protein and that the NLS may be operably linked to the C′ terminal fragment of the split nucleic acid modifying protein. However, the arrangement where the NLS is operably linked to the N′ terminal fragment of the split nucleic acid modifying protein and that the NES is operably linked to the C′ terminal fragment of the split nucleic acid modifying protein may be preferred.

The NES functions to localize the second nucleic acid modifying protein fusion construct outside of the nucleus, at least until the inducer energy source is provided (e.g., at least until an energy source is provided to the inducer to perform its function). The presence of the inducer stimulates dimerization of the two nucleic acid modifying protein fusions within the cytoplasm and makes it thermodynamically worthwhile for the dimerized, first and second, nucleic acid modifying protein fusions to localize to the nucleus. Without being bound by theory, Applicants believe that the NES sequesters the second nucleic acid modifying protein fusion to the cytoplasm (i.e., outside of the nucleus). The NLS on the first nucleic acid modifying protein fusion localizes it to the nucleus. In both cases, Applicants use the NES or NLS to shift an equilibrium (the equilibrium of nuclear transport) to a desired direction. The dimerization typically occurs outside of the nucleus (a very small fraction might happen in the nucleus) and the NLSs on the dimerized complex shift the equilibrium of nuclear transport to nuclear localization, so the dimerized and hence reconstituted nucleic acid modifying protein enters the nucleus.

Beneficially, Applicants are able to reconstitute function in the split nucleic acid modifying protein. Transient transfection is used to prove the concept and dimerization occurs in the background in the presence of the inducer energy source. No activity is seen with separate fragments of the nucleic acid modifying protein. Stable expression through lentiviral delivery is then used to develop this and show that a split nucleic acid modifying protein approach can be used.

This present split nucleic acid modifying protein approach is beneficial as it allows the nucleic acid modifying protein activity to be inducible, thus allowing for temporal control. Furthermore, different localization sequences may be used (i.e., the NES and NLS as preferred) to reduce background activity from auto-assembled complexes. Tissue specific promoters, for example one for each of the first and second nucleic acid modifying protein fusion constructs, may also be used for tissue-specific targeting, thus providing spatial control. Two different tissue specific promoters may be used to exert a finer degree of control if required. The same approach may be used in respect of stage-specific promoters or there may a mixture of stage and tissue specific promoters, where one of the first and second nucleic acid modifying protein fusion constructs is under the control of (i.e. operably linked to or comprises) a tissue-specific promoter, whilst the other of the first and second nucleic acid modifying protein fusion constructs is under the control of (i.e. operably linked to or comprises) a stage-specific promoter.

The inducible nucleic acid modifying protein nucleic acid modifying system comprises one or more nuclear localization sequences (NLSs), as described herein, for example as operably linked to the first nucleic acid modifying protein fusion construct. These nuclear localization sequences are ideally of sufficient strength to drive accumulation of said first nucleic acid modifying protein fusion construct in a detectable amount in the nucleus of a eukaryotic cell. Without wishing to be bound by theory, it is believed that a nuclear localization sequence is not necessary for nucleic acid modifying complex activity in eukaryotes, but that including such sequences enhances activity of the system, especially as to targeting nucleic acid molecules in the nucleus, and assists with the operation of the present 2-part system.

Equally, the second nucleic acid modifying protein fusion construct is operably linked to a nuclear export sequence (NES). Indeed, it may be linked to one or more nuclear export sequences. In other words, the number of export sequences used with the second nucleic acid modifying protein fusion construct is preferably 1 or 2 or 3. Typically 2 is preferred, but 1 is enough and so is preferred in some embodiments. Suitable examples of NLS and NES are known in the art. For example, a preferred nuclear export signal (NES) is human protein tyrosin kinase 2. Preferred signals will be species specific.

Where the FRB and FKBP system are used, the FKBP is preferably flanked by nuclear localization sequences (NLSs). Where the FRB and FKBP system are used, the preferred arrangement is N′ terminal nucleic acid modifying protein—FRB—NES:C′ terminal nucleic acid modifying protein-FKBP-NLS. Thus, the first nucleic acid modifying protein fusion construct would comprise the C′ terminal nucleic acid modifying protein part and the second DNA modifying protein fusion construct would comprise the N′ terminal nucleic acid modifying protein part.

Another beneficial aspect to the present invention is that it may be turned on quickly, i.e. that is has a rapid response. It is believed, without being bound by theory, that nucleic acid modifying protein activity can be induced through dimerization of existing (already present) fusion constructs (through contact with the inducer energy source) more rapidly than through the expression (especially translation) of new fusion constructs. As such, the first and second nucleic acid modifying protein fusion constructs may be expressed in the target cell ahead of time, i.e. before nucleic acid modifying protein activity is required. nucleic acid modifying protein activity can then be temporally controlled and then quickly constituted through addition of the inducer energy source, which ideally acts more quickly (to dimerize the heterodimer and thereby provide nucleic acid modifying protein activity) than through expression (including induction of transcription) of nucleic acid modifying protein delivered by a vector, for example.

Applicants demonstrate that nucleic acid modifying protein can be split into two components, which reconstitute a functional nuclease when brought back together. Employing rapamycin sensitive dimerization domains, Applicants generate a chemically inducible nucleic acid modifying protein for temporal control of nucleic acid modifying protein-mediated genome editing and transcription modulation. Put another way, Applicants demonstrate that nucleic acid modifying protein can be rendered chemically inducible by being split into two fragments and that rapamycin-sensitive dimerization domains may be used for controlled reassembly of the nucleic acid modifying protein. Applicants show that the re-assembled nucleic acid modifying protein may be used to mediate genome editing (through nuclease/nickase activity) as well as transcription modulation (as a DNA-binding domain, the so-called “dead nucleic acid modifying protein”).

As such, the use of rapamycin-sensitive dimerization domains is preferred. Reassembly of the nucleic acid modifying protein is preferred. Reassembly can be determined by restoration of binding activity. Where the nucleic acid modifying protein is a nickase or induces a double-strand break, suitable comparison percentages compared to a wildtype are described herein.

Rapamycin treatments can last 12 days. The dose can be 200 nM. This temporal and/or molar dosage is an example of an appropriate dose for Human embryonic kidney 293FT (HEK293FT) cell lines and this may also be used in other cell lines. This figure can be extrapolated out for therapeutic use in vivo into, for example, mg/kg. However, it is also envisaged that the standard dosage for administering rapamycin to a subject is used here as well. By the “standard dosage”, it is meant the dosage under rapamycin's normal therapeutic use or primary indication (i.e. the dose used when rapamycin is administered for use to prevent organ rejection).

It is noteworthy that the preferred arrangement of nucleic acid modifying protein-FRB/FKBP pieces are separate and inactive until rapamycin-induced dimerization of FRB and FKBP results in reassembly of a functional full-length nucleic acid modifying protein nuclease. Thus, it is preferred that first nucleic acid modifying protein fusion construct attached to a first half of an inducible heterodimer is delivered separately and/or is localized separately from the second nucleic acid modifying protein fusion construct attached to a first half of an inducible heterodimer.

To sequester the nucleic acid modifying protein (N)-FRB fragment in the cytoplasm, where it is less likely to dimerize with the nuclear-localized nucleic acid modifying protein-(C)-FKBP fragment, it is preferable to use on nucleic acid modifying protein (N)-FRB a single nuclear export sequence (NES) from the human protein tyrosin kinase 2 (nucleic acid modifying protein (N)-FRB-NES). In the presence of rapamycin, nucleic acid modifying protein (N)-FRB-NES dimerizes with nucleic acid modifying protein (C)-FKBP-2×NLS to reconstitute a complete nucleic acid modifying protein, which shifts the balance of nuclear trafficking toward nuclear import and allows DNA targeting.

With respect to general information on nucleic acid modifying systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); European Patents EP 2 784 162 B1 and EP 2 771 468 B1; European Patent Applications EP 2 771 468 (EP13818570.7), EP 2 764 103 (EP13824232.6), and EP 2 784 162 (EP14170383.5); and PCT Patent Publications PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809). Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/836,127, 61/836,101, 61/836,080 and 61/835,973, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT Patent applications Nos: PCT/US2014/041803, PCT/US2014/041800, PCT/US2014/041809, PCT/US2014/041804 and PCT/US2014/041806, each filed Jun. 10, 2014 Jun. 10, 2014; PCT/US2014/041808 filed Jun. 11, 2014; and PCT/US2014/62558 filed Oct. 28, 2014, and U.S. Provisional Patent Applications Ser. Nos. 61/915,150, 61/915,301, 61/915,267 and 61/915,260, each filed Dec. 12, 2013; 61/757,972 and 61/768,959, filed on Jan. 29, 2013 and Feb. 25, 2013; 61/835,936, 61/836,127, 61/836,101, 61/836,080, 61/835,973, and 61/835,931, filed Jun. 17, 2013; 62/010,888 and 62/010,879, both filed Jun. 11, 2014; 62/010,329 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/054,490, 62/055,484, 62/055,460 and 62/055,487, each filed Sep. 25, 2014; and 62/069,243, filed Oct. 27, 2014. Reference is also made to U.S. provisional patent applications Nos. 62/055,484, 62/055,460, and 62/055,487, filed Sep. 25, 2014; U.S. provisional patent application 61/980,012, filed Apr. 15, 2014; and U.S. provisional patent application 61/939,242 filed Feb. 12, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013. Reference is made to US provisional patent application U.S. Ser. No. 61/980,012 filed Apr. 15, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013.

Mention is also made of U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. application 62/091,462, 12 Dec. 2014, DEAD GUIDES FOR CRISPR TRANSCRIPTION FACTORS; U.S. application 62/096,324, 23 Dec. 2014, DEAD GUIDES FOR CRISPR TRANSCRIPTION FACTORS; U.S. application 62/091,456, 12 Dec. 2014, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, RNA-TARGETING SYSTEM; U.S. application 62/096,656, 24 Dec. 2014, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/054,675, 24 Sep. 2014, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

The subject invention may be used as part of a research program wherein there is transmission of results or data. A computer system (or digital device) may be used to receive, transmit, display and/or store results, analyze the data and/or results, and/or produce a report of the results and/or data and/or analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present invention can be transmitted over such networks or connections (or any other suitable means for transmitting information, including but not limited to mailing a physical report, such as a print-out) for reception and/or for review by a receiver. The receiver can be but is not limited to an individual, or electronic system (e.g. one or more computers, and/or one or more servers). In some embodiments, the computer system comprises one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc. A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users. A machine readable medium comprising computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution. Accordingly, the invention comprehends performing any method herein-discussed and storing and/or transmitting data and/or results therefrom and/or analysis thereof, as well as products from performing any method herein-discussed, including intermediates.

### Target Sequences

Throughout this disclosure there has been mention of nucleic acid modifying protein or nucleic acid modifying complexes or systems. Nucleic acid modifying systems or complexes can target nucleic acid molecules, e.g., nucleic acid modifying complexes can target and cleave or nick or simply sit upon a target DNA molecule (depending if the nucleic acid modifying protein has mutations that render it a nickase or “dead”). Such systems or complexes are amenable for achieving tissue-specific and temporally controlled targeted deletion of candidate disease genes. Examples include but are not limited to genes involved in cholesterol and fatty acid metabolism, amyloid diseases, dominant negative diseases, latent viral infections, among other disorders. Accordingly, target sequences for such systems or complexes, can be in candidate disease genes, e.g.:

| Disease | GENE | SPACER | PAM | Mechanism | References |
|---|---|---|---|---|---|
| Hypercholesterolemia | HMGCR | GCCAAATTGG | CGG | Knockout | Fluvastatin: a review of its |
| | | ACGACCCTCG | | | pharmacology and use in the |
| | | (SEQ ID NO: 10) | | | management of |
| | | | | | hypercholesterolaemia. |
| | | | | | (Plosker GL et al. Drugs |
| | | | | | 1996, 51(3):433-459) |
| Hypercholesterolemia | SQLE | CGAGGAGACC | TGG | Knockout | Potential role of nonstatin |
| | | CCCGTTTCGG | | | cholesterol lowering agents |
| | | (SEQ ID NO: 11) | | | (Trapani et al. IUBMB Life, |
| | | | | | Volume 63, Issue 11, pages |
| | | | | | 964-971, November 2011) |
| Hyperlipidemia | DGAT1 | CCCGCCGCCG | AGG | Knockout | DGAT1 inhibitors as anti- |
| | | CCGTGGCTCG | | | obesity and anti-diabetic |
| | | (SEQ ID NO: 12) | | | agents. (Birch AM et al. |
| | | | | | Current Opinion in Drug |
| | | | | | Discovery & Development |
| | | | | | [2010, 13(4):489-496) |
| Leukemia | BCR-ABL | TGAGCTCTAC | AGG | Knockout | Killing of leukemic cells |
| | | GAGATCCACA | | | with a BCR/ABL fusion gene |
| | | SEQ ID NO: 14) | | | by RNA interference (RNAi). |
| | | | | | (Fuchs et al. Oncogene 2002, |
| | | | | | 21(37):5716-5724) |

Thus, the present invention, with regard to nucleic acid modifying protein or nucleic acid modifying complexes contemplates correction of hematopoietic disorders. For example, Severe Combined Immune Deficiency (SCID) results from a defect in lymphocytes T maturation, always associated with a functional defect in lymphocytes B (Cavazzana-Calvo et al., Annu. Rev. Med., 2005, 56, 585-602; Fischer et al., Immunol. Rev., 2005, 203, 98-109). In the case of Adenosine Deaminase (ADA) deficiency, one of the SCID forms, patients can be treated by injection of recombinant Adenosine Deaminase enzyme. Since the ADA gene has been shown to be mutated in SCID patients (Giblett et al., Lancet, 1972, 2, 1067-1069), several other genes involved in SCID have been identified (Cavazzana-Calvo et al., Annu. Rev. Med., 2005, 56, 585-602; Fischer et al., Immunol. Rev., 2005, 203, 98-109). There are four major causes for SCID: (i) the most frequent form of SCID, SCID-X1 (X-linked SCID or X-SCID), is caused by mutation in the IL2RG gene, resulting in the absence of mature T lymphocytes and NK cells. IL2RG encodes the gamma C protein (Noguchi, et al., Cell, 1993, 73, 147-157), a common component of at least five interleukin receptor complexes. These receptors activate several targets through the JAK3 kinase (Macchi et al., Nature, 1995, 377, 65-68), which inactivation results in the same syndrome as gamma C inactivation; (ii) mutation in the ADA gene results in a defect in purine metabolism that is lethal for lymphocyte precursors, which in turn results in the quasi absence of B, T and NK cells; (iii) V(D)J recombination is an essential step in the maturation of immunoglobulins and T lymphocytes receptors (TCRs). Mutations in Recombination Activating Gene 1 and 2 (RAG1 and RAG2) and Artemis, three genes involved in this process, result in the absence of mature T and B lymphocytes; and (iv) Mutations in other genes such as CD45, involved in T cell specific signaling have also been reported, although they represent a minority of cases (Cavazzana-Calvo et al., Annu. Rev. Med., 2005, 56, 585-602; Fischer et al., Immunol. Rev., 2005, 203, 98-109). In aspect of the invention, relating to CRISPR or CRISPR-Cas complexes contemplates system, the invention contemplates that it may be used to correct ocular defects that arise from several genetic mutations further described in Genetic Diseases of the Eye, Second Edition, edited by Elias I. Traboulsi, Oxford University Press, 2012. Non-limiting examples of ocular defects to be corrected include macular degeneration (MD), retinitis pigmentosa (RP). Non-limiting examples of genes and proteins associated with ocular defects include but are not limited to the following proteins: (ABCA4) ATP-binding cassette, sub-family A (ABC1), member 4 ACHM1 achromatopsia (rod monochromacy) 1 ApoE Apolipoprotein E (ApoE) C1QTNF5 (CTRP5) C1q and tumor necrosis factor related protein 5 (C1QTNF5) C2 Complement component 2 (C2) C3 Complement components (C3) CCL2 Chemokine (C-C motif) Ligand 2 (CCL2) CCR2 Chemokine (C-C motif) receptor 2 (CCR2) CD36 Cluster of Differentiation 36 CFB Complement factor B CFH Complement factor CFH H CFHR1 complement factor H-related 1 CFHR3 complement factor H-related 3 CNGB3 cyclic nucleotide gated channel beta 3 CP ceruloplasmin (CP) CRP C reactive protein (CRP) CST3 cystatin C or cystatin 3 (CST3) CTSD Cathepsin D (CTSD) CX3CR1 chemokine (C-X3-C motif) receptor 1 ELOVL4 Elongation of very long chain fatty acids 4 ERCC6 excision repair cross-complementing rodent repair deficiency, complementation group 6 FBLN5 Fibulin-5 FBLN5 Fibulin 5 FBLN6 Fibulin 6 FSCN2 fascin (FSCN2) HMCN1 Hemicentrin 1 HMCN1 hemicentin 1 HTRA1 HtrA serine peptidase 1 (HTRA1) HTRA1 HtrA serine peptidase 1 IL-6 Interleukin 6 IL-8 Interleukin 8 LOC387715 Hypothetical protein PLEKHA1 Pleckstrin homology domain-containing family A member 1 (PLEKHA1) PROMI Prominin 1 (PROM1 or CD133) PRPH2 Peripherin-2 RPGR retinitis pigmentosa GTPase regulator SERPING1 serpin peptidase inhibitor, clade G, member 1 (C1—inhibitor) TCOF1 Treacle TIMP3 Metalloproteinase inhibitor 3 (TIMP3) TLR3 Toll-like receptor 3 The present invention, with regard to CRISPR or CRISPR-Cas complexes contemplates also contemplates delivering to the heart. For the heart, a myocardium tropic adena-associated virus (AAVM) is preferred, in particular AAVM41 which showed preferential gene transfer in the heart (see, e.g., Lin-Yanga et al., PNAS, Mar. 10, 2009, vol. 106, no. 10). For example, US Patent Publication No. 20110023139, describes use of zinc finger nucleases to genetically modify cells, animals and proteins associated with cardiovascular disease. Cardiovascular diseases generally include high blood pressure, heart attacks, heart failure, and stroke and TIA. By way of example, the chromosomal sequence may comprise, but is not limited to, IL1B (interleukin 1, beta), XDH (xanthine dehydrogenase), TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), CTSK (cathepsin K), PTGIR (prostaglandin 12 (prostacyclin) receptor (IP)), KCNJ11 (potassium inwardly-rectifying channel, subfamily J, member 11), INS (insulin), CRP (C-reactive protein, pentraxin-related), PDGFRB (platelet-derived growth factor receptor, beta polypeptide), CCNA2 (cyclin A2), PDGFB (platelet-derived growth factor beta polypeptide (simian sarcoma viral (v-sis) oncogene homolog)), KCNJ5 (potassium inwardly-rectifying channel, subfamily J, member 5), KCNN3 (potassium intermediate/small conductance calcium-activated channel, subfamily N, member 3), CAPN10 (calpain 10), PTGES (prostaglandin E synthase), ADRA2B (adrenergic, alpha-2B-, receptor), ABCG5 (ATP-binding cassette, sub-family G (WHITE), member 5), PRDX2 (peroxiredoxin 2), CAPN5 (calpain 5), PARP14 (poly (ADP-ribose) polymerase family, member 14), MEX3C (mex-3 homolog C (C. elegans)), ACE angiotensin I converting enzyme (peptidyl-dipeptidase A) 1), TNF (tumor necrosis factor (TNF superfamily, member 2)), IL6 (interleukin 6 (interferon, beta 2)), STN (statin), SERPINEl (serpin peptidase inhibitor, clade E (nexin, plasminogen activator inhibitor type 1), member 1), ALB (albumin), ADIPOQ (adiponectin, C1Q and collagen domain containing), APOB (apolipoprotein B (including Ag(x) antigen)), APOE (apolipoprotein E), LEP (leptin), MTHFR (5,10-methylenetetrahydrofolate reductase (NADPH)), APOA1 (apolipoprotein A-I), EDN1 (endothelin 1), NPPB (natriuretic peptide precursor B), NOS3 (nitric oxide synthase 3 (endothelial cell)), PPARG (peroxisome proliferator-activated receptor gamma), PLAT (plasminogen activator, tissue), PTGS2 (prostaglandin-endoperoxide synthase 2 (prostaglandin G/H synthase and cyclooxygenase)), CETP (cholesteryl ester transfer protein, plasma), AGTR1 (angiotensin II receptor, type 1), HMGCR (3-hydroxy-3-methylglutaryl-Coenzyme A reductase), IGF1 (insulin-like growth factor 1 (somatomedin C)), SELE (selectin E), REN (renin), PPARA (peroxisome proliferator-activated receptor alpha), PON1 (paraoxonase 1), KNG1 (kininogen 1), CCL2 (chemokine (C-C motif) ligand 2), LPL (lipoprotein lipase), VWF (von Willebrand factor), F2 (coagulation factor II (thrombin)), ICAM1 (intercellular adhesion molecule 1), TGFB1 (transforming growth factor, beta 1), NPPA (natriuretic peptide precursor A), IL10 (interleukin 10), EPO (erythropoietin), SOD1 (superoxide dismutase 1, soluble), VCAM1 (vascular cell adhesion molecule 1), IFNG (interferon, gamma), LPA (lipoprotein, Lp(a)), MPO (myeloperoxidase), ESRI (estrogen receptor 1), MAPK1 (mitogen-activated protein kinase 1), HP (haptoglobin), F3 (coagulation factor III (thromboplastin, tissue factor)), CST3 (cystatin C), COG2 (component of oligomeric golgi complex 2), MMP9 (matrix metallopeptidase 9 (gelatinase B, 92 kDa gelatinase, 92 kDa type IV collagenase)), SERPINC1 (serpin peptidase inhibitor, clade C (antithrombin), member 1), F8 (coagulation factor VIII, procoagulant component), HMOX1 (heme oxygenase (decycling) 1), APOC3 (apolipoprotein C-III), IL8 (interleukin 8), PROK1 (prokineticin 1), CBS (cystathionine-beta-synthase), NOS2 (nitric oxide synthase 2, inducible), TLR4 (toll-like receptor 4), SELP (selectin P (granule membrane protein 140 kDa, antigen CD62)), ABCA1 (ATP-binding cassette, sub-family A (ABC1), member 1), AGT (angiotensinogen (serpin peptidase inhibitor, clade A, member 8)), LDLR (low density lipoprotein receptor), GPT (glutamic-pyruvate transaminase (alanine aminotransferase)), VEGFA (vascular endothelial growth factor A), NR3C2 (nuclear receptor subfamily 3, group C, member 2), IL18 (interleukin 18 (interferon-gamma-inducing factor)), NOS1 (nitric oxide synthase 1 (neuronal)), NR3C1 (nuclear receptor subfamily 3, group C, member 1 (glucocorticoid receptor)), FGB (fibrinogen beta chain), HGF (hepatocyte growth factor (hepapoietin A; scatter factor)), IL1A (interleukin 1, alpha), RETN (resistin), AKT1 (v-akt murine thymoma viral oncogene homolog 1), LIPC (lipase, hepatic), HSPD1 (heat shock 60 kDa protein 1 (chaperonin)), MAPK14 (mitogen-activated protein kinase 14), SPP1 (secreted phosphoprotein 1), ITGB3 (integrin, beta 3 (platelet glycoprotein 111a, antigen CD61)), CAT (catalase), UTS2 (urotensin 2), THBD (thrombomodulin), F10 (coagulation factor X), CP (ceruloplasmin (ferroxidase)), TNFRSF11 B (tumor necrosis factor receptor superfamily, member 11b), EDNRA (endothelin receptor type A), EGFR (epidermal growth factor receptor (erythroblastic leukemia viral (v-erb-b) oncogene homolog, avian)), MMP2 (matrix metallopeptidase 2 (gelatinase A, 72 kDa gelatinase, 72 kDa type IV collagenase)), PLG (plasminogen), NPY (neuropeptide Y), RHOD (ras homolog gene family, member D), MAPK8 (mitogen-activated protein kinase 8), MYC (v-myc myelocytomatosis viral oncogene homolog (avian)), FN1 (fibronectin 1), CMA1 (chymase 1, mast cell), PLAU (plasminogen activator, urokinase), GNB3 (guanine nucleotide binding protein (G protein), beta polypeptide 3), ADRB2 (adrenergic, beta-2-, receptor, surface), APOA5 (apolipoprotein A-V), SOD2 (superoxide dismutase 2, mitochondrial), F5 (coagulation factor V (proaccelerin, labile factor)), VDR (vitamin D (1,25-dihydroxyvitamin D3) receptor), ALOX5 (arachidonate 5-lipoxygenase), HLA-DRB1 (major histocompatibility complex, class II, DR beta 1), PARP1 (poly (ADP-ribose) polymerase 1), CD40LG (CD40 ligand), PON2 (paraoxonase 2), AGER (advanced glycosylation end product-specific receptor), IRS1 (insulin receptor substrate 1), PTGS1 (prostaglandin-endoperoxide synthase 1 (prostaglandin G/H synthase and cyclooxygenase)), ECE1 (endothelin converting enzyme 1), F7 (coagulation factor VII (serum prothrombin conversion accelerator)), URN (interleukin 1 receptor antagonist), EPHX2 (epoxide hydrolase 2, cytoplasmic), IGFBP1 (insulin-like growth factor binding protein 1), MAPK10 (mitogen-activated protein kinase 10), FAS (Fas (TNF receptor superfamily, member 6)), ABCB1 (ATP-binding cassette, sub-family B (MDR/TAP), member 1), JUN (jun oncogene), IGFBP3 (insulin-like growth factor binding protein 3), CD14 (CD14 molecule), PDE5A (phosphodiesterase 5A, cGMP-specific), AGTR2 (angiotensin II receptor, type 2), CD40 (CD40 molecule, TNF receptor superfamily member 5), LCAT (lecithin-cholesterol acyltransferase), CCR5 (chemokine (C-C motif) receptor 5), MMP1 (matrix metallopeptidase 1 (interstitial collagenase)), TIMP1 (TIMP metallopeptidase inhibitor 1), ADM (adrenomedullin), DYT10 (dystonia 10), STAT3 (signal transducer and activator of transcription 3 (acute-phase response factor)), MMP3 (matrix metallopeptidase 3 (stromelysin 1, progelatinase)), ELN (elastin), USF1 (upstream transcription factor 1), CFH (complement factor H), HSPA4 (heat shock 70 kDa protein 4), MMP12 (matrix metallopeptidase 12 (macrophage elastase)), MME (membrane metallo-endopeptidase), F2R (coagulation factor II (thrombin) receptor), SELL (selectin L), CTSB (cathepsin B), ANXA5 (annexin A5), ADRB1 (adrenergic, beta-1-, receptor), CYBA (cytochrome b-245, alpha polypeptide), FGA (fibrinogen alpha chain), GGT1 (gamma-glutamyltransferase 1), LIPG (lipase, endothelial), HIF1A (hypoxia inducible factor 1, alpha subunit (basic helix-loop-helix transcription factor)), CXCR4 (chemokine (C-X-C motif) receptor 4), PROC (protein C (inactivator of coagulation factors Va and VIIIa)), SCARB1 (scavenger receptor class B, member 1), CD79A (CD79a molecule, immunoglobulin-associated alpha), PLTP (phospholipid transfer protein), ADD1 (adducin 1 (alpha)), FGG (fibrinogen gamma chain), SAA1 (serum amyloid A1), KCNH2 (potassium voltage-gated channel, subfamily H (eag-related), member 2), DPP4 (dipeptidyl-peptidase 4), G6PD (glucose-6-phosphate dehydrogenase), NPR1 (natriuretic peptide receptor A/guanylate cyclase A (atrionatriuretic peptide receptor A)), VTN (vitronectin), KIAA0101 (KIAA0101), FOS (FBJ murine osteosarcoma viral oncogene homolog), TLR2 (toll-like receptor 2), PPIG (peptidylprolyl isomerase G (cyclophilin G)), IL1R1 (interleukin 1 receptor, type I), AR (androgen receptor), CYP1A1 (cytochrome P450, family 1, subfamily A, polypeptide 1), SERPINA1 (serpin peptidase inhibitor, clade A (alpha-1 antiproteinase, antitrypsin), member 1), MTR (5-methyltetrahydrofolate-homocysteine methyltransferase), RBP4 (retinol binding protein 4, plasma), APOA4 (apolipoprotein A-IV), CDKN2A (cyclin-dependent kinase inhibitor 2A (melanoma, p16, inhibits CDK4)), FGF2 (fibroblast growth factor 2 (basic)), EDNRB (endothelin receptor type B), ITGA2 (integrin, alpha 2 (CD49B, alpha 2 subunit of VLA-2 receptor)), CABINI (calcineurin binding protein 1), SHBG (sex hormone-binding globulin), HMGB1 (high-mobility group box 1), HSP90B2P (heat shock protein 90 kDa beta (Grp94), member 2 (pseudogene)), CYP3A4 (cytochrome P450, family 3, subfamily A, polypeptide 4), GJA1 (gap junction protein, alpha 1, 43 kDa), CAV1 (caveolin 1, caveolae protein, 22 kDa), ESR2 (estrogen receptor 2 (ER beta)), LTA (lymphotoxin alpha (TNF superfamily, member 1)), GDF15 (growth differentiation factor 15), BDNF (brain-derived neurotrophic factor), CYP2D6 (cytochrome P450, family 2, subfamily D, polypeptide 6), NGF (nerve growth factor (beta polypeptide)), SP1 (Sp1 transcription factor), TGIF1 (TGFB-induced factor homeobox 1), SRC (v-src sarcoma (Schmidt-Ruppin A-2) viral oncogene homolog (avian)), EGF (epidermal growth factor (beta-urogastrone)), PIK3CG (phosphoinositide-3-kinase, catalytic, gamma polypeptide), HLA-A (major histocompatibility complex, class I, A), KCNQ1 (potassium voltage-gated channel, KQT-like subfamily, member 1), CNR1 (cannabinoid receptor 1 (brain)), FBN1 (fibrillin 1), CHKA (choline kinase alpha), BEST1 (bestrophin 1), APP (amyloid beta (A4) precursor protein), CTNNB1 (catenin (cadherin-associated protein), beta 1, 88 kDa), IL2 (interleukin 2), CD36 (CD36 molecule (thrombospondin receptor)), PRKAB1 (protein kinase, AMP-activated, beta 1 non-catalytic subunit), TPO (thyroid peroxidase), ALDH7A1 (aldehyde dehydrogenase 7 family, member A1), CX3CR1 (chemokine (C-X3-C motif) receptor 1), TH (tyrosine hydroxylase), F9 (coagulation factor IX), GH1 (growth hormone 1), TF (transferrin), HFE (hemochromatosis), IL17A (interleukin 17A), PTEN (phosphatase and tensin homolog), GSTM1 (glutathione S-transferase mu 1), DMD (dystrophin), GATA4 (GATA binding protein 4), F13A1 (coagulation factor XIII, A1 polypeptide), TTR (transthyretin), FABP4 (fatty acid binding protein 4, adipocyte), PON3 (paraoxonase 3), APOC1 (apolipoprotein C-I), INSR (insulin receptor), TNFRSF1B (tumor necrosis factor receptor superfamily, member 1B), HTR2A (5-hydroxytryptamine (serotonin) receptor 2A), CSF3 (colony stimulating factor 3 (granulocyte)), CYP2C9 (cytochrome P450, family 2, subfamily C, polypeptide 9), TXN (thioredoxin), CYP111B2 (cytochrome P450, family 11, subfamily B, polypeptide 2), PTH (parathyroid hormone), CSF2 (colony stimulating factor 2 (granulocyte-macrophage)), KDR (kinase insert domain receptor (a type III receptor tyrosine kinase)), PLA2G2A (phospholipase A2, group IIA (platelets, synovial fluid)), B2M (beta-2-microglobulin), THBS1 (thrombospondin 1), GCG (glucagon), RHOA (ras homolog gene family, member A), ALDH2 (aldehyde dehydrogenase 2 family (mitochondrial)), TCF7L2 (transcription factor 7-like 2 (T-cell specific, HMG-box)), BDKRB2 (bradykinin receptor B2), NFE2L2 (nuclear factor (erythroid-derived 2)-like 2), NOTCHI (Notch homolog 1, translocation-associated (Drosophila)), UGT1A1 (UDP glucuronosyltransferase 1 family, polypeptide A1), IFNA1 (interferon, alpha 1), PPARD (peroxisome proliferator-activated receptor delta), SIRT1 (sirtuin (silent mating type information regulation 2 homolog) 1 (S. cerevisiae)), GNRH1 (gonadotropin-releasing hormone 1 (luteinizing-releasing hormone)), PAPPA (pregnancy-associated plasma protein A, pappalysin 1), ARR3 (arrestin 3, retinal (X-arrestin)), NPPC (natriuretic peptide precursor C), AHSP (alpha hemoglobin stabilizing protein), PTK2 (PTK2 protein tyrosine kinase 2), IL13 (interleukin 13), MTOR (mechanistic target of rapamycin (serine/threonine kinase)), ITGB2 (integrin, beta 2 (complement component 3 receptor 3 and 4 subunit)), GSTT1 (glutathione S-transferase theta 1), IL6ST (interleukin 6 signal transducer (gp130, oncostatin M receptor)), CPB2 (carboxypeptidase B2 (plasma)), CYP1A2 (cytochrome P450, family 1, subfamily A, polypeptide 2), HNF4A (hepatocyte nuclear factor 4, alpha), SLC6A4 (solute carrier family 6 (neurotransmitter transporter, serotonin), member 4), PLA2G6 (phospholipase A2, group VI (cytosolic, calcium-independent)), TNFSF11 (tumor necrosis factor (ligand) superfamily, member 11), SLC8A1 (solute carrier family 8 (sodium/calcium exchanger), member 1), F2RL1 (coagulation factor II (thrombin) receptor-like 1), AKR1A1 (aldo-keto reductase family 1, member A1 (aldehyde reductase)), ALDH9A1 (aldehyde dehydrogenase 9 family, member A1), BGLAP (bone gamma-carboxyglutamate (gla) protein), MTTP (microsomal triglyceride transfer protein), MTRR (5-methyltetrahydrofolate-homocysteine methyltransferase reductase), SULT1A3 (sulfotransferase family, cytosolic, 1A, phenol-preferring, member 3), RAGE (renal tumor antigen), C4B (complement component 4B (Chido blood group), P2RY12 (purinergic receptor P2Y, G-protein coupled, 12), RNLS (renalase, FAD-dependent amine oxidase), CREB1 (cAMP responsive element binding protein 1), POMC (proopiomelanocortin), RAC1 (ras-related C3 botulinum toxin substrate 1 (rho family, small GTP binding protein Rac1)), LMNA (lamin NC), CD59 (CD59 molecule, complement regulatory protein), SCN5A (sodium channel, voltage-gated, type V, alpha subunit), CYP1B1 (cytochrome P450, family 1, subfamily B, polypeptide 1), MIF (macrophage migration inhibitory factor (glycosylation-inhibiting factor)), MMP13 (matrix metallopeptidase 13 (collagenase 3)), TIMP2 (TIMP metallopeptidase inhibitor 2), CYP19A1 (cytochrome P450, family 19, subfamily A, polypeptide 1), CYP21A2 (cytochrome P450, family 21, subfamily A, polypeptide 2), PTPN22 (protein tyrosine phosphatase, non-receptor type 22 (lymphoid)), MYH14 (myosin, heavy chain 14, non-muscle), MBL2 (mannose-binding lectin (protein C) 2, soluble (opsonic defect)), SELPLG (selectin P ligand), AOC3 (amine oxidase, copper containing 3 (vascular adhesion protein 1)), CTSL1 (cathepsin L1), PCNA (proliferating cell nuclear antigen), IGF2 (insulin-like growth factor 2 (somatomedin A)), ITGB1 (integrin, beta 1 (fibronectin receptor, beta polypeptide, antigen CD29 includes MDF2, MSK12)), CAST (calpastatin), CXCL12 (chemokine (C-X-C motif) ligand 12 (stromal cell-derived factor 1)), IGHE (immunoglobulin heavy constant epsilon), KCNE1 (potassium voltage-gated channel, Isk-related family, member 1), TFRC (transferrin receptor (p90, CD71)), COL1A1 (collagen, type I, alpha 1), COL1A2 (collagen, type I, alpha 2), IL2RB (interleukin 2 receptor, beta), PLA2G10 (phospholipase A2, group X), ANGPT2 (angiopoietin 2), PROCR (protein C receptor, endothelial (EPCR)), NOX4 (NADPH oxidase 4), HAMP (hepcidin antimicrobial peptide), PTPN11 (protein tyrosine phosphatase, non-receptor type 11), SLC2A1 (solute carrier family 2 (facilitated glucose transporter), member 1), IL2RA (interleukin 2 receptor, alpha), CCL5 (chemokine (C-C motif) ligand 5), IRF1 (interferon regulatory factor 1), CFLAR (CASP8 and FADD-like apoptosis regulator), CALCA (calcitonin-related polypeptide alpha), EIF4E (eukaryotic translation initiation factor 4E), GSTP1 (glutathione S-transferase pi 1), JAK2 (Janus kinase 2), CYP3A5 (cytochrome P450, family 3, subfamily A, polypeptide 5), HSPG2 (heparan sulfate proteoglycan 2), CCL3 (chemokine (C-C motif) ligand 3), MYD88 (myeloid differentiation primary response gene (88)), VIP (vasoactive intestinal peptide), SOAT1 (sterol O-acyltransferase 1), ADRBK1 (adrenergic, beta, receptor kinase 1), NR4A2 (nuclear receptor subfamily 4, group A, member 2), MMP8 (matrix metallopeptidase 8 (neutrophil collagenase)), NPR2 (natriuretic peptide receptor B/guanylate cyclase B (atrionatriuretic peptide receptor B)), GCH1 (GTP cyclohydrolase 1), EPRS (glutamyl-prolyl-tRNA synthetase), PPARGC1A (peroxisome proliferator-activated receptor gamma, coactivator 1 alpha), F12 (coagulation factor XII (Hageman factor)), PECAM1 (platelet/endothelial cell adhesion molecule), CCL4 (chemokine (C-C motif) ligand 4), SERPINA3 (serpin peptidase inhibitor, clade A (alpha-1 antiproteinase, antitrypsin), member 3), CASR (calcium-sensing receptor), GJA5 (gap junction protein, alpha 5, 40 kDa), FABP2 (fatty acid binding protein 2, intestinal), TTF2 (transcription termination factor, RNA polymerase II), PROS1 (protein S (alpha)), CTF1 (cardiotrophin 1), SGCB (sarcoglycan, beta (43 kDa dystrophin-associated glycoprotein)), YME1L1 (YME1-like 1 (S. cerevisiae)), CAMP (cathelicidin antimicrobial peptide), ZC3H12A (zinc finger CCCH-type containing 12A), AKR1B1 (aldo-keto reductase family 1, member B1 (aldose reductase)), DES (desmin), MMP7 (matrix metallopeptidase 7 (matrilysin, uterine)), AHR (aryl hydrocarbon receptor), CSF1 (colony stimulating factor 1 (macrophage)), HDAC9 (histone deacetylase 9), CTGF (connective tissue growth factor), KCNMA1 (potassium large conductance calcium-activated channel, subfamily M, alpha member 1), UGT1A (UDP glucuronosyltransferase 1 family, polypeptide A complex locus), PRKCA (protein kinase C, alpha), COMT (catechol-.beta.-methyltransferase), S100B (S100 calcium binding protein B), EGRI (early growth response 1), PRL (prolactin), IL15 (interleukin 15), DRD4 (dopamine receptor D4), CAMK2G (calcium/calmodulin-dependent protein kinase II gamma), SLC22A2 (solute carrier family 22 (organic cation transporter), member 2), CCL11 (chemokine (C-C motif) ligand 11), PGF (B321 placental growth factor), THPO (thrombopoietin), GP6 (glycoprotein VI (platelet)), TACRI (tachykinin receptor 1), NTS (neurotensin), HNF1A (HNF1 homeobox A), SST (somatostatin), KCND1 (potassium voltage-gated channel, Shal-related subfamily, member 1), LOC646627 (phospholipase inhibitor), TBXAS1 (thromboxane A synthase 1 (platelet)), CYP2J2 (cytochrome P450, family 2, subfamily J, polypeptide 2), TBXA2R (thromboxane A2 receptor), ADH1C (alcohol dehydrogenase 1C (class I), gamma polypeptide), ALOX12 (arachidonate 12-lipoxygenase), AHSG (alpha-2-HS-glycoprotein), BHMT (betaine-homocysteine methyltransferase), GJA4 (gap junction protein, alpha 4, 37 kDa), SLC25A4 (solute carrier family 25 (mitochondrial carrier; adenine nucleotide translocator), member 4), ACLY (ATP citrate lyase), ALOX5AP (arachidonate 5-lipoxygenase-activating protein), NUMA1 (nuclear mitotic apparatus protein 1), CYP27B1 (cytochrome P450, family 27, subfamily B, polypeptide 1), CYSLTR2 (cysteinyl leukotriene receptor 2), SOD3 (superoxide dismutase 3, extracellular), LTC4S (leukotriene C4 synthase), UCN (urocortin), GHRL (ghrelin/obestatin prepropeptide), APOC2 (apolipoprotein C-II), CLEC4A (C-type lectin domain family 4, member A), KBTBD10 (kelch repeat and BTB (POZ) domain containing 10), TNC (tenascin C), TYMS (thymidylate synthetase), SHCl (SHC (Src homology 2 domain containing) transforming protein 1), LRP1 (low density lipoprotein receptor-related protein 1), SOCS3 (suppressor of cytokine signaling 3), ADH1B (alcohol dehydrogenase 1B (class I), beta polypeptide), KLK3 (kallikrein-related peptidase 3), HSD11B1 (hydroxysteroid (11-beta) dehydrogenase 1), VKORCl (vitamin K epoxide reductase complex, subunit 1), SERPINB2 (serpin peptidase inhibitor, clade B (ovalbumin), member 2), TNS1 (tensin 1), RNF19A (ring finger protein 19A), EPOR (erythropoietin receptor), ITGAM (integrin, alpha M (complement component 3 receptor 3 subunit)), PITX2 (paired-like homeodomain 2), MAPK7 (mitogen-activated protein kinase 7), FCGR3A (Fc fragment of IgG, low affinity 111a, receptor (CD16a)), LEPR (leptin receptor), ENG (endoglin), GPX1 (glutathione peroxidase 1), GOT2 (glutamic-oxaloacetic transaminase 2, mitochondrial (aspartate aminotransferase 2)), HRH1 (histamine receptor H1), NR112 (nuclear receptor subfamily 1, group I, member 2), CRH (corticotropin releasing hormone), HTR1A (5-hydroxytryptamine (serotonin) receptor IA), VDACi (voltage-dependent anion channel 1), HPSE (heparanase), SFTPD (surfactant protein D), TAP2 (transporter 2, ATP-binding cassette, sub-family B (MDR/TAP)), RNF123 (ring finger protein 123), PTK2B (PTK2B protein tyrosine kinase 2 beta), NTRK2 (neurotrophic tyrosine kinase, receptor, type 2), IL6R (interleukin 6 receptor), ACHE (acetylcholinesterase (Yt blood group)), GLP1R (glucagon-like peptide 1 receptor), GHR (growth hormone receptor), GSR (glutathione reductase), NQO1 (NAD(P)H dehydrogenase, quinone 1), NR5A1 (nuclear receptor subfamily 5, group A, member 1), GJB2 (gap junction protein, beta 2, 26 kDa), SLC9A1 (solute carrier family 9 (sodium/hydrogen exchanger), member 1), MAOA (monoamine oxidase A), PCSK9 (proprotein convertase subtilisin/kexin type 9), FCGR2A (Fc fragment of IgG, low affinity IIa, receptor (CD32)), SERPINF1 (serpin peptidase inhibitor, clade F (alpha-2 antiplasmin, pigment epithelium derived factor), member 1), EDN3 (endothelin 3), DHFR (dihydrofolate reductase), GAS6 (growth arrest-specific 6), SMPD1 (sphingomyelin phosphodiesterase 1, acid lysosomal), UCP2 (uncoupling protein 2 (mitochondrial, proton carrier)), TFAP2A (transcription factor AP-2 alpha (activating enhancer binding protein 2 alpha)), C4BPA (complement component 4 binding protein, alpha), SERPINF2 (serpin peptidase inhibitor, clade F (alpha-2 antiplasmin, pigment epithelium derived factor), member 2), TYMP (thymidine phosphorylase), ALPP (alkaline phosphatase, placental (Regan isozyme)), CXCR2 (chemokine (C-X-C motif) receptor 2), SLC39A3 (solute carrier family 39 (zinc transporter), member 3), ABCG2 (ATP-binding cassette, sub-family G (WHITE), member 2), ADA (adenosine deaminase), JAK3 (Janus kinase 3), HSPAIA (heat shock 70 kDa protein 1A), FASN (fatty acid synthase), FGF1 (fibroblast growth factor 1 (acidic)), F11 (coagulation factor XI), ATP7A (ATPase, Cu++ transporting, alpha polypeptide), CR1 (complement component (3b/4b) receptor 1 (Knops blood group)), GFAP (glial fibrillary acidic protein), ROCK1 (Rho-associated, coiled-coil containing protein kinase 1), MECP2 (methyl CpG binding protein 2 (Rett syndrome)), MYLK (myosin light chain kinase), BCHE (butyrylcholinesterase), LIPE (lipase, hormone-sensitive), PRDX5 (peroxiredoxin 5), ADORAl (adenosine A1 receptor), WRN (Werner syndrome, RecQ helicase-like), CXCR3 (chemokine (C-X-C motif) receptor 3), CD81 (CD81 molecule), SMAD7 (SMAD family member 7), LAMC2 (laminin, gamma 2), MAP3K5 (mitogen-activated protein kinase kinase kinase 5), CHGA (chromogranin A (parathyroid secretory protein 1)), IAPP (islet amyloid polypeptide), RHO (rhodopsin), ENPP1 (ectonucleotide pyrophosphatase/phosphodiesterase 1), PTHLH (parathyroid hormone-like hormone), NRG1 (neuregulin 1), VEGFC (vascular endothelial growth factor C), ENPEP (glutamyl aminopeptidase (aminopeptidase A)), CEBPB (CCAAT/enhancer binding protein (C/EBP), beta), NAGLU (N-acetylglucosaminidase, alpha-), F2RL3 (coagulation factor II (thrombin) receptor-like 3), CX3CL1 (chemokine (C-X3-C motif) ligand 1), BDKRB1 (bradykinin receptor B1), ADAMTS13 (ADAM metallopeptidase with thrombospondin type 1 motif, 13), ELANE (elastase, neutrophil expressed), ENPP2 (ectonucleotide pyrophosphatase/phosphodiesterase 2), CISH (cytokine inducible SH2-containing protein), GAST (gastrin), MYOC (myocilin, trabecular meshwork inducible glucocorticoid response), ATP1A2 (ATPase, Na+/K+transporting, alpha 2 polypeptide), NF1 (neurofibromin 1), GJB1 (gap junction protein, beta 1, 32 kDa), MEF2A (myocyte enhancer factor 2A), VCL (vinculin), BMPR2 (bone morphogenetic protein receptor, type II (serine/threonine kinase)), TUBB (tubulin, beta), CDCl42 (cell division cycle 42 (GTP binding protein, 25 kDa)), KRT18 (keratin 18), HSF1 (heat shock transcription factor 1), MYB (v-myb myeloblastosis viral oncogene homolog (avian)), PRKAA2 (protein kinase, AMP-activated, alpha 2 catalytic subunit), ROCK2 (Rho-associated, coiled-coil containing protein kinase 2), TFPI (tissue factor pathway inhibitor (lipoprotein-associated coagulation inhibitor)), PRKG1 (protein kinase, cGMP-dependent, type I), BMP2 (bone morphogenetic protein 2), CTNND1 (catenin (cadherin-associated protein), delta 1), CTH (cystathionase (cystathionine gamma-lyase)), CTSS (cathepsin S), VAV2 (vav 2 guanine nucleotide exchange factor), NPY2R (neuropeptide Y receptor Y2), IGFBP2 (insulin-like growth factor binding protein 2, 36 kDa), CD28 (CD28 molecule), GSTA1 (glutathione S-transferase alpha 1), PPIA (peptidylprolyl isomerase A (cyclophilin A)), APOH (apolipoprotein H (beta-2-glycoprotein I)), S100A8 (S100 calcium binding protein A8), IL11 (interleukin 11), ALOX15 (arachidonate 15-lipoxygenase), FBLN1 (fibulin 1), NR1H3 (nuclear receptor subfamily 1, group H, member 3), SCD (stearoyl-CoA desaturase (delta-9-desaturase)), GIP (gastric inhibitory polypeptide), CHGB (chromogranin B (secretogranin 1)), PRKCB (protein kinase C, beta), SRD5A1 (steroid-5-alpha-reductase, alpha polypeptide 1 (3-oxo-5 alpha-steroid delta 4-dehydrogenase alpha 1)), HSD11B2 (hydroxysteroid (11-beta) dehydrogenase 2), CALCRL (calcitonin receptor-like), GALNT2 (UDP-N-acetyl-alpha-D-galactosamine:polypeptide N-acetylgalactosaminyltransferase 2 (GalNAc-T2)), ANGPTL4 (angiopoietin-like 4), KCNN4 (potassium intermediate/small conductance calcium-activated channel, subfamily N, member 4), PIK3C2A (phosphoinositide-3-kinase, class 2, alpha polypeptide), HBEGF (heparin-binding EGF-like growth factor), CYP7A1 (cytochrome P450, family 7, subfamily A, polypeptide 1), HLA-DRB5 (major histocompatibility complex, class II, DR beta 5), BNIP3 (BCL2/adenovirus E1B 19 kDa interacting protein 3), GCKR (glucokinase (hexokinase 4) regulator), S100A12 (S100 calcium binding protein A12), PADI4 (peptidyl arginine deiminase, type IV), HSPA14 (heat shock 70 kDa protein 14), CXCR1 (chemokine (C-X-C motif) receptor 1), H19 (H19, imprinted maternally expressed transcript (non-protein coding)), KRTAP19-3 (keratin associated protein 19-3), IDDM2 (insulin-dependent diabetes mellitus 2), RAC2 (ras-related C3 botulinum toxin substrate 2 (rho family, small GTP binding protein Rac2)), RYRI (ryanodine receptor 1 (skeletal)), CLOCK (clock homolog (mouse)), NGFR (nerve growth factor receptor (TNFR superfamily, member 16)), DBH (dopamine beta-hydroxylase (dopamine beta-monooxygenase)), CHRNA4 (cholinergic receptor, nicotinic, alpha 4), CACNAlC (calcium channel, voltage-dependent, L type, alpha 1C subunit), PRKAG2 (protein kinase, AMP-activated, gamma 2 non-catalytic subunit), CHAT (choline acetyltransferase), PTGDS (prostaglandin D2 synthase 21 kDa (brain)), NR1H2 (nuclear receptor subfamily 1, group H, member 2), TEK (TEK tyrosine kinase, endothelial), VEGFB (vascular endothelial growth factor B), MEF2C (myocyte enhancer factor 2C), MAPKAPK2 (mitogen-activated protein kinase-activated protein kinase 2), TNFRSF11A (tumor necrosis factor receptor superfamily, member 11a, NFKB activator), HSPA9 (heat shock 70 kDa protein 9 (mortalin)), CYSLTR1 (cysteinyl leukotriene receptor 1), MAT1A (methionine adenosyltransferase I, alpha), OPRL1 (opiate receptor-like 1), IMPA1 (inositol(myo)-1(or 4)-monophosphatase 1), CLCN2 (chloride channel 2), DLD (dihydrolipoamide dehydrogenase), PSMA6 (proteasome (prosome, macropain) subunit, alpha type, 6), PSMB8 (proteasome (prosome, macropain) subunit, beta type, 8 (large multifunctional peptidase 7)), CHI3L1 (chitinase 3-like 1 (cartilage glycoprotein-39)), ALDH1B1 (aldehyde dehydrogenase 1 family, member B1), PARP2 (poly (ADP-ribose) polymerase 2), STAR (steroidogenic acute regulatory protein), LBP (lipopolysaccharide binding protein), ABCC6 (ATP-binding cassette, sub-family C(CFTR/MRP), member 6), RGS2 (regulator of G-protein signaling 2, 24 kDa), EFNB2 (ephrin-B2), GJB6 (gap junction protein, beta 6, 30 kDa), APOA2 (apolipoprotein A-II), AMPD1 (adenosine monophosphate deaminase 1), DYSF (dysferlin, limb girdle muscular dystrophy 2B (autosomal recessive)), FDFT1 (farnesyl-diphosphate farnesyltransferase 1), EDN2 (endothelin 2), CCR6 (chemokine (C-C motif) receptor 6), GJB3 (gap junction protein, beta 3, 31 kDa), IL1RL1 (interleukin 1 receptor-like 1), ENTPD1 (ectonucleoside triphosphate diphosphohydrolase 1), BBS4 (Bardet-Biedl syndrome 4), CELSR2 (cadherin, EGF LAG seven-pass G-type receptor 2 (flamingo homolog, Drosophila)), F11R (F11 receptor), RAPGEF3 (Rap guanine nucleotide exchange factor (GEF) 3), HYAL1 (hyaluronoglucosaminidase 1), ZNF259 (zinc finger protein 259), ATOX1 (ATX1 antioxidant protein 1 homolog (yeast)), ATF6 (activating transcription factor 6), KHK (ketohexokinase (fructokinase)), SAT1 (spermidine/spermine N1-acetyltransferase 1), GGH (gamma-glutamyl hydrolase (conjugase, folylpolygammaglutamyl hydrolase)), TIMP4 (TIMP metallopeptidase inhibitor 4), SLC4A4 (solute carrier family 4, sodium bicarbonate cotransporter, member 4), PDE2A (phosphodiesterase 2A, cGMP-stimulated), PDE3B (phosphodiesterase 3B, cGMP-inhibited), FADS1 (fatty acid desaturase 1), FADS2 (fatty acid desaturase 2), TMSB4X (thymosin beta 4, X-linked), TXNIP (thioredoxin interacting protein), LIMS1 (LIM and senescent cell antigen-like domains 1), RHOB (ras homolog gene family, member B), LY96 (lymphocyte antigen 96), FOXO1 (forkhead box 01), PNPLA2 (patatin-like phospholipase domain containing 2), TRH (thyrotropin-releasing hormone), GJC1 (gap junction protein, gamma 1, 45 kDa), SLC17A5 (solute carrier family 17 (anion/sugar transporter), member 5), FTO (fat mass and obesity associated), GJD2 (gap junction protein, delta 2, 36 kDa), PSRC1 (proline/serine-rich coiled-coil 1), CASP12 (caspase 12 (gene/pseudogene)), GPBAR1 (G protein-coupled bile acid receptor 1), PXK (PX domain containing serine/threonine kinase), IL33 (interleukin 33), TRIB1 (tribbles homolog 1 (Drosophila)), PBX4 (pre-B-cell leukemia homeobox 4), NUPR1 (nuclear protein, transcriptional regulator, 1), 15 September (15 kDa selenoprotein), CILP2 (cartilage intermediate layer protein 2), TERC (telomerase RNA component), GGT2 (gamma-glutamyltransferase 2), MT-CO1 (mitochondrially encoded cytochrome c oxidase I), and UOX (urate oxidase, pseudogene). In an additional embodiment, the chromosomal sequence may further be selected from Pon1 (paraoxonase 1), LDLR (LDL receptor), ApoE (Apolipoprotein E), Apo B-100 (Apolipoprotein B-100), ApoA (Apolipoprotein(a)), ApoA1 (Apolipoprotein A1), CBS (Cystathione B-synthase), Glycoprotein IIb/IIb, MTHRF (5,10-methylenetetrahydrofolate reductase (NADPH), and combinations thereof. In one iteration, the chromosomal sequences and proteins encoded by chromosomal sequences involved in cardiovascular disease may be chosen from CacnalC, Sod1, Pten, Ppar(alpha), Apo E, Leptin, and combinations thereof. The text herein accordingly provides exemplary targets as to CRISPR or CRISPR-Cas systems or complexes.

The following are incorporated by reference.
Biosecurity Implications of Gene Drive Research (http://nas-sites.org/gene-drives/2015/10/07/implications-of-gene-drive-research-on-biosecurity-webinar/). You, E.
Biotechnology. A prudent path forward for genomic engineering and germline gene modification. Baltimore, D.; Berg, P.; Botchan, M.; Carroll, D.; Charo, R. A.; Church, G.; Corn, J. E.; Daley, G. Q.; Doudna, J. A.; Fenner, M.; Greely, H. T.; Jinek, M.; Martin, G. S.; Penhoet, E.; Puck, J.; Sternberg, S. H.; Weissman, J. S.; Yamamoto, K. R. Science 2015, 348, 36-8. PMC4394183
BIOSAFETY. Safeguarding gene drive experiments in the laboratory. Akbari, O. S.; Bellen, H. J.; Bier, E.; Bullock, S. L.; Burt, A.; Church, G. M.; Cook, K. R.; Duchek, P.; Edwards, O. R.; Esvelt, K. M.; Gantz, V. M.; Golic, K. G.; Gratz, S. J.; Harrison, M. M.; Hayes, K. R.; James, A. A.; Kaufman, T. C.; Knoblich, J.; Malik, H. S.; Matthews, K. A.; O'Connor-Giles, K. M.; Parks, A. L.; Perrimon, N.; Port, F.; Russell, S.; Ueda, R.; Wildonger, J. Science 2015, 349, 927-9. PMC4692367
Gene drive overdrive. Nat Biotech 2015, 33, 1019-1021
Opinion: Is CRISPR-based gene drive a biocontrol silver bullet or global conservation threat? Webber, B. L.; Raghu, S.; Edwards, O. R. Proceedings of the National Academy of Sciences 2015, 112, 10565-10567
Gene drives spread their wings. Saey, T. H. Science News 2015, 188, 16
Concerning RNA-guided gene drives for the alteration of wild populations. Esvelt, K. M.; Smidler, A. L.; Catteruccia, F.; Church, G. M. eLife 2014, 3, e03401
The dawn of active genetics. Gantz, V. M.; Bier, E. Bioessays 2016, 38, 50-63
Cheating evolution: engineering gene drives to manipulate the fate of wild populations. Champer, J.; Buchman, A.; Akbari, O. S. Nat Rev Genet 2016, 17, 146-159
Entomological terrorism: a tactic in assymmetrical warfare. Monthei, D.; Mueller, S.; Lockwood, J.; Debboun, M. US Army Med Dep J 2010, 11-21
CRISPR-mediated direct mutation of cancer genes in the mouse liver. Xue, W.; Chen, S.; Yin, H.; Tammela, T.; Papagiannakopoulos, T.; Joshi, N. S.; Cai, W.; Yang, G.; Bronson, R.; Crowley, D. G.; Zhang, F.; Anderson, D. G.; Sharp, P. A.; Jacks, T. Nature 2014, 514, 380-4. PMC4199937
Rapid modelling of cooperating genetic events in cancer through somatic genome editing. Sanchez-Rivera, F. J.; Papagiannakopoulos, T.; Romero, R.; Tammela, T.; Bauer, M. R.; Bhutkar, A.; Joshi, N. S.; Subbaraj, L.; Bronson, R. T.; Xue, W.; Jacks, T. Nature 2014, 516, 428-31. PMC4292871
CRISPR-Cas9 knockin mice for genome editing and cancer modeling. Platt, R. J.; Chen, S.; Zhou, Y.; Yim, M. J.; Swiech, L.; Kempton, H. R.; Dahlman, J. E.; Parnas, O.; Eisenhaure, T. M.; Jovanovic, M.; Graham, D. B.; Jhunjhunwala, S.; Heidenreich, M.; Xavier, R. J.; Langer, R.; Anderson, D. G.; Hacohen, N.; Regev, A.; Feng, G.; Sharp, P. A.; Zhang, F. Cell 2014, 159, 440-55. PMC4265475
Global microRNA depletion suppresses tumor angiogenesis. Chen, S.; Xue, Y.; Wu, X.; Le, C.; Bhutkar, A.; Bell, E. L.; Zhang, F.; Langer, R.; Sharp, P. A. Genes Dev 2014, 28, 1054-67. PMC4035535
Applications of the CRISPR-Cas9 system in cancer biology. Sánchez-Rivera, F. J.; Jacks, T. Nat Rev Cancer 2015, 15, 387-95
Genome-wide CRISPR screen in a mouse model of tumor growth and metastasis.
Chen, S.; Sanjana, N. E.; Zheng, K.; Shalem, O.; Lee, K.; Shi, X.; Scott, D. A.; Song, J.; Pan, J. Q.; Weissleder, R.; Lee, H.; Zhang, F.; Sharp, P. A. Cell 2015, 160, 1246-60. PMC4380877
Increasing the efficiency of precise genome editing with CRISPR-Cas9 by inhibition of nonhomologous end joining. Maruyama, T.; Dougan, S. K.; Truttmann, M. C.; Bilate, A. M.; Ingram, J. R.; Ploegh, H. L. Nat Biotech 2015, 33, 538-542
Increasing the efficiency of homology-directed repair for CRISPR-Cas9-induced precise gene editing in mammalian cells. Chu, V. T.; Weber, T.; Wefers, B. Nat. Biotech 2015, 33, 543-8
Enhancing homology-directed genome editing by catalytically active and inactive CRISPR-Cas9 using asymmetric donor DNA. Richardson, C. D.; Ray, G. J.; DeWitt, M. A.; Curie, G. L.; Corn, J. E. Nat Biotech 2016, 34, 339-344
The cytotoxicity of (−)-lomaiviticin A arises from induction of double-strand breaks in DNA. Colis, L. C.; Woo, C. M.; Hegan, D. C.; Li, Z.; Glazer, P. M.; Herzon, S. B. Nature chemistry 2014, 6, 504-510
Structural basis for DNA cleavage by the potent antiproliferative agent (−)-lomaiviticin A. Woo, C. M.; Li, Z.; Paulson, E. K. 2016, 113, 2851-6
Rational design of human DNA ligase inhibitors that target cellular DNA replication and repair. Chen, X.; Zhong, S.; Zhu, X.; Dziegielewska, B.; Ellenberger, T.; Wilson, G. M.; MacKerell, A. D., Jr.; Tomkinson, A. E. Cancer Res 2008, 68, 3169-77. PMC2734474
An inhibitor of nonhomologous end-joining abrogates double-strand break repair and impedes cancer progression. Srivastava, M.; Nambiar, M.; Sharma, S.; Karki, S. S.; Goldsmith, G.; Hegde, M.; Kumar, S.; Pandey, M.; Singh, R. K.; Ray, P.; Natarajan, R.; Kelkar, M.; De, A.; Choudhary, B.; Raghavan, S. C. Cell 2012, 151, 1474-87
Increasing the efficiency of precise genome editing with CRISPR-Cas9 by inhibition of nonhomologous end joining. Maruyama, T.; Dougan, S. K.; Truttmann, M. C.; Bilate, A. M.; Ingram, J. R.; Ploegh, H. L. Nat Biotechnol 2015, 33, 538-42. PMC4618510
Increasing the efficiency of homology-directed repair for CRISPR-Cas9-induced precise gene editing in mammalian cells. Chu, V. T.; Weber, T.; Wefers, B.; Wurst, W.; Sander, S.; Rajewsky, K.; Kuhn, R. Nat Biotechnol 2015, 33, 543-8
SCR7 is neither a selective nor a potent inhibitor of human DNA ligase IV. Greco, G. E.; Matsumoto, Y.; Brooks, R. C.; Lu, Z.; Lieber, M. R.; Tomkinson, A. E. DNA Repair (Amst) 2016, 43, 18-23. PMC5042453
A chemical compound that stimulates the human homologous recombination protein RAD51. Jayathilaka, K.; Sheridan, S. D.; Bold, T. D.; Bochenska, K.; Logan, H. L.; Weichselbaum, R. R.; Bishop, D. K.; Connell, P. P. Proc Natl Acad Sci USA 2008, 105, 15848-53. PMC2572930
Dual and Opposite Effects of hRAD51 Chemical Modulation on HIV-1 Integration. Thierry, S.; Benleulmi, M. S.; Sinzelle, L.; Thierry, E.; Calmels, C.; Chaignepain, S.; Waffo-Teguo, P.; Merillon, J. M.; Budke, B.; Pasquet, J. M.; Litvak, S.; Ciuffi, A.; Sung, P.; Connell, P.; Hauber, I.; Hauber, J.; Andreola, M. L.; Delelis, O.; Parissi, V. Chem Biol 2015, 22, 712-23. PMC4889029
Nuclear domain ‘knock-in’ screen for the evaluation and identification of small molecule enhancers of CRISPR-based genome editing. Pinder, J.; Salsman, J.; Dellaire, G. Nucleic Acids Res 2015, 43, 9379-92. PMC4627099
A guide to genome engineering with programmable nucleases. Kim, H.; Kim, J. S. Nat Rev Genet 2014, 15, 321-34
Synthetic mimetics of protein secondary structure domains. Ross, N. T.; Katt, W. P.; Hamilton, A. D. Philosophical transactions. Series A, Mathematical, physical, and engineering sciences 2010, 368, 989-1008
The crystal structure of TAL effector PthXo1 bound to its DNA target. Mak, A. N.-S.; Bradley, P.; Cernadas, R. A.; Bogdanove, A. J.; Stoddard, B. L. Science (New York, N.Y.) 2012, 335, 716-719
Calculating structures and free energies of complex molecules: combining molecular mechanics and continuum models. Kollman, P. A.; Massova, I.; Reyes, C.; Kuhn, B.; Huo, S.; Chong, L.; Lee, M.; Lee, T.; Duan, Y.; Wang, W. Accounts of chemical research 2000, 33, 889-897%@0001-4842
Antechamber: an accessory software package for molecular mechanical calculations. Wang, J.; Wang, W.; Kollman, P. A.; Case, D. A. J. Am. Chem. Soc 2001, 222, U403
Boronate-mediated biologic delivery. Ellis, G. A.; Palte, M. J.; Raines, R. T. J Am Chem Soc 2012, 134, 3631-4. Pmc3304437
Cell-penetrating peptides: 20 years later, where do we stand? Bechara, C.; Sagan, S. FEBS Lett 2013, 587, 1693-702
Click chemistry in complex mixtures: bioorthogonal bioconjugation. McKay, C. S.; Finn, M. G. Chem Biol 2014, 21, 1075-101. Pmc4331201
High-frequency off-target mutagenesis induced by CRISPR-Cas nucleases in human cells. Fu, Y.; Foden, J. A.; Khayter, C.; Maeder, M. L.; Reyon, D.; Joung, J. K.; Sander, J. D. Nat Biotechnol 2013, 31, 822-6. Pmc3773023
Multidimensional chemical control of CRISPR-Cas9. Maji, B.; Moore, C. L.; Zetsche, B.; Volz, S. E.; Zhang, F.; Shoulders, M. D.; Choudhary, A. Nat Chem Biol 2016, advance online publication
Evolution and classification of the CRISPR-Cas systems. Makarova, K. S.; Haft, D. H.; Barrangou, R.; Brouns, S. J.; Charpentier, E.; Horvath, P.; Moineau, S.; Mojica, F. J.; Wolf, Y. I.; Yakunin, A. F.; van der Oost, J.; Koonin, E. V. Nat Rev Microbiol 2011, 9, 467-77. PMC3380444
An updated evolutionary classification of CRISPR-Cas systems. Makarova, K. S.; Wolf, Y. I.; Alkhnbashi, O. S.; Costa, F.; Shah, S. A.; Saunders, S. J.; Barrangou, R.; Brouns, S. J.; Charpentier, E.; Haft, D. H.; Horvath, P.; Moineau, S.; Mojica, F. J.; Terns, R. M.; Terns, M. P.; White, M. F.; Yakunin, A. F.; Garrett, R. A.; van der Oost, J.; Backofen, R.; Koonin, E. V. Nat Rev Microbiol 2015
Advances in CRISPR-Cas9 genome engineering: lessons learned from RNA interference. Barrangou, R.; Birmingham, A.; Wiemann, S.; Beijersbergen, R. L.; Hornung, V.; Smith, A. Nucleic Acids Res 2015, 43, 3407-19. PMC4402539
The mechanism of double-strand DNA break repair by the nonhomologous DNA end-joining pathway. Lieber, M. R. Annu Rev Biochem 2010, 79, 181-211. Pmc3079308
Reduced ciliary polycystin-2 in induced pluripotent stem cells from polycystic kidney disease patients with PKD1 mutations. Freedman, B. S.; Lam, A. Q.; Sundsbak, J. L.; Iatrino, R.; Su, X.; Koon, S. J.; Wu, M.; Daheron, L.; Harris, P. C.; Zhou, J.; Bonventre, J. V. J Am Soc Nephrol 2013, 24, 1571-86. Pmc3785271
Polycystic kidney disease. Wilson, P. D. N Engl J Med 2004, 350, 151-64
Fibrocystin/polyductin, found in the same protein complex with polycystin-2, regulates calcium responses in kidney epithelia. Wang, S.; Zhang, J.; Nauli, S. M.; Li, X.; Starremans, P. G.; Luo, Y.; Roberts, K. A.; Zhou, J. Mol Cell Biol 2007, 27, 3241-52. Pmc1899915
Site-specific C-terminal and internal loop labeling of proteins using sortase-mediated reactions. Guimaraes, C. P.; Witte, M. D.; Theile, C. S.; Bozkurt, G.; Kundrat, L.; Blom, A. E.; Ploegh, H. L. Nat Protoc 2013, 8, 1787-99. PMC3943461
Site-specific N-terminal labeling of proteins using sortase-mediated reactions. Theile, C. S.; Witte, M. D.; Blom, A. E.; Kundrat, L.; Ploegh, H. L.; Guimaraes, C. P. Nat Protoc 2013, 8, 1800-7. PMC3941705
Sortase-mediated ligations for the site-specific modification of proteins. Schmohl, L.; Schwarzer, D. Curr Opin Chem Biol 2014, 22, 122-8
A split-Cas9 architecture for inducible genome editing and transcription modulation. Zetsche, B.; Volz, S. E.; Zhang, F. Nat Biotechnol 2015, 33, 139-42. PMC4503468
Rational design of a split-Cas9 enzyme complex. Wright, A. V.; Sternberg, S. H.; Taylor, D. W.; Staahl, B. T.; Bardales, J. A.; Komfeld, J. E.; Doudna, J. A. Proc Natl Acad Sci USA 2015, 112, 2984-9. PMC4364227
Rationally engineered Cas9 nucleases with improved specificity. Slaymaker, I. M.; Gao, L.; Zetsche, B.; Scott, D. A.; Yan, W. X.; Zhang, F. Science (New York, N.Y.) 2016, 351, 84-88
RNA-programmed genome editing in human cells. Jinek, M.; East, A.; Cheng, A.; Lin, S.; Ma, E.; Doudna, J. Elife 2013, 2, e00471. PMC3557905
Cationic lipid-mediated delivery of proteins enables efficient protein-based genome editing in vitro and in vivo. Zuris, J. A.; Thompson, D. B.; Shu, Y.; Guilinger, J. P.; Bessen, J. L.; Hu, J. H.; Maeder, M. L.; Joung, J. K.; Chen, Z. Y.; Liu, D. R. Nat Biotechnol 2015, 33, 73-80. PMC4289409
High-frequency off-target mutagenesis induced by CRISPR-Cas nucleases in human cells. Fu, Y.; Foden, J. A.; Khayter, C.; Maeder, M. L.; Reyon, D.; Joung, J. K.; Sander, J. D. Nat Biotechnol 2013, 31, 822-6. Pmc3773023
DNA repair targeted therapy: The past or future of cancer treatment? Gavande, N. S.; VanderVere-Carozza, P. S.; Hinshaw, H. D.; Jalal, S. I.; Sears, C. R.; Pawelczak, K. S.; Turchi, J. J. Pharmacol Ther 2016, 160, 65-83. PMC4811676

Although the present invention and its advantages have been described in detail, it should be understood that various changes, substitutions and alterations can be made herein without departing from the spirit and scope of the invention as defined in the appended claims. The present invention will be further illustrated in the following Examples which are given for illustration purposes only and are not intended to limit the invention in any way.

### WORKING EXAMPLES

In one example of the usefulness of the multivalent display of small molecules, NHEJ inhibitors conjugated to Cas9 could localize inhibition to the strand break site, enhancing precise editing while drastically reducing toxicity. Local inhibition of uracil DNA glycosylase would also be helpful for the development of efficient base editors, and the local inhibition of p53 pathway activation will increase the efficiency of precision genome editing in many primary cells where Cas9-induced double-strand breaks lead to apoptosis via activation of the p53 pathway. Finally, tissue-specific ligands displayed on Cas9 will enable cell-specific genome editing.

### Example 1—a Synthetic all in One Genome Editor

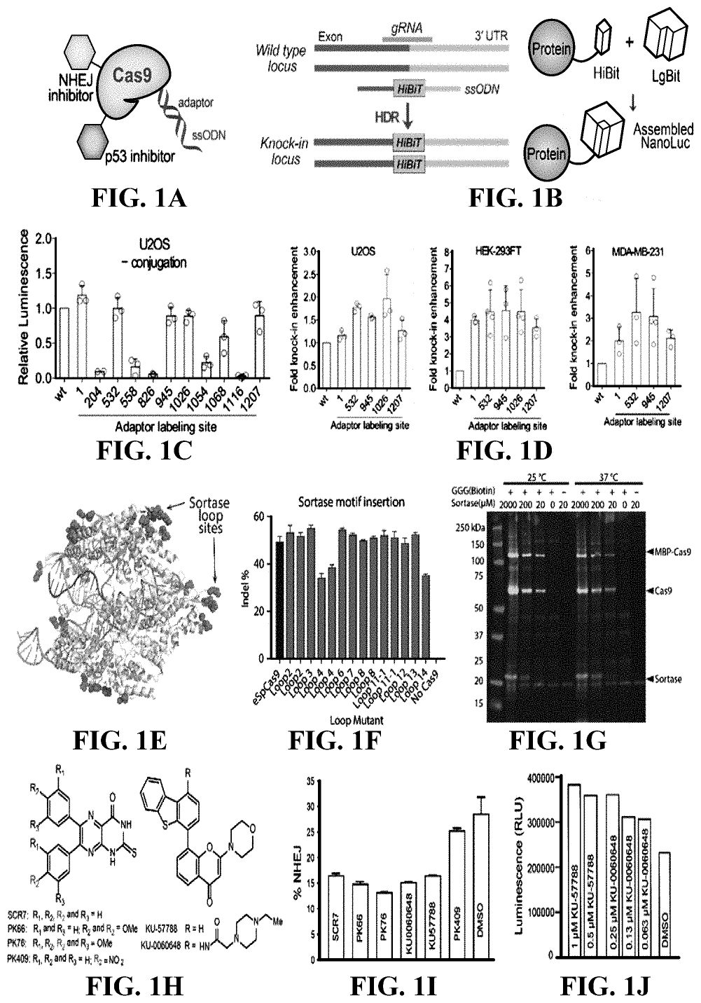

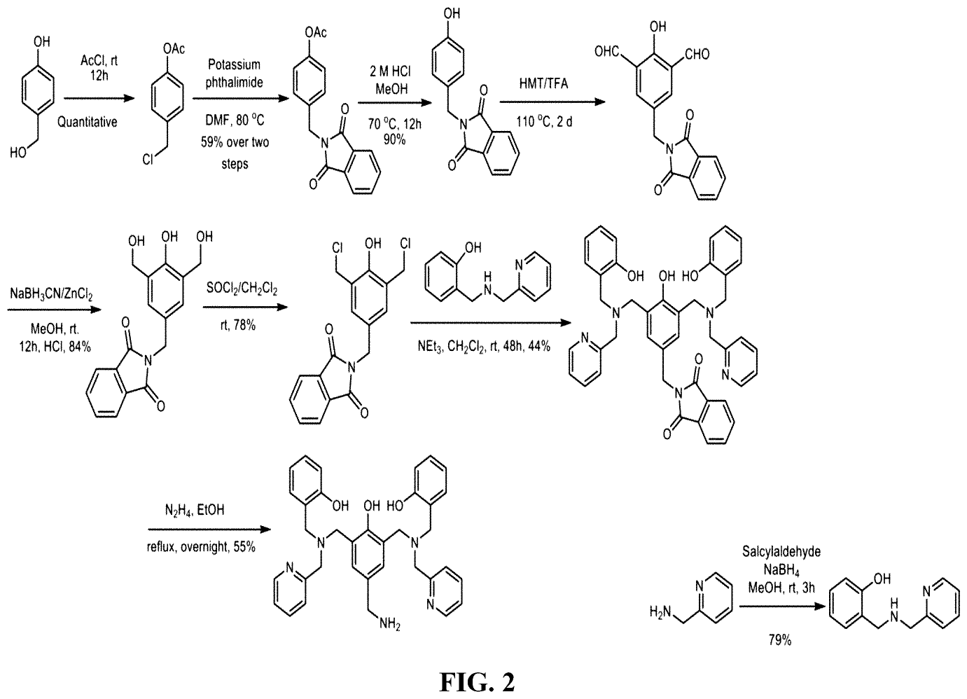

Applicants sought to establish a general platform for attaching various molecules to Cas9 due to the diverse nature of our desired conjugates (i.e., nucleic acids, nanoparticles, antibodies, small molecules). This platform relies on thiol-maleimide chemistry and DNA base pairing, which are both simple and well established and are amenable to a wide range of substrates. Following a structure-guided approach, Applicants systematically scanned the domains of Cas9 to choose residues replaceable with engineered cysteines to which molecules of any size could be efficiently appended without the loss of Cas9 activity. Because many possible conjugates such as long oligonucleotides are prohibitively expensive for or unamenable to the direct thiol-maleimide conjugation, Applicants next developed a more general conjugation platform. To do this, Applicants designed a short oligonucleotide handle named ‘adaptor’ that is attached to Cas9 via thiol-maleimide chemistry and uses base pairing to anchor any molecule naturally containing or appended with nucleic acids (FIG. 1a). As an example, Applicants linked ssODNs to Cas9 using the adapter strategy, because they are large, expensive, and not available in large enough quantities for efficient thiol-maleimide conjugation. The conjugation enhanced the HDR-mediated incorporation of the desired sequence from ssODN at the break site by more than four-fold due to the increased local concentration. Applicants further demonstrate the robustness of the method with knock-in enhancements in multiple cell types and genomic sites using multiple assay readouts. The chemical conjugation strategy enabled multivalent display of ssODNs, which further enhanced the knock-in efficiency. Applicants also confirmed that the Cas9-DNA conjugate did not alter Cas9 specificity.

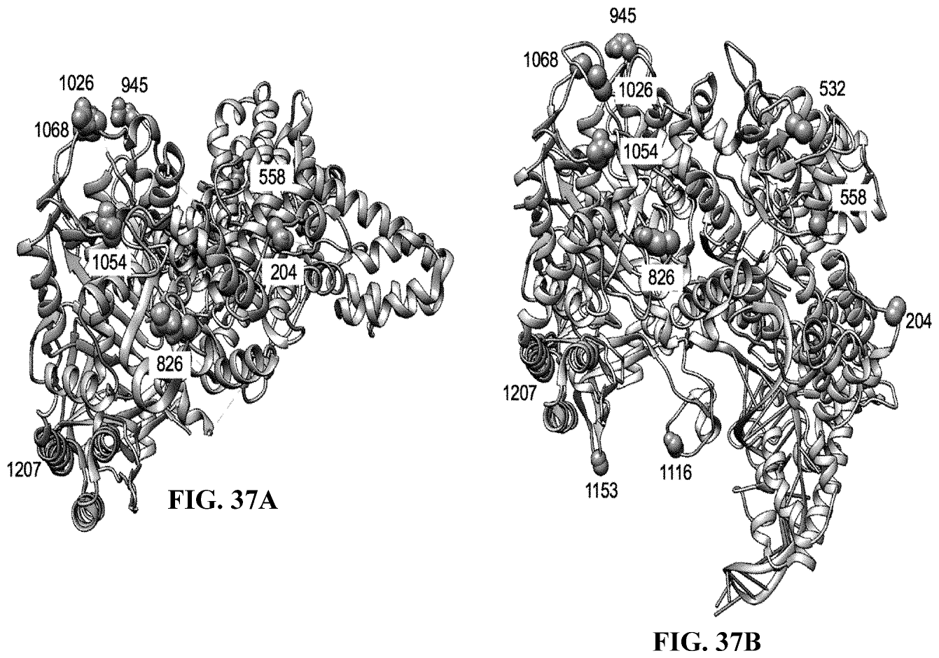

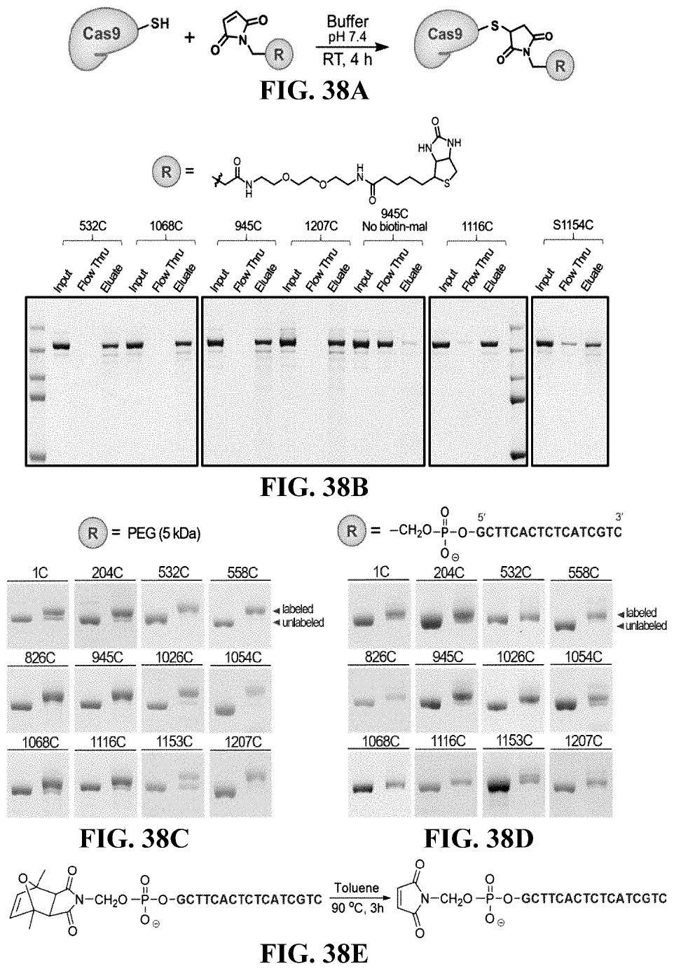

To choose the conjugation sites, Applicants analyzed the structures of apo-Cas9,²⁰ guide RNA (gRNA)-bound Cas9,²¹ and gRNA- and DNA-bound Cas9²² and selected residues that could provide a high labeling yield, could tolerate chemical modifications, were located on surface-exposed loop regions in all crystal structures, and spanned all the domains of Cas9. Also, Applicants selected residues 558 and 1116 as controls, since modifications at 558 will impede the Cas9:gRNA interaction and at 1116 will impede protospacer adjacent motif recognition by Cas9 (FIGS. 12b and 37A-37B). Applicants first optimized the conjugation conditions for Cas9 variants using biotin-maleimide and PEG (5 kDa)-maleimide and as model compounds (FIG. 38A-38E). The reactions were all fast and high yielding except for the 1153C mutant-sites proximal to 1153C (i.e., 1154C) also yielded low conjugation efficiencies (FIG. 38A-38E). The location of these residues was not assigned at the crystal structure of apo-Cas9, but Applicants assumed that they are amenable to efficient conjugation because the loop they belong to was predicted to be surface-exposed and very flexible. The labeling results, however, indicate that the loop may have higher order structures to prevent efficient chemical reactions. Therefore, those sites were not used in future experiments. Applicants next utilized these optimized conditions to label Cas9 with a 17-nucleotide (nt) DNA adaptor (5′-GCTTCACTCTCATCGTC-3′) (SEQ ID NO: 14) and found conversion rates comparable to those of PEG labeling (FIG. 38A-38E), attesting that efficient conjugation of multiple cargo types can occur at these sites.

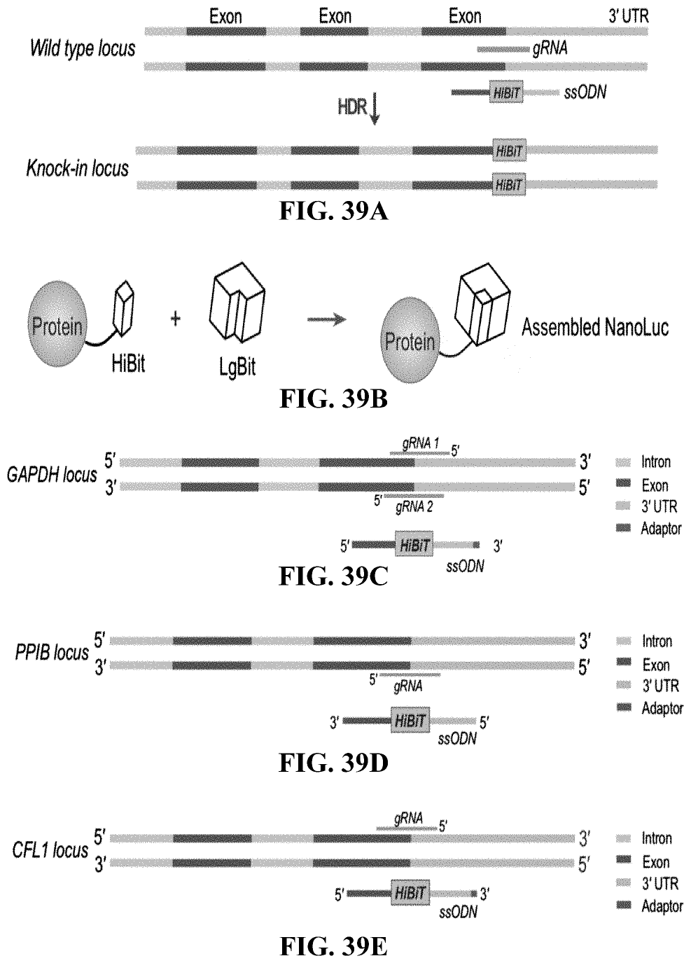

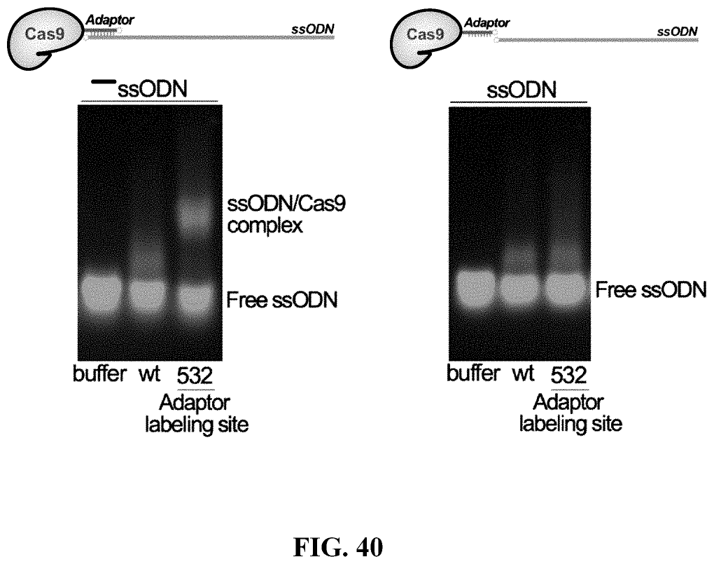

Applicants then designed an ssODN that would insert a 33-nt DNA fragment (HiBiT sequence) at a gene of interest by HDR pathway (FIG. 39A). This insertion results in the expression of a fusion protein with a C-terminal HiBiT tag, which is a small fragment of the NanoLuc luciferase. When complemented by LgBiT, the remainder of NanoLuc, the full-length luciferase is reconstituted to generate a luminescence signal proportional to the degree of ssODN incorporation, providing an easy readout for measuring the level of HDR (FIG. 39B). Applicants chose GAPDH as the first target gene (FIG. 39C) owing to its abundant expression in many cell types, which should allow for the reliable detection of the luminescence signal. Applicants designed two ssODNs that had the same homology arms and insertion sequence, one with a sequence complementary to the Cas9-adaptor conjugates and one without to serve as a negative control (FIG. 13a), and Applicants confirmed the conjugation of the ssODN bearing the complementary sequence to Cas9-adaptor using a gel-shift assay (FIG. 40). Using the negative control ssODN without the complementary sequence, Applicants determined if appending the DNA adaptor to the cysteine affected Cas9 activity using the HiBiT insertion assay (FIG. 13b). As expected, much of the enzyme activity was lost by modifications at residues 558 and 1116, indicating site-specificity of the labeling and reliability of the measurement. Using this assay, Applicants were able to identify five mutants (1C, 532C, 945C, 1026C, 1207C) whose activity was largely maintained (>85% of wildtype in U2OS.eGFP.PEST cells) even after labeling with the 17-nt adaptor, and further characterized the behavior of these five mutants in multiple cell lines (U2OS, HEK-293FT, MDA-MB-231) using conjugated ssODN. Using the luminescence signals from unconjugated ssODN as normalization controls (left panels, FIGS. 13c-13e), Applicants demonstrated an enhancement in knock-in efficiency in all cell lines tested, with more than four-fold increase in HEK-293FT cells. Applicants were able to rank the efficiencies of the mutants in each cell line, with two internal conjugation sites (532C, 945C) generally performing better than the terminal conjugation site (1C). An examination of the crystal structure indicates that cargos on the two internal residues are expected to align with substrate DNA while cargos on the terminal residue project outward from the DNA, which may explain the differences in the HDR-enhancing capacities of different mutants. Applicants note that in contrast to the multi-site internal fusions, genetic fusions of cargos are limited to the termini, restricting the options for generating optimal Cas9-sODN conjugates.

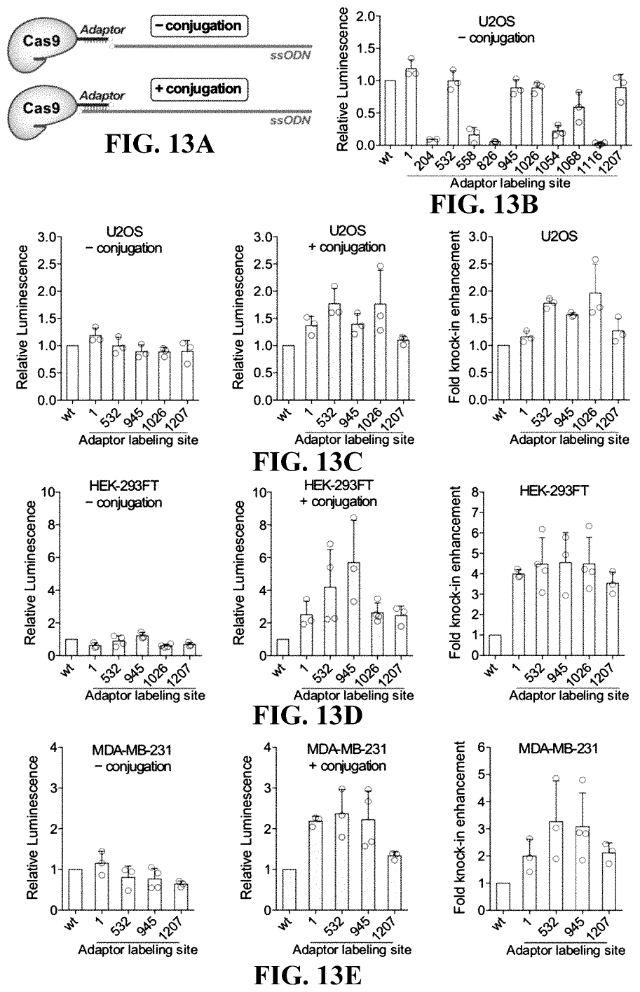

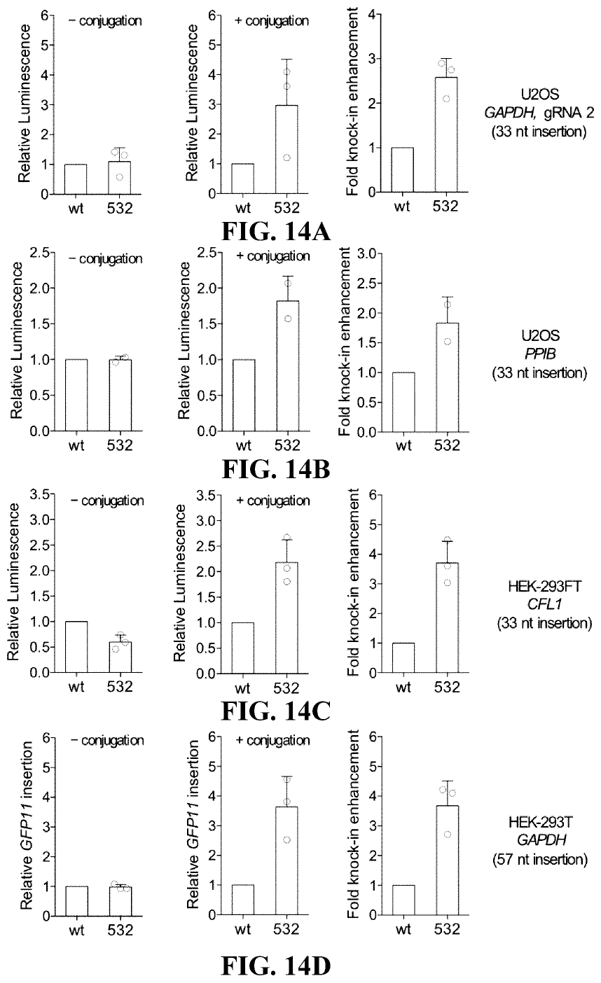

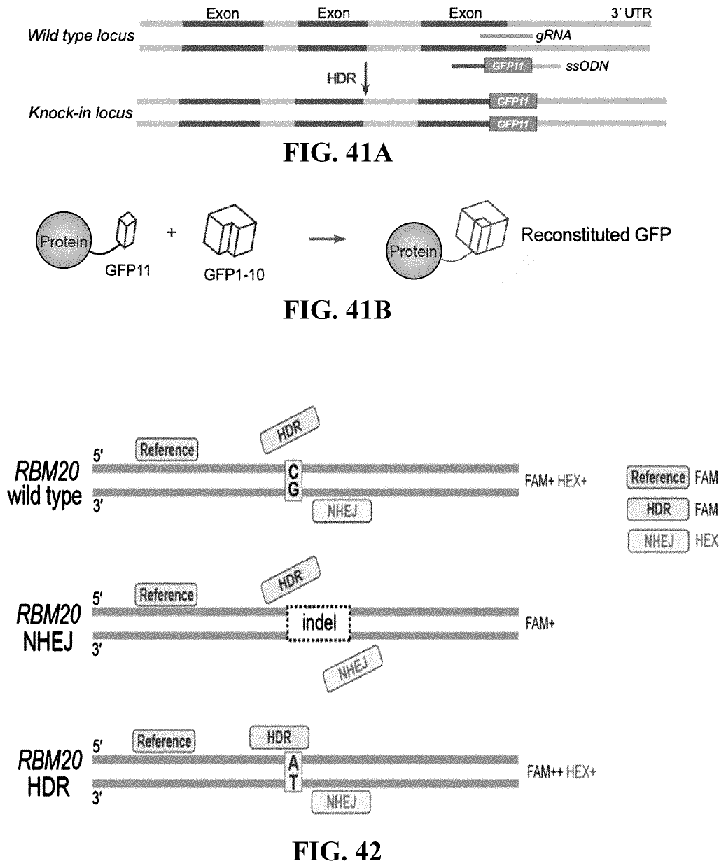

Applicants confirmed the enhancement of knock-in efficiency at another cleavage site of the GAPDH locus (FIGS. 14a and 39c) as well as at multiple genomic loci (PPIB, CFL1; FIGS. 14b, 14c, and 39d, 39e). Applicants demonstrated enhanced knock-in using a longer DNA fragment (GFP11, 57 nt) at the GAPDH locus, whose correct incorporation expresses a fusion protein with a C-terminal GFP11 tag that can form a fully functional GFP when complemented by GFP1-10 for easy fluorescence detection in cells (FIG. 41A-41B). Here as well, the Cas9-ssODN conjugation increased the knock-in efficiency by more than three-fold (FIG. 14D).

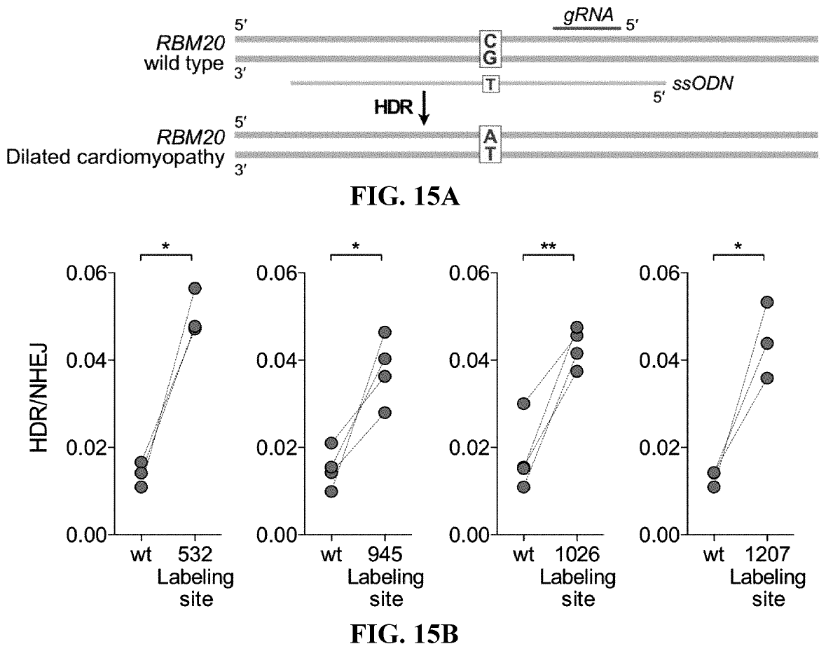

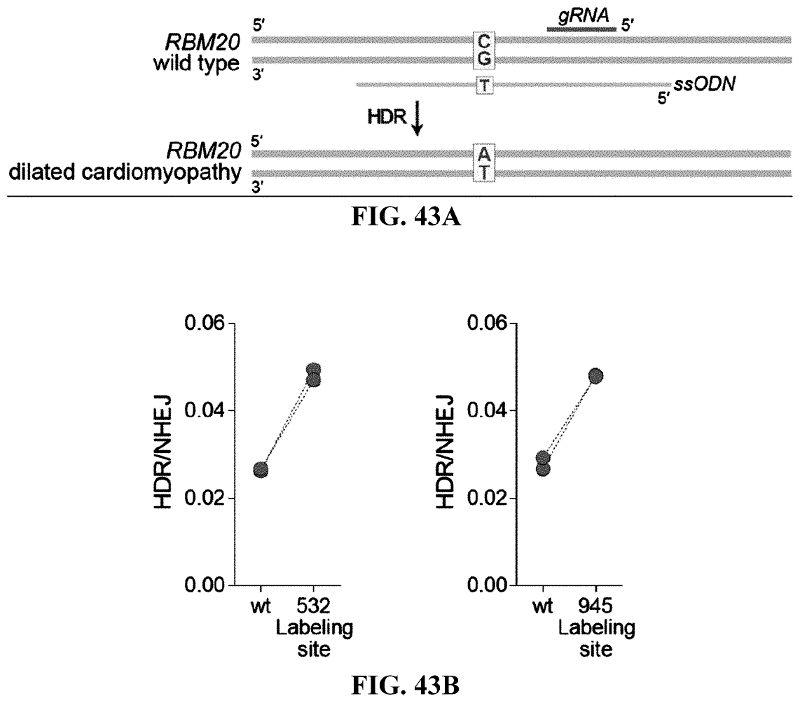

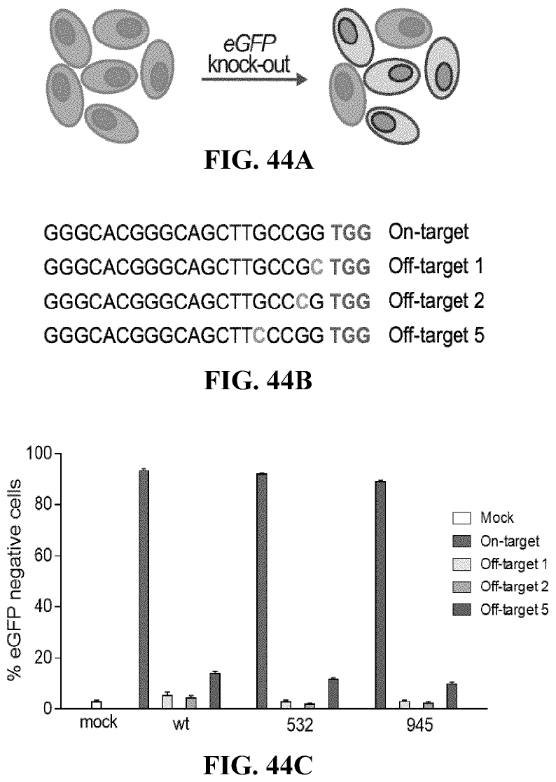

Next, Applicants directly measured the ratio of HDR-mediated single nucleotide exchange to a random indel generated by NHEJ (FIG. 15a) using a reported droplet digital PCR (ddPCR) assay that employs probes to distinguish between wildtype, NHEJ-edited, and HDR-edited sequences at RBM20 locus (FIG. 42). All Cas9-ssODN conjugates increased the ratio of HDR over NHEJ, again indicating the generality of the platform. The conjugates also enhanced HDR rate when another gRNA/ssODN pair was employed to introduce the same mutation (FIG. 43A-43B). Finally, Applicants investigated the off-target profile of the Cas9-adaptor conjugate in an eGFP disruption assay using a perfect match gRNA and off-target gRNAs targeting the eGFP gene stably integrated into the genome of U2OS cells.²⁸ As shown in FIG. 41A-41B, the Cas9-adaptor conjugate retained the target specificity while maintaining the on-target activity.

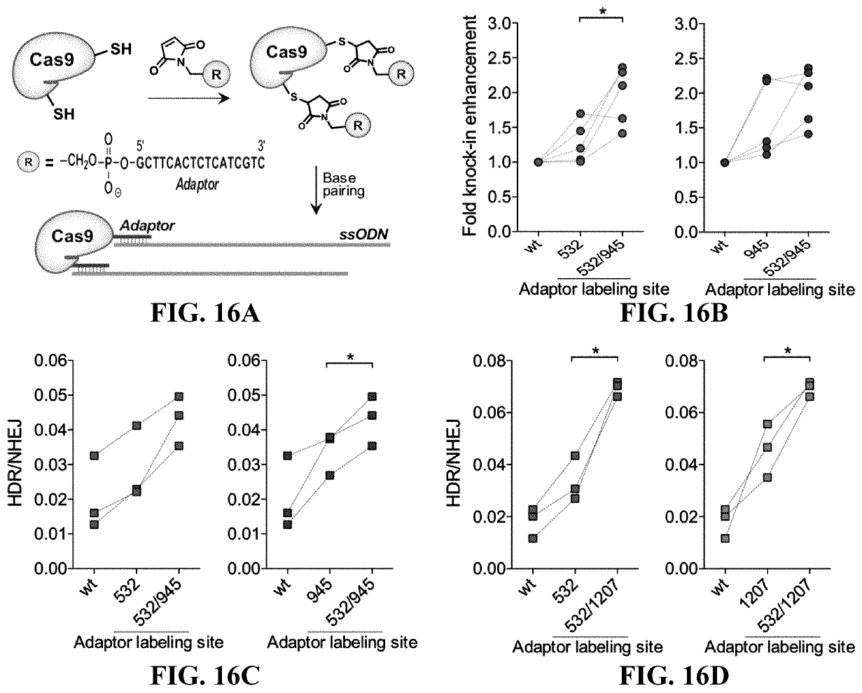

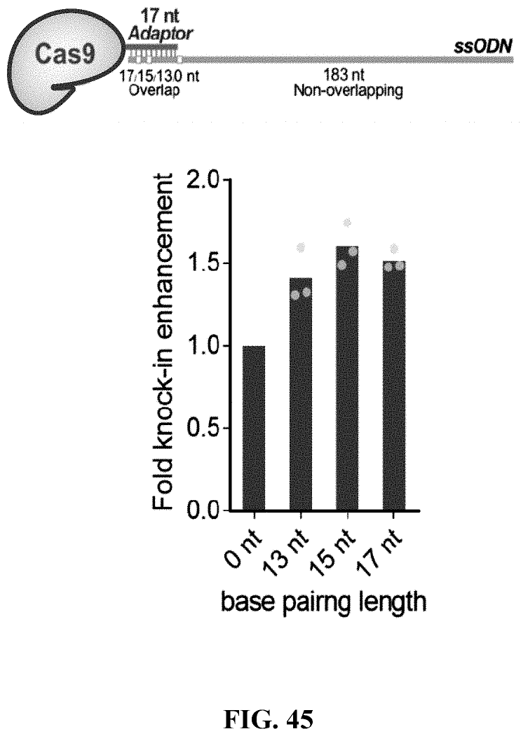

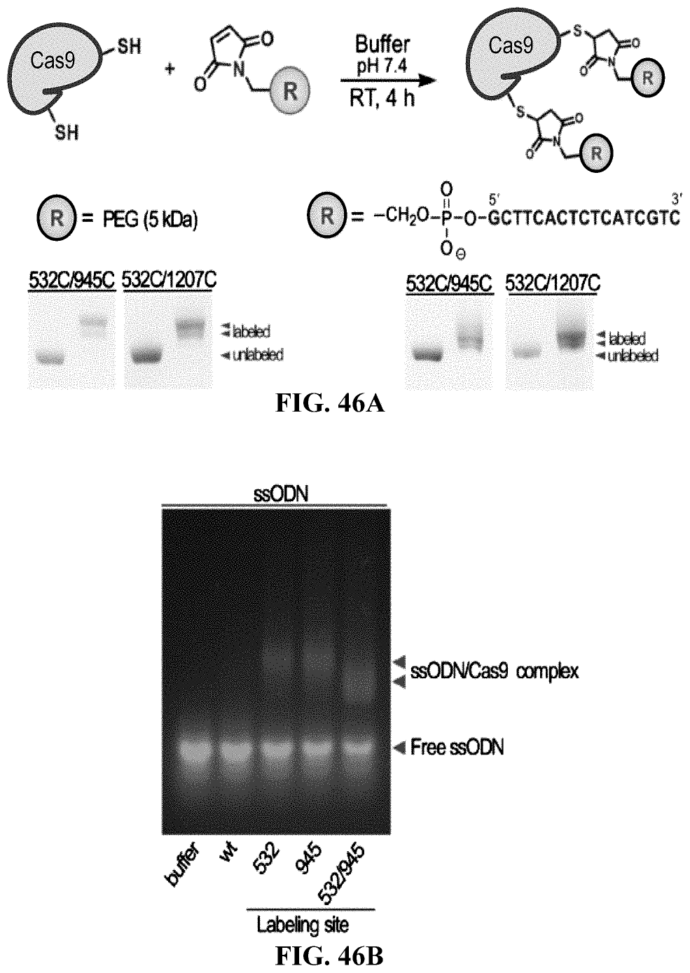

While these studies were underway, reports of genetic fusions of Cas9 to avidin, SNAP, or PCV, which in turn can bind to ssODN, have appeared in the literature,^{12,29-30} and the current studies complement these approaches in multiple ways. First, the current Cas9-adaptor constructs are much smaller than the reported constructs. Applicants also investigated the possibility of further decreasing the length of the adaptor, and found that hybridization by 13 nt or 15 nt showed similar HDR-enhancing effect as the standard 17 nt pairing (FIG. 45). Second, while the genetic fusions were mostly tested at the N- and C-term of Cas9, Applicants have systematically investigated terminal and internal conjugation sites and found that internal conjugation sites yielded higher knock-in efficiencies. Third, the adaptor-based conjugation strategy does not require chemical modification of ssODNs in comparison to avidin- or SNAP-based methods. In addition, adaptor sequence can readily be opted depending on the ssODN sequence for preventing secondary structure formation while PCV recognition sequence cannot be changed. More importantly, owing to the small size of the adaptor and chemical nature of the platform, multivalent displays are feasible (FIG. 16A). To demonstrate this, Applicants produced Cas9 double-cysteine mutants (532C/945C and 532C/1207C) and labeled them with the adaptor (FIG. 46A). Next, Applicants confirmed the binding of ssODNs to Cas9 (FIG. 46B) and observed a boost in HDR efficiency for both the 33-nt HiBiT insertion and a single nucleotide exchange (FIGS. 16B, 16C, 16D).

In summary, Applicants present a simple, scalable, and modular chemical platform for site-specific Cas9 labeling with a wide range of functional molecules. Applicants first identified multiple internal residues which are compatible with modification by thiol-maleimide reaction without compromising the enzyme function. As model labels, small molecule (biotin) and medium-sized molecule (PEG) were efficiently linked to Cas9. Then, Applicants conjugated a short oligonucleotide handle as a universal anchoring point for any kind of oligonucleotide-containing functional molecules, making this platform amenable to nearly every type of desired conjugate. When ssODN was attached to this anchor, HDR efficiency was significantly increased in all genome editing cases Applicants tested, indicating the desirability for Cas9 conjugation systems in gene editing applications. The Cas9-adaptor design was modular in that the same construct could be used for multiple HDR cases (seven examples in this study) without a need for additional experimental steps. Moreover, the chemical platform enabled multivalent display of ssODN, which further enhanced HDR efficiency. Therefore, our Cas9-ssODN conjugation system will serve as a tool for safe and practical genome editing in diverse applications. Beyond ssODN, the adaptor handle can hybridize to any type of cargos bearing the complementary DNA, providing a new method for the practical application of genome engineering technology.

### Example 2—NHEJ Inhibitor/HDR Actovator Small-Molecules

In earlier work disclosed in PCT/US2018/057182, several known NHEJ inhibitors were synthesized or acquired, and test, including SCR7 analogs. Presented here are additional NHEJ inhibitor molecules for use in the Synthetic All in One Genome Editors. SCR6 and SCR7 analogs were investigated. Particularly preferred analogs include the SCR6 analogs

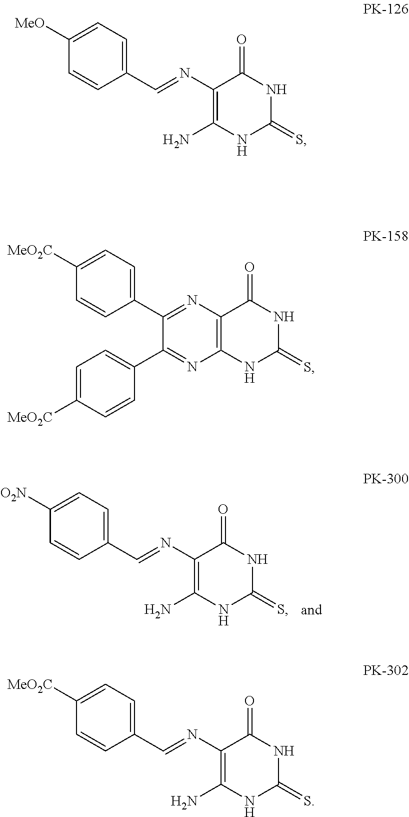 [SMILES: COc1ccc(/C=N/c2c(N)[nH]c(=S)[nH]c2=O)cc1 ; COC(=O)c1ccc(-c2nc3[nH]c(=S)[nH]c(=O)c3nc2-c2ccc(C(=O)OC)cc2)cc1 ; Nc1[nH]c(=S)[nH]c(=O)c1/N=C/c1ccc([N+](=O)[O-])cc1 ; COC(=O)c1ccc(/C=N/c2c(N)[nH]c(=S)[nH]c2=O)cc1]

Novel SCR7 analogs were also investigated and include

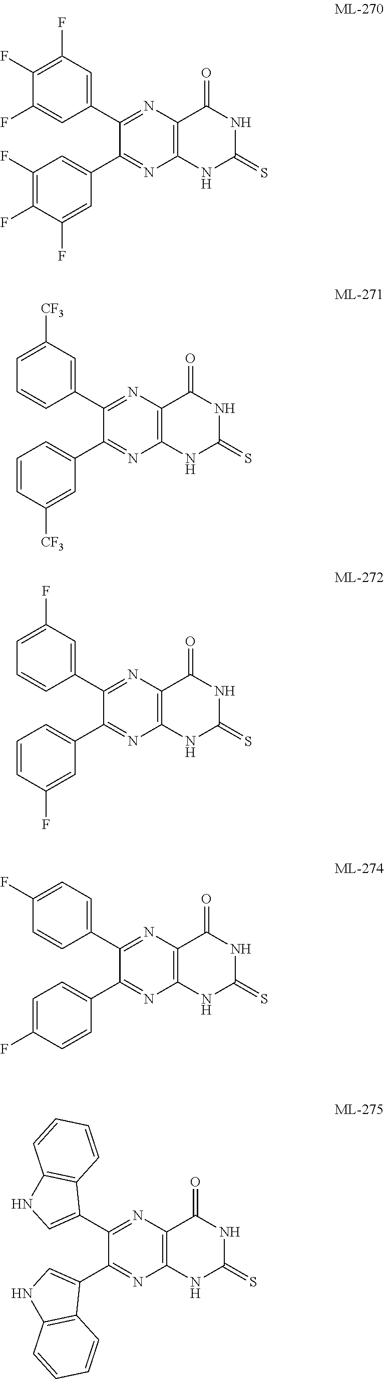 [SMILES: O=c1[nH]c(=S)[nH]c2nc(-c3cc(F)c(F)c(F)c3)c(-c3cc(F)c(F)c(F)c3)nc12 ; O=c1[nH]c(=S)[nH]c2nc(-c3cccc(C(F)(F)F)c3)c(-c3cccc(C(F)(F)F)c3)nc12 ; O=c1[nH]c(=S)[nH]c2nc(-c3cccc(F)c3)c(-c3cccc(F)c3)nc12 ; O=c1[nH]c(=S)[nH]c2nc(-c3ccc(F)cc3)c(-c3ccc(F)cc3)nc12 ; O=c1[nH]c(=S)[nH]c2nc(-c3c[nH]c4ccccc34)c(-c3c[nH]c4ccccc34)nc12]

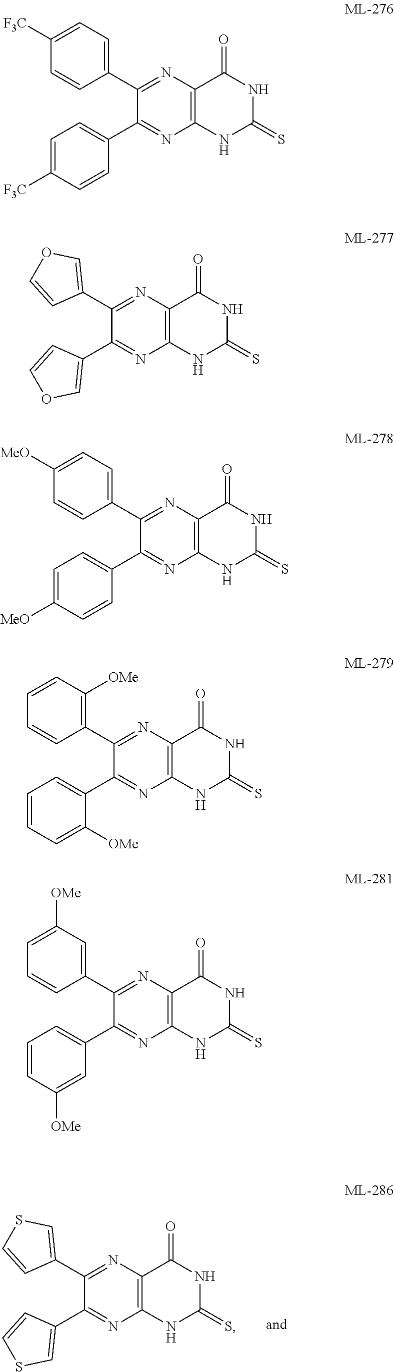 [SMILES: O=c1[nH]c(=S)[nH]c2nc(-c3ccc(C(F)(F)F)cc3)c(-c3ccc(C(F)(F)F)cc3)nc12 ; O=c1[nH]c(=S)[nH]c2nc(-c3ccoc3)c(-c3ccoc3)nc12 ; COc1ccc(-c2nc3[nH]c(=S)[nH]c(=O)c3nc2-c2ccc(OC)cc2)cc1 ; COc1ccccc1-c1nc2[nH]c(=S)[nH]c(=O)c2nc1-c1ccccc1OC ; COc1cccc(-c2nc3[nH]c(=S)[nH]c(=O)c3nc2-c2cccc(OC)c2)c1 ; O=c1[nH]c(=S)[nH]c2nc(-c3ccsc3)c(-c3ccsc3)nc12]

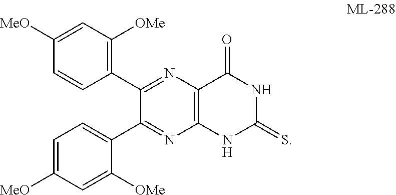 [SMILES: COc1ccc(-c2nc3[nH]c(=S)[nH]c(=O)c3nc2-c2ccc(OC)cc2OC)c(OC)c1]

Applicants additionally investigated HDR Activators

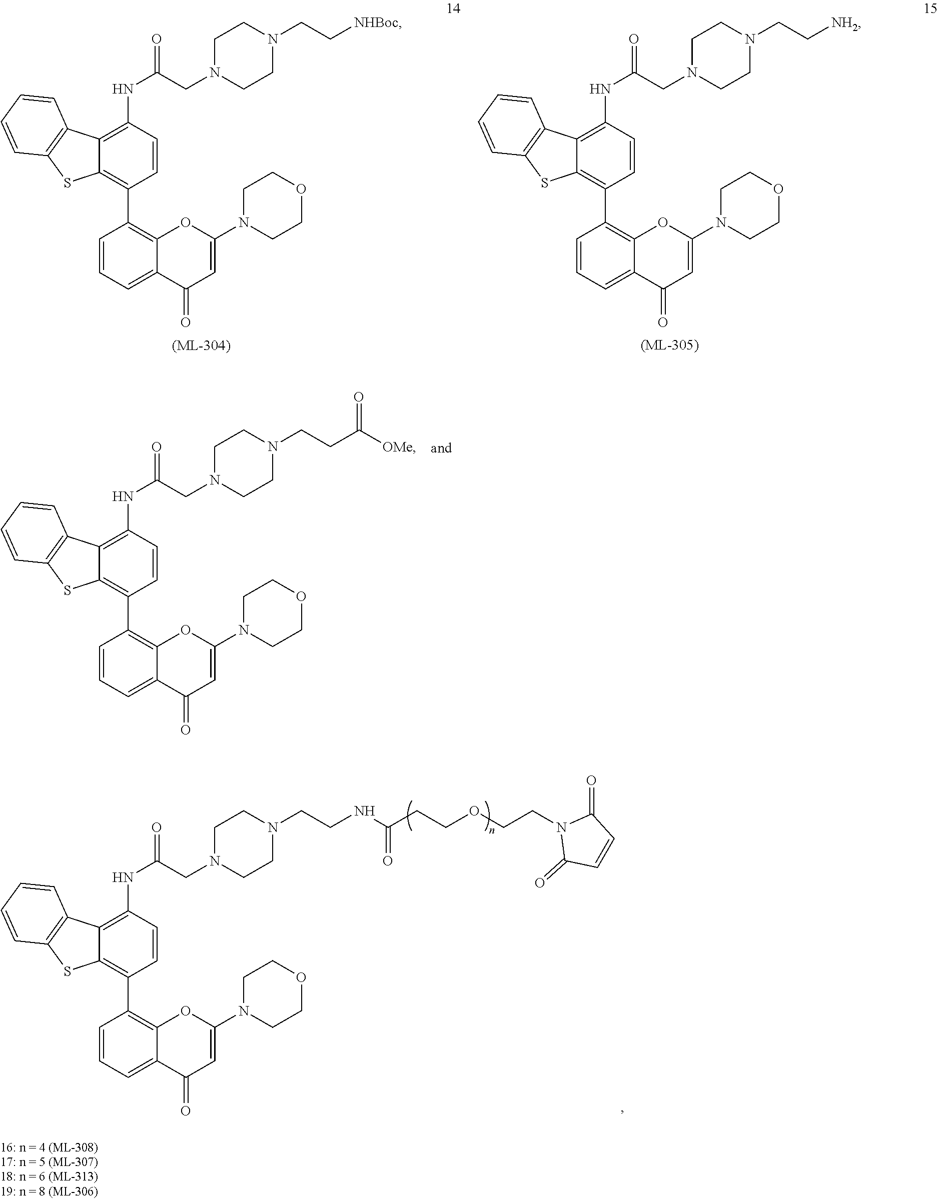

wherein n=4, 5, 6 or 8.

### Example 3—Small Molecule Strand Breakers for Conjugation

Applicants prepared additional small molecule strand breakers that can be conjugated to the SAGE as disclosed herein.

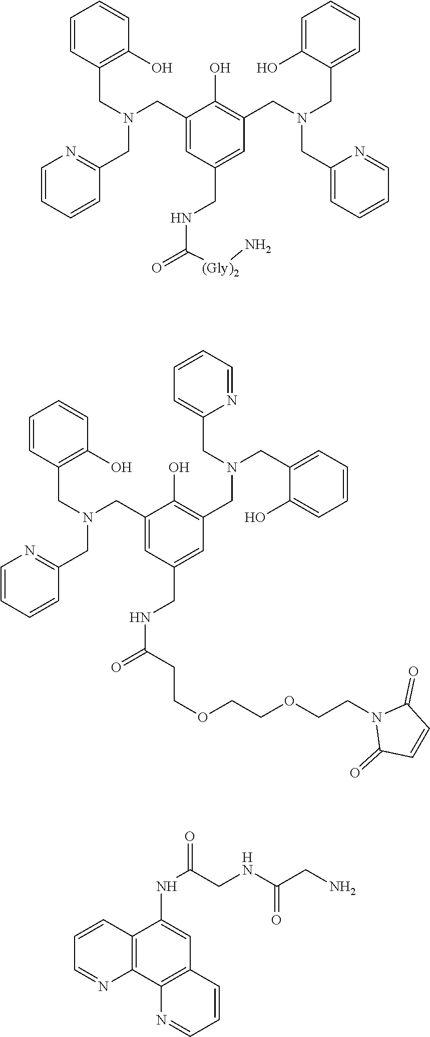

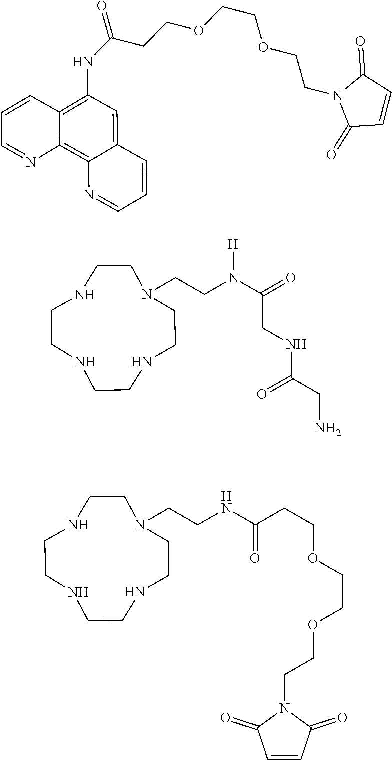 [SMILES: O=C(CCOCCOCCN1C(=O)C=CC1=O)Nc1cc2cccnc2c2ncccc12 ; NCC(=O)NCC(=O)NCCN1CCNCCNCCNCC1 ; O=C(CCOCCOCCN1C(=O)C=CC1=O)NCCN1CCNCCNCCNCC1]

### Example 4—Cell Engineering

β-cell transplantation shows promise, however poor graft survival due to alloimmune and autoimmune rejection and engraftment inefficiency prevents sustained therapeutic effects, and it suffers from a striking initial graft loss of 55% to 70%2,3. Global immunosuppressants can decrease islet rejection, but there is increased risk for opportunistic infections. Applicants proposed to engineer islets with CRISPR-Cas9 based technology for long-term, self-mediated secretion of low-level immunosuppressive cytokines to abate chronic immune attacks and inhibit fibrosis formation. The first challenge was to address the genome editing and the pre-disposition of NHEJ repair which can lead to the p53 apoptosis pathway.

The SAGE “all-in-one” approach is to be used by pioneering the development of a multifunctional Cas9 whose capacities are augmented using small molecules and donor DNAs as disclosed in this application. Chemical biologists have developed a powerful array of cell-compatible chemical conjugation techniques that can enhance the endogenous function of a given protein. Applicants studies will, for the first time, apply these powerful approaches toward the building of a suite of active Cas9 proteins capable of multivalent, orthogonal, and novel chemical conjugation. Current approaches toward increasing high knock-in involves global inhibition of NHEJ repair pathways across the cell, which requires high concentrations of exogenously supplied inhibitor that can lead to undesired toxicity and mutagenicity. Through chemical conjugation, Applicants will develop strategies to locally enhance the concentration of cell repair-biasing molecules at the target site, leading to high fidelity and nontoxic repair.

Base-editors display uracil DNA glycosylase inhibitors for local inhibition of these glycosylases, which is a key requirement for efficient base editing. Inspired by the mechanism-of action of antibody-drug conjugates and base editors, Applicants have proposed to display ssODN, NHEJ and p53 pathway modulators on Cas9 to generate a semi-synthetic, multifunctional genome editor, which Applicant scall SynGEM (FIG. 1A). Locating ssODNs close to the DNA break site would enhance the rate of precision genome editing due to the increased local concentration. In addition, inhibiting NHEJ pathways can be a viable strategy to direct DNA repair process toward HDR pathway. Cas9-induced double-strand break leads to activation of p53 pathway followed by apoptosis, greatly reducing the efficiency of precision genome editing in primary cells and stem cells. Selection process to enrich HDR-edited cells may enrich p53-impared cells, which will increase the risk of tumor development when used in clinic. Therefore, temporarily inhibiting p53 pathway by small molecules will be another viable strategy for increasing HDR efficiency while lowering genotoxicity. Based on these assumptions, SynGEMs will be developed. Long ssODNs will be attached to Cas9 by developing a modular conjugation strategy that enables tethering of any ssODN without extra steps. Known inhibitors of the NHEJ and p53 pathway can be appended to Cas9. Local display of small molecules would minimize the toxicity and mutagenesis due to global NHEJ/p53 pathway inhibition. Towards these goals, Applicants have optimized orthogonal conjugation chemistries to Cas9 and demonstrated HDR enhancement by ssODN tethering to Cas9. Applicants have also validated IL-10 secretion from INS1E cells using Cas9-mediated genome editing.

Applicants first developed a platform for site-specific cysteine conjugation on Cas9. Guided by structure of Cas9, cysteines were engineered on solvent-exposed loops of various Cas9 domains, and mutated polar residues to minimize potential structure disruption. Eleven single cysteine mutants were recombinantly expressed and using PEG (5 kDa)-maleimide conjugation, Applicants confirmed that conjugation at 10 sites (M1C, S204C, E532C, K558C, Q826C, E945C, E1026C, E1068C, S1116C, E1207C) was efficient (data now shown). Next, the single-cysteine mutants were labeled with short oligonucleotide named ‘universal adaptor’ that can be an anchoring point for any kind of functional molecules based on DNA hybridization (FIG. 1A). Applicants designed a ssODN that would insert a 33-nt DNA fragment (HiBiT sequence) at the GAPDH locus by HDR (FIG. 1B). This insertion would result in the expression of a fusion protein containing a C-terminal HiBiT tag, a small fragment of the NanoLuc luciferase. Upon cell lysis and complementation with the remainder of NanoLuc, LgBiT, intact NanoLuc is reconstituted eliciting a robust luminescent signal that is proportional to the degree of ssODN insertion (FIG. 1B). Applicants designed two ssODNs that had the same homology arms and insertion sequence, one with a sequence complementary to the adaptor for conjugation and one without for a negative control. Using the negative control ssODN without the complementary sequence, and determined whether appending the DNA adaptor to Cas9 affected the enzyme activity in the HiBiT sequence knock-in assay (FIG. 1C). Five mutants (1C, 532C, 945C, 1026C, 1207C) were identified whose activity was largely maintained (>85% of wildtype in U2OS cells) even after labeling with the 17-nt adaptor. Next, Applicants proceeded to use Cas9 labeled at those sites and using the luminescence signals from unconjugated ssODN as normalization controls (FIG. 1D), and demonstrated an enhancement in knock-in efficiency by Cas9-ssODN conjugation in multiple cell lines. Finally, appendage of two ssODN significantly improved the degree of HDR (data not shown). Applicants have now confirmed these enhancements in HDR using multiple readouts (e.g., ddPCR) and at several genomic loci without altering the specificity of Cas9. Applicants have also engineered Cas9 to accommodate a single sortase recognition sequence (Leu-Pro-Xxx-Thr-Gly, where Xxx is any amino acid) in collaboration with Feng Zhang's laboratory at the Broad Institute (FIG. 1E). Expression of these sortase loop-containing Cas9 variants (Cas9-SortLoop) in mammalian cells verified that most retained activity compared to wtCas9, as validated by next-generation sequencing assays quantifying insertion/deletion (indel) mutations events against EMX1 (FIG. 1F). Applicants confirmed sortase-mediated labeling of a model biotin-containing poly-Gly peptide for SortLoop #7 (FIG. 1G). These studies confirm that sortase chemistry can be used for labeling of Cas9 without perturbing activity. Following the demonstration of HDR enhancement using conjugated ssODN to Cas9, Applicants optimized the structure of small-molecule modulators of NHEJ pathway for their activity in cells. Here, Applicants synthesized or acquired several known NHEJ inhibitors, and performed preliminary structure-activity relationship studies to determine potential sites for linker attachment (FIG. 1H). For example, for SCR7 Applicants envisioned that the aryl rings as potential linker attachment site. Applicants further synthesized and tested analogs with various aryl rings using a droplet digital (ddPCR) assay which can detect wildtype and genome-edited alleles in RBM20 locus. Dose-dependent inhibition of NHEJ was seen (FIG. 1I) as previously reported and DNA-PK inhibitors (KU-57788 and KU-0060648) enhanced HDR enhancement in the HiBIT assay (FIG. 1J)

Development and optimization of SynGEM in the context of beta cells. Applicants have already identified potent small-molecule inhibitors of the NHEJ pathway proteins, and there exist several inhibitors of p53 pathway that act by inhibiting ATM kinases. Applicants will append these inhibitors to Cas9. Based on medicinal chemistry studies done by us and others, Applicants have identified sites on these small molecules for linker attachment (FIG. 1H). For NHEJ inhibitors, Applicants will synthesize NHEJ inhibitors tested above (e.g., SCR7 analogs, KU-0060648) to bear linkers (e.g., PEG) that will be conjugated to Cas9, and propose to generate ˜7 conjugates for each Cas9-ssODN, Cas9-NHEJ inhibitor, and Cas9-p53 pathway inhibitor. Applicants will test these conjugates in the ddPCR assay described above to identify the top two conjugates for each category that significantly enhance HDR and prevent genotoxicity (for p53 inhibitor). ssODN attachment will be through adaptors as described in section C.1.2. Following the identification of the most optimized systems for ssODN, NHEJ/p53 pathway inhibition, Applicants will generate a synthetic Cas9 bearing all the three components. Simultaneous orthogonal conjugation of the three components can be challenging, and Applicants propose multiple orthogonal conjugation strategies: cysteine-maleimide, sortase chemistry, and unnatural amino acids bearing groups with orthogonal reactivity to cysteine and sortase. For unnatural amino acid mutagenesis59, Applicants will utilize genetic code expansion by adding an engineered pyrrolysyl tRNA (PylT)/tRNA synthetase pair to the translational machinery of cells to enable the site-specific incorporation of p-azido Phenylalanine (pAzF) into CRISPR/Cas9.60 This method relies on a unique codon-tRNA pair and corresponding aminoacyl tRNA synthetase (aaRS) for each unnatural amino acid that does not cross-react with any of the endogenous tRNAs, aaRSs, amino acids or codons in the host organism. The ribosome translates mRNA into a polypeptide by complementing triplet codons with matching aminoacylated tRNAs.59 Three of the 64 different triplet codons do not code for an amino acid, but cause recruitment of a release factor resulting in disengagement of the ribosome and termination of the synthesis of the growing polypeptide. These codons are called; ochre (TAA), opal (TGA), and amber (TAG). Of the three stop codons, the amber codon is the least used in E. coli (˜7%) and rarely terminates essential genes. Applicants will place amber suppression codons at the optimal sites identified above. While Applicants propose to use pAzF as the unnatural amino acid that can react with cyclooctyne group, Applicants will also explore tetrazine chemistries which are also high yielding and orthogonal to the reactivity of cysteines, and sortase. Applicants note that multiple reports for incorporation of unnatural amino acids in Cas9 exists61 and members of the PIs laboratory62,63 have deep expertise in unnatural amino acid mutagenesis.

Efficient Knock-In of Immunomodulatory Genes in Human Islets and Human Stem Cell Derived f-Cells.

Applicants will synthesize and optimize SynGEMs in the context of human islets and human stem cell derived beta cells (hSC β-cells). Human islets from cadaver pancreases will be obtained from JDRF human islet consortium and will be used as such for the knock-in experiments. Multiple methods have now been described for efficient delivery of Cas9:gRNA:ssODN in primary cells, including nucleofection which will be explored. In vitro cytokine release profile from cells as well as manipulation of cell density, device design and reiterate on details of gene insertion such as the insertion site, gRNA and ssODN design to recapitulate the secretion profile required for the desired anti-inflammatory and anti-fibrosis effect in vivo will be performed.

### Example 5—Genome Editing of Pancreatic p-Cells to Secrete Functional Molecules

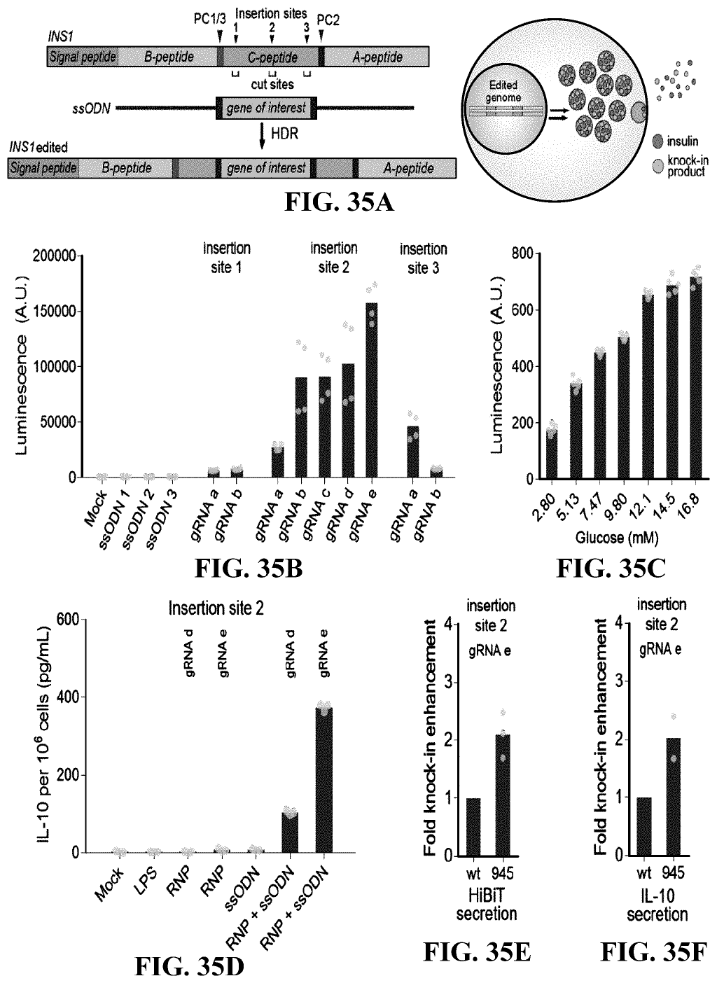

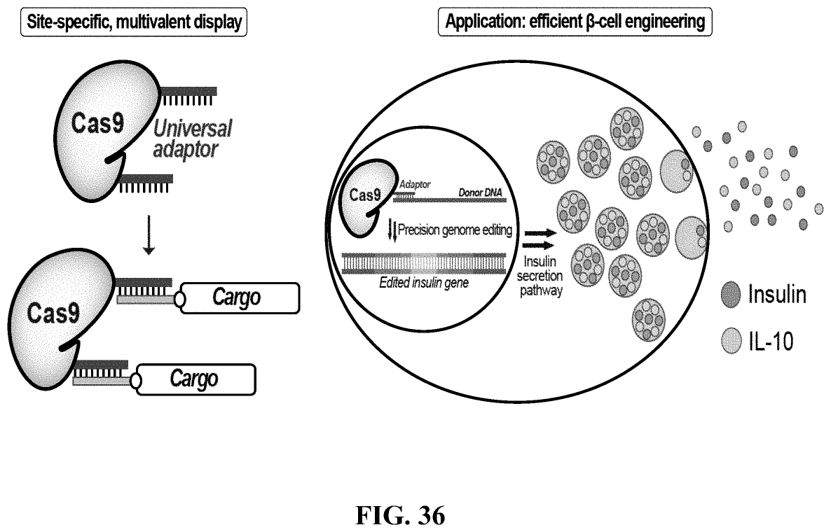

Applicants employed the chemically modified Cas9 to establish a general platform for 1-cell genome editing given the urgent need for providing 1-cells with immunomodulatory functions, such that cure for the diabetes can be achieved by cell-based therapies. Since c-peptide is cleaved off during insulin processing and secreted, we hypothesized that knocking in a desired gene into the c-peptide portion of the proinsulin locus would enable the secretion of the inserted gene product. Indeed, a lentiviral vector encoding proinsulin-luciferase fusion construct, which has a Gaussia luciferase in the middle of the c-peptide portion, expressed functional luciferase when stably integrated into the INS-1E β-cell line. Here, the expression level of luciferase was directly proportional to that of insulin, and responded sensitively to external stimuli such as glucose concentration. However, β-cells engineered with viral vectors poses safety issues such as immunogenicity. Thus, precise genome editing can be a powerful way to insert a desired gene fragment into the c-peptide region, which will allow co-secretion of the target gene product with insulin (FIG. 35A).

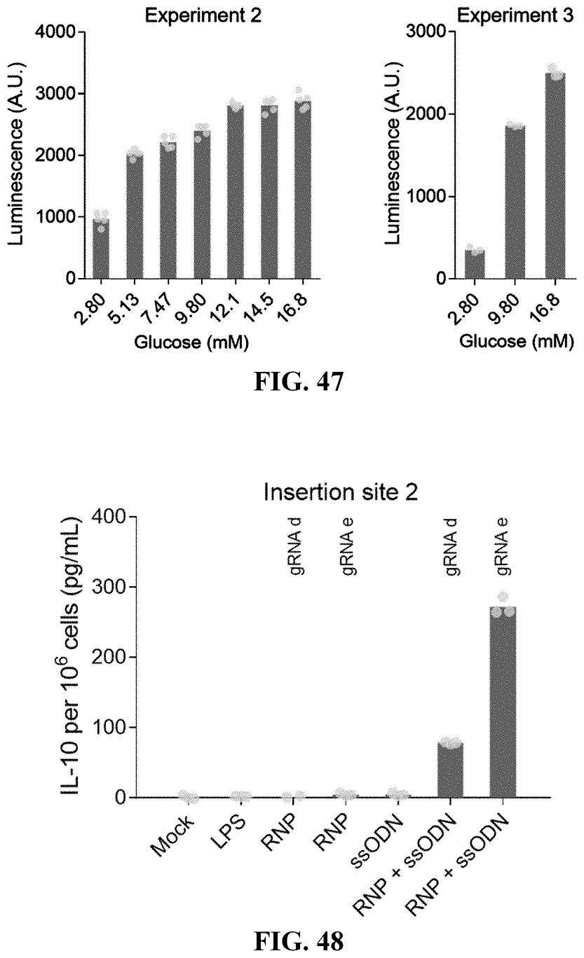

As a proof-of-concept, Applicants first demonstrated the HDR-mediated knock-in of the HiBiT sequence at the c-peptide portion of INS1 locus in INS-1E cells. Target HiBiT sequence was flanked by additional prohormone convertase 2 (PC2) cleavage sites. Therefore, no extra amino acids would be present at each end of the knock-in product after processing (FIG. 35A). To identify the best gene insertion site and DNA cleavage site, three gene insertion sites were chosen at the start, middle, and end regions of the c-peptide locus, and designed several gRNAs to target these sites such that insertion sites and DNA cleavage sites are close enough to obtain high HDR efficiency (FIG. 35A). In addition, genome-wide off-target profiles of gRNAs were considered so that potential off-target sites have mismatches at the seed sequences or have at least three mismatches in the gene-encoding regions. When genome editing was performed at these target sites using Cas9 ribonucleoprotein (RNP) and ssODNs, HiBiT peptide was secreted from INS-1E cells, which could be readily detected through luminescence signals from the cell culture supernatant after complementation by the LgBiT protein. The highest knock-in efficiency was achieved when the c-peptide middle region (site 2) was targeted (FIG. 35B). Therefore, this insertion site was used for the following experiments. HiBiT peptide secretion was stimulated by glucose, indicating that the knock-in product is secreted thought the insulin processing and secretion pathways (FIGS. 35C and 47).

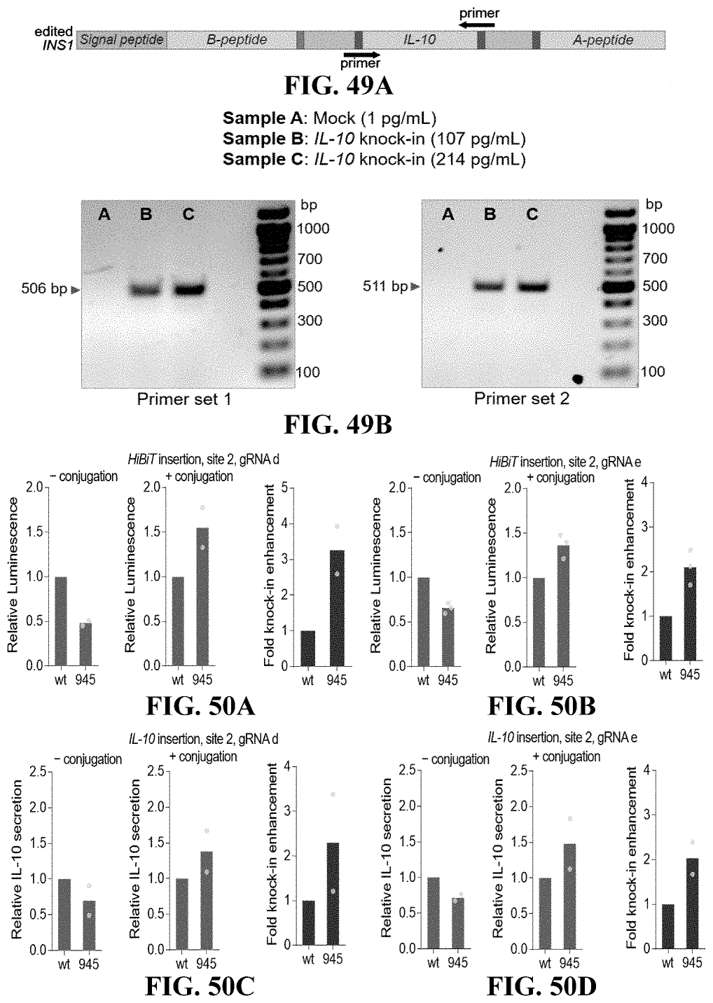

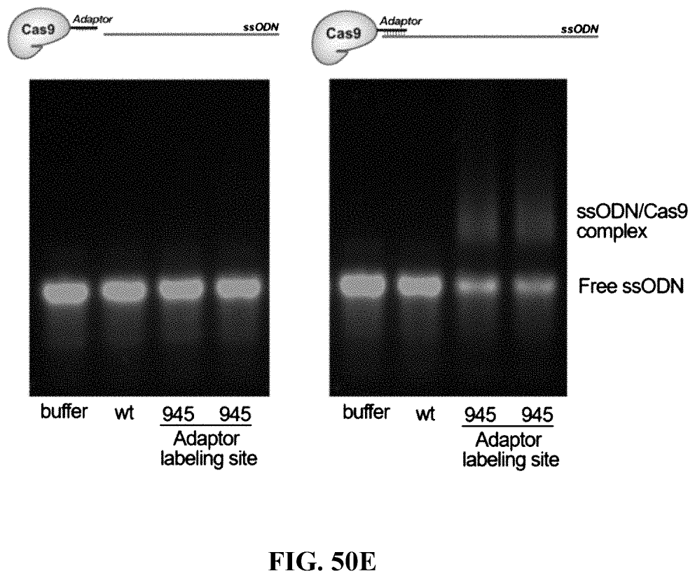

Based on this optimized design, we set out to knock-in a long gene fragment to secret an anti-inflammatory cytokine IL-10 that can protect β-cell from the immune-triggered destruction.³⁰ The ssODNs for IL-10 insertion had longer homology arms (150 nt left arm and 155 nt right arm) for efficient incorporation of the long gene fragment. Secretory signal peptide sequence present in IL-10 gene was omitted, and only mature protein portion was used because insulin secretion pathway is responsible for the IL-10 secretion at the engineered P-cells. PC2 cleavage sites were added at each end of IL-10 for obtaining intact IL-10 as the knock-in product, and the corresponding ssODN was synthesized by reverse transcription. When INS-1E cells were transfected with both Cas9 RNP and ssODN, IL-10 was secreted to the cell culture media as determined by enzyme-linked immunosorbent assay (ELISA). RNP only nor ssODN only did not induce IL-10 secretion. Moreover, treatment of the cells with lipopolysaccharides (LPS) was not enough to induce IL-10 secretion (FIGS. 35D and 48). Then, we extracted genomic DNA from unedited and edited cells, and amplified the knock-in sequence using the knock-in-specific primer pairs. The IL-10 secretion level correlated with the amount of the edited genomic DNA (FIG. 49), and Sanger sequencing confirmed the correct insertion of the IL-10 gene at the INS1 c-peptide region. Finally, we employed our chemical Cas9 modification system for this β-cell genome editing, and found that both HiBiT secretion and IL-10 secretion were promoted by Cas9-ssODN conjugates (FIGS. 35E, 35F and 50).

### Experimental Methods for Examples 1-5

Cas9 Expression and Purification

A plasmid for SpCas9 expression (2×NLS and C-terminal His tag, pET-28a) was a gift from the Gao group (Addgene #98158).¹ E. coli Rosette2 (DE3)-expressing wildtype Cas9, single-cysteine Cas9 mutants, or double-cysteine Cas9 mutants were grown overnight at 18° C. with 0.5 mM of IPTG supplemented when the OD_{600 nm} reached 0.8-1.2. The protein was purified by successive Ni-NTA affinity chromatography, cation exchange chromatography, and size-exclusion chromatography. Purified proteins were snap-frozen in liquid nitrogen and stored at −80° C. in Cas9 storage buffer (20 mM Tris HCl, 0.1 M KCl, 1 mM TCEP, 20% glycerol, pH 7.5).

Site-Directed Mutagenesis

Two cysteine residues in SpCas9 (C80, C574) were replaced by serine to give a cysteine-free mutant. Based on this construct, multiple single-cysteine and double-cysteine mutants were generated by introducing cysteines at the designated residues. Mutagenesis was performed using the partial overlapping primer design method² or using a Q5 Site-Directed Mutagenesis Kit (New England Biolabs).

Cas9 Labeling by Thiol-Maleimide Conjugation

Adaptor oligonucleotide (GCTTCACTCTCATCGTC) (SEQ ID NO: 15) modified with protected maleimide (maleimide-2,5-dimethylfuran cycloadduct) at the 5′ terminus was synthesized by Gene Link. Prior to thiol-maleimide conjugation, the maleimide group was deprotected via retro-Diels-Alder reaction by heating the DNA in toluene for 3 h at 90° C. Solvent was removed under the reduced pressure, and the resulting DNA in solid form was dissolved in water to give 2 mM solution. Cas9 cysteine mutants (4 μM) were mixed with 300 μM of PEG (5 kDa)-maleimide or adaptor oligonucleotide-maleimide in reaction buffer (20 mM Tris HCl, 0.1 M KCl, 1 mM TCEP, pH 7.5). The reaction proceeded for 3 h at room temperature (RT) with mild shaking. The resulting mixture was diluted with a high-salt buffer (20 mM Tris HCl, 1 M KCl, 1 mM TCEP, 20% glycerol, pH 7.5) and incubated with Ni-NTA agarose beads at 4° C. The beads were extensively washed with the high-salt buffer to completely remove non-specifically bound oligonucleotide molecules. Labeled Cas9 was eluted with an elution buffer (20 mM Tris HCl, 0.1 M KCl, 1 mM TCEP, 250 mM imidazole, 10% glycerol, pH 7.5). Finally, buffer exchange was conducted using Amicon Ultra-0.5 mL centrifugal filters with a 100 kDa cut-off (Millipore) to give Cas9-adaptor conjugates in storage buffer (20 mM Tris HCl, 0.1 M KCl, 1 mM TCEP, 10% glycerol, pH 7.5).

Cas9 Biotin Labeling and Pull-Down by Streptavidin Beads

Cas9 with enhanced specificity [eSpCas9(1.1)]³ was used for biotin labeling. Cas9 cysteine mutants (7 μM) were mixed with 500 μM of EZ-Link™ Maleimide-PEG2-Biotin (Thermo) in a reaction buffer (20 mM Tris HCl, 0.1 M KCl, 1 mM TCEP, pH 7.5). The reaction proceeded for 4 h at room temperature (RT) with mild rotation. Excess compounds were removed by Bio-Gel P-6 columns (Biorad) according to the manufacturer's protocol. Next, 30 pmol of Cas9 from the above step was incubated with 30 μL of Pierce Streptavidin Magnetic Beads (Thermo) overnight at 4° C. Flow-through was collected and the beads were washed twice with a washing buffer (20 mM Tris HCl, 0.15 M NaCl, 0.1% Tween20, pH 7.4; 300 μL) and once with the reaction buffer (300 μL). The beads were heated to 95° C. for 5 min in the presence of SDS-PAGE buffer, and the resulting bead-bound fraction (eluate) and flow-through were subjected to SDS-PAGE followed by Coomassie staining.

Electrophoretic Mobility Shift Assay

For this assay, 300 nM of Cas9 was mixed with 300 nM of ssODNs in a binding buffer (20 mM Tris HCl, 0.1 M KCl, 1 mM TCEP, 10% glycerol, pH 7.5). For the Cas9 double-adaptor conjugates, 200 nM of protein and 400 nM of ssODN were used. For testing long ssODNs (Figure S14), 80 nM Cas9 and 60 nM ssODN were used. The mixture was incubated for 30 min at RT and resolved by 1% agarose gel. DNA was stained using SYBR Gold, and fluorescence images were obtained using an Azure c600 (Azure Biosystems) with the Cy3 channel.

In Vitro Transcription to Synthesize Single-Guide RNAs

Sequences of target-specific forward primers and universal reverse primers are listed in Table 4. Polymerase chain reactions (PCR) were conducted using Q5 High-Fidelity 2x Master Mix (New England Biolabs) in the presence of 0.5 μM of forward and reverse primers in a volume of 25 μL. The PCR program was as follows: Initial denaturation at 95° C. for 1 min; 25 cycles of 95° C. for 15 s, 58° C. for 30 s, and 72° C. for 15 s; final extension at 72° C. for 2 min and cooling to 25° C. using a 1% ramp. The resulting mixture was used for in vitro transcription without purification. The reaction was performed using the HiScribe T7 Quick High Yield RNA Synthesis Kit (New England Biolabs). The mixture contained 10 μL of NTP buffer mix, 2 μL of the above crude PCR product, 2 μL of T7 RNA polymerase mix, and 0.75 μL of recombinant RNase inhibitor (New England Biolabs) in a final volume of 30 μL. The reaction was conducted for 10 h at 37° C. DNase treatment was performed to remove template DNA according to the manual. RNAs were purified using the MEGAclear Transcription Clean-Up Kit (Invitrogen) according to the manual.

Short Single-Stranded Oligonucleotides

Single-stranded donor DNAs for HiBiT insertion, GFP11 insertion, and single nucleotide exchange at the RBM20 locus were Ultramer DNA oligonucleotides synthesized by Integrated DNA Technology. Their sequences are listed in Table 5.

Long Single-Stranded Oligonucleotides for IL-10 Insertion

Single-stranded donor DNAs for IL-10 insertion were synthesize by reverse transcription.⁴ First, double-stranded gBlocks DNAs were synthesized by Integrated DNA Technology. The DNAs have the T7 promoter sequences followed by the reverse complementary sequences of the final ssODN sequences. DNAs were produced in large quantities by PCR, followed by gel electrophoresis and gel extraction. Next, in vitro transcription was performed using the HiScribe T7 Quick High Yield RNA Synthesis Kit (New England Biolabs). The mixture contained 10 μL of NTP buffer mix, 400 ng of the double-stranded DNA template, 2 μL of T7 RNA polymerase mix, and 0.4 μL of recombinant RNase inhibitor (New England Biolabs) in a final volume of 20 μL. The reaction was conducted for 5 h at 37° C. DNase treatment was performed to remove template DNA according to the manual. The resulting RNAs were purified using the MEGAclear Transcription Clean-Up Kit (Invitrogen) according to the manual. Finally, reverse transcription was performed to obtain single-stranded donor DNAs. Approximately 200-250 pmol of RNA was mixed with 400 pmol of reverse primer and 6 μL of dNTP mix (25 mM each, New England Biolabs) in nuclease-free water at a final volume of 35 μL. The mixture was incubated at 65° C. for 5 min, then immediately placed on ice for 5 min to induce RNA—primer annealing. Then, 10 μL of 5x RT buffer (250 mM Tris HCl, 375 mM KCl, 15 mM MgCl₂, pH 8.3), 2.5 μL of 0.1 M dithiothreitol solution, 0.5 μL of RNase inhibitor (New England Biolabs), and 2.5 μL of TGIRT-III reverse transcriptase (InGex) were added to the RNA-primer solution. The reaction was proceed at 58° C. for 3 h. Next, RNA was hydrolyzed by adding 21 μL of 0.5 M NaOH solution and heating at 95° C. for 10 min. The basic solution was quenched by the addition of 21 μL of 0.5 M HCl solution. The resulting single-stranded DNAs were purified using MinElute PCR Purification Kit (Qiagen) according to the manual. The purity of the ssDNA was confirmed by 6% TBE-Urea gel electrophoresis followed by SYBR Gold staining. All DNA sequences are listed in Table 6.

### HiBiT Sequence Knock-In by Nucleofection

U2OS.eGFP.PEST cells or MDA-MB-231 cells were transfected with Cas9 ribonucleoprotein (RNP) and ssODN using the SE Cell Line 4D-Nucleofector kit (Lonza) following the pulse program of DN-130 (U2OS.eGFP.PEST) or CH-125 (MDA-MB-231). For Cas9-ssODN conjugates, 10 pmol of Cas9-adaptor were pre-mixed with 10 pmol of ssODN and incubated at RT for 15-30 min prior to RNP formation to ensure Cas9-ssODN conjugate formation. Then 10 pmol of gRNA was added, and the final mixture was incubated for 5-10 min at RT. In cases where Cas9 did not specifically bind ssODNs, the RNP was formed first because it is known that nonspecific Cas9-DNA interactions hamper the RNP formation. After incubating Cas9 and gRNA at RT for 5-10 min, 10 pmol of ssODN were added to the mixture. Approximately 200,000 cells were transfected with the above mixtures in a well of the nucleofection kit, and 20,000 transfected cells were seeded in each well of a 96-well plate. Cells were incubated for 24 h at 37° C., and cell viability was measured using the PrestoBlue Cell Viability Reagent (Thermo). Next, the HiBiT assay was performed using the Nano-Glo HiBiT Lytic Detection System (Promega) according to the manufacturer's protocol. The resulting luminescence signals were normalized based on the cell viability.

### HiBiT Sequence Knock-In by Lipofection

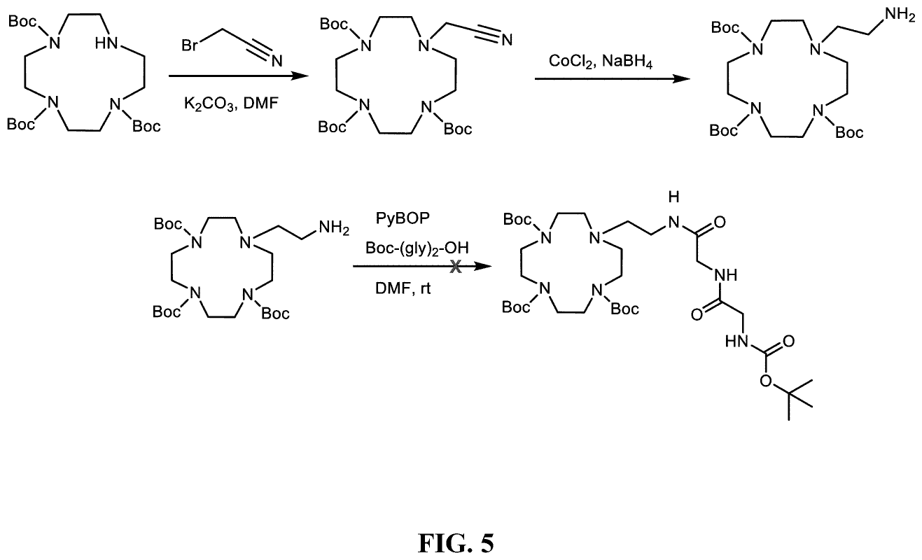

HEK-293FT cells were seeded in a 96-well plate at a density of 10,000 cells per well. The next day, Lipofectamine CRISPRMAX (Invitrogen) was used to transfect the cells with Cas9 RNP and ssODN, with final concentrations of 25 nM of both reagents in 110 μL of medium per well in a 96-well plate. For Cas9-ssODN conjugates, the Cas9-adaptor was pre-mixed with the ssODN in Opti-MEM (Gibco) and incubated at RT for 15-30 min prior to RNP formation. Next, gRNA was added, and the mixture was incubated for 5-10 min at RT. Then, Plus reagent (Thermo; 0.17 μL per well) was added, and the mixture was incubated for an additional 5 min. Finally, Lipofectamine CRISPRMAX (0.3 μL per well) in Opti-MEM was added, and the mixture was incubated at RT for 5 min. The final transfection mixture was transferred to each well. In cases where Cas9 did not specifically bind ssODNs, the RNP was first formed by incubating Cas9 and gRNA at RT for 5-10 min in Opti-MEM. Then, Plus reagent (0.17 μL per well) was added, and the mixture was incubated for an additional 5 min. Next, the ssODN was added to the mixture. Finally, Lipofectamine CRISPRMAX (0.3 μL per well) in Opti-MEM was added, and the mixture was incubated at RT for 5 min. The final transfection mixture was transferred to each well. The transfections were performed in three technical replicates for each biological replicate. For knock-in experiments using Cas9 double-ssODN conjugates (FIG. 5), RNP formation was performed first to prevent the non-specific Cas9-ssODN interaction that blocks RNP formation and decreases the genome editing efficiency. A luminescence detection assay was performed as described above at 24 h post transfection.

### GFP11 Sequence Knock-In by Lipofection

HEK-293T cells were seeded in a 96-well plate at a density of 8,000 cells per well. The next day, Lipofectamine CRISPRMAX was used to transfect the cells with Cas9 RNP (30 nM) and ssODN (30 nM) using the same procedures as described above for the HiBiT knock-in assay. Approximately 20-22 hours post transfection, the media was exchanged, and the cells were incubated for an additional 2-4 hours. Then, a plasmid encoding for a GFP1-10 fragment (Addgene #70219, a kind gift from Prof. Bo Huang)⁵ was delivered to the cells using Lipofectamine 2000 (Invitrogen) (120 ng plasmid and 0.4 μL Lipofectamine per well). A total of 50 h after RNP and ssODN transfection, the cells were fixed using 4% paraformaldehyde. Nuclei were stained by HCS NuclearMask Blue Stain (Invitrogen), and fluorescence images were obtained using the ImageXpress Micro (Molecular Devices) with the DAPI and GFP channels.

### Droplet Digital PCR-Based Assay to Quantify NHEJ and HDR

HEK-293FT cells were seeded in a 96-well plate at a density of 10,000 cells per well. The next day, the cells were transfected with Cas9 RNP (35 nM) and ssODN (35 nM) using Lipofectamine RNAiMAX (Invitrogen) in 110 μL of media per well in a 96-well plate. For Cas9-ssODN conjugates, the Cas9-adaptor was pre-mixed with ssODN in Opti-MEM (Gibco) and incubated at RT for 15-30 min prior to RNP formation. Next, gRNA was added, and the mixture was incubated for 5-10 min at RT. Finally, Lipofectamine RNAiMAX (0.3 μL per well) in Opti-MEM was added, and the mixture was incubated at RT for 5 min. The final transfection mixture was transferred to each well. In cases where Cas9 does not specifically bind to the ssODNs, the RNP was first formed by incubating Cas9 and gRNA at RT for 5-10 min in Opti-MEM. Then, the ssODN was added to the mixture. Finally, Lipofectamine RNAiMAX (0.3 μL per well) in Opti-MEM was added, and the mixture was incubated at RT for 5 min. The final transfection mixture was transferred to each well. The transfections were performed in three technical replicates for each biological replicate. Two days post transfection, the cells were harvested, and the genomic DNA was extracted using a DNeasy Blood & Tissue Kit (Qiagen). Genomic sequences were read by droplet digital PCR as previously reported.⁶⁻⁷ For the single-nucleotide exchange experiments using Cas9 double-ssODN conjugates (FIG. 5), RNP formation was performed first because more ssODN was used in comparison to RNP.

eGFP Disruption Assay to Confirm the Target Specificity of the Cas9-Adaptor Conjugate

Cas9 (10 pmol) and sgRNA (10 pmol) were mixed and incubated at RT for 5 min. U2OS.eGFP.PEST cells⁸ were transfected with the RNP complex using the SE Cell Line 4D-Nucleofector kit (Lonza) following the pulse program of DN-130. After transfection, cells were suspended in the culture media and transferred to a 96-well plate (200,000 cell/well). Forty-eight hours after transfection, cells were fixed with 4% paraformaldehyde solution and nuclei were stained with HCS NuclearMask Blue Stain (Invitrogen). The resulting fluorescence signals from eGFP and nuclei were measured using an ImageXpress Micro High Content Analysis System (Molecular Devices).

### HiBiT Sequence Knock-In by Nucleofection in INS-1E Cells

INS-1E cells were transfected with Cas9 ribonucleoprotein (RNP) and ssODN using the SF Cell Line 4D-Nucleofector kit (Lonza) following the pulse program of DE-130. For Cas9-ssODN conjugates, 20 pmol of Cas9-adaptor were pre-mixed with 20 pmol of ssODN and incubated at RT for 15-30 min prior to RNP formation to ensure Cas9-ssODN conjugate formation. Then 20 pmol of gRNA was added, and the final mixture was incubated for 5-10 min at RT. In cases where Cas9 did not specifically bind ssODNs, the RNP was formed first because nonspecific Cas9-DNA interactions can hamper the RNP formation. After incubating Cas9 and gRNA at RT for 5-10 min, 20 pmol of ssODN were added to the mixture. Approximately 200,000 cells were transfected with the above mixtures in a well of the nucleofection kit, and cells were seeded in a well of a 24-well plate. Cells were incubated at 37° C. for 48 h, and the supernatant was taken to measure the amount of secreted HiBiT peptide using the Nano-Glo HiBiT Extracellular Detection System (Promega). The resulting luminescence signals were normalized based on the cell viability.

### IL-10 Knock-In by Nucleofection in INS-1E Cells

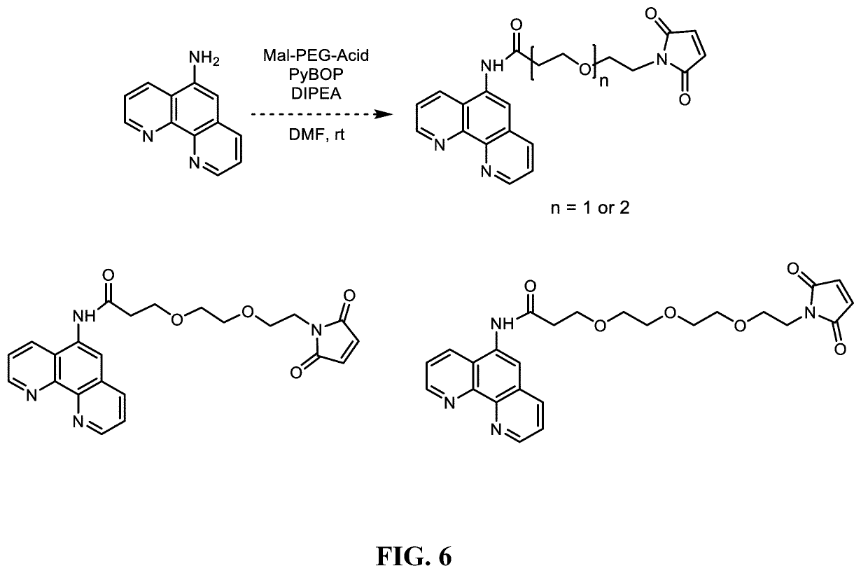

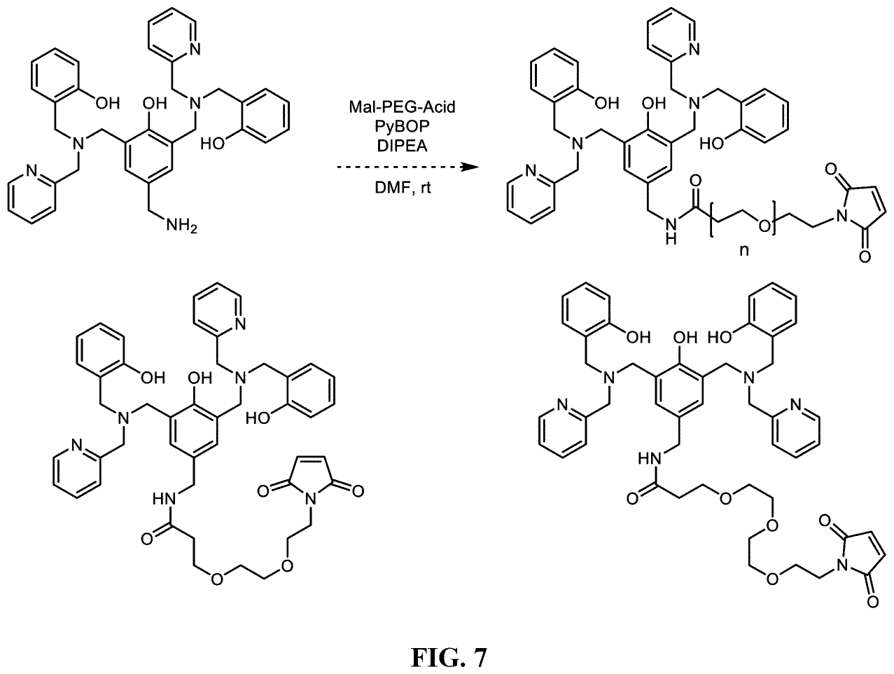

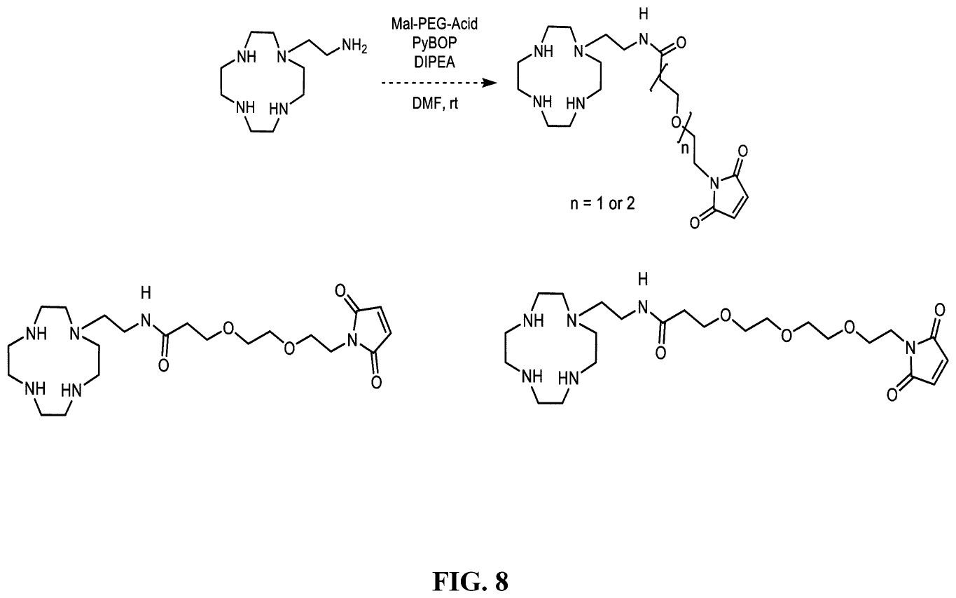

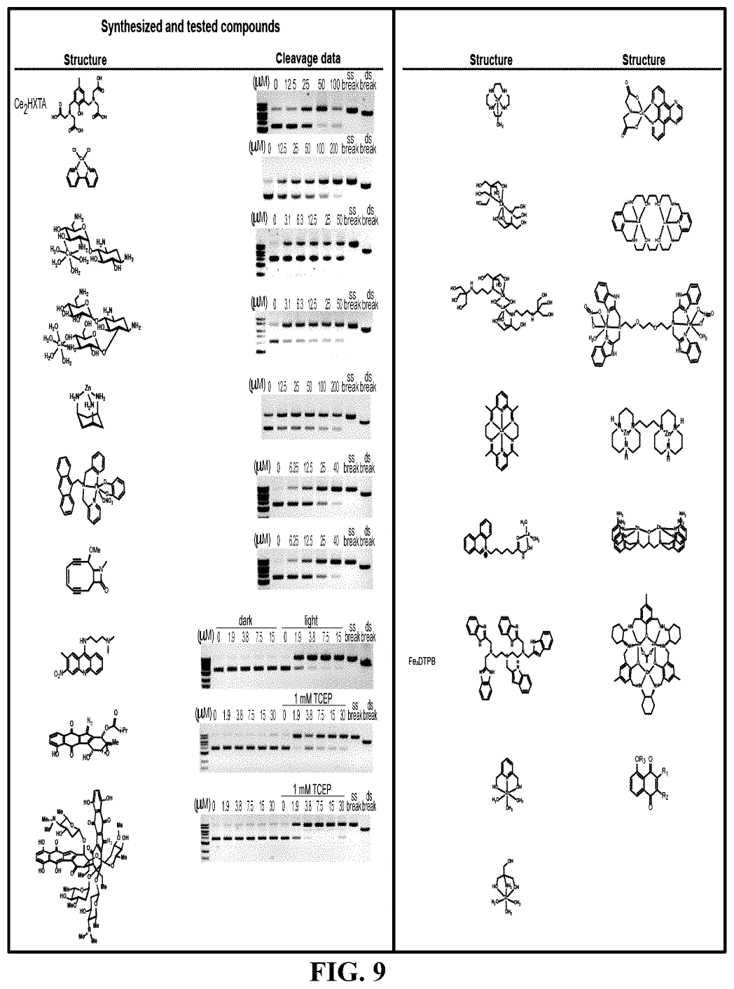

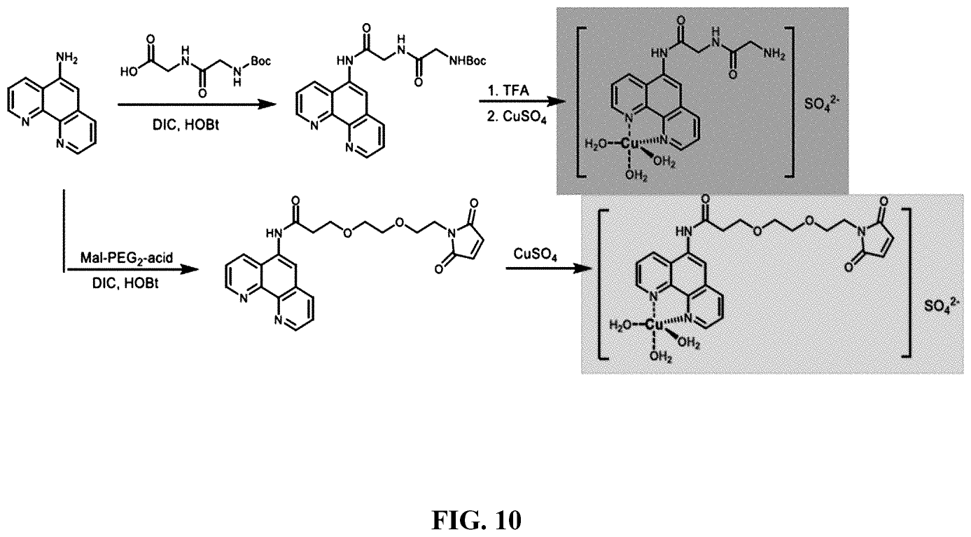

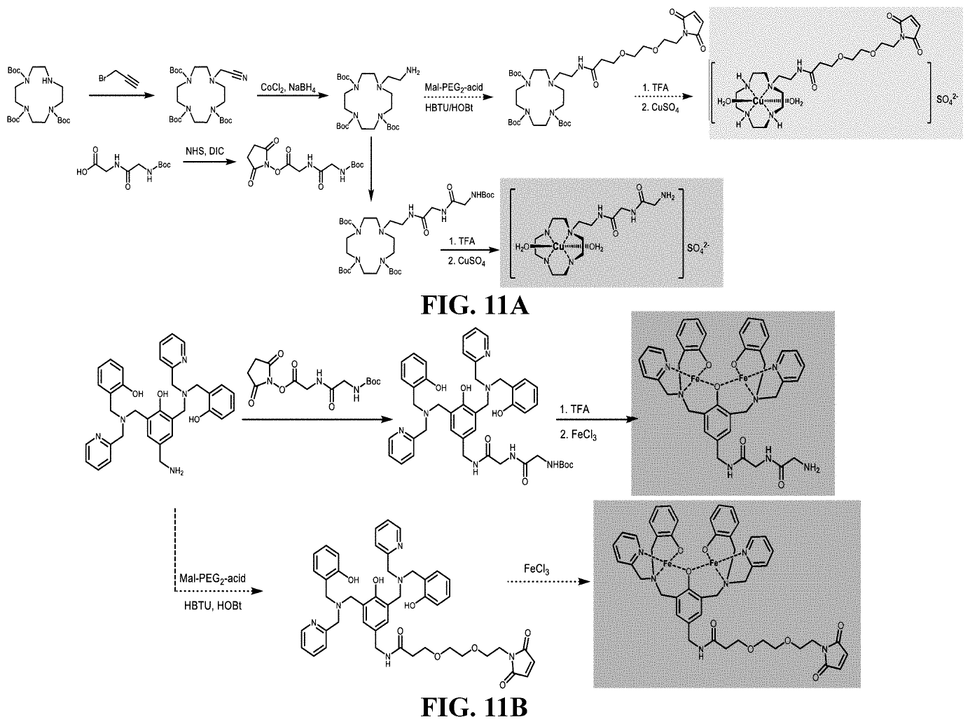

INS-1E cells were transfected with Cas9 ribonucleoprotein (RNP) and ssODN using the SF Cell Line 4D-Nucleofector kit (Lonza) following the pulse program of DE-130. For Cas9-ssODN conjugates, 20 pmol of Cas9-adaptor were pre-mixed with 12 pmol of ssODN and incubated at RT for 15-30 min prior to RNP formation to ensure Cas9-ssODN conjugate formation. Then 20 pmol of gRNA was added, and the final mixture was incubated for 5-10 min at RT. In cases where Cas9 did not specifically bind ssODNs, the RNP was formed first because nonspecific Cas9-DNA interactions can hamper the RNP formation. After incubating Cas9 and gRNA at RT for 5-10 min, 12 pmol of ssODN were added to the mixture. Approximately 200,000 cells were transfected with the above mixtures in a well of the nucleofection kit, and cells were seeded in a well of a 24-well plate. Cells were incubated at 37° C. for 72 h, and the supernatant was taken to measure the amount of secreted IL-10 using the IL-10 Rat ELISA Kit (Invitrogen, catalog #BMS629). The resulting values were normalized based on the cell viability. LPS was used at the concentration of 10 pg/mL (FIGS. 6d and S12).

### Glucose-Stimulated Peptide Secretion

INS-1E cells knocked in with the HiBiT sequence were grown in a large scale. Then, cells were seeded in a 24-well plate at the density of 150,000 cells per well. The next day, cell were washed with and incubated in Krebs-Ringer bicarbonate buffer (138 mM NaCl, 5.4 mM KCl, 5 mM NaHCO₃, 2.6 mM MgCl₂, 2.6 mM CaCl₂), 10 mM HEPES, pH 7.4, 0.5% BSA) without glucose for 2 h. Cells were subsequently incubated with Krebs-Ringer bicarbonate buffer containing glucose (from 2.8 mM to 16.8 mM) for 1 h. The supernatant was taken to measure the amount of secreted HiBiT peptide using the Nano-Glo HiBiT Extracellular Detection System (Promega).

### PCR to Amplify the IL-10 Knock-In Sequence

Genomic DNAs from the edited INS-1E cells were extracted using a DNeasy Blood & Tissue Kit (Qiagen). Fifty ng of genomic DNA was mixed with 1.25 uM of forward primer, 1.25 uM of reverse primer, and Q5 Hot Start High-Fidelity 2× Master Mix (New England Biolabs) in a final volume of 25 μL. Primer set 1 (forward: CCCGGAGAAGCGTAGCAAA, reverse: CCCCGGCACGCTTATTTTTC, (SEQ ID NOs: 16 and 17) Ta=68° C., 36 temperature cycles) and primer set 2 (forward: CCCGGAGAAGCGTAGCAAAG, reverse: AAGATCCCCGGCACGCTTATTT, (SEQ ID NOs: 18 and 19) Ta=70° C., 40 temperature cycles) were used.

TABLE 3
Primer sequences for mutagenesis.

| Mutation | |
|---|---|
| site | Primer sequence |
| C80S | F: CGTATTAGCTATCTACAGGAGATTTTTTCAAATGAG |
| | R: GTAGATAGCTAATACGATTCTTCCGACGTG |
| | (SEQ ID NOs: 20 and 21) |
| C574S | F: AAAAATAGAAAGCTTTGATAGTGTTGAAATTTC |
| | R: TTGAAATAATCTTCTTTTAATTGC |
| | (SEQ ID NOs: 22 and 23) |
| M1C | F: GAGGAAGGTGTGCGATAAGAAATACTCAATAGG |
| | R: TTCTTCTTGGGCATAAAC |
| | (SEQ ID NOs: 24 and 25) |
| S204C | F: TATTAACGCATGCGGAGTAGATGC |
| | R: GGGTTTTCTTCAAATAATTGATTG |
| | (SEQ ID NOs: 26 and 27) |
| E532C | F: GTTACTTGCGGAATGCGAAAACCAGCATTTC |
| | R: CGCATTCCGCAAGTAACATATTTGACCTTTGTC |
| | (SEQ ID NOs: 28 and 29) |
| K558C | F: AACAAATCGATGCGTAACCGTTAAGCAATTAAAAG |
| | R: TTGAAGAGTAAATCAACAATG |
| | (SEQ ID NOs: 30 and 31) |
| Q826C | F: GTATGTGGACTGCGAATTAGATATTAATCGTTTAAG |
| | R: ATGTCTCTTCCATTTTGG |
| | (SEQ ID NOs: 32 and 33) |
| E945C | F: TAAATACGATTGCAATGATAAACTTATTCGAG |
| | R: GTATTCATGCGACTATCC |
| | (SEQ ID NOs: 34 and 35) |
| E1026C | F: TGCTAAGTCTTGCCAAGAAATAGGC |
| | R: ATCATTTTACGAACATCATAAAC |
| | (SEQ ID NOs: 36 and 37) |
| N1054C | F: TACACTTGCATGCGGAGAGATTCGC |
| | R: ATTTCTGTTTTGAAGAAGTTC |
| | (SEQ ID NOs: 38 and 39) |
| E1068C | F: CTAATGGGTGCACTGGAGAAATTGTCTGGG |
| | R: CTCCAGTGCACCCATTAGTTTCGATTAGAGGG |
| | (SEQ ID NOs: 40 and 41) |
| S1116C | F: AAAAAGAAATTGCGACAAGCTTATTGCTC |
| | R: GGTAAAATTGACTCCTTGG |
| | (SEQ ID NOs: 42 and 43) |
| K1153C | F: AAAAGGGTGCTCGAAGAAGTTAAAATCCGTTAAAGAG |
| | R: CTTCGAGCACCCTTTTTCCACCTTAGCAAC |
| | (SEQ ID NOs: 44 and 45) |
| E1207C | F: TTTTGAGTTATGCAACGGTCGTAAACG |
| | R: AGACTATATTTAGGTAGTTTAATG |
| | (SEQ ID NOs: 46 and 47) |

TABLE 4
Primer sequences for gRNA synthesis.

| Primer | |
|---|---|
| name | Primer sequence |
| Universal | AAAAGCACCGACTCGGTGCCACTTTTTCAAGTTGATAA |
| reverse | CGGACTAGCCTTATTTT (SEQ ID NO: 48) |
| | AACTTGCTATTTCTAGCTCTAAAAC (SEQ ID NO: |
| | 49) |
| GAPDH 1 | TAATACGACTCACTATAGGTCCAGGGGTCTTACTCCTG |
| forward | TTTTAGAGCTAGAAAT (SEQ ID NO: 50) |
| GAPDH 2 | TAATACGACTCACTATAGCCTCCAAGGAGTAAGACCCC |
| forward | GTTTTAGAGCTAGAAAT (SEQ ID NO: 51) |
| PPIB | TAATACGACTCACTATAGCGCCAAGGAGTAGGGCACAG |
| forward | TTTTAGAGCTAGAAAT (SEQ ID NO: 52) |
| CFL1 | TAATACGACTCACTATAGGGCCAGAAGGGGCTCACAAG |
| forward | TTTTAGAGCTAGAAAT (SEQ ID NO: 53) |
| RBM20 1 | TAATACGACTCACTATAGGGACCTCGGGGAGAGTGACG |
| forward | TTTTAGAGCTAGAAAT (SEQ ID NO: 54) |
| RBM20 2 | TAATACGACTCACTATAGGGGAGAGTGACCGGCTCACG |
| forward | TTTTAGAGCTAGAAAT (SEQ ID NO: 55) |
| INS1 1a | TAATACGACTCACTATAGCCCAAGTCCCGTCGTGAAGG |
| forward | TTTTAGAGCTAGAAAT (SEQ ID NO: 56) |
| INS1 1b | TAATACGACTCACTATAGCTCCAGTTGTGGCACTTGCG |
| forward | TTTTAGAGCTAGAAAT (SEQ ID NO: 57) |
| INS1 2a | TAATACGACTCACTATAGGGTGGAGGCCCGGAGGCCGT |
| forward | TTTAGAGCTAGAAAT (SEQ ID NO: 58) |
| INS1 2b | TAATACGACTCACTATAGGGTGGAGGCCCGGAGGCCGG |
| forward | TTTTAGAGCTAGAAAT (SEQ ID NO: 59) |
| INS1 2c | TAATACGACTCACTATAGTCTGAAGATCCCCGGCCTCG |
| forward | TTTTAGAGCTAGAAAT (SEQ ID NO: 60) |
| INS1 2d | TAATACGACTCACTATAGTGGGTGGAGGCCCGGAGGCG |
| forward | TTTTAGAGCTAGAAAT (SEQ ID NO: 61) |
| INS1 2e | TAATACGACTCACTATAGCTGAAGATCCCCGGCCTCCG |
| forward | TTTTAGAGCTAGAAAT (SEQ ID NO: 62) |
| INS1 3a | TAATACGACTCACTATAGACAATGCCACGCTTCTGCCG |
| forward | TTTTAGAGCTAGAAAT (SEQ ID NO: 63) |
| INS1 3b | TAATACGACTCACTATAGCTTCAGACCTTGGCACTGGG |
| forward | TTTTAGAGCTAGAAAT (SEQ ID NO: 64) |
| eGFP on- | TAATACGACTCACTATAGGGCACGGGCAGCTTGCCGGG |
| target | TTTTAGAGCTAGAAAT (SEQ ID NO: 65) |
| forward | |
| eGFP off- | TAATACGACTCACTATAGGGCACGGGCAGCTTGCCGCG |
| target 1 | TTTTAGAGCTAGAAAT (SEQ ID NO: 66) |
| forward | |
| eGFP off- | TAATACGACTCACTATAGGGCACGGGCAGCTTGCCCGG |
| target 2 | TTTTAGAGCTAGAAAT (SEQ ID NO: 67) |
| forward | |
| eGFP off- | TAATACGACTCACTATAGGGCACGGGCAGCTTCCCGGG |
| target 5 | TTTTAGAGCTAGAAAT (SEQ ID NO: 68) |
| forward | |

TABLE 5
Sequences of short ssODNs (<200 nt).

| ssODN name | Assay | ssODN sequence |
|---|---|---|
| GAPDH | NanoLuc | TCTTCTAGGTATGACAACGAATTTGGCTACAGCAA |
| adaptor | luciferase | CAGGGTGGTGGACCTCATGGCCCACATGGCCTCCA |
| | complementation | AGGAGGTGAGCGGCTGGCGGCTGTTCAAGAAGATT |
| | | AGCTAAGACCCCTGGACCACCAGCCCCAGCAAGAG |
| | | CACAAGAGGAAGAGAGAGACCCTCACTGCTGGGGA |
| | | GTCCCTGCGACGATGAGAGTGAAGC |
| | | (SEQ ID NO: 69) |
| GAPDH | NanoLuc | TCTTCTAGGTATGACAACGAATTTGGCTACAGCAA |
| adaptor- | luciferase | CAGGGTGGTGGACCTCATGGCCCACATGGCCTCCA |
| free | complementation | AGGAGGTGAGCGGCTGGCGGCTGTTCAAGAAGATT |
| | | AGCTAAGACCCCTGGACCACCAGCCCCAGCAAGAG |
| | | CACAAGAGGAAGAGAGAGACCCTCACTGCTGGGGA |
| | | GTCCCTGC |
| | | (SEQ ID NO: 70) |
| GAPDH | NanoLuc | TCTTCTAGGTATGACAACGAATTTGGCTACAGCAA |
| 15-nt | luciferase | CAGGGTGGTGGACCTCATGGCCCACATGGCCTCCA |
| adaptor | complementation | AGGAGGTGAGCGGCTGGCGGCTGTTCAAGAAGATT |
| | | AGCTAAGACCCCTGGACCACCAGCCCCAGCAAGAG |
| | | CACAAGAGGAAGAGAGAGACCCTCACTGCTGGGGA |
| | | GTCCCTGCGACGATGAGAGTGAA |
| | | (SEQ ID NO: 71) |
| GAPDH | NanoLuc | TCTTCTAGGTATGACAACGAATTTGGCTACAGCAA |
| 13-nt | luciferase | CAGGGTGGTGGACCTCATGGCCCACATGGCCTCCA |
| adaptor | complementation | AGGAGGTGAGCGGCTGGCGGCTGTTCAAGAAGATT |
| | | AGCTAAGACCCCTGGACCACCAGCCCCAGCAAGAG |
| | | CACAAGAGGAAGAGAGAGACCCTCACTGCTGGGGA |
| | | GTCCCTGCGACGATGAGAGTG |
| | | (SEQ ID NO: 72) |
| PPIB | NanoLuc | CAGCTCAGAGCCCTGTGGCGGACTACAGGGCCTGC |
| adaptor | luciferase | ACAGACGGTCACTCAAAGAAAGATGTCCCTGTGCC |
| | complementation | CTAGCTAATCTTCTTGAACAGCCGCCAGCCGCTCA |
| | | CCTCCTTGGCGATGGCAAAGGGCTTCTCCACCTCG |
| | | ATCTTGCCGCAGTCTGCGATGATCACATCCTTCAG |
| | | GGGTGACGATGAGAGTGAAGC |
| | | (SEQ ID NO: 73) |
| PPIB | NanoLuc | CAGCTCAGAGCCCTGTGGCGGACTACAGGGCCTGC |
| adaptor- | luciferase | ACAGACGGTCACTCAAAGAAAGATGTCCCTGTGCC |
| free | complementation | CTAGCTAATCTTCTTGAACAGCCGCCAGCCGCTCA |
| | | CCTCCTTGGCGATGGCAAAGGGCTTCTCCACCTCG |
| | | ATCTTGCCGCAGTCTGCGATGATCACATCCTTCAG |
| | | GGGT |
| | | (SEQ ID NO: 74) |
| CFL1 | NanoLuc | GAGGTCAAGGACCGCTGCACCCTGGCAGAGAAGCT |
| adaptor | luciferase | GGGGGGCAGTGCCGTCATCTCCCTGGAGGGCAAGC |
| | complementation | CTTTGGTGAGCGGCTGGCGGCTGTTCAAGAAGATT |
| | | AGCTGAGCCCCTTCTGGCCCCCTGCCTGGAGCATC |
| | | TGGCAGCCCCACACCTGCCCTTGGGGGTTGCAGGC |
| | | TGCCCCCTGACGATGAGAGTGAAGC |
| | | (SEQ ID NO: 75) |
| CFL1 | NanoLuc | GAGGTCAAGGACCGCTGCACCCTGGCAGAGAAGCT |
| adaptor- | luciferase | GGGGGGCAGTGCCGTCATCTCCCTGGAGGGCAAGC |
| free | complementation | CTTTGGTGAGCGGCTGGCGGCTGTTCAAGAAGATT |
| | | AGCTGAGCCCCTTCTGGCCCCCTGCCTGGAGCATC |
| | | TGGCAGCCCCACACCTGCCCTTGGGGGTTGCAGGC |
| | | TGCCCCCT |
| | | (SEQ ID NO: 76) |
| INS1 | NanoLuc | GGAGGCTCTGTACCTGGTGTGTGGGGAACGTGGTT |
| site 1 | luciferase | TCTTCTACACACCCAAGTCCCGTCGTGAAGTGGAG |
| adaptor- | complementation | AAGCGTGTGAGCGGCTGGCGGCTGTTCAAGAAGAT |
| free | | TAGCAAGCGTGACCCGCAAGTGCCACAACTGGAGC |
| | | TGGGTGGAGGCCCGGAGGCCGGGGATCTTCAGACC |
| | | TTGGCACTGG |
| | | (SEQ ID NO: 77) |
| INS1 | NanoLuc | ACACCCAAGTCCCGTCGTGAAGTGGAGGACCCGCA |
| site 2 | luciferase | AGTGCCACAACTGGAGCTGGGTGGAGGCCCGGAGA |
| adaptor | complementation | AGCGTGTGAGCGGCTGGCGGCTGTTCAAGAAGATT |
| | | AGCAAGCGTGCCGGGGATCTTCAGACCTTGGCACT |
| | | GGAGGTTGCCCGGCAGAAGCGTGGCATTGTGGATC |
| | | AGTGCTGC |
| | | (SEQ ID NO: 78) |
| INS1 | NanoLuc | ACACCCAAGTCCCGTCGTGAAGTGGAGGACCCGCA |
| site 2 | luciferase | AGTGCCACAACTGGAGCTGGGTGGAGGCCCGGAGA |
| adaptor- | complementation | AGCGTGTGAGCGGCTGGCGGCTGTTCAAGAAGATT |
| free | | AGCAAGCGTGCCGGGGATCTTCAGACCTTGGCACT |
| | | GGAGGTTGCCCGGCAGAAGCGTGGCATTGTGGATC |
| | | AGTGCTGCGACGATGAGAGTGAAGC |
| | | (SEQ ID NO: 79) |
| INS1 | NanoLuc | GCAAGTGCCACAACTGGAGCTGGGTGGAGGCCCGG |
| site 3 | luciferase | AGGCCGGGGATCTTCAGACCTTGGCACTGGAGGTT |
| adaptor- | complementation | AAGCGTGTGAGCGGCTGGCGGCTGTTCAAGAAGAT |
| free | | TAGCAAGCGTGCCCGGCAGAAGCGTGGCATTGTGG |
| | | ATCAGTGCTGCACCAGCATCTGCTCCCTCTACCAA |
| | | CTGGAGAACT |
| | | (SEQ ID NO: 80) |
| GAPDH | GFP | GACAACGAATTTGGCTACAGCAACAGGGTGGTGGA |
| adaptor | complementation | CCTCATGGCCCACATGGCCTCCAAGGAGGGTGGCG |
| | | GCCGTGACCACATGGTCCTTCATGAGTATGTAAAT |
| | | GCTGCTGGGATTACATAAGACCCCTGGACCACCAG |
| | | CCCCAGCAAGAGCACAAGAGGAAGAGAGAGACCCT |
| | | CACTGCTGGACGATGAGAGTGAAGC |
| | | (SEQ ID NO: 81) |
| GAPDH | GFP | GACAACGAATTTGGCTACAGCAACAGGGTGGTGGA |
| adaptor- | complementation | CCTCATGGCCCACATGGCCTCCAAGGAGGGTGGCG |
| free | | GCCGTGACCACATGGTCCTTCATGAGTATGTAAAT |
| | | GCTGCTGGGATTACATAAGACCCCTGGACCACCAG |
| | | CCCCAGCAAGAGCACAAGAGGAAGAGAGAGACCCT |
| | | CACTGCTG |
| | | (SEQ ID NO: 82) |
| RBM20 1 | Droplet digital | GTGGGAAGAGCTGCAGGAGGTGAAGCTGGGAGTGT |
| adaptor | PCR | GGGACCTCGGTGAGAGTGACCGGCTCACCGGACTA |
| | | CTAGACCGCGGCCTTTCTGGGCCATATCTGTGAGG |
| | | GAGCCAAGGAGCAGGGACGATGAGAGTGAAGC |
| | | (SEQ ID NO: 83) |
| RBM20 2 | Droplet digital | ACAGATATGGCCCAGAAAGGCCGCGGTCTAGTAGT |
| adaptor | PCR | CCGGTGAGCCGGTCACTGTCCCCGAGGTCCCACAC |
| | | ACCCAGCGACGATGAGAGTGAAGC |
| | | (SEQ ID NO: 84) |

TABLE 6
Sequences of gBlocks DNAs and primers
for generating ssODNs for IL-10 knock-in.

| DNA name | DNA sequence |
|---|---|
| gBlocks | TAATACGACTCACTATAGCTTCACTCTCATCGTCGGCT |
| INS1- | TTATTCATTGCAGAGGGGTGGGCGGGGAGTGGTGGACT |
| IL-10 | CAGTTGCAGTAGTTCTCCAGTTGGTAGAGGGAGCAGAT |
| adaptor | GCTGGTGCAGCACTGATCCACAATGCCACGCTTCTGCC |
| | GGGCAACCTCCAGTGCCAAGGTCTGAAGATCCCCGGCA |
| | CGCTTATTTTTCATTTTGAGTGTCACGTAGGCTTCTAT |
| | GCAGTTGATGAAGATGTCAAACTCATTCATGGCCTTGT |
| | AGACACCTTTGTCTTGGAGCTTATTAAAATCATTCTTC |
| | ACCTGCTCCACTGCCTTGCTTTTATTCTCACAGGGGAG |
| | AAATCGATGACAGCGTCGCAGCTGTATCCAGAGGGTCT |
| | TCAGCTTCTCTCCCAGGGAATTCAAATGCTCCTTGATT |
| | TCTGGGCCATGGTTCTCTGCCTGGGGCATCACTTCTAC |
| | CAGGTAAAACTTGATCATTTCTGACAAGGCTTGGCAAC |
| | CCAAGTAACCCTTAAAGTCCTGCAGTAAGGAATCTGTC |
| | AGCAGTATGTTGTCCAGCTGGTCCTTCTTTTGAAAGAA |
| | AGTCTTCACTTGACTGAAGGCAGCCCTCAGCTCTCGGA |
| | GCATGTGGGTCTGGCTGACTGGGAAGTGGGTGCAGTTA |
| | TTGTCACCCCGGATGGAATGGCCTTTGCTACGCTTCTC |
| | CGGGCCTCCACCCAGCTCCAGTTGTGGCACTTGCGGGT |
| | CCTCCACTTCACGACGGGACTTGGGTGTGTAGAAGAAA |
| | CCACGTTCCCCACACACCAGGTACAGAGCCTCCACCAG |
| | GTGAGGACCACAAAGGTGCTGTTTGACAAAAGC |
| | (SEQ ID NO: 85) |
| gBlocks | TAATACGACTCACTATAGGCTTTATTCATTGCAGAGGG |
| INS1- | GTGGGCGGGGAGTGGTGGACTCAGTTGCAGTAGTTCTC |
| IL-10 | CAGTTGGTAGAGGGAGCAGATGCTGGTGCAGCACTGAT |
| adaptor- | CCACAATGCCACGCTTCTGCCGGGCAACCTCCAGTGCC |
| free | AAGGTCTGAAGATCCCCGGCACGCTTATTTTTCATTTT |
| | GAGTGTCACGTAGGCTTCTATGCAGTTGATGAAGATGT |
| | CAAACTCATTCATGGCCTTGTAGACACCTTTGTCTTGG |
| | AGCTTATTAAAATCATTCTTCACCTGCTCCACTGCCTT |
| | GCTTTTATTCTCACAGGGGAGAAATCGATGACAGCGTC |
| | GCAGCTGTATCCAGAGGGTCTTCAGCTTCTCTCCCAGG |
| | GAATTCAAATGCTCCTTGATTTCTGGGCCATGGTTCTC |
| | TGCCTGGGGCATCACTTCTACCAGGTAAAACTTGATCA |
| | TTTCTGACAAGGCTTGGCAACCCAAGTAACCCTTAAAG |
| | TCCTGCAGTAAGGAATCTGTCAGCAGTATGTTGTCCAG |
| | CTGGTCCTTCTTTTGAAAGAAAGTCTTCACTTGACTGA |
| | AGGCAGCCCTCAGCTCTCGGAGCATGTGGGTCTGGCTG |
| | ACTGGGAAGTGGGTGCAGTTATTGTCACCCCGGATGGA |
| | ATGGCCTTTGCTACGCTTCTCCGGGCCTCCACCCAGCT |
| | CCAGTTGTGGCACTTGCGGGTCCTCCACTTCACGACGG |
| | GACTTGGGTGTGTAGAAGAAACCACGTTCCCCACACAC |
| | CAGGTACAGAGCCTCCACCAGGTGAGGACCACAAAGGT |
| | GCTGTTTGACAAAAGC |
| | (SEQ ID NO: 86) |
| INS1 | TAATACGACTCACTATAGCTTCACTCTCATCG |
| forward | (SEQ ID NO: 88) |
| adaptor | |
| INS1 | TAATACGACTCACTATAGGCTTTATTCATTGCAGAGGG |
| forward | GTGG |
| adaptor- | (SEQ ID NO: 88) |
| free | |
| INS1 | GCTTTTGTCAAACAGCACCTT (SEQ ID NO: 89) |
| reverse | |
| universal | |

TABLE 7
Sequences of long ssODNs for IL-10 knock-in.

| ssODN | | |
|---|---|---|
| name | Assay | ssODN sequence |
| INS1- | IL-10 | GCTTTTGTCAAACAGCACCTTTGTGGTCC |
| IL-10 | ELISA | TCACCTGGTGGAGGCTCTGTACCTGGTGT |
| adaptor | | GTGGGGAACGTGGTTTCTTCTACACACCC |
| | | AAGTCCCGTCGTGAAGTGGAGGACCCGCA |
| | | AGTGCCACAACTGGAGCTGGGTGGAGGCC |
| | | CGGAGAAGCGTAGCAAAGGCCATTCCATC |
| | | CGGGGTGACAATAACTGCACCCACTTCCC |
| | | AGTCAGCCAGACCCACATGCTCCGAGAGC |
| | | TGAGGGCTGCCTTCAGTCAAGTGAAGACT |
| | | TTCTTTCAAAAGAAGGACCAGCTGGACAA |
| | | CATACTGCTGACAGATTCCTTACTGCAGG |
| | | ACTTTAAGGGTTACTTGGGTTGCCAAGCC |
| | | TTGTCAGAAATGATCAAGTTTTACCTGGT |
| | | AGAAGTGATGCCCCAGGCAGAGAACCATG |
| | | GCCCAGAAATCAAGGAGCATTTGAATTCC |
| | | CTGGGAGAGAAGCTGAAGACCCTCTGGAT |
| | | ACAGCTGCGACGCTGTCATCGATTTCTCC |
| | | CCTGTGAGAATAAAAGCAAGGCAGTGGAG |
| | | CAGGTGAAGAATGATTTTAATAAGCTCCA |
| | | AGACAAAGGTGTCTACAAGGCCATGAATG |
| | | AGTTTGACATCTTCATCAACTGCATAGAA |
| | | GCCTACGTGACACTCAAAATGAAAAATAA |
| | | GCGTGCCGGGGATCTTCAGACCTTGGCAC |
| | | TGGAGGTTGCCCGGCAGAAGCGTGGCATT |
| | | GTGGATCAGTGCTGCACCAGCATCTGCTC |
| | | CCTCTACCAACTGGAGAACTACTGCAACT |
| | | GAGTCCACCACTCCCCGCCCACCCCTCTG |
| | | CAATGAATAAAGCCGACGATGAGAGTGAA |
| | | GC (SEQ ID NO: 90) |
| INS1- | IL-10 | GCTTTTGTCAAACAGCACCTTTGTGGTCC |
| IL-10 | ELISA | TCACCTGGTGGAGGCTCTGTACCTGGTGT |
| adaptor- | | GTGGGGAACGTGGTTTCTTCTACACACCC |
| free | | AAGTCCCGTCGTGAAGTGGAGGACCCGCA |
| | | AGTGCCACAACTGGAGCTGGGTGGAGGCC |
| | | CGGAGAAGCGTAGCAAAGGCCATTCCATC |
| | | CGGGGTGACAATAACTGCACCCACTTCCC |
| | | AGTCAGCCAGACCCACATGCTCCGAGAGC |
| | | TGAGGGCTGCCTTCAGTCAAGTGAAGACT |
| | | TTCTTTCAAAAGAAGGACCAGCTGGACAA |
| | | CATACTGCTGACAGATTCCTTACTGCAGG |
| | | ACTTTAAGGGTTACTTGGGTTGCCAAGCC |
| | | TTGTCAGAAATGATCAAGTTTTACCTGGT |
| | | AGAAGTGATGCCCCAGGCAGAGAACCATG |
| | | GCCCAGAAATCAAGGAGCATTTGAATTCC |
| | | CTGGGAGAGAAGCTGAAGACCCTCTGGAT |
| | | ACAGCTGCGACGCTGTCATCGATTTCTCC |
| | | CCTGTGAGAATAAAAGCAAGGCAGTGGAG |
| | | CAGGTGAAGAATGATTTTAATAAGCTCCA |
| | | AGACAAAGGTGTCTACAAGGCCATGAATG |
| | | AGTTTGACATCTTCATCAACTGCATAGAA |
| | | GCCTACGTGACACTCAAAATGAAAAATAA |
| | | GCGTGCCGGGGATCTTCAGACCTTGGCAC |
| | | TGGAGGTTGCCCGGCAGAAGCGTGGCATT |
| | | GTGGATCAGTGCTGCACCAGCATCTGCTC |
| | | CCTCTACCAACTGGAGAACTACTGCAACT |
| | | GAGTCCACCACTCCCCGCCCACCCCTCTG |
| | | CAATGAATAAAGCC (SEQ ID NO: 91) |

### Example 6

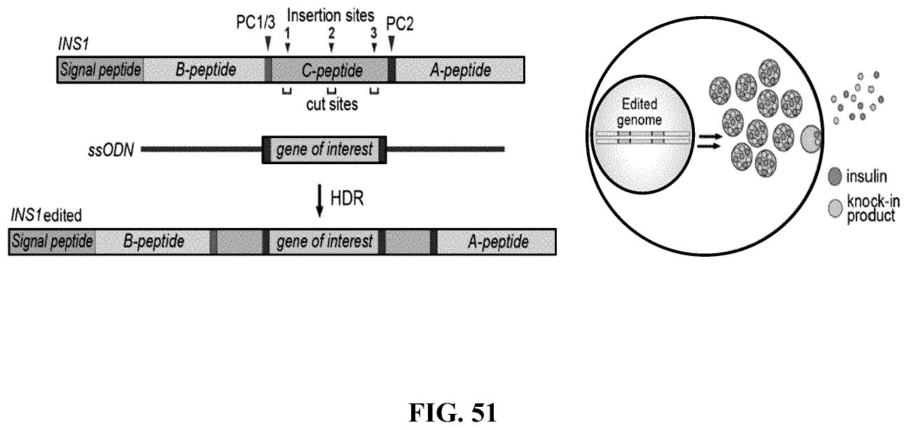

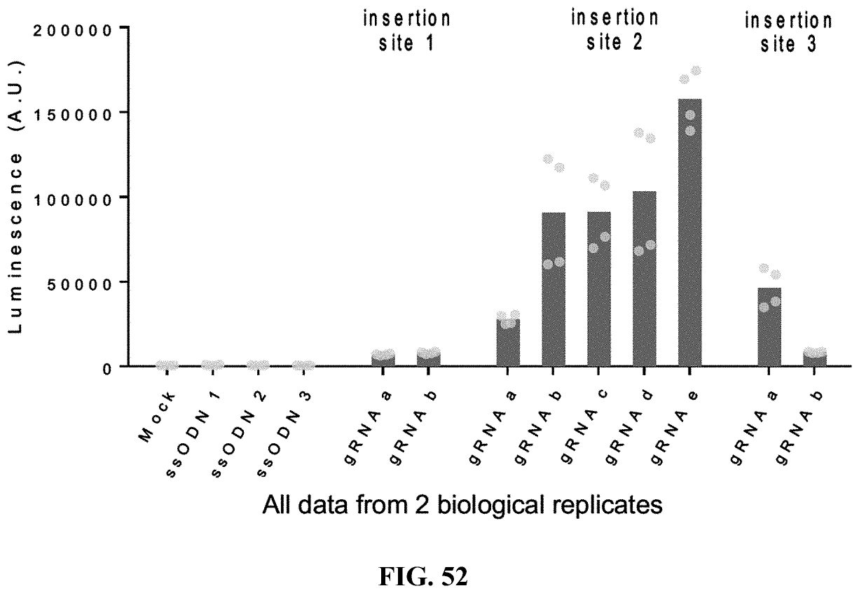

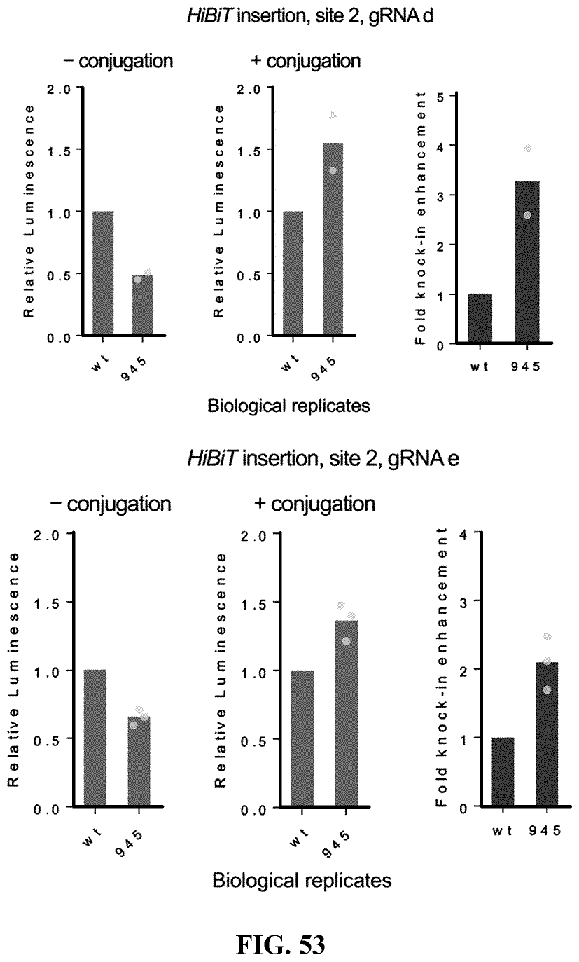

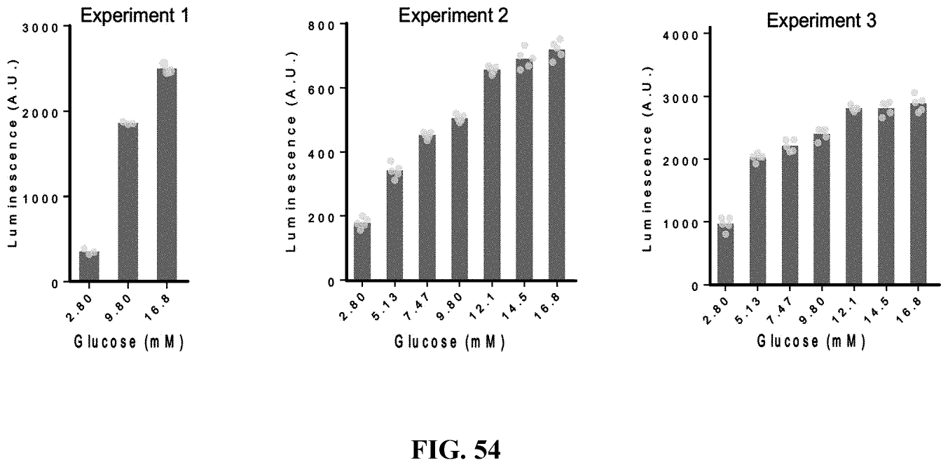

Insertion of a gene of interest at different insertion sites on an insulin gene was tested. The general approach is shown in FIG. 51. Various gRNA molecules were selected (FIG. 52). Effects of Cas9-ssODN conjugation on HiBiT insertion were tested (FIG. 53). Edited cells were grown in a large scale and glucose-stimulated secretion of inserted peptides were tested in 24-well plates (FIG. 54).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An engineered, non-naturally occurring molecule comprising a nucleic acid binding domain, one or more effector domains, and one or more activator of homology-directed repair (HDR) and/or one or more inhibitor of non-homologous end joining (NHEJ).

2. The engineered, non-naturally occurring molecule of claim 1, wherein the one or more inhibitor of NHEJ is selected from

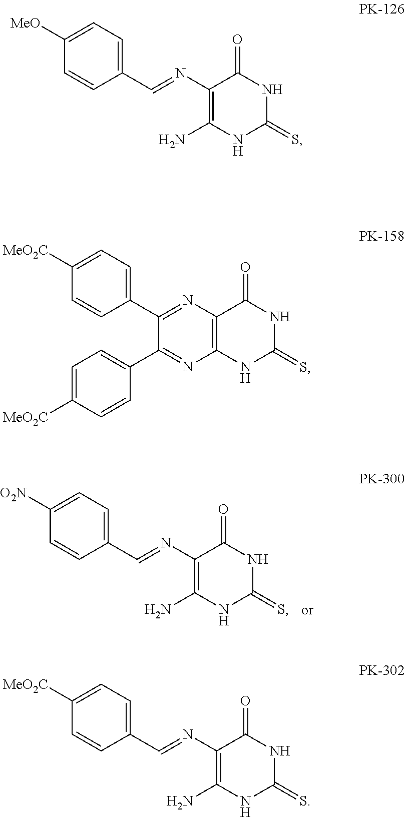 [SMILES: COc1ccc(/C=N/c2c(N)[nH]c(=S)[nH]c2=O)cc1 ; COC(=O)c1ccc(-c2nc3[nH]c(=S)[nH]c(=O)c3nc2-c2ccc(C(=O)OC)cc2)cc1 ; Nc1[nH]c(=S)[nH]c(=O)c1/N=C/c1ccc([N+](=O)[O-])cc1 ; COC(=O)c1ccc(/C=N/c2c(N)[nH]c(=S)[nH]c2=O)cc1]

3. The engineered, non-naturally occurring composition of claim 3, wherein the one or more inhibitor of NHEJ is an SCR7 analog selected from:

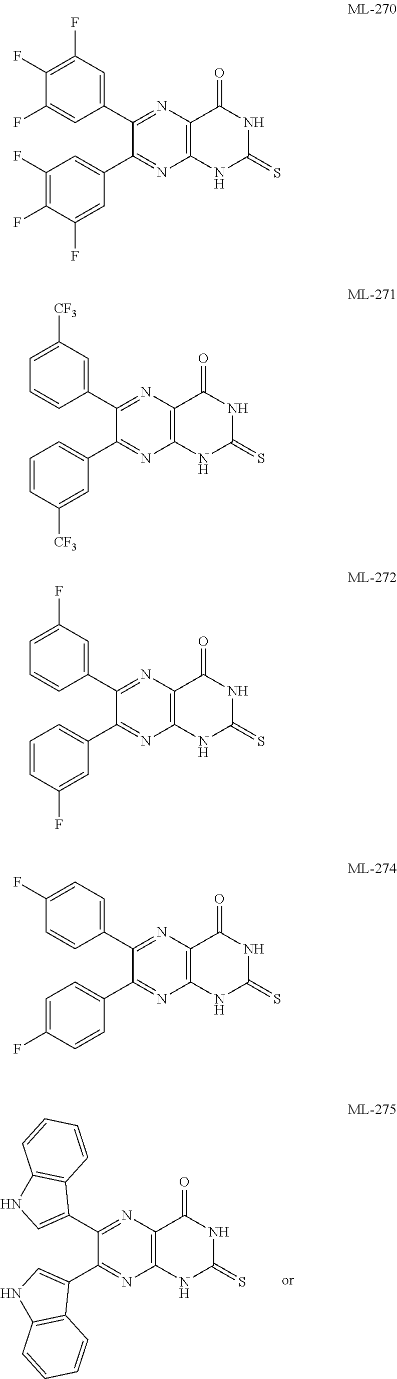 [SMILES: O=c1[nH]c(=S)[nH]c2nc(-c3cc(F)c(F)c(F)c3)c(-c3cc(F)c(F)c(F)c3)nc12 ; O=c1[nH]c(=S)[nH]c2nc(-c3cccc(C(F)(F)F)c3)c(-c3cccc(C(F)(F)F)c3)nc12 ; O=c1[nH]c(=S)[nH]c2nc(-c3cccc(F)c3)c(-c3cccc(F)c3)nc12 ; O=c1[nH]c(=S)[nH]c2nc(-c3ccc(F)cc3)c(-c3ccc(F)cc3)nc12 ; O=c1[nH]c(=S)[nH]c2nc(-c3c[nH]c4ccccc34)c(-c3c[nH]c4ccccc34)nc12]

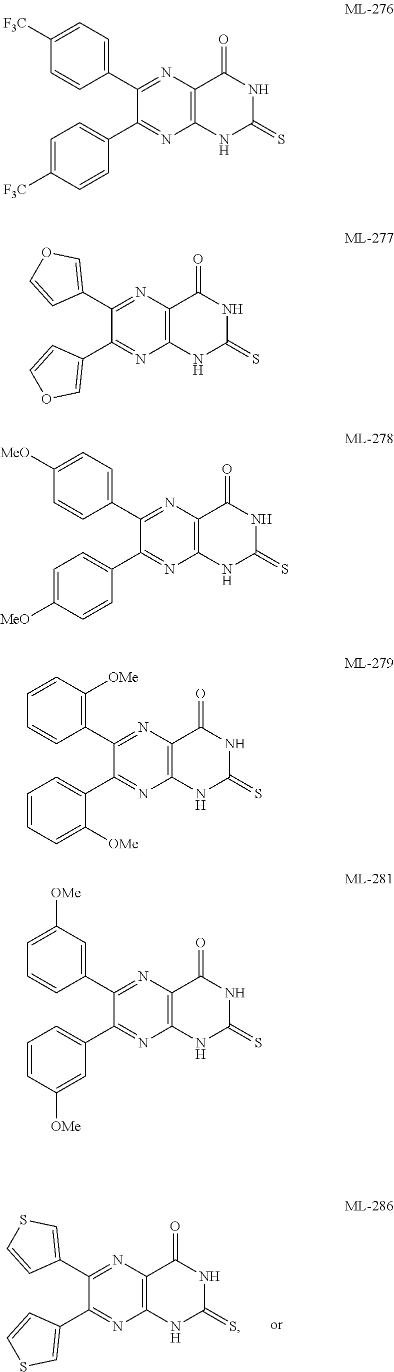 [SMILES: O=c1[nH]c(=S)[nH]c2nc(-c3ccc(C(F)(F)F)cc3)c(-c3ccc(C(F)(F)F)cc3)nc12 ; O=c1[nH]c(=S)[nH]c2nc(-c3ccoc3)c(-c3ccoc3)nc12 ; COc1ccc(-c2nc3[nH]c(=S)[nH]c(=O)c3nc2-c2ccc(OC)cc2)cc1 ; COc1ccccc1-c1nc2[nH]c(=S)[nH]c(=O)c2nc1-c1ccccc1OC ; COc1cccc(-c2nc3[nH]c(=S)[nH]c(=O)c3nc2-c2cccc(OC)c2)c1 ; O=c1[nH]c(=S)[nH]c2nc(-c3ccsc3)c(-c3ccsc3)nc12]

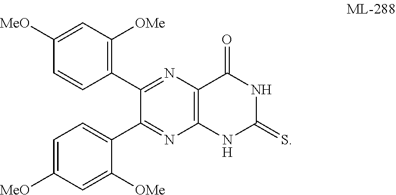 [SMILES: COc1ccc(-c2nc3[nH]c(=S)[nH]c(=O)c3nc2-c2ccc(OC)cc2OC)c(OC)c1]

4. The engineered, non-naturally occurring complex of claim 1, wherein the one or more activator of HDR are selected from

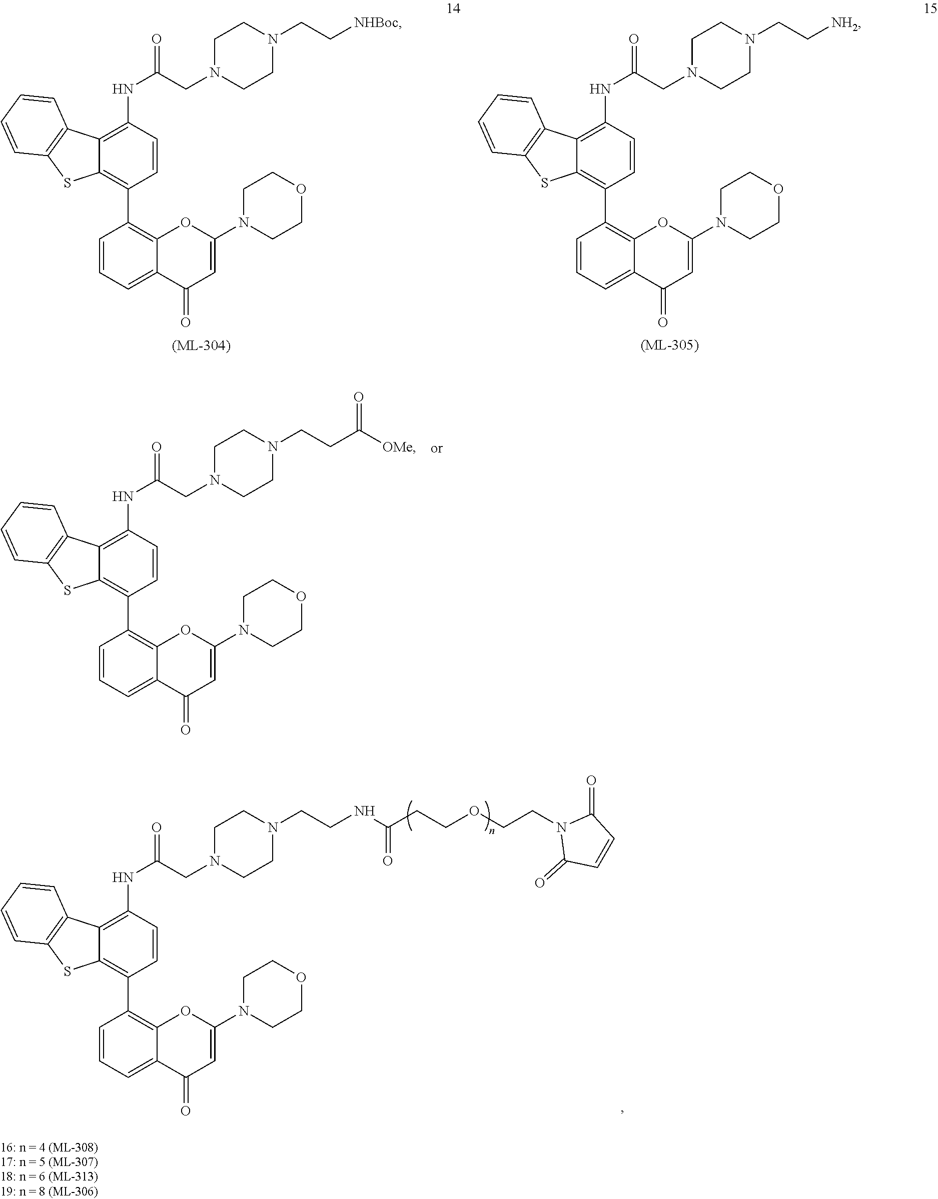

5. An engineered, non-naturally occurring composition, comprising:
the engineered, non-naturally occurring molecule of any of claims 1-4, wherein the molecule is nucleic acid-guided molecule comprising a nucleic acid binding domain which complexes with a guide comprising a polynucleotide
wherein the guide directs sequence specific binding of the nucleic acid-guided molecule to a target nucleic acid, and as to an analogous naturally-occurring nucleic acid-guided molecule, the engineered, non-naturally-occurring nucleic acid-guided molecule is truncated, optionally wherein the one or more effector domains is heterologous.

6. The engineered, non-naturally occurring composition of claim 5, wherein the nucleic acid binding domain comprises a truncated CRISPR protein.

7. The engineered, non-naturally occurring composition of claim 1, wherein the one or more inhibitor of NHEJ is an inhibitor of DNA ligase IV, KU70, or KU80, an SCR7, SCR6, or an analog thereof.

8. The engineered, non-naturally occurring composition of claim 6, comprising a p53 inhibitor, optionally α pifthrin, or an ATM kinase inhibitor, optionally KU-55933.

9. The engineered, non-naturally occurring composition of claim 6, comprising a uracil DNA glycosylase inhibitor (UGI) or functional fragment thereof.

10. The engineered, non-naturally occurring complex of claim 6, wherein the nucleic acid binding domain comprises amino acids of the RuvC, bridge helix, REC1, and PI domains of SpCas9 that interact with SpCas9 guide RNAs.

11. The engineered, non-naturally occurring composition of claim 6, wherein the nucleic acid binding domain comprises binding residues which correspond to all or a subset of the following amino acids of SpCas9: Lys30, Lys33, Arg40, Lys44, Asn46, Glu57, Thr62, Arg69, Asn77, Leu101, Ser104, Phe105, Arg115, His116, Ile135, His160, Lys163, Arg165, Gly166, Tyr325, His328, Arg340, Phe351, Asp364, Gln402, Arg403, Thr404, Asn407, Arg447, Ile448, Leu455, Ser460, Arg467, Thr472, Ile473, Lys510, Tyr515, Trp659, Arg661, Met694, Gln695, His698, His721, Ala728, Lys742, Gln926, Val1009, Lys1097, Val1100, Gly1103, Thr1102, Phe1105, Ile1110, Tyr1113, Arg1122, Lys1123, Lys1124, Tyr1131, Glu1225, Ala1227, Gln1272, His1349, Ser1351, and Tyr1356.

12. The engineered, non-naturally occurring composition of claim 11, wherein the nucleic acid binding domain further comprises binding residues which correspond to all or a subset of Ala59, Arg63, Arg66, Arg70, Arg74, Arg78, Lys50, Tyr515, Arg661, Gln926, and Val1009 of SpCas9, and/or further comprises binding residues which correspond to all or a subset of Leu169, Tyr450, Met495, Asn497, Trp659, Arg661, Met694, Gln695, His698, Ala728, Gln926, and Glu1108 of SpCas9.

13. The engineered, non-naturally occurring composition of claim 6, wherein the nucleic acid binding domain is truncated as to all or part of the NUC lobe of SpCas9.

14. The engineered, non-naturally occurring composition of claim 6, wherein the nucleic acid binding domain is truncated as to one or more of the RuvCI, RuvC II, RuvC III, HNH and PI domains of SpCas9.

15. The engineered, non-naturally occurring composition of claim 6, wherein the nucleic acid binding domain comprises amino acids of the RuvC, bridge helix, REC, WED, phosphate lock loop (PLL), and PI domains of SaCas9 that interact with SaCas9 guide RNAs.

16. The engineered, non-naturally occurring composition of claim 6, wherein the nucleic acid binding domain comprises binding residues which correspond to all or a subset of the following amino acids of SaCas9: Asn47, Lys50, Arg54, Lys57, Arg58, Arg61, His62, His111, Lys114, Gly162, Val164, Arg165, Arg209, Glu213, Gly216, Ser219, Asn780, Arg781, Leu783, Leu788, Ser790, Arg792, Asn804, Lys867, Tyr868, Lys870, Lys878, Lys879, Lys881, Leu891, Tyr897, Arg901, and Lys906.

17. The engineered, non-naturally occurring composition of claim 16, wherein the nucleic acid binding domain further comprises binding residues which correspond to all or a subset of Asn44, Arg48, Arg51, Arg55, Arg59, Arg60, Arg116, Gly117, Arg165, Gly166, Arg208, Arg209, Tyr211, Thr238, Tyr239, Lys248, Tyr256, Arg314, and Asn394, of SaCas9 and/or all or a subset of Tyr211, Trp229, Tyr230, Gly235, Arg245, Gly391, Thr392, Asn419, Leu446, Tyr651, and Arg654 of SaCas9.

18. The engineered, non-naturally occurring composition of claim 6, wherein the nucleic acid binding domain is truncated as to all or part of the NUC lobe of SaCas9.

19. The engineered, non-naturally occurring composition of claim 6, wherein the nucleic acid binding domain is truncated as to one or more of the RuvCI, RuvC II, RuvC III, HNH, WED, and PI domains of SaCas9.

20. The engineered, non-naturally occurring composition of claim 6, wherein the nucleic acid binding domain comprises amino acids of WED, REC1, REC2, PI, bridge helix, and RuvC domains of AsCpf1 that interact with AsCpf1 guide RNAs.

21. The engineered, non-naturally occurring composition of claim 6, wherein the nucleic acid binding domain comprises binding residues which correspond to all or a subset of the following amino acids of AsCpf1: Lys15, Arg18, Lys748, Gly753, His755, Gly756, Lys757, Asn759, His761, Arg790, Met806, Leu807, Asn808, Lys809, Lys810, Lys852, His856, Ile858, Arg863, Tyr940, Lys943, Asp966, His977, Lys1022 and Lys1029.

22. The engineered, non-naturally occurring composition of claim 21, wherein the nucleic acid binding domain further comprises binding residues which correspond to all or a subset of Tyr47, Lys51, Arg176, Arg192, Gly270, Gln286, Lys273, Lys307, Leu310, Lys369, Lys414, His 479, Asn515, Arg518, Lys530, Glu786, His872, Arg955, and Gln956 of AsCpf1 and/or all or a subset of Asn178, Ser186, Asn278, Arg301, Thr315, Ser376, Lys524, Lys603, Lys780, Gly783, Gln784, Arg951, Ile964, Lys965, Gnl1014, Phe1052, and Ala1053 of AsCpf1.

23. The engineered, non-naturally occurring composition of claim 6, wherein the nucleic acid binding domain is truncated as to all or part of the NUC lobe of AsCpf1.

24. The engineered, non-naturally occurring composition of claim 6, wherein the nucleic acid binding domain is truncated as to one or more of the WED-I, WED-II, WED-III, PI, RuvC I, RuvC II, RuvC III, Nuc, BH, and PI domains of AsCpf1.

25. The engineered, non-naturally occurring composition of any one of claims 6 to 19, wherein the nucleic acid binding domain lacks one or more amino acid positions K169, Y450, N497, R661, Q695, Q926, K810, K848, K1003, R1060, or D1135, or corresponding amino acids of an SpCas9 ortholog.

26. The engineered, non-naturally occurring composition of any one of claims 6 to 19, wherein the nucleic acid binding domain lacks one or more of RuvCI, RuvCII, RuvCIII, NUC, PI, or BH.

27. The engineered, non-naturally occurring composition of claim 5, further comprising a guide, wherein the guide comprises RNA.

28. The engineered, non-naturally occurring composition of claim 27, wherein the guide comprises a nucleotide analog.

29. The engineered, non-naturally occurring composition of claim 5, wherein the nucleic acid binding domain and the one or more effector domains are covalently linked with a linker.

30. The engineered, non-naturally occurring composition of claim 29, wherein the linker comprises a chemical linker or an amino acid linker.

31. The engineered, non-naturally occurring composition of claim 29, wherein the linker comprises Gly-Gly-Gly-Gly-Ser (GGGGS) (SEQ ID NO: 92).

32. The engineered, non-naturally occurring composition of claim 29, wherein the linker comprises PEG.

33. The engineered, non-naturally occurring composition of claim 29, wherein the linker is cleavable in vivo.

34. The engineered, non-naturally occurring composition of claim 5, wherein the binding domain and one or more effector domains are non-covalently associated.

35. The engineered, non-naturally occurring composition of claim 34, wherein the composition is inducible.

36. The engineered, non-naturally occurring composition of claim 34, wherein the composition is switchable.

37. The engineered, non-naturally occurring composition of claim 34, wherein the guide comprises an aptamer that associates with the one or more effector domains.
The engineered, non-naturally-occurring molecule of claim 1, wherein as to an analogous naturally-occurring molecule, the engineered, non-naturally-occurring molecule is truncated and the one or more effector domains is heterologous.

38. The engineered, non-naturally-occurring molecule of claim 37, wherein the nucleic acid-binding domain comprises at least five or more transcript activator-like effector (TALE) monomers and at least one or more half-monomers specifically ordered to a target locus of interest.

39. The engineered, non-naturally-occurring molecule of claim 38, wherein the one or more monomers or half-monomers comprise one or more peptidomimetics.

40. The engineered, non-naturally-occurring molecule of claim 38 or 39, wherein the one or more monomers or half-monomers ere further modified to be proteolytically and chemically stable.

41. The engineered, non-naturally-occurring molecule of claim 40, wherein the further modifications comprise one or more of stapling, side-chain cross-linking, and hydrogen-bond surrogating.

42. The engineered, non-naturally occurring molecule or composition of any one of claims 1 to 41, wherein the one or more effector domain comprises one or more of a single stranded nuclease, a double strand nuclease, a helicase, a methylase, a demethylase, an acetylase, a deacetylase, a deaminase, an integrase, a recombinase, of a cellular uptake activity associated domain.

43. The engineered, non-naturally occurring molecule or composition of any one of claims 1 to 41, wherein the one or more effector domain comprises a small molecule that induces single- or double-strand breaks in the nucleic acid target.

44. The engineered, non-naturally occurring molecule or composition of any one of claims 1 to 43, wherein the composition comprises one or more nuclear localization signals (NLSs).

45. The engineered, non-naturally occurring molecule or composition of claim 44, wherein the one or more NLSs is linked to the nucleic acid-binding domain.

46. The engineered, non-naturally occurring molecule or composition of claim 44, wherein the one or more NLSs is linked to the one or more effector domains.

47. The engineered non-naturally occurring molecule or composition of any one of claims 1 to 43, wherein the molecule or composition comprises a delivery enhancer.

48. The engineered non-naturally occurring molecule or composition of any one of claims 1 to 43, wherein the molecule or composition comprises a cellular permeability enhancer.

49. The composition of any of claims 5 to 48, further comprising a guide which directs sequence specific binding of the nucleic acid-guided molecule to a target nucleic acid.

50. The composition of claim 49, wherein the guide nucleic acid is RNA.

51. The composition of claim 50, wherein the RNA is a single guide RNA (sgRNA).

52. The composition of any of claims 49 to 51, wherein the composition is provided as a complex.

53. The composition of any one of claims 49 to 52, wherein the composition further comprises one or more effector domains that are heterologous to the engineered, non-naturally occurring nucleic acid-guided molecule.

54. The composition of any one of claims 49 to 53, wherein the composition further comprises a recombination template.

55. The composition of claim 49, wherein the activator of HDR is a small molecule.

56. The composition of claim 49, wherein the HDR activator is RS1.

57. The composition of claim 49, wherein the activator of HDR stimulates RAD51.

58. The composition of claim 49, wherein the activator of HDR is linked to the nucleic acid binding molecule.

59. The composition of claim 49, wherein the inhibitor of NHEJ is an inhibitor of DNA ligase IV, KU70, or KU80.

60. The composition of claim 49, wherein the inhibitor of NHEJ is a small molecule.

61. The composition of claim 49, wherein the inhibitor of NHEJ is linked to the nucleic acid binding molecule.

62. The complex or composition of any one of the preceding claims, wherein the guide nucleic acid is in a duplex with a target nucleic acid.

63. The complex or composition of any one of the preceding claims, wherein the target nucleic acid comprises chromosomal DNA.

64. The complex or composition of any one of the preceding claims, wherein the target nucleic acid comprises mitochondrial DNA.

65. The complex or composition of any one of the preceding claims, wherein the target nucleic acid comprises viral, bacterial, or fungal DNA or RNA.

66. A method of repairing DNA damage in a cell or tissue, which comprises contacting the damaged DNA of the cell or tissue with the complex or composition of any one of claims 1 to 65.

67. A DNA repair kit comprising the complex or composition of any one of claims 1 to 65.

68. A vector system for delivering to a mammalian cell or tissue comprising the complex or composition of any one of claims 1 to 65.

69. An engineered, non-naturally occurring nucleic acid modifying system, comprising:
the composition of claim 6;
wherein the one or more effector components facilitate DNA repair by homology directed repair (HDR).

70. The system of claim 69, wherein the one or more effector components comprise one or more single stranded oligo donors.

71. The system of claim 69, wherein the one or more effector components comprise a single-stranded oligo donor (ssODN), one or more NHEJ inhibitors, and one or more HDR activators.

72. The system of claim 69 to 71 wherein the HDR activator stimulates RAD51 activity.

73. The system of claim 69, further comprising a p53 inhibitor, optionally α pifthrin or an ATM kinase inhibitor, optionally KU-5593.

74. The system of claim 69, further comprising a uracil DNA glycosylase inhibitor (UGI) or functional fragment thereof.

75. The system of claim 69, wherein the CRISPR/Cas protein is selected from the group consisting of an engineered Cas9, Cpf1, Cas12b, Cas12c, Cas13a, Cas13b, Cas13c, and Cas13d protein.

76. The system of claim 69, wherein the CRISPR/Cas protein is an engineered Cas9 protein.

77. The system of claim 69, wherein the CRISPR/Cas protein comprises one or more engineered cysteine amino acids.

78. The system of claim 69, wherein the CRISPR/Cas protein is an SpCas9 protein comprising C80S and C574S mutations and one or more mutations selected from the group consisting of M1C, S204C, D435C, E532C, Q674C, Q826C, S867C, E945C, S1025C, E1026C, N1054C, E1068C, S1116C, K1153C, E1207C.

79. The system of claim 95, comprising two or more mutations comprising E532 C and E945C, or E532C and E1207C.

80. The system of claim 79, further comprising two ssODN.

81. The system of claim 69, wherein the CRISPR/Cas protein comprises a sortase recognition sequence Leu-Pro-Xxx-Thr-Gly.

82. The system of claim 69, wherein the CRISPR/Cas protein comprises one or more unnatural amino acid p-Acetyl Phenylalanine (pAcF), or one or more unnatural amino acid comprising tetrazine.

83. The system of claim 70, wherein the one or more effector components further comprise one or more adaptor oligonucleotides, wherein one adaptor oligonucleotide hybridizes with one ssODN.

84. The system of claim 83, wherein the one or more adaptor oligonucleotides are at least 10 nucleotides, at least 13 nucleotides, at least 15 nucleotides, or at least 17 nucleotides.

85. The system of claim 84, wherein each adaptor oligonucleotide and the hybridizing ssODN have at least 13 overlapping nucleotides.

86. The system of claim 69, wherein the guide nucleic acid is a guide RNA molecule.

87. The system of claim 69 to 71, wherein the NHEJ inhibitor is an inhibitor of DNA ligase IV, KU70, or KU80.

88. The system of claim 69 to 71, wherein the NHEJ inhibitor is selected from the group consisting of SCR7, SCR6, KU inhibitor, and analogs thereof.

89. The system of claim 69, wherein the one or more effector components are linked to the CRISPR/Cas protein.

90. The system of claim 89, wherein the one or more effector components are covalently linked to the CRISPR/Cas protein.

91. The system of claim 90, wherein the one or more effector components are linked to the CRISPR/Cas protein via cysteines, sortase chemistry, or unnatural amino acids.

92. The system of claim 69, wherein the one or more effector components are linker modified.

93. The system of claim 92, wherein the linker comprises a maleimide group.

94. The system of claim 92, wherein the linker comprises PEG.

95. The system of claim 92, wherein the linker comprises a poly-Gly peptide.

96. The system of claim 70, wherein the CRISPR/Cas protein is an SpCas9 protein comprising C80S and C574S mutations and one or more mutations selected from the group consisting of M1C, S204C, D435C, E532C, Q674C, Q826C, S867C, E945C, S1025C, E1026C, N1054C, E1068C, S1116C, K1153C, E1207C.

97. The system of claim 83, wherein the one or more adaptor oligonucleotides are linked to the CRISPR/Cas protein via thiol-maleimide chemistry.

98. The system of any one of the preceding claims, wherein the guide nucleic acid is in a duplex with the target nucleic acid.

99. The system of any of claims 69-98, wherein the target nucleic acid comprises chromosomal DNA.

100. The system of any of claims 69-98, wherein the target nucleic acid comprises mitochondrial DNA.

101. The system of any of claims 69-98, wherein the target nucleic acid comprises viral, bacterial, or fungal DNA.

102. The system of any of claims 69-98, wherein the target nucleic acid comprises viral, bacterial, or fungal RNA.

103. A method for enhancing HDR at one or more target loci in a target cell, comprising delivering the system of any of claims 69-102 to the target cell.

104. The method of claim 03, wherein the system is delivered to the target cell via electroporation.

105. The method of claim 103, wherein the system is delivered to the target cell via lipid-mediated delivery.

106. An engineered, non-naturally occurring nucleic acid modifying system, comprising:
a) a first engineered, non-naturally occurring DNA reader, wherein the first DNA reader binds a target nucleic acid;
b) a first effector component, wherein the first effector is a small molecule and modifies the target nucleic acid.

107. The system of claim 106, wherein the first DNA reader is a peptide nucleic acid (PNA) polymer, or transcript activator-like effector (TALE).

108. The system of claim 106, further comprising one or more Non-Homologous End Joining (NHEJ) inhibitors and/or one or more Homology-Directed Repair (HDR) activators.

109. The system of claim 108, wherein the NHEJ inhibitor is selected from the group consisting of SCR7, SCR6, KU inhibitor, and analogs thereof.

110. The system of claim 108, wherein the NHEJ inhibitor is selected from

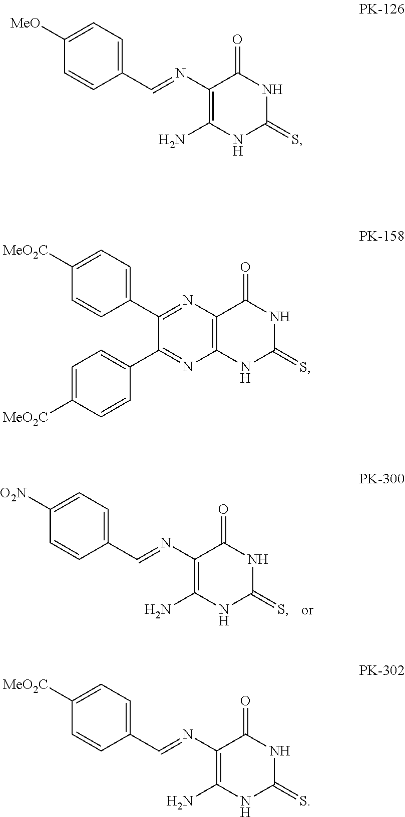 [SMILES: COc1ccc(/C=N/c2c(N)[nH]c(=S)[nH]c2=O)cc1 ; COC(=O)c1ccc(-c2nc3[nH]c(=S)[nH]c(=O)c3nc2-c2ccc(C(=O)OC)cc2)cc1 ; Nc1[nH]c(=S)[nH]c(=O)c1/N=C/c1ccc([N+](=O)[O-])cc1 ; COC(=O)c1ccc(/C=N/c2c(N)[nH]c(=S)[nH]c2=O)cc1]

111. The system of claim 108, wherein the NHEJ inhibitor is an SCR7 analog selected from:

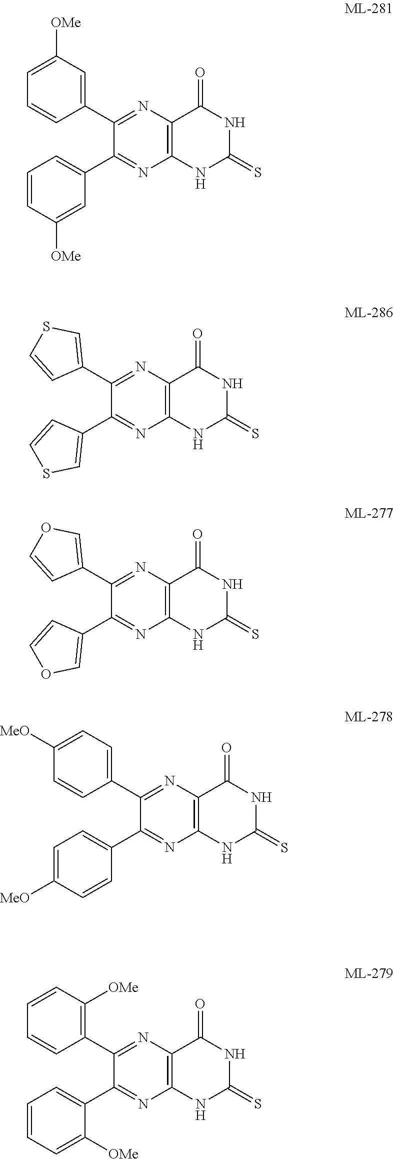 [SMILES: COc1cccc(-c2nc3[nH]c(=S)[nH]c(=O)c3nc2-c2cccc(OC)c2)c1 ; O=c1[nH]c(=S)[nH]c2nc(-c3ccsc3)c(-c3ccsc3)nc12 ; O=c1[nH]c(=S)[nH]c2nc(-c3ccoc3)c(-c3ccoc3)nc12 ; COc1ccc(-c2nc3[nH]c(=S)[nH]c(=O)c3nc2-c2ccc(OC)cc2)cc1 ; COc1ccccc1-c1nc2[nH]c(=S)[nH]c(=O)c2nc1-c1ccccc1OC]

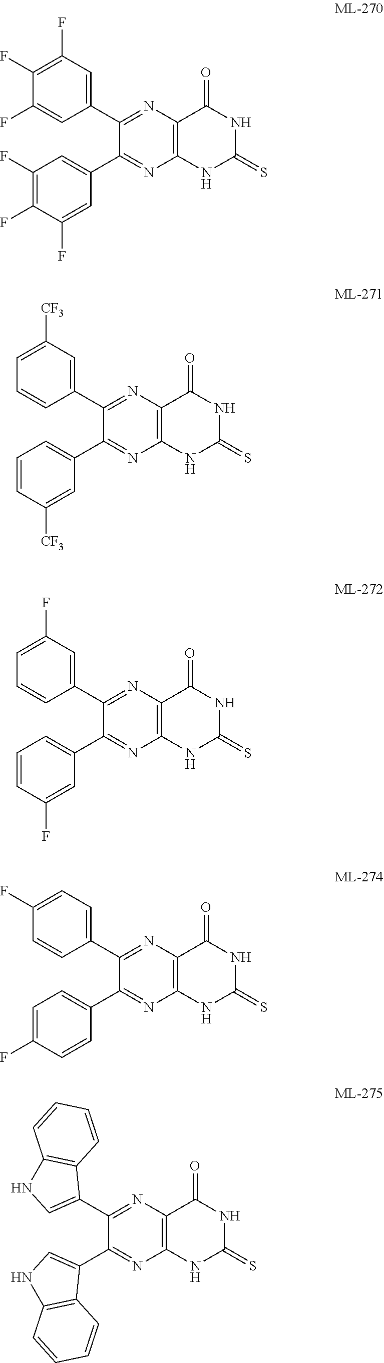

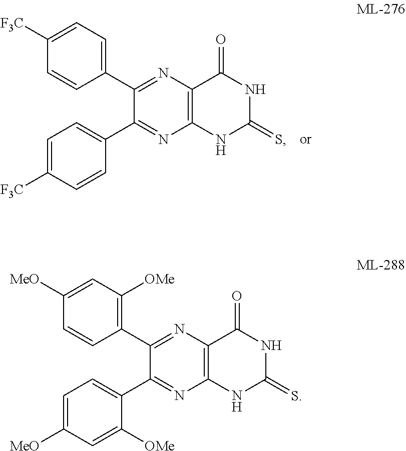 [SMILES: O=c1[nH]c(=S)[nH]c2nc(-c3ccc(C(F)(F)F)cc3)c(-c3ccc(C(F)(F)F)cc3)nc12 ; COc1ccc(-c2nc3[nH]c(=S)[nH]c(=O)c3nc2-c2ccc(OC)cc2OC)c(OC)c1]

112. The system of claim 108, wherein the HDR activator is a small molecule.

113. The system of claim 108, wherein the HDR activator is selected from 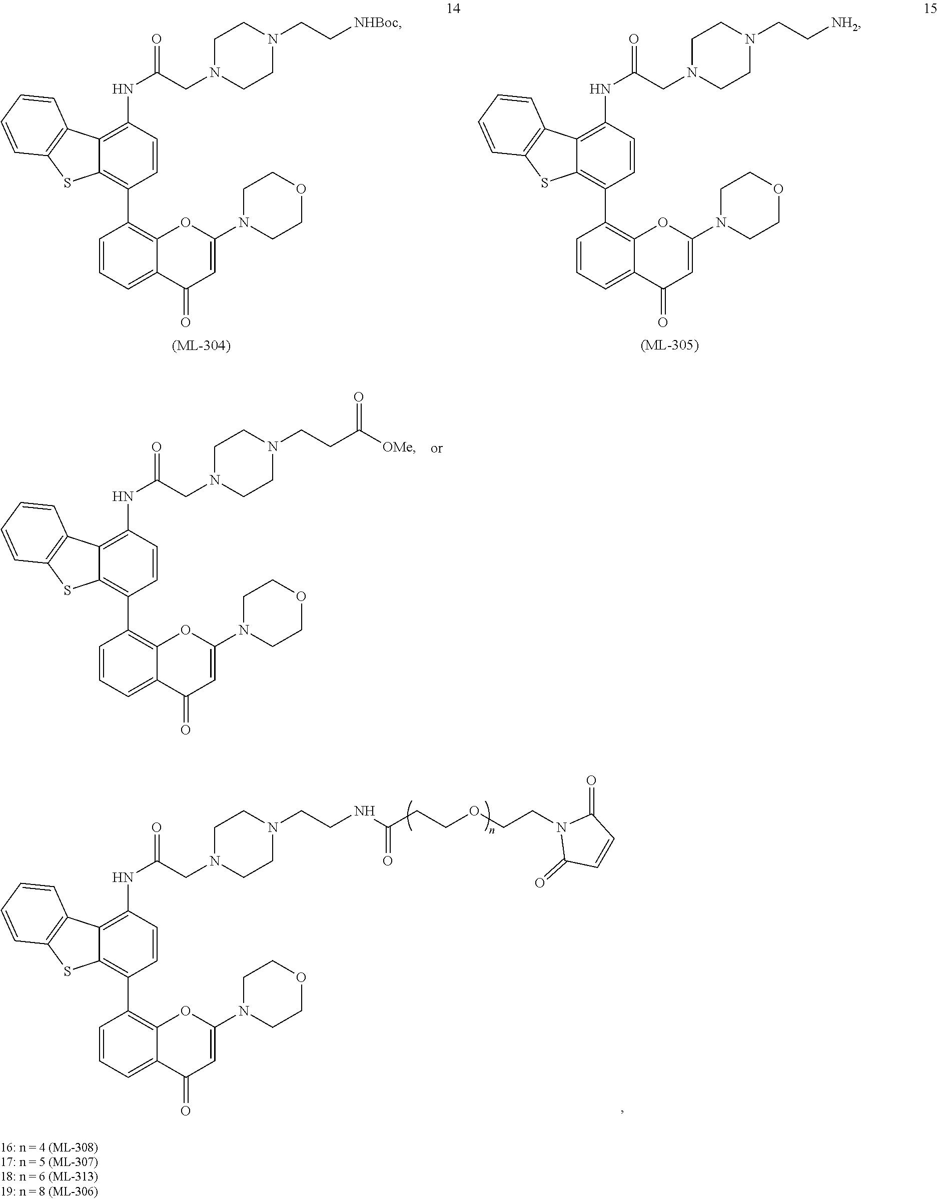
wherein n=4, 5, 6 or 8.

114. The system of claim 106, wherein the first DNA reader is a PNA polymer.

115. The system of claim 106, wherein the first effector component is a small molecule synthetic nuclease.

116. The system of claim 115, wherein the first effector component is selected from the group consisting of diazofluorenes, nitracrines, metal complexes, enediyenes, methoxsalen derivatives, daunorubicin derivatives, and juglones.

117. The system of claim 115, wherein the small synthetic nuclease is selected from

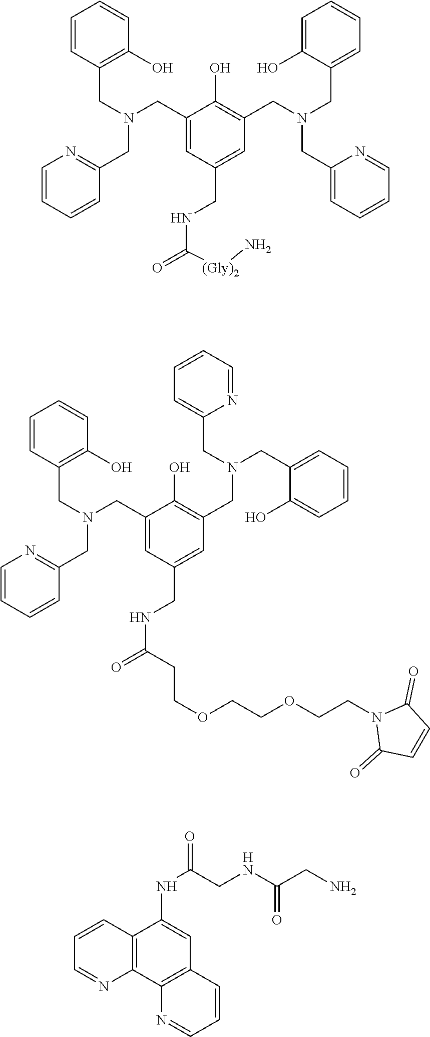

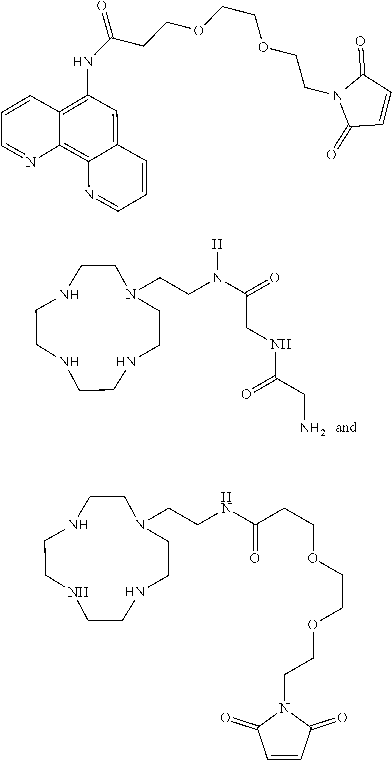 [SMILES: O=C(CCOCCOCCN1C(=O)C=CC1=O)Nc1cc2cccnc2c2ncccc12 ; NCC(=O)NCC(=O)NCCN1CCNCCNCCNCC1 ; O=C(CCOCCOCCN1C(=O)C=CC1=O)NCCN1CCNCCNCCNCC1]

118. The system of claim 115, wherein the synthetic nuclease is a single strand breaking small molecule.

119. The system of claim 115, wherein the synthetic nuclease is a double strand breaking small molecule.

120. The system of claim 115, wherein the first effector component is linked to the first DNA reader.

121. The system of claim 120, wherein the first effector component is covalently linked to the first DNA reader.

122. The system of claim 121, wherein the first effector component comprises one or more maleimide, azide, or alkyne functional groups and the first DNA reader comprises a PEG linker comprising one or more thiol, alkyne, or azide functional groups.

123. The system of claim 106, further comprising a second DNA reader and a second effector component.

124. The system of claim 123, wherein the first effector component is covalently linked to the first DNA reader and the second effector component is covalently linked to the second DNA reader.

125. The system of claim 124, wherein both the first and second DNA readers are PNA polymers.

126. The system of claim 125, wherein the first effector component is an inactive small molecule synthetic nuclease and the second effector component is a trigger reagent, wherein the trigger reagent activates the small molecule synthetic nuclease.

127. The system of claim 126, wherein the first effector component is Kinamycin C and the second effector component is a reducing agent.

128. The system of claim 126, wherein the first effector component is dynemicin and the second effector component is a reducing agent.

129. The system of claim 128, wherein the first effector component comprises a first fragment of a reactive group of a small molecule synthetic nuclease and the second effector component comprises a second fragment of the reactive group of the small molecule synthetic nuclease, wherein the small molecule synthetic nuclease is only active when the first fragment and the second fragment are together.

130. The system of claim 123, further comprising a third and a fourth effector component.

131. The system of claim 130, wherein both the first and second DNA readers are PNA polymers, and the first, second, third, and fourth effector component are small molecule single strand breaking synthetic nucleases.

132. The system of claim 131, wherein the first and second synthetic nucleases are linked to the first PNA polymer, and the third and fourth synthetic nucleases are linked to the second PNA polymer.

133. The system of claim 132, further comprising one or more single-stranded oligo donors (ssODNs).

134. The system of claim 106, further comprising one or more NHEJ inhibitors and/or one or more HDR activators.

135. The system of claim 114, wherein the NHEJ inhibitor is an inhibitor of DNA ligase IV, KU70, or KU80.

136. The system of claim 114, wherein the NHEJ inhibitor is a small molecule.

137. The system of claim 114, wherein the NHEJ inhibitor is selected from the group consisting of SCR7, SCR6, KU inhibitor, and analogs thereof.

138. The system of claim 114, wherein the HDR activator is a small molecule.

139. The system of claim 114, wherein the HDR activator is RS1 or analogs thereof.

140. The system of claim 114, wherein the HDR activator stimulates RAD51 activity.

141. The system of any of claims 93-140, wherein the target nucleic acid comprises chromosomal DNA, mitochondrial DNA, viral DNA or RNA, bacterial DNA or RNA, or fungal DNA or RNA.

142. The system of any one of the preceding claims, further comprising a delivery enhancer.

143. The system of claim 142, wherein the delivery enhancer is a cellular permeability enhancer.

144. The system or complex of any of the preceding claims, comprising a p53 inhibitor, optionally α-pefthrin, or an ATM kinase inhibitor, optionally KU-5593.

145. The system of complex of any of the preceding claims comprising a synthetic nuclease selected from

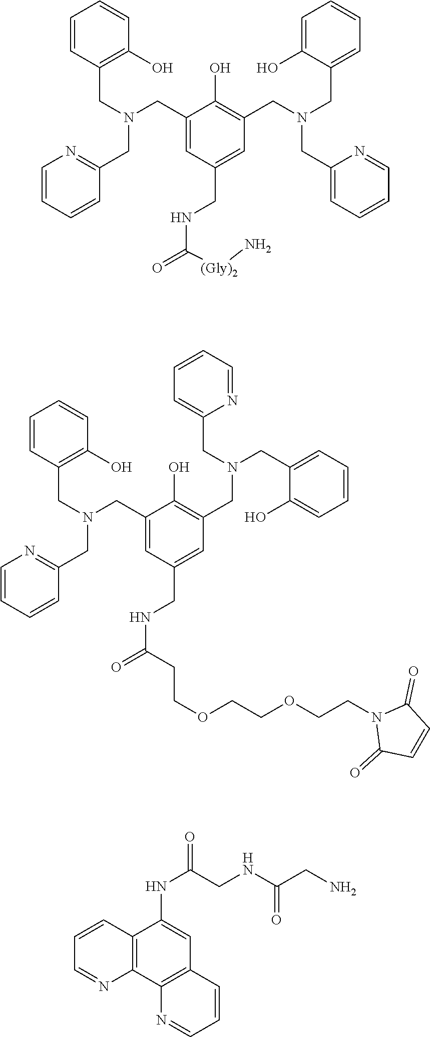

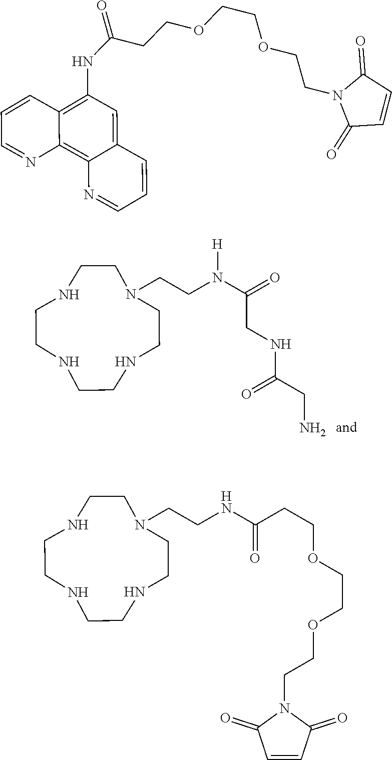 [SMILES: O=C(CCOCCOCCN1C(=O)C=CC1=O)Nc1cc2cccnc2c2ncccc12 ; NCC(=O)NCC(=O)NCCN1CCNCCNCCNCC1 ; O=C(CCOCCOCCN1C(=O)C=CC1=O)NCCN1CCNCCNCCNCC1]

146. A method of precise genome editing in a cell or tissue, comprising delivering the system of any one of claims to the cell or tissue.

147. The method of claim 146, wherein the system is delivered using Poly(lactic co-glycolic acids) (PLGA) nanoparticles.
